SEQUENCE LISTING (86 sequences)

SEQ ID NO: 1 - DNA, 384 bp, Artificial Sequence
  Strep-Tactin comprising Caf
atggaagcaggtatcaccggcacctggtacaaccagctcggctcgaccttcatcgtgaccgcgggtgcagacggagctctgaccggtacctacgtcacggcgcgtggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtagctgctgacctccggcaccaccgaggccaacgcctggaagtccacgctggtcggccacgacaccttcaccaaggtgaagccgtccgccgcctcctaa

SEQ ID NO: 2 - protein, 127 aa, Artificial Sequence
  Strep-Tactin comprising Caf
  UNSURE 96: modified amino acid Caf
MEAGITGTWYNQLGSTFIVTAGADGALTGTYVTARGNAESRYVLTGRYDSAPATDGSGTALGWTVAWKNNYRNAHSATTWSGQYVGGAEARINTQXLLTSGTTEANAWKSTLVGHDTFTKVKPSAAS

SEQ ID NO: 3 - DNA, 384 bp, Artificial Sequence
  Core streptavidin comprising Caf
atggaagcaggtatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtagctgctgacctccggcaccaccgaggccaacgcctggaagtccacgctggtcggccacgacaccttcaccaaggtgaagccgtccgccgcctcctaa

SEQ ID NO: 4 - protein, 127 aa, Artificial Sequence
  Core streptavidin comprising Caf
  UNSURE 96: modified amino acid Caf
MEAGITGTWYNQLGSTFIVTAGADGALTGTYESAVGNAESRYVLTGRYDSAPATDGSGTALGWTVAWKNNYRNAHSATTWSGQYVGGAEARINTQXLLTSGTTEANAWKSTLVGHDTFTKVKPSAAS

SEQ ID NO: 5 - DNA, 552 bp, Artificial Sequence
  Unprocessed streptavidin (i.e. pre- streptavidin) comprising Caf
atgcgcaagatcgtcgttgcagccatcgccgtttccctgaccacggtctcgattacggccagcgcttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtagctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtag

SEQ ID NO: 6 - protein, 183 aa, Artificial Sequence
  Unprocessed streptavidin (i.e. pre- streptavidin) comprising Caf
  UNSURE 132: modified amino acid Caf
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQXLLTSGTTEANAWKSTLVGHDTFTKVKPSAASIDAAKKAGVNNGNPLDAVQQ

SEQ ID NO: 7 - DNA, 384 bp, Artificial Sequence
  Strep-Tactin
atggaagcaggtatcaccggcacctggtacaaccagctcggctcgaccttcatcgtgaccgcgggtgcagacggagctctgaccggtacctacgtcacggcgcgtggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctcctaa

SEQ ID NO: 8 - protein, 127 aa, Artificial Sequence
  Strep-Tactin
MEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 9 - DNA, 384 bp, Streptomyces avidinii
  Core streptavidin
atggaagcaggtatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctcctaa

SEQ ID NO: 10 - protein, 127 aa, Streptomyces avidinii
  Core streptavidin
MEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 11 - DNA, 552 bp, Streptomyces avidinii
  Unprocessed streptavidin (pre-streptavidin)
atgcgcaagatcgtcgttgcagccatcgccgtttccctgaccacggtctcgattacggccagcgcttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtag

SEQ ID NO: 12 - protein, 183 aa, Streptomyces avidinii
  Pre-streptavidin
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 13 - protein, 9 aa, Artificial Sequence
  Strep-tag
AWRHPQFGG

SEQ ID NO: 14 - protein, 8 aa, Artificial Sequence
  Strep-tag II
WSHPQFEK

SEQ ID NO: 15 - protein, 10 aa, Artificial Sequence
  myc-tag
EQKLISEEDL

SEQ ID NO: 16 - protein, 58 aa, Artificial Sequence [sequence omitted]

SEQ ID NO: 17 - protein, 55 aa, Artificial Sequence
  C1 domain of protein G
  VARIANT 3: Caf
  VARIANT 5: Caf
  VARIANT 10: Caf
  VARIANT 16: Caf
  VARIANT 28: Caf
  VARIANT 32: Caf
  VARIANT 35: Caf
TYKLILNGKTLKGETTTEAVDAATAEKVFKQYANDNGVDGEWTYDDATKTFTVTE

SEQ ID NO: 18 - protein, 55 aa, Artificial Sequence
  C2 domain of protein G
  VARIANT 3: Caf
  VARIANT 5: Caf
  VARIANT 10: Caf
  VARIANT 16: Caf
  VARIANT 28: Caf
  VARIANT 32: Caf
  VARIANT 35: Caf
TYKLVINGKTLKGETTTEAVDAATAEKVFKQYANDNGVDGEWTYDDATKTFTVTE

SEQ ID NO: 19 - protein, 55 aa, Artificial Sequence
  C3 domain of protein G
  VARIANT 3: Caf
  VARIANT 5: Caf
  VARIANT 10: Caf
  VARIANT 16: Caf
  VARIANT 28: Caf
  VARIANT 32: Caf
  VARIANT 35: Caf
TYKLVINGKTLKGETTTKAVDAETAEKAFKQYANDNGVDGVWTYDDATKTFTVTE

SEQ ID NO: 20 - protein, 64 aa, Artificial Sequence
  Domain B1 of protein L
  VARIANT 5: Caf
  VARIANT 9: Caf
  VARIANT 11: Caf
  VARIANT 12: Caf
  VARIANT 16: Caf
  VARIANT 22: Caf
  VARIANT 26: Caf
  VARIANT 32: Caf
  VARIANT 35: Caf
  VARIANT 39: Caf
  VARIANT 43: Caf
  VARIANT 44: Caf
  VARIANT 47: Caf
KEEVTIKVNLIFADGKTQTAEFKGTFEEATAKAYAYADLLAKENGEYTADLEDGGNTINIKFAG

SEQ ID NO: 21 - protein, 451 aa, Artificial Sequence
  Heavy chain of the anti-myc-tag monoclonal antibody clone 9E10
  VARIANT 57: Caf
  VARIANT 102: Caf
  VARIANT 103: Caf
  VARIANT 104: Caf
  VARIANT 105: Caf
  VARIANT 109: Caf
  VARIANT 110: Caf
  VARIANT 111: Caf
EVHLVESGGDLVKPGGSLKLSCAASGFTFSHYGMSWVRQTPDKRLEWVATIGSRGTYTHYPDSVKGRFTISRDNDKNALYLQMNSLKSEDTAMYYCARRSEFYYYGNTYYYSAMDYWGQGASVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWPNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 22 - protein, 218 aa, Artificial Sequence
  Light chain of the anti-myc-tag monoclonal antibody clone 9E10
DIVLTQSPASLAVSLGQRATISCRASESVDNYGFSFMNWFQQKPGQPPKLLIYAISNRGSGVPARFSGSGSGTDFSLNIHPVEEDDPAMYFCQQTKEVPWTFGGGTKLEIKRADAAPTVSIFPPSSEQLTSGGASVVCFLNNLYPKDINVKWKIDGSERQNGVLNSWTDQDSKDSTYSMSSTLTLTKDEYERHNSYTCEATHKTSTSPIVKSFNRNEC

SEQ ID NO: 23 - DNA, 5798 bp, Artificial Sequence
  pSBX8.101d58
tttttacggttcctggccttttgctggccttttgctcacatgacccgacaccataacgctcggttgccgccgggcgttttttattggccagatgattaattcctaatttttgttgacactctatcattgatagagttattttaccactccctatcagtgatagagaaaagtgaaatgaatagttcgacaaaatctagataacgagggcaaaaaatgtctaaaggtgaagaacttttcactggagttgtcccaattcttgttgaattagatggtgatgttaatgggcacaaattttctgtcagtggagagggtgaaggtgatgcaacatagggaaaacttacccttaaatttatttgcactactggaaaactacctgttccatggccaacacttgtcactactttgacttatggtgttcaatgcttttcaagatacccggatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagggaccacatggtccttcttgagtttgtaacagctgctgggattacacatggcatggatgaactgtaccaaagcgcttggagccacccgcagttcgaaaaataataagcttgacctgtgaagtgaaaaatggcgcacattgtgcgacattttttttgtctgccgtttaccgctactgcgcggcagtacgcctttggtttatccattttatacaatccatgtaaaaaagggccctgaaattcaggaccctttctgagctcattacaggttggtgctaataccattatagtagctctcggaacggcttgcacgtttaatgtttttgaaaccgtgcattactttcagcagacgttcgagaccaaaacctgcaccaatccagggtttatcaataccccattcacgatccaggctaaccggaccaacaactgcgctactcagttccagatcaccgtgcataatatccagggtatcaccataaaccatgcagctatcaccaacaatttcaaagtcgatttccaggtaatccagaaactctttaatcagtgcttccagattttcacgggtacaaccgctacccatctgacaaaagttcaccattgtaaattcttccaggtgttctttaccatcactttctttacgatagcacggaccaacttcaaagattttgataggaccaggcagaatacgatccagtttccgcaggtagttatacagtgtcggtgccagcataggacgcagacacaggtttttatcaacgcgaaagatttgtttgctcagttcggtatcattgttaatgcccatacgttcaacatattctgccggaatcagaatcgggcttttgatttccagaaaaccgcgatccacgaaaaatttggtaatatcacgttccagtttacccagataatcttcgcggtcgttggtatataagcgttgaaaatcatttttacgacgagtaaccagttccggttccagttcacgaaacggttttgccatattcaggctgattttatcttcaggactcagcagtgcttcaacacgatctaactgagaccgggttaagctcggtgccggtgcgcttgccggaacgctgctattcggggtgctttttgccggactcggaacgctacggctggtattggtgcttgcttttgcgctaacgctattttccagaggtttcggtgcgcgactaacgcttttcggcattgcttttttaactttaggtgcgctcacaacacgaactttaactgaatttttgctttcggtgctgcgtgtcagaaaattgttaatatcttcatcactcacacggcagcgtttacaggttttacgatatttgtgatgacgaaatgcgcgtgcggtacgacagctacggctattattcacaaccagatgatcgccacaggccatttcaatatagattttgctgcggctaacttcgtgatgtttgattttatgcagggtgccggtacggctcatccacagacctgttgcgctaatcagaacatccagcggttttttatccatatcgtacctccttaaatttctaggttgtgacctaggtgatttagtttaccagtgcaaaagaaatgtcaaaagagaagggcgtgaatttaacgcggttccagcgcaaagacttcaaaacctgcgtcggtgccgatttcggcctattggttaaaaaatgagctgagttctagtaaaaaaaatccttagctttcgctaaggatctgcagtggcggaaaccccgggaatctaacccggctgaacggatttagagtccattcgatctacatgatcaggtttccgaattcagcgttacaagtattacacaaagttttttatgttgagaatatttttttgatggggcatggcgcaaaacctttcgcggtatggcatgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaagaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaatgagaatattcgttttcacccaaggaatagaggatatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgactagttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatagcttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 24 - DNA, 660 bp, Artificial Sequence
  CatUAG119
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgactagttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 25 - DNA, 714 bp, Artificial Sequence
  eGFPUAG39
atgtctaaaggtgaagaacttttcactggagttgtcccaattcttgttgaattagatggtgatgttaatgggcacaaattttctgtcagtggagagggtgaaggtgatgcaacatagggaaaacttacccttaaatttatttgcactactggaaaactacctgttccatggccaacacttgtcactactttgacttatggtgttcaatgcttttcaagatacccggatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagggaccacatggtccttcttgagtttgtaacagctgctgggattacacatggcatggatgaactgtaccaa

SEQ ID NO: 26 - DNA, 1257 bp, Artificial Sequence
  wt PylRS
atggataaaaaaccgctggatgttctgattagcgcaacaggtctgtggatgagccgtaccggcaccctgcataaaatcaaacatcacgaagttagccgcagcaaaatctatattgaaatggcctgtggcgatcatctggttgtgaataatagccgtagctgtcgtaccgcacgcgcatttcgtcatcacaaatatcgtaaaacctgtaaacgctgccgtgtgagtgatgaagatattaacaattttctgacacgcagcaccgaaagcaaaaattcagttaaagttcgtgttgtgagcgcacctaaagttaaaaaagcaatgccgaaaagcgttagtcgcgcaccgaaacctctggaaaatagcgttagcgcaaaagcaagcaccaataccagccgtagcgttccgagtccggcaaaaagcaccccgaatagcagcgttccggcaagcgcaccggcaccgagcttaacccggtctcagttagatcgtgttgaagcactgctgagtcctgaagataaaatcagcctgaatatggcaaaaccgtttcgtgaactggaaccggaactggttactcgtcgtaaaaatgattttcaacgcttatataccaacgaccgcgaagattatctgggtaaactggaacgtgatattaccaaatttttcgtggatcgcggttttctggaaatcaaaagcccgattctgattccggcagaatatgttgaacgtatgggcattaacaatgataccgaactgagcaaacaaatctttcgcgttgataaaaacctgtgtctgcgtcctatgctggcaccgacactgtataactacctgcggaaactggatcgtattctgcctggtcctatcaaaatctttgaagttggtccgtgctatcgtaaagaaagtgatggtaaagaacacctggaagaatttacaatggtgaacttttgtcagatgggtagcggttgtacccgtgaaaatctggaagcactgattaaagagtttctggattacctggaaatcgactttgaaattgttggtgatagctgcatggtttatggtgataccctggatattatgcacggtgatctggaactgagtagcgcagttgttggtccggttagcctggatcgtgaatggggtattgataaaccctggattggtgcaggttttggtctcgaacgtctgctgaaagtaatgcacggtttcaaaaacattaaacgtgcaagccgttccgagagctactataatggtattagcaccaacctg

SEQ ID NO: 27 - DNA, 29 bp, Artificial Sequence
  Primer
tagctgcatggtttttggtgataccctgg

SEQ ID NO: 28 - DNA, 29 bp, Artificial Sequence
  Primer
ccagggtatcaccaaaaaccatgcagcta

SEQ ID NO: 29 - DNA, 1257 bp, Artificial Sequence
  PylRS#1 (Y349F)
atggataaaaaaccgctggatgttctgattagcgcaacaggtctgtggatgagccgtaccggcaccctgcataaaatcaaacatcacgaagttagccgcagcaaaatctatattgaaatggcctgtggcgatcatctggttgtgaataatagccgtagctgtcgtaccgcacgcgcatttcgtcatcacaaatatcgtaaaacctgtaaacgctgccgtgtgagtgatgaagatattaacaattttctgacacgcagcaccgaaagcaaaaattcagttaaagttcgtgttgtgagcgcacctaaagttaaaaaagcaatgccgaaaagcgttagtcgcgcaccgaaacctctggaaaatagcgttagcgcaaaagcaagcaccaataccagccgtagcgttccgagtccggcaaaaagcaccccgaatagcagcgttccggcaagcgcaccggcaccgagcttaacccggtctcagttagatcgtgttgaagcactgctgagtcctgaagataaaatcagcctgaatatggcaaaaccgtttcgtgaactggaaccggaactggttactcgtcgtaaaaatgattttcaacgcttatataccaacgaccgcgaagattatctgggtaaactggaacgtgatattaccaaatttttcgtggatcgcggttttctggaaatcaaaagcccgattctgattccggcagaatatgttgaacgtatgggcattaacaatgataccgaactgagcaaacaaatctttcgcgttgataaaaacctgtgtctgcgtcctatgctggcaccgacactgtataactacctgcggaaactggatcgtattctgcctggtcctatcaaaatctttgaagttggtccgtgctatcgtaaagaaagtgatggtaaagaacacctggaagaatttacaatggtgaacttttgtcagatgggtagcggttgtacccgtgaaaatctggaagcactgattaaagagtttctggattacctggaaatcgactttgaaattgttggtgatagctgcatggtttttggtgataccctggatattatgcacggtgatctggaactgagtagcgcagttgttggtccggttagcctggatcgtgaatggggtattgataaaccctggattggtgcaggttttggtctcgaacgtctgctgaaagtaatgcacggtttcaaaaacattaaacgtgcaagccgttccgagagctactataatggtattagcaccaacctg

SEQ ID NO: 30 - DNA, 27 bp, Artificial Sequence
  Primer
gagcttaacccggtctcagttagatcg

SEQ ID NO: 31 - DNA, 19 bp, Artificial Sequence
  Primer
ggtagcggttgtacccgtg

SEQ ID NO: 32 - DNA, 57 bp, Artificial Sequence
  Primer
  variation 18: /replace=“a,c,g,t”
  variation 19: /replace=“a,c,g,t”
  variation 24: /replace=“a,c,g,t”
  variation 25: /replace=“a,c,g,t”
  variation 30: /replace=“a,c,g,t”
  variation 31: /replace=“a,c,g,t”
  variation 36: /replace=“a,c,g,t”
  variation 37: /replace=“a,c,g,t”
gggtacaaccgctaccsnnctgsnnaaasnncacsnntgtaaattcttccaggtgtt

SEQ ID NO: 33 - DNA, 61 bp, Artificial Sequence
  Primer
  variation 39: /replace=“a,c,g,t”
  variation 40: /replace=“a,c,g,t”
ctttcagcagacgttcgagaccaaaacctgcaccaatsnngggtttatcaataccccattc

SEQ ID NO: 34 - DNA, 21 bp, Artificial Sequence
  Primer
ctttcagcagacgttcgagac

SEQ ID NO: 35 - DNA, 1257 bp, Artificial Sequence
  CafRS#7
atggataaaaaaccgctggatgttctgattagcgcaacaggtctgtggatgagccgtaccggcaccctgcataaaatcaaacatcacgaagttagccgcagcaaaatctatattgaaatggcctgtggcgatcatctggttgtgaataatagccgtagctgtcgtaccgcacgcgcatttcgtcatcacaaatatcgtaaaacctgtaaacgctgccgtgtgagtgatgaagatattaacaattttctgacacgcagcaccgaaagcaaaaattcagttaaagttcgtgttgtgagcgcacctaaagttaaaaaagcaatgccgaaaagcgttagtcgcgcaccgaaacctctggaaaatagcgttagcgcaaaagcaagcaccaataccagccgtagcgttccgagtccggcaaaaagcaccccgaatagcagcgttccggcaagcgcaccggcaccgagcttaacccggtctcagttagatcgtgttgaagcactgctgagtcctgaagataaaatcagcctgaatatggcaaaaccgtttcgtgaactggaaccggaactggttactcgtcgtaaaaatgattttcaacgcttatataccaacgaccgcgaagattatctgggtaaactggaacgtgatattaccaaatttttcgtggatcgcggttttctggaaatcaaaagcccgattctgattccggcagaatatgttgaacgtatgggcattaacaatgataccgaactgagcaaacaaatctttcgcgttgataaaaacctgtgtctgcgtcctatgctggcaccgacactgtataactacctgcggaaactggatcgtattctgcctggtcctatcaaaatctttgaagttggtccgtgctatcgtaaagaaagtgatggtaaagaacacctggaagaatttacacaggtgtcctttggccagatgggtagcggttgtacccgtgaaaatctggaagcactgattaaagagtttctggattacctggaaatcgactttgaaattgttggtgatagctgcatggtttttggtgataccctggatattatgcacggtgatctggaactgagtagcgcagttgttggtccggttagcctggatcgtgaatggggtattgataaaccctggattggtgcaggttttggtctcgaacgtctgctgaaagtaatgcacggtttcaaaaacattaaacgtgcaagccgttccgagagctactataatggtattagcaccaacctg

SEQ ID NO: 36 - DNA, 1257 bp, Artificial Sequence
  CafRS#7-R6
  variation 799: /replace=“a,c,g,t”
  variation 800: /replace=“a,c,g,t”
  variation 801: /replace=“a,c,g,t”
  variation 808: /replace=“a,c,g,t”
  variation 809: /replace=“a,c,g,t”
  variation 811: /replace=“a,c,g,t”
  variation 812: /replace=“a,c,g,t”
  variation 820: /replace=“a,c,g,t”
  variation 821: /replace=“a,c,g,t”
  variation 822: /replace=“a,c,g,t”
  variation 853: /replace=“a,c,g,t”
  variation 854: /replace=“a,c,g,t”
  variation 859: /replace=“a,c,g,t”
  variation 860: /replace=“a,c,g,t”
atggataaaaaaccgctggatgttctgattagcgcaacaggtctgtggatgagccgtaccggcaccctgcataaaatcaaacatcacgaagttagccgcagcaaaatctatattgaaatggcctgtggcgatcatctggttgtgaataatagccgtagctgtcgtaccgcacgcgcatttcgtcatcacaaatatcgtaaaacctgtaaacgctgccgtgtgagtgatgaagatattaacaattttctgacacgcagcaccgaaagcaaaaattcagttaaagttcgtgttgtgagcgcacctaaagttaaaaaagcaatgccgaaaagcgttagtcgcgcaccgaaacctctggaaaatagcgttagcgcaaaagcaagcaccaataccagccgtagcgttccgagtccggcaaaaagcaccccgaatagcagcgttccggcaagcgcaccggcaccgagcttaacccggtctcagttagatcgtgttgaagcactgctgagtcctgaagataaaatcagcctgaatatggcaaaaccgtttcgtgaactggaaccggaactggttactcgtcgtaaaaatgattttcaacgcttatataccaacgaccgcgaagattatctgggtaaactggaacgtgatattaccaaatttttcgtggatcgcggttttctggaaatcaaaagcccgattctgattccggcagaatatgttgaacgtatgggcattaacaatgataccgaactgagcaaacaaatctttcgcgttgataaaaacctgtgtctgcgtcctatgctgnnnccgacannsnnsaactacnnncggaaactggatcgtattctgcctggtcctnnsaaannstttgaagttggtccgtgctatcgtaaagaaagtgatggtaaagaacacctggaagaatttacacaggtgtcctttggccagatgggtagcggttgtacccgtgaaaatctggaagcactgattaaagagtttctggattacctggaaatcgactttgaaattgttggtgatagctgcatggtttttggtgataccctggatattatgcacggtgatctggaactgagtagcgcagttgttggtccggttagcctggatcgtgaatggggtattgataaaccctggattggtgcaggttttggtctcgaacgtctgctgaaagtaatgcacggtttcaaaaacattaaacgtgcaagccgttccgagagctactataatggtattagcaccaacctg

SEQ ID NO: 37 - DNA, 66 bp, Artificial Sequence
  Primer
  variation 25: /replace=“a,c,g,t”
  variation 26: /replace=“a,c,g,t”
  variation 34: /replace=“a,c,g,t”
  variation 35: /replace=“a,c,g,t”
  variation 37: /replace=“a,c,g,t”
  variation 38: /replace=“a,c,g,t”
  variation 46: /replace=“a,c,g,t”
  variation 47: /replace=“a,c,g,t”
ggcagaatacgatccagtttccgsnngtagttsnnsnntgtcggsnncagcataggacgcagacac

SEQ ID NO: 38 - DNA, 63 bp, Artificial Sequence
  Primer
  variation 31: /replace=“a,c,g,t”
  variation 32: /replace=“a,c,g,t”
  variation 37: /replace=“a,c,g,t”
  variation 38: /replace=“a,c,g,t”
cggaaactggatcgtattctgcctggtcctnnsaaannstttgaagttggtccgtgctatcgt

SEQ ID NO: 39 - DNA, 1386 bp, Artificial Sequence
  CafRS#29
atggataaaaaaccgctggatgttctgattagcgcaacaggtctgtggatgagccgtaccggcaccctgcataaaatcaaacatcacgaagttagccgcagcaaaatctatattgaaatggcctgtggcgatcatctggttgtgaataatagccgtagctgtcgtaccgcacgcgcatttcgtcatcacaaatatcgtaaaacctgtaaacgctgccgtgtgagtgatgaagatattaacaattttctgacacgcagcaccgaaagcaaaaattcagttaaagttcgtgttgtgagcgcacctaaagttaaaaaagcaatgccgaaaagcgttagtcgcgcaccgaaacctctggaaaatagcgttagcgcaaaagcaagcaccaataccagccgtagcgttccgagtccggcaaaaagcaccccgaatagcagcgttccggcaagcgcaccggcaccgagcttaacccggtctcagttagatcgtgttgaagcactgctgagtcctgaagataaaatcagcctgaatatggcaaaaccgtttcgtgaactggaaccggaactggttactcgtcgtaaaaatgattttcaacgcttatataccaacgaccgcgaagattatctgggtaaactggaacgtgatattaccaaatttttcgtggatcgcggttttctggaaatcaaaagcccgattctgattccggcagaatatgttgaacgtatgggcattaacaatgataccgaactgagcaaacaaatctttcgcgttgataaaaacctgtgtctgcgtcctatgctgaccccgacattgttcaactacgcgcggaaactggatcgtattctgcctggtcctaacaagagctttgaagttggtccgtgctatcgtaaagaaagtgatggtaaagaacacctggaagaatttacacaggtgtcctttggccagatgggtagcggttgtacccgtgaaaatctggaagcactgattaaagagtttctggattacctggaaatcgactttgaaattgttggtgatagctgcatggtttttggtgataccctggatattatgcacggtgatctggaactgagtagcgcagttgttggtccggttagcctggatcgtgaatggggtattgataaaccctggattggtgcaggttttggtctcgaacgtctgctgaaagtaatgcacggtttcaaaaacattaaacgtgcaagccgttccgagagctactataatggtattagcaccaacctgtaatgagctcagagagggtcctgattttcagggccctttttttacgtggtattgtataaaatggataaaccaaaggcgtactgccgcgcagtagcggtaaacggcagacaaaaaaaatgtcgcacagtg

SEQ ID NO: 40 - DNA, 1386 bp, Artificial Sequence
  CafRS#29-R5
  variation 820: /replace=“a,c,g,t”
  variation 821: /replace=“a,c,g,t”
  variation 853: /replace=“a,c,g,t”
  variation 854: /replace=“a,c,g,t”
  variation 859: /replace=“a,c,g,t”
  variation 860: /replace=“a,c,g,t”
  variation 925: /replace=“a,c,g,t”
  variation 926: /replace=“a,c,g,t”
  variation 931: /replace=“a,c,g,t”
  variation 932: /replace=“a,c,g,t”
atggataaaaaaccgctggatgttctgattagcgcaacaggtctgtggatgagccgtaccggcaccctgcataaaatcaaacatcacgaagttagccgcagcaaaatctatattgaaatggcctgtggcgatcatctggttgtgaataatagccgtagctgtcgtaccgcacgcgcatttcgtcatcacaaatatcgtaaaacctgtaaacgctgccgtgtgagtgatgaagatattaacaattttctgacacgcagcaccgaaagcaaaaattcagttaaagttcgtgttgtgagcgcacctaaagttaaaaaagcaatgccgaaaagcgttagtcgcgcaccgaaacctctggaaaatagcgttagcgcaaaagcaagcaccaataccagccgtagcgttccgagtccggcaaaaagcaccccgaatagcagcgttccggcaagcgcaccggcaccgagcttaacccggtctcagttagatcgtgttgaagcactgctgagtcctgaagataaaatcagcctgaatatggcaaaaccgtttcgtgaactggaaccggaactggttactcgtcgtaaaaatgattttcaacgcttatataccaacgaccgcgaagattatctgggtaaactggaacgtgatattaccaaatttttcgtggatcgcggttttctggaaatcaaaagcccgattctgattccggcagaatatgttgaacgtatgggcattaacaatgataccgaactgagcaaacaaatctttcgcgttgataaaaacctgtgtctgcgtcctatgctgaccccgacattgttcaactacnnscggaaactggatcgtattctgcctggtcctnnsaagnnstttgaagttggtccgtgctatcgtaaagaaagtgatggtaaagaacacctggaagaatttacannsgtgnnstttggccagatgggtagcggttgtacccgtgaaaatctggaagcactgattaaagagtttctggattacctggaaatcgactttgaaattgttggtgatagctgcatggtttttggtgataccctggatattatgcacggtgatctggaactgagtagcgcagttgttggtccggttagcctggatcgtgaatggggtattgataaaccctggattggtgcaggttttggtctcgaacgtctgctgaaagtaatgcacggtttcaaaaacattaaacgtgcaagccgttccgagagctactataatggtattagcaccaacctgtaatgagctcagagagggtcctgattttcagggccctttttttacgtggtattgtataaaatggataaaccaaaggcgtactgccgcgcagtagcggtaaacggcagacaaaaaaaatgtcgcacagtg

SEQ ID NO: 41 - DNA, 42 bp, Artificial Sequence
  Primer
  variation 23: /replace=“a,c,g,t”
  variation 24: /replace=“a,c,g,t”
cagaatacgatccagtttccgsnngtagttatacagtgtcgg

SEQ ID NO: 42 - DNA, 57 bp, Artificial Sequence
  Primer
  variation 30: /replace=“a,c,g,t”
  variation 31: /replace=“a,c,g,t”
  variation 36: /replace=“a,c,g,t”
  variation 37: /replace=“a,c,g,t”
gggtacaaccgctacccatctggccaaasnncacsnntgtaaattcttccaggtgtt

SEQ ID NO: 43 - DNA, 1260 bp, Artificial Sequence
  CafRS#30
atggataaaaaaccgctggatgttctgattagcgcaacaggtctgtggatgagccgtaccggcaccctgcataaaatcaaacatcacgaagttagccgcagcaaaatctatattgaaatggcctgtggcgatcatctggttgtgaataatagccgtagctgtcgtaccgcacgcgcatttcgtcatcacaaatatcgtaaaacctgtaaacgctgccgtgtgagtgatgaagatattaacaattttctgacacgcagcaccgaaagcaaaaattcagttaaagttcgtgttgtgagcgcacctaaagttaaaaaagcaatgccgaaaagcgttagtcgcgcaccgaaacctctggaaaatagcgttagcgcaaaagcaagcaccaataccagccgtagcgttccgagtccggcaaaaagcaccccgaatagcagcgttccggcaagcgcaccggcaccgagcttaacccggtctcagttagatcgtgttgaagcactgctgagtcctgaagataaaatcagcctgaatatggcaaaaccgtttcgtgaactggaaccggaactggttactcgtcgtaaaaatgattttcaacgcttatataccaacgaccgcgaagattatctgggtaaactggaacgtgatattaccaaatttttcgtggatcgcggttttctggaaatcaaaagcccgattctgattccggcagaatatgttgaacgtatgggcattaacaatgataccgaactgagcaaacaaatctttcgcgttgataaaaacctgtgtctgcgtcctatgctgaccccgacattgtataactacagccggaaactggatcgtattctgcctggtccttccaaagtctttgaagttggtccgtgctatcgtaaagaaagtgatggtaaagaacacctggaagaatttacaatggtggtgtttggccagatgggtagcggttgtacccgtgaaaatctggaagcactgattaaagagtttctggattacctggaaatcgactttgaaattgttggtgatagctgcatggtttttggtgataccctggatattatgcacggtgatctggaactgagtagcgcagttgttggtccggttagcctggatcgtgaatggggtattgataaaccctggattggtgcaggttttggtctcgaacgtctgctgaaagtaatgcacggtttcaaaaacattaaacgtgcaagccgttccgagagctactataatggtattagcaccaacctgtaa

SEQ ID NO: 44 - DNA, 3139 bp, Artificial Sequence
  pSAm1
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggatctcgatcccgcgaaattaatacgactcactatagggagaccacaacggtttccctctagaaataattttgtttaactttaagaaggagatatacatatggaagcaggtatcaccggcacctggtacaaccagctcggctcgaccttcatcgtgaccgcgggtgcagacggagctctgaccggtacctacgtcacggcgcgtggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctcctaataagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 45 - DNA, 40 bp, Artificial Sequence
  Primer
gcagacggagccctgaccggtacctactagacggcgcgtg

SEQ ID NO: 46 - DNA, 40 bp, Artificial Sequence
  Primer
cacgcgccgtctagtaggtaccggtcagggctccgtctgc

SEQ ID NO: 47 - DNA, 384 bp, Artificial Sequence
  SAm1UAG44
atggaagcaggtatcaccggcacctggtacaaccagctcggctcgaccttcatcgtgaccgcgggtgcagacggagccctgaccggtacctactagacggcgcgtggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctcctaa

SEQ ID NO: 48 - DNA, 28 bp, Artificial Sequence
  Primer
gatcaacacccagtagctgctgacctcc

SEQ ID NO: 49 - DNA, 28 bp, Artificial Sequence
  Primer
ggaggtcagcagctactgggtgttgatc

SEQ ID NO: 50 - DNA, 28 bp, Artificial Sequence
  Primer
gaggccaacgcctagaagtccacgctgg

SEQ ID NO: 51 - DNA, 28 bp, Artificial Sequence
  Primer
ccagcgtggacttctaggcgttggcctc

SEQ ID NO: 52 - DNA, 384 bp, Artificial Sequence
  SAm1UAG120
atggaagcaggtatcaccggcacctggtacaaccagctcggctcgaccttcatcgtgaccgcgggtgcagacggagctctgaccggtacctacgtcacggcgcgtggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctagaagtccacgctggtcggccacgacaccttcaccaaggtgaagccgtccgccgcctcctaa

SEQ ID NO: 53 - DNA, 5798 bp, Artificial Sequence
  pSBX8.CafRS#30.d58
tttttacggttcctggccttttgctggccttttgctcacatgacccgacaccataacgctcggttgccgccgggcgttttttattggccagatgattaattcctaatttttgttgacactctatcattgatagagttattttaccactccctatcagtgatagagaaaagtgaaatgaatagttcgacaaaatctagataacgagggcaaaaaatgtctaaaggtgaagaacttttcactggagttgtcccaattcttgttgaattagatggtgatgttaatgggcacaaattttctgtcagtggagagggtgaaggtgatgcaacatagggaaaacttacccttaaatttatttgcactactggaaaactacctgttccatggccaacacttgtcactactttgacttatggtgttcaatgcttttcaagatacccggatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagggaccacatggtccttcttgagtttgtaacagctgctgggattacacatggcatggatgaactgtaccaaagcgcttggagccacccgcagttcgaaaaataataagcttgacctgtgaagtgaaaaatggcgcacattgtgcgacattttttttgtctgccgtttaccgctactgcgcggcagtacgcctttggtttatccattttatacaatccatgtaaaaaagggccctgaaattcaggaccctttctgagctcattacaggttggtgctaataccattatagtagctctcggaacggcttgcacgtttaatgtttttgaaaccgtgcattactttcagcagacgttcgagaccaaaacctgcaccaatccagggtttatcaataccccattcacgatccaggctaaccggaccaacaactgcgctactcagttccagatcaccgtgcataatatccagggtatcaccaaaaaccatgcagctatcaccaacaatttcaaagtcgatttccaggtaatccagaaactctttaatcagtgcttccagattttcacgggtacaaccgctacccatctggccaaacaccaccattgtaaattcttccaggtgttctttaccatcactttctttacgatagcacggaccaacttcaaagactttggaaggaccaggcagaatacgatccagtttccggctgtagttatacaatgtcggggtcagcataggacgcagacacaggtttttatcaacgcgaaagatttgtttgctcagttcggtatcattgttaatgcccatacgttcaacatattctgccggaatcagaatcgggcttttgatttccagaaaaccgcgatccacgaaaaatttggtaatatcacgttccagtttacccagataatcttcgcggtcgttggtatataagcgttgaaaatcatttttacgacgagtaaccagttccggttccagttcacgaaacggttttgccatattcaggctgattttatcttcaggactcagcagtgcttcaacacgatctaactgagaccgggttaagctcggtgccggtgcgcttgccggaacgctgctattcggggtgctttttgccggactcggaacgctacggctggtattggtgcttgcttttgcgctaacgctattttccagaggtttcggtgcgcgactaacgcttttcggcattgcttttttaactttaggtgcgctcacaacacgaactttaactgaatttttgctttcggtgctgcgtgtcagaaaattgttaatatcttcatcactcacacggcagcgtttacaggttttacgatatttgtgatgacgaaatgcgcgtgcggtacgacagctacggctattattcacaaccagatgatcgccacaggccatttcaatatagattttgctgcggctaacttcgtgatgtttgattttatgcagggtgccggtacggctcatccacagacctgttgcgctaatcagaacatccagcggttttttatccatatcgtacctccttaaatttctaggttgtgacctaggtgatttagtttaccagtgcaaaagaaatgtcaaaagagaagggcgtgaatttaacgcggttccagcgcaaagacttcaaaacctgcgtcggtgccgatttcggcctattggttaaaaaatgagctgagttctagtaaaaaaaatccttagctttcgctaaggatctgcagtggcggaaaccccgggaatctaacccggctgaacggatttagagtccattcgatctacatgatcaggtttccgaattcagcgttacaagtattacacaaagttttttatgttgagaatatttttttgatggggcatggcgcaaaacctttcgcggtatggcatgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaagaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaatgagaatattcgttttcacccaaggaatagaggatatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgactagttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatagcttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 54 - DNA, 5435 bp, Artificial Sequence
  pSBX8.CafRS#30.d47
tttttacggttcctggccttttgctggccttttgctcacatgacccgacaccataacgctcggttgccgccgggcgttttttattggccagatgattaattcctaatttttgttgacactctatcattgatagagttattttaccactccctatcagtgatagagaaaagtgaaatgaatagttcgacaaaatctagataacgagggcaaaaaatggaagcaggtatcaccggcacctggtacaaccagctcggctcgaccttcatcgtgaccgcgggtgcagacggagccctgaccggtacctactagacggcgcgtggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctcctaataagcttgacctgtgaagtgaaaaatggcgcacattgtgcgacattttttttgtctgccgtttaccgctactgcgcggcagtacgcctttggtttatccattttatacaatccatgtaaaaaagggccctgaaattcaggaccctttctgagctcattacaggttggtgctaataccattatagtagctctcggaacggcttgcacgtttaatgtttttgaaaccgtgcattactttcagcagacgttcgagaccaaaacctgcaccaatccagggtttatcaataccccattcacgatccaggctaaccggaccaacaactgcgctactcagttccagatcaccgtgcataatatccagggtatcaccaaaaaccatgcagctatcaccaacaatttcaaagtcgatttccaggtaatccagaaactctttaatcagtgcttccagattttcacgggtacaaccgctacccatctggccaaacaccaccattgtaaattcttccaggtgttctttaccatcactttctttacgatagcacggaccaacttcaaagactttggaaggaccaggcagaatacgatccagtttccggctgtagttatacaatgtcggggtcagcataggacgcagacacaggtttttatcaacgcgaaagatttgtttgctcagttcggtatcattgttaatgcccatacgttcaacatattctgccggaatcagaatcgggcttttgatttccagaaaaccgcgatccacgaaaaatttggtaatatcacgttccagtttacccagataatcttcgcggtcgttggtatataagcgttgaaaatcatttttacgacgagtaaccagttccggttccagttcacgaaacggttttgccatattcaggctgattttatcttcaggactcagcagtgcttcaacacgatctaactgagaccgggttaagctcggtgccggtgcgcttgccggaacgctgctattcggggtgctttttgccggactcggaacgctacggctggtattggtgcttgcttttgcgctaacgctattttccagaggtttcggtgcgcgactaacgcttttcggcattgcttttttaactttaggtgcgctcacaacacgaactttaactgaatttttgctttcggtgctgcgtgtcagaaaattgttaatatcttcatcactcacacggcagcgtttacaggttttacgatatttgtgatgacgaaatgcgcgtgcggtacgacagctacggctattattcacaaccagatgatcgccacaggccatttcaatatagattttgctgcggctaacttcgtgatgtttgattttatgcagggtgccggtacggctcatccacagacctgttgcgctaatcagaacatccagcggttttttatccatatcgtacctccttaaatttctaggttgtgacctaggtgatttagtttaccagtgcaaaagaaatgtcaaaagagaagggcgtgaatttaacgcggttccagcgcaaagacttcaaaacctgcgtcggtgccgatttcggcctattggttaaaaaatgagctgagttctagtaaaaaaaatccttagctttcgctaaggatctgcagtggcggaaaccccgggaatctaacccggctgaacggatttagagtccattcgatctacatgatcaggtttccgaattcagcgttacaagtattacacaaagttttttatgttgagaatatttttttgatggggcatggcgcaaaacctttcgcggtatggcatgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaagaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaatgagaatattcgttttcacccaaggaatagaggatatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgactagttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatagcttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 55 - DNA, 5435 bp, Artificial Sequence
  pSBX8.CafRS#30.d53
tttttacggttcctggccttttgctggccttttgctcacatgacccgacaccataacgctcggttgccgccgggcgttttttattggccagatgattaattcctaatttttgttgacactctatcattgatagagttattttaccactccctatcagtgatagagaaaagtgaaatgaatagttcgacaaaatctagataacgagggcaaaaaatggaagcaggtatcaccggcacctggtacaaccagctcggctcgaccttcatcgtgaccgcgggtgcagacggagctctgaccggtacctacgtcacggcgcgtggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtagctgctgacctccggcaccaccgaggccaacgcctggaagtccacgctggtcggccacgacaccttcaccaaggtgaagccgtccgccgcctcctaataagcttgacctgtgaagtgaaaaatggcgcacattgtgcgacattttttttgtctgccgtttaccgctactgcgcggcagtacgcctttggtttatccattttatacaatccatgtaaaaaagggccctgaaattcaggaccctttctgagctcattacaggttggtgctaataccattatagtagctctcggaacggcttgcacgtttaatgtttttgaaaccgtgcattactttcagcagacgttcgagaccaaaacctgcaccaatccagggtttatcaataccccattcacgatccaggctaaccggaccaacaactgcgctactcagttccagatcaccgtgcataatatccagggtatcaccaaaaaccatgcagctatcaccaacaatttcaaagtcgatttccaggtaatccagaaactctttaatcagtgcttccagattttcacgggtacaaccgctacccatctggccaaacaccaccattgtaaattcttccaggtgttctttaccatcactttctttacgatagcacggaccaacttcaaagactttggaaggaccaggcagaatacgatccagtttccggctgtagttatacaatgtcggggtcagcataggacgcagacacaggtttttatcaacgcgaaagatttgtttgctcagttcggtatcattgttaatgcccatacgttcaacatattctgccggaatcagaatcgggcttttgatttccagaaaaccgcgatccacgaaaaatttggtaatatcacgttccagtttacccagataatcttcgcggtcgttggtatataagcgttgaaaatcatttttacgacgagtaaccagttccggttccagttcacgaaacggttttgccatattcaggctgattttatcttcaggactcagcagtgcttcaacacgatctaactgagaccgggttaagctcggtgccggtgcgcttgccggaacgctgctattcggggtgctttttgccggactcggaacgctacggctggtattggtgcttgcttttgcgctaacgctattttccagaggtttcggtgcgcgactaacgcttttcggcattgcttttttaactttaggtgcgctcacaacacgaactttaactgaatttttgctttcggtgctgcgtgtcagaaaattgttaatatcttcatcactcacacggcagcgtttacaggttttacgatatttgtgatgacgaaatgcgcgtgcggtacgacagctacggctattattcacaaccagatgatcgccacaggccatttcaatatagattttgctgcggctaacttcgtgatgtttgattttatgcagggtgccggtacggctcatccacagacctgttgcgctaatcagaacatccagcggttttttatccatatcgtacctccttaaatttctaggttgtgacctaggtgatttagtttaccagtgcaaaagaaatgtcaaaagagaagggcgtgaatttaacgcggttccagcgcaaagacttcaaaacctgcgtcggtgccgatttcggcctattggttaaaaaatgagctgagttctagtaaaaaaaatccttagctttcgctaaggatctgcagtggcggaaaccccgggaatctaacccggctgaacggatttagagtccattcgatctacatgatcaggtttccgaattcagcgttacaagtattacacaaagttttttatgttgagaatatttttttgatggggcatggcgcaaaacctttcgcggtatggcatgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaagaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaatgagaatattcgttttcacccaaggaatagaggatatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgactagttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatagcttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 56 - DNA, 5435 bp, Artificial Sequence
  pSBX8.CafRS#30.d51
tttttacggttcctggccttttgctggccttttgctcacatgacccgacaccataacgctcggttgccgccgggcgttttttattggccagatgattaattcctaatttttgttgacactctatcattgatagagttattttaccactccctatcagtgatagagaaaagtgaaatgaatagttcgacaaaatctagataacgagggcaaaaaatggaagcaggtatcaccggcacctggtacaaccagctcggctcgaccttcatcgtgaccgcgggtgcagacggagctctgaccggtacctacgtcacggcgcgtggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctagaagtccacgctggtcggccacgacaccttcaccaaggtgaagccgtccgccgcctcctaataagcttgacctgtgaagtgaaaaatggcgcacattgtgcgacattttttttgtctgccgtttaccgctactgcgcggcagtacgcctttggtttatccattttatacaatccatgtaaaaaagggccctgaaattcaggaccctttctgagctcattacaggttggtgctaataccattatagtagctctcggaacggcttgcacgtttaatgtttttgaaaccgtgcattactttcagcagacgttcgagaccaaaacctgcaccaatccagggtttatcaataccccattcacgatccaggctaaccggaccaacaactgcgctactcagttccagatcaccgtgcataatatccagggtatcaccaaaaaccatgcagctatcaccaacaatttcaaagtcgatttccaggtaatccagaaactctttaatcagtgcttccagattttcacgggtacaaccgctacccatctggccaaacaccaccattgtaaattcttccaggtgttctttaccatcactttctttacgatagcacggaccaacttcaaagactttggaaggaccaggcagaatacgatccagtttccggctgtagttatacaatgtcggggtcagcataggacgcagacacaggtttttatcaacgcgaaagatttgtttgctcagttcggtatcattgttaatgcccatacgttcaacatattctgccggaatcagaatcgggcttttgatttccagaaaaccgcgatccacgaaaaatttggtaatatcacgttccagtttacccagataatcttcgcggtcgttggtatataagcgttgaaaatcatttttacgacgagtaaccagttccggttccagttcacgaaacggttttgccatattcaggctgattttatcttcaggactcagcagtgcttcaacacgatctaactgagaccgggttaagctcggtgccggtgcgcttgccggaacgctgctattcggggtgctttttgccggactcggaacgctacggctggtattggtgcttgcttttgcgctaacgctattttccagaggtttcggtgcgcgactaacgcttttcggcattgcttttttaactttaggtgcgctcacaacacgaactttaactgaatttttgctttcggtgctgcgtgtcagaaaattgttaatatcttcatcactcacacggcagcgtttacaggttttacgatatttgtgatgacgaaatgcgcgtgcggtacgacagctacggctattattcacaaccagatgatcgccacaggccatttcaatatagattttgctgcggctaacttcgtgatgtttgattttatgcagggtgccggtacggctcatccacagacctgttgcgctaatcagaacatccagcggttttttatccatatcgtacctccttaaatttctaggttgtgacctaggtgatttagtttaccagtgcaaaagaaatgtcaaaagagaagggcgtgaatttaacgcggttccagcgcaaagacttcaaaacctgcgtcggtgccgatttcggcctattggttaaaaaatgagctgagttctagtaaaaaaaatccttagctttcgctaaggatctgcagtggcggaaaccccgggaatctaacccggctgaacggatttagagtccattcgatctacatgatcaggtttccgaattcagcgttacaagtattacacaaagttttttatgttgagaatatttttttgatggggcatggcgcaaaacctttcgcggtatggcatgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaagaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaatgagaatattcgttttcacccaaggaatagaggatatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgactagttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatagcttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 57 - DNA, 4568 bp, Artificial Sequence
  pASK75-PhoA-strepII
ccatcgaatggccagatgattaattcctaatttttgttgacactctatcattgatagagttattttaccactccctatcagtgatagagaaaagtgaaatgaatagttcgacaaaaatctagaacatggagaaaataaagtgaaacaaagcactattgcactggcactcttaccgttactgtttacccctgtgacaaaagcccggacaccagaaatgcctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaaccgcctagcgcttggtctcacccgcagttcgaaaaataataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 58 - DNA, 138 bp, Homo sapiens
  Albumin binding domain (ABD)
ctggcagaagcaaaagttctggcaaatcgtgaactggataaatatggtgtgagcgactattacaagaacctgattaataacgcgaaaaccgtggaaggtgttaaagcactgattgatgaaattctggcagcactgccg

SEQ ID NO: 59 - protein, 46 aa, Homo sapiens
  Albumin binding domain (ABD)
LAEAKVLANRELDKYGVSDYYKNLINNAKTVEGVKALIDEILAALP

SEQ ID NO: 60 - DNA, 357 bp, Artificial Sequence
  ProtL-ABD fusion protein
atgaaagaagaagttaccattaaagttaatctgattttcgccgatggtaaaacccagaccgcagaatttaaaggcacctttgaagaagcaaccgcaaaagcctatgcctatgccgatctgctggcaaaagaaaatggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaa

SEQ ID NO: 61 - protein, 118 aa, Artificial Sequence
  ProtL-ABD fusion protein
MKEEVTIKVNLIFADGKTQTAEFKGTFEEATAKAYAYADLLAKENGEYTADLEDGGNTINIKFAGGAVDANSLAEAKVLANRELDKYGVSDYYKNLINNAKTVEGVKALIDEILAALP

SEQ ID NO: 62 - DNA, 3493 bp, Artificial Sequence
  pASK75-ProtL-ABD
ccatcgaatggccagatgattaattcctaatttttgttgacactctatcattgatagagttattttaccactccctatcagtgatagagaaaagtgaaatgaatagttcgacaaaaatctagaaataattttgtttaactttaagaaggagatatacatatgaaagaagaagttaccattaaagttaatctgattttcgccgatggtaaaacccagaccgcagaatttaaaggcacctttgaagaagcaaccgcaaaagcctatgcctatgccgatctgctggcaaaagaaaatggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatttgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 63 - DNA, 36 bp, Artificial Sequence
  AJR_ProtL_PHE337TAG_fw
cattaaagttaatctgatttaggccgatggtaaaac

SEQ ID NO: 64 - DNA, 36 bp, Artificial Sequence
  AJR_ProtL_PHE337TAG_rv
gttttaccatcggcctaaatcagattaactttaatg

SEQ ID NO: 65 - DNA, 45 bp, Artificial Sequence
  AJR_ProtL_Y361N_L365S_fw
caaaagcctatgccaacgccgatctgagcgcaaaagaaaatggtg

SEQ ID NO: 66 - DNA, 45 bp, Artificial Sequence
  AJR_ProtL_Y361N_L365S_rv
caccattttcttttgcgctcagatcggcgttggcataggcttttg

SEQ ID NO: 67 - DNA, 357 bp, Artificial Sequence
  ProtLUAG337-ABD
atgaaagaagaagttaccattaaagttaatctgatttaggccgatggtaaaacccagaccgcagaatttaaaggcacctttgaagaagcaaccgcaaaagcctatgccaacgccgatctgagcgcaaaagaaaatggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaa

SEQ ID NO: 68 - DNA, 28 bp, Artificial Sequence
  AJR_ProtL_PHE347TAG_fw
cagaccgcagaatagaaaggcacctttg

SEQ ID NO: 69 - DNA, 28 bp, Artificial Sequence
  AJR_ProtL_PHE347TAG_rv
caaaggtgcctttctattctgcggtctg

SEQ ID NO: 70 - DNA, 34 bp, Artificial Sequence
  AJR_ProtL_TYR361ALA_fw
ccgcaaaagcctatgccgcggccgatctgctggc

SEQ ID NO: 71 - DNA, 34 bp, Artificial Sequence
  AJR_ProtL_TYR361ALA_rv
gccagcagatcggccgcggcataggcttttgcgg

SEQ ID NO: 72 - DNA, 357 bp, Artificial Sequence
  ProtLUAG347-ABD
atgaaagaagaagttaccattaaagttaatctgattttcgccgatggtaaaacccagaccgcagaatagaaaggcacctttgaagaagcaaccgcaaaagcctatgccgcggccgatctgctggcaaaagaaaatggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaa

SEQ ID NO: 73 - DNA, 30 bp, Artificial Sequence
  AJR_ProtL_ALA360TAG_fw
ccgcaaaagcctattagtatgccgatctgc

SEQ ID NO: 74 - DNA, 30 bp, Artificial Sequence
  AJR_ProtL_ALA360TAG_rv
gcagatcggcatactaataggcttttgcgg

SEQ ID NO: 75 - DNA, 357 bp, Artificial Sequence
  ProtLUAG360-ABD
atgaaagaagaagttaccattaaagttaatctgattttcgccgatggtaaaacccagaccgcagaatttaaaggcacctttgaagaagcaaccgcaaaagcctattagtatgccgatctgctggcaaaagaaaatggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaa

SEQ ID NO: 76 - DNA, 36 bp, Artificial Sequence
  AJR_ProtL_LEU364TAG_fw
ctatgcctatgccgattagctggcaaaagaaaatgg

SEQ ID NO: 77 - DNA, 36 bp, Artificial Sequence
  AJR_ProtL_LEU364TAG_rv
ccattttcttttgccagctaatcggcataggcatag

SEQ ID NO: 78 - DNA, 357 bp, Artificial Sequence
  ProtLUAG364-ABD
atgaaagaagaagttaccattaaagttaatctgattttcgccgatggtaaaacccagaccgcagaatttaaaggcacctttgaagaagcaaccgcaaaagcctatgcctatgccgattagctggcaaaagaaaatggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaa

SEQ ID NO: 79 - DNA, 34 bp, Artificial Sequence
  AJR_ProtL_GLU368TAG_fw
gatctgctggcaaaatagaatggtgaatataccg

SEQ ID NO: 80 - DNA, 34 bp, Artificial Sequence
  AJR_ProtL_GLU368TAG_rv
cggtatattcaccattctattttgccagcagatc

SEQ ID NO: 81 - DNA, 357 bp, Artificial Sequence
  ProtLUAG368-ABD
atgaaagaagaagttaccattaaagttaatctgattttcgccgatggtaaaacccagaccgcagaatttaaaggcacctttgaagaagcaaccgcaaaagcctatgcctatgccgatctgctggcaaaatagaatggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaa

SEQ ID NO: 82 - DNA, 32 bp, Artificial Sequence
  AJR_ProtL_ASN369TAG_fw
ctgctggcaaaagaatagggtgaatataccgc

SEQ ID NO: 83 - DNA, 32 bp, Artificial Sequence
  AJR_ProtL_ASN369TAG_rv
gcggtatattcaccctattcttttgccagcag

SEQ ID NO: 84 - DNA, 357 bp, Artificial Sequence
  ProtLUAG369-ABD
atgaaagaagaagttaccattaaagttaatctgattttcgccgatggtaaaacccagaccgcagaatttaaaggcacctttgaagaagcaaccgcaaaagcctatgcctatgccgatctgctggcaaaagaatagggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaa

SEQ ID NO: 85 - DNA, 5429 bp, Artificial Sequence
  pSBX8.CafRS#30d71
tttttacggttcctggccttttgctggccttttgctcacatgacccgacaccataacgctcggttgccgccgggcgttttttattggccagatgattaattcctaatttttgttgacactctatcattgatagagttattttaccactccctatcagtgatagagaaaagtgaaatgaatagttcgacaaaatctagaaataattttgtttaactttaagaaggagatatacatatgaaagaagaagttaccattaaagttaatctgatttaggccgatggtaaaacccagaccgcagaatttaaaggcacctttgaagaagcaaccgcaaaagcctatgccaacgccgatctgagcgcaaaagaaaatggtgaatataccgcagatctggaagatggtggtaataccatcaatatcaaatttgccggtggtgccgttgatgcaaatagcctggcagaagcaaaagttctggcaaatcgtgaactggataaatatggtgtgagcgactattacaagaacctgattaataacgcgaaaaccgtggaaggtgttaaagcactgattgatgaaattctggcagcactgccgtaataagcttgacctgtgaagtgaaaaatggcgcacattgtgcgacattttttttgtctgccgtttaccgctactgcgcggcagtacgcctttggtttatccattttatacaatccatgtaaaaaagggccctgaaattcaggaccctttctgagctcattacaggttggtgctaataccattatagtagctctcggaacggcttgcacgtttaatgtttttgaaaccgtgcattactttcagcagacgttcgagaccaaaacctgcaccaatccagggtttatcaataccccattcacgatccaggctaaccggaccaacaactgcgctactcagttccagatcaccgtgcataatatccagggtatcaccaaaaaccatgcagctatcaccaacaatttcaaagtcgatttccaggtaatccagaaactctttaatcagtgcttccagattttcacgggtacaaccgctacccatctggccaaacaccaccattgtaaattcttccaggtgttctttaccatcactttctttacgatagcacggaccaacttcaaagactttggaaggaccaggcagaatacgatccagtttccggctgtagttatacaatgtcggggtcagcataggacgcagacacaggtttttatcaacgcgaaagatttgtttgctcagttcggtatcattgttaatgcccatacgttcaacatattctgccggaatcagaatcgggcttttgatttccagaaaaccgcgatccacgaaaaatttggtaatatcacgttccagtttacccagataatcttcgcggtcgttggtatataagcgttgaaaatcatttttacgacgagtaaccagttccggttccagttcacgaaacggttttgccatattcaggctgattttatcttcaggactcagcagtgcttcaacacgatctaactgagaccgggttaagctcggtgccggtgcgcttgccggaacgctgctattcggggtgctttttgccggactcggaacgctacggctggtattggtgcttgcttttgcgctaacgctattttccagaggtttcggtgcgcgactaacgcttttcggcattgcttttttaactttaggtgcgctcacaacacgaactttaactgaatttttgctttcggtgctgcgtgtcagaaaattgttaatatcttcatcactcacacggcagcgtttacaggttttacgatatttgtgatgacgaaatgcgcgtgcggtacgacagctacggctattattcacaaccagatgatcgccacaggccatttcaatatagattttgctgcggctaacttcgtgatgtttgattttatgcagggtgccggtacggctcatccacagacctgttgcgctaatcagaacatccagcggttttttatccatatcgtacctccttaaatttctaggttgtgacctaggtgatttagtttaccagtgcaaaagaaatgtcaaaagagaagggcgtgaatttaacgcggttccagcgcaaagacttcaaaacctgcgtcggtgccgatttcggcctattggttaaaaaatgagctgagttctagtaaaaaaaatccttagctttcgctaaggatctgcagtggcggaaaccccgggaatctaacccggctgaacggatttagagtccattcgatctacatgatcaggtttccgaattcagcgttacaagtattacacaaagttttttatgttgagaatatttttttgatggggcatggcgcaaaacctttcgcggtatggcatgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaagaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaatgagaatattcgttttcacccaaggaatagaggatatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgactagttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatagcttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 86 - protein, 118 aa, Artificial Sequence
  ProtLUAG337-ABD
  UNSURE 13: modified amino acid Caf
MKEEVTIKVNLIXADGKTQTAEFKGTFEEATAKAYANADLSAKENGEYTADLEDGGNTINIKFAGGAVDANSLAEAKVLANRELDKYGVSDYYKNLINNAKTVEGVKALIDEILAALP

(19) United States
(12) Patent Application Publication
(10) Pub. No.: US 2021/0079037 A1
(43) Pub. Date: Mar. 18, 2021

(54) LIGHT-SWITCHABLE POLYPEPTIDE AND USES THEREOF

(71) Applicant: TECHNISCHE UNIVERSITÄT MÜNCHEN, München (DE)

(72) Inventors: Arne SKERRA, Dachau (DE); Andreas REICHERT, Freising (DE); Martin DAUNER, Halle (DE)

(73) Assignee: TECHNISCHE UNIVERSITÄT MÜNCHEN, München (DE)

(21) Appl. No.: 16/611,801

(22) PCT Filed: May 9, 2018
(86) PCT No.: PCT/EP2018/062160

§ 371 Date: Nov. 7, 2019

(30) Foreign Application Priority Data
May 10, 2017 (EP) 17170516.3

(51) Int. Cl. C07K 1/22; G01N 33/542; G01N 33/58; C07K 14/36

(52) U.S. Cl. CPC C07K 1/22; C07K 14/36; G01N 33/582; G01N 33/542

(57) ABSTRACT

The present invention relates to a light-switchable polypeptide. In particular, the present invention relates to a polypeptide comprising a light-responsive element, wherein the configuration (i.e. the configurational state) of the light-responsive element can be switched between a trans and cis isomer by irradiating the polypeptide with (a) particular wavelength(s) of light, and wherein the switch of said configuration alters the conformation and binding activity of said polypeptide to a ligand (e.g. molecule of interest). Also, the present invention comprises using said light-switchable polypeptide for isolating and/or purifying a molecule of interest. The present invention further provides an affinity matrix, an affinity chromatography column, and an affinity chromatography apparatus comprising the light-switchable polypeptide of the invention.

The present invention relates to a light-switchable polypeptide. In particular, the present invention relates to a polypeptide comprising a light-responsive element, wherein the configuration (i.e. the configurational state) of the light-responsive element can be switched between a trans and cis isomer by irradiating the polypeptide with (a) particular wavelength(s) of light, and wherein the switch of said configuration alters the conformation and binding activity of said polypeptide to a ligand (e.g. molecule of interest). Also, the present invention comprises using said light-switchable polypeptide for isolating and/or purifying a molecule of interest. The present invention further provides an affinity matrix, an affinity chromatography column, and an affinity chromatography apparatus comprising the light-switchable polypeptide of the invention.

Affinity chromatography is a high resolution and high capacity separation method that has become increasingly important for separating and purifying proteins and other biological molecules. Since the inception of affinity chromatography over 50 years ago (Cuatrecasas et al. 1968 Proc. Natl. Acad. Sci. USA 61: 636-643), traditional purification techniques based on pH, ionic strength, or temperature have been replaced by this technology in many cases.

Today, affinity chromatography represents one of the most powerful techniques available for purification of biologically active compounds. The method is also a valuable tool for studying a variety of biological processes such as enzymatic activity, physiological regulation by hormones, protein-protein or cell-cell interactions among others (Wilchek 2004 Protein Sci. 13: 3066-3070). The wide applicability of affinity chromatography is based on a highly specific, reversible biological interaction between two molecules: an affinity molecule and a molecule of interest (i.e. a target molecule or ligand). The affinity molecule is attached to a solid matrix, the so-called solid phase or stationary phase (also called affinity support). The molecule of interest to be purified is present in a liquid phase (also called mobile phase) (Hage 2012 J. Pharm. Biomed. Anal. 69: 93-105).

Typically, affinity purification involves 3 steps: (i) incubation of a liquid crude sample with the affinity support to allow the target molecule of interest (ligand) in the sample to bind to the immobilized affinity molecule, (ii) washing away of non-bound sample components from the chromatography matrix and (iii) dissociation and recovery of the target molecule of interest from the affinity support (i.e., elution) by altering the buffer conditions such that the binding interaction between the affinity molecule and the ligand no longer occurs (Magdeldin & Moser 2012 Affinity chromatography: Principles and applications, In: Affinity Chromatography, Ed. S. Magdeldin, InTech, pp. 1-28). Because of the highly selective binding function of many affinity molecules, the method can be used to isolate, measure, or study specific molecules of interest even when they are present in complex biological samples and/or in minute quantities (Hage 2012 J. Pharm. Biomed. Anal. 69: 93-105).

In particular, purification of recombinant proteins can be simplified by fusion of the target protein of interest with a distinct amino acid sequence, commonly referred to as affinity tag. This tag can range from a short sequence of amino acids to domains or even entire proteins (Terpe 2003 Appl. Microbiol. Biotechnol. 60: 523-533). Furthermore, some tags increase protein solubility and, thus, enhance yield and facilitate purification. An overview of some common tags used for affinity chromatography is shown in Table 1, below.

One example for a highly useful affinity tag is the Strep-tag, which was developed as a generic tool for the purification and detection of recombinant proteins. This affinity tag was initially selected from a genetic random library as a nine amino acid peptide (AWRHPQFGG, SEQ ID NO: 13) that binds specifically and reversibly to streptavidin (Schmidt & Skerra 1993 Protein Eng. 6: 109-122). Hence, the Strep-tag can serve for the efficient purification of corresponding fusion proteins on streptavidin affinity columns. Elution of the bound recombinant protein is effected under mild buffer conditions in a biochemically active state by competition with natural streptavidin ligands, like D-biotin or D-desthiobiotin. The Strep-tag can be directly fused to a recombinant polypeptide during subcloning of its cDNA or gene and it usually does not interfere with protein function, folding or secretion.

The Strep-tag/streptavidin system was systematically optimized over the years, including engineering of streptavidin itself (resulting in the streptavidin mutant 1, also known as “Strep-Tactin”) and X-ray crystallographic analysis of the streptavidin-peptide complexes, revealing a conformationally driven binding mechanism (Schmidt & Skerra 1994 J. Chromatogr. A 676: 337-345; Schmidt & Skerra 1996 J. Mol. Biol. 255: 753-766; Voss & Skerra 1997 Protein Eng. 10: 975-982.; Korndoerfer & Skerra 2002 Protein Sci. 11: 883-893; Schmidt & Skerra 2007 Nat. Protoc. 2: 1528-1535). As result, the Strep-tag—or its improved version Strep-tag II—provides a reliable tool for the parallel isolation and functional analysis of multiple gene products in biopharmaceutical drug development, industrial biotechnology and protein/proteome research.

Most significantly, the well-characterized interaction between the Strep-tag ligand and the streptavidin affinity molecule enables one-step purification of tagged proteins of interest, which makes this kind of affinity chromatography a superior purification technique. Unlike conventional chromatographic procedures, such as gel filtration or ion-exchange chromatography, affinity chromatography is able to selectively isolate one molecule of interest at a time, whereas those conventional methods usually enrich molecules with similar biophysical characteristics (size, shape, charge, hydrophobicity and the like) (Bruemmer 1979 J. Solid-Phase Biochem. 4: 171-187).

However, affinity chromatography procedures known in the art also have disadvantages. After a sample has been loaded onto an affinity column under conditions that allow strong binding of the molecule of interest, as well as subsequent depletion of host cell components, an elution buffer is required to dissociate the target molecule (ligand) from the affinity matrix/support in the final step. This elution, often viewed as the most delicate step of an affinity chromatography protocol, should ideally be carried out in a way that keeps the affinity matrix intact, allowing regeneration and multiple use of the column (Firer 2001 J. Biochem. Biophys. Methods 49: 433-442). While binding of the target molecule to the affinity molecule occurs under conditions that mimic the native environment with regard to pH and ionic strength, the elution step often requires a drastic change of the mobile phase, for example by strongly altering the pH, polarity or ionic strength (Hage 2012 J. Pharm. Biomed. Anal. 69: 93-105).

Alternatively, a competitor can be added to the mobile phase in order to displace the target molecule bound to the affinity molecule that is immobilized on the column (Hage 2012 J. Pharm. Biomed. Anal. 69: 93-105), for example D-desthiobiotin in the case of the Strep-tag (Schmidt & Skerra 2007 Nat. Protoc. 2: 1528-1535) or imidazole in the case of the His(6)-tag (Skerra et al. 1991 Biotechnology (N Y) 9: 273-278). Evidently, using such a small molecule as competing agent for elution results in contamination of the solution comprising the purified molecule of interest. Consequently, these reagents must be removed in time-consuming additional purification steps, for example by dialysis or gel filtration, if incompatible with subsequent experiments or applications. On the other hand, unspecific elution conditions like altered pH, high concentrations of salts, organic cosolvents, detergents, metal ions, chelators or reducing agents are often detrimental to the target molecule. Particularly if the target molecule is a protein, such elution conditions can result in denaturation, aggregation or chemical modification, e.g. deamidation, thus hampering the functional activity.

Furthermore, after elution of the target molecule, the affinity column must be regenerated in a time-consuming procedure prior to the next round of sample application. In the case of the Strep-tag affinity chromatography this step involves washing of the column with HABA (4′-hydroxyazobenzene-2-carboxylic acid) to efficiently remove the competing agent D-desthiobiotin from the immobilized affinity molecule (e.g. streptavidin or a mutant thereof), followed by depletion of HABA by extensive washing with buffer.

Thus, the technical problem underlying the present invention is the provision of means and methods that allow a fast isolation and/or purification of a molecule of interest, wherein contamination and biochemical modification of the eluted molecule of interest is reduced.

This technical problem is solved by provision of the embodiments as defined herein and as characterized in the claims.

Accordingly, the present invention relates to a polypeptide comprising a light-responsive element (e.g. a light-responsive group or a light-responsive amino acid side chain), wherein the configuration of the light-responsive element can be switched by irradiating the polypeptide with (a) particular wavelength(s) of light, and wherein the switch of said configuration alters the binding activity of the polypeptide to a ligand.

Thus, the present invention provides a polypeptide comprising a light-responsive element, which is also termed “light-switchable polypeptide” herein. This light-switchable polypeptide paves the way for a fast and economic isolation and purification method with less contamination of the eluted molecule of interest as compared to conventional purification methods.

In particular, the light-switchable polypeptide of the invention (affinity polypeptide) may be comprised in a matrix of an affinity chromatography column. In the ground state (in the dark) or if the inventive light-switchable polypeptide is irradiated with particular wavelengths of light (e.g. visible light of about 400 to 530 nm, e.g. 400 to 500 nm), then the light-switchable polypeptide has a configuration which has binding activity to the ligand, such as a molecule of interest (in one embodiment, via binding to an affinity tag that is fused with the molecule of interest). If the light-responsive element has this configuration, then the light-switchable polypeptide specifically catches the molecule of interest (e.g. a recombinant protein) from a mixture (such as a cell extract or culture supernatant or other kind of mixture). Subsequently, the undesired components of the mixture (such as the undesired biomolecules of the cell extract or of the culture supernatant) may be removed, e.g. by washing the column with a buffer solution. In order to subsequently elute the molecule of interest, the light-switchable polypeptide is just irradiated with particular (different) wavelengths of light (e.g. with ultraviolet (UV) light having wavelengths of 300 to 390 nm). Consequently, the light-switchable polypeptide switches into a conformation which does not have binding activity to the molecule of interest. Thus, the molecule of interest can be eluted with any desired buffer or solution, and the eluted molecule of interest will not be contaminated with any aggressive chemical.

Accordingly, the light-switchable polypeptide provided herein has the advantages that binding of a molecule of interest to the light-switchable polypeptide (e.g. within a matrix of an affinity chromatography column), and elution of the molecule of interest can be easily and inexpensively achieved by irradiating the light-switchable polypeptide with particular wavelengths of light. In addition, the light-switchable polypeptide enables an affinity chromatography procedure under physiological purification conditions, wherein no specialized elution buffer is required. Therefore, using the light-switchable (affinity) polypeptide provided herein allows the purification of bioactive recombinant proteins of interest. Accordingly, the light-switchable polypeptide provided herein is an affinity polypeptide which can be used for the purification of proteins of interest, e.g. under physiological purification conditions. In addition, using the inventive light-switchable polypeptide enables a sharp and easily controllable elution of the molecule of interest and results in a pure sample without small molecule or solvent contamination. Especially if the molecule of interest is used for therapeutic purposes, the avoidance of contaminations is of high importance. In particular, the reduction of contaminations within the solution comprising an eluted therapeutic molecule may improve tolerability and avoid side effects of the therapeutic molecule. Also, contaminations interfere with many assays or measurements of biomolecules of interest in basic research. Furthermore, an affinity chromatography column that is functionalized with the light-switchable polypeptide provided herein has a short regeneration time, which can significantly fasten the purification of one or several target molecule(s). This is of particular interest for automated high throughput isolation and/or purification of molecules of interest, e.g. in the screening for a desired therapeutic protein.

Thus, advantages of the means and methods provided herein are, e.g.: (a) elution of the molecule of interest in the desired buffer, suitable for subsequent use, without contamination by agents that are conventionally used for achieving elution of the target molecule; (b) quick and optionally automated chromatography cycles; and (c) high concentration of the molecule of interest in the elution fraction due to the very sharp elution peak (since the light-switching of the light-switchable polypeptide provided herein is more efficient and much faster than conventional re-buffering of affinity columns via liquid flow).

A further advantage of the light-switchable polypeptide of the invention is that it is devoid of a covalently or non-covalently bound prosthetic group (cofactor or coenzyme), for example flavin mononucleotide (FMN) or retinal, as they are found in photoactive proteins or light-sensing domains in nature.

One aspect of the present invention relates to the use of the light-switchable polypeptide provided herein (i.e. the polypeptide comprising a light-responsive element) for isolating and/or purifying a molecule of interest.

The terms “isolating a molecule of interest” and “purifying a molecule of interest” as well as grammatical variations thereof are used interchangeably herein and mean that the amount of molecules other than the molecule of interest is decreased. These terms include that many, most or all substances other than the molecule of interest are reduced, minimized or removed. As described below in more detail, the molecule of interest may be any molecule. For example, the molecule of interest may be selected from the group consisting of a peptide, an oligopeptide, a polypeptide, a protein, an antibody or a fragment thereof, an immunoglobulin or a fragment thereof, an enzyme, a hormone, a cytokine, a complex, an oligonucleotide, a polynucleotide, a nucleic acid, a carbohydrate, a liposome, a nanoparticle, a cell, a biomacromolecule, a biomolecule and a small molecule. Herein the terms “isolating a molecule of interest”, “separating a molecule of interest” and “purifying a molecule of interest” include that cellular material other than the molecule of interest such as, for example, components of the cell extract or culture media are reduced, minimized or removed. Thus, the term “isolating/purifying a molecule of interest” also includes that a molecule of interest is separated from (a) component(s) of its natural environment (including, for example, other proteins, nucleic acids, carbohydrates. lipids, cofactors, metabolites and the like). According to the present invention the molecule of interest may be purified to at least 70%, more preferably at least 80%, and most preferably at least 90% purity as determined, for example, by electrophoresis (e.g., agarose gel electrophoresis, starch gel electrophoresis, polyacrylamide gel electrophoresis, SDS-PAGE, isoelectric focusing (IEF), capillary electrophoresis), chromatography (e.g., ion exchange, size exclusion or reverse phase HPLC) or other methods (e.g., mass spectroscopy, MS, enzyme-linked immunosorbent assay, ELISA, flow cytometry such as FACS). Such methods for determining the purity of a molecule of interest are commonly known in the art. Preferably, isolation/purification of a given molecule of interest means rendering the molecule of interest substantially pure.

The light-switchable polypeptide provided herein may be part of (i.e. comprised in) a solid phase. For example, the light-switchable polypeptide may be part of a solid phase of an affinity chromatography system, and a molecule of interest may be part of the corresponding liquid phase.

Thus, a further aspect of the present invention relates to a method for isolating and/or purifying a molecule of interest, the method comprises the steps of
(i) contacting a liquid phase comprising the molecule of interest with the light-switchable polypeptide of the invention,
   wherein the light-switchable polypeptide is part of (i.e. comprised in) a solid phase, and wherein the light-responsive element is in a first configuration so that the polypeptide (i.e. the light-switchable polypeptide) has high affinity to the molecule of interest; and
(ii) irradiating the light-switchable polypeptide with (a) wavelength(s) that change(s) the light-responsive element to a second configuration so that the polypeptide (i.e. the light-switchable polypeptide) has a decreased affinity to the molecule of interest as compared to the affinity of step (i) and eluting the molecule of interest.

In step (ii) of the method described above, elution of the molecule of interest is preferably performed while irradiating the light-switchable polypeptide with (a) particular wavelength(s) of light. However, due to the slow relaxation of the light-responsive element, step (ii) may also be performed in a gradual manner, that is, more specifically, the light-switchable polypeptide may be irradiated in a first step; and elution of the molecule of interest may be performed in a second step, e.g. in the dark.

In the context of the present invention a light-switchable variant of a known streptavidin mutein (particularly of Strep-Tactin) has been designed and prepared as a recombinant protein. Therefore, the light-switchable polypeptide of the invention may be streptavidin comprising a light-responsive element or a variant or mutein of streptavidin comprising a light-responsive element. Accordingly, one aspect of the present invention relates to the light-switchable polypeptide, use, or method provided herein, wherein the light-switchable polypeptide is streptavidin comprising a light-responsive element or a variant or mutein of streptavidin comprising a light-response element.

The light-controllable streptavidin mutein provided herein paves the way for light-controlled chromatography also with other protein-based affinity molecules. Thus, it is envisaged in the context of the present invention to integrate a light-responsive element (e.g. a light-responsive amino acid side chain) into other proteins that are capable of binding a defined ligand (molecule of interest, for example a protein or an immunoglobulin), such as protein A, protein G, protein L, or an anti-myc-tag antibody (such as the antibody fragment Fab 9E10). Accordingly, the light-switchable polypeptide of the present invention may be any polypeptide selected from:
(i) streptavidin or a variant or mutein thereof, comprising a light-responsive element;
(ii) protein A or a fragment, variant or mutein thereof, comprising a light-responsive element;
(iii) protein G or a fragment, variant or mutein thereof, comprising a light-responsive element;
(iv) protein L or a fragment, variant or mutein thereof, comprising a light-responsive element; or
(v) an anti-myc-tag antibody or a fragment, variant or mutein thereof, comprising a light-responsive element.

Streptavidin is an extracellular protein produced by Streptomyces avidinii that tightly binds D-biotin. The unprocessed protein consists of 159 amino acids and has a molecular weight of about 16 kDa. The processed protein (i.e. core streptavidin) consists of about 127 amino acids. Functional streptavidin has a tetrameric structure comprising four streptavidin subunits. The high affinity of streptavidin to biotin is the basis for many biological and biotechnological labeling and binding experiments. Indeed, with a K_{d} value of 10⁻¹⁴ mol/l, the binding of streptavidin to biotin represents one of the strongest non-covalent affinities known (Green 1975 Adv. Protein Chem. 29: 85-133). The term “K_{d}” (also called “K_{D}”) refers to the equilibrium dissociation constant (the reciprocal of the equilibrium binding constant) and is used herein according to the definitions provided in the art.

Strep-tag and Strep-tag II are artificial peptide ligands of streptavidin (Schmidt & Skerra 1993 Protein Eng. 6: 109-122). Strep-tag and Strep-tag II bind competitively with biotin to streptavidin. Streptavidin and its variants and muteins are commonly used to isolate and/or purify molecules that comprise the Strep-tag, Strep-tag II, or biotin. A known mutein of streptavidin is Strep-Tactin. The amino acid sequences of core streptavidin and Strep-Tactin are provided herein as SEQ ID NOs: 10 and 8, respectively.

Protein G, protein A and protein L are immunoglobulin-binding bacterial proteins that can be used to isolate and/or purify immunoglobulins or antibodies.

Protein A is a 42 kDa surface protein originally found in the cell wall of Staphylococcus aureus. Protein A has an ability to bind immunoglobulins (Ig), including antibodies (such as monoclonal antibodies, MAb) and fragments thereof. Protein A comprises five homologous Ig-binding domains that each fold into a three-helix bundle. Each of these five domains is able to bind antibodies from many mammalian species, most notably those belonging to the class of immunoglobulin G (IgG). For affinity purification purposes often a recombinant fragment comprising residues 212 to 269 (UniProt database entry P38507) of protein A is used. This fragment comprises or consists of domain B of protein A. More specifically, protein A binds to the heavy chain within the Fc region of most immunoglobulins, and also within the Fab region, especially in the case of the human VH3 family. In order to increase the tolerance of the domain B towards site-specific chemical cleavage of fusion proteins using hydroxylamine, the sensitive Asn-Gly dipeptide at its residues 28-29 was changed by site-directed mutagenesis to Asn-Ala, resulting in the so-called engineered Z domain (Haber 2008 J. Chromatogr. B 848: 40-47). This Z domain of protein A, coupled to a chromatography support, can be used for the affinity purification of antibodies. The amino acid sequence of the domain Z of protein A is provided herein as SEQ ID NO: 16. Amino acid positions within this sequence suitable for incorporation of a light-responsive element, e.g. 4′-carboxyphenylazophenylalanine or 3′-carboxyphenylazophenylalanine, are Phe5, Gln9, Phe13, Tyr14, Glu25, Gln26, Arg27, Asn28 Ala29, Phe30, Ile31, Gln32, Lys35, Asp36, Asp37, Gln40, Asn43, Leu45, Glu47, Leu51, and/or Asn52 of SEQ ID NO: 16 (corresponding to positions 216, 220, 224, 225, 236, 237, 238, 239, 240, 241, 242, 243, 246, 247, 248, 251, 254, 256, 258, 262 and 263, respectively, in UniProt database entry P38507). The light-responsive element may be incorporated into protein A at one or more of these amino acid positions. Ala29 corresponds to Gly29 in the wild-type B domain of protein A.

Thus, if the light-switchable polypeptide provided herein is protein A (or a variant, mutein, fusion protein or fragment thereof, in particular comprising the Z domain) comprising a light-responsive element, then the molecule of interest (ligand) is preferably an antibody or a fragment thereof, and more preferably an IgG (e.g. a human IgG, such as a human IgG1, IgG2, or IgG4; or a murine IgG, such as a murine IgG2a, IgG2, or IgG3) or a fragment thereof. In such a case the molecule of interest (ligand) may also be a human IgG3 or a murine IgG1; or a fragment thereof. Accordingly, if the light-switchable polypeptide provided herein is protein A (or a variant, mutein, fusion protein or fragment thereof, preferably a fragment that comprises the Z domain) comprising a light-responsive element, then the molecule of interest (ligand) is preferably an antibody or a fragment thereof, and more preferably an IgG (e.g. a human IgG, such as a human IgG1, IgG2, IgG3 or IgG4; or a murine IgG, such as a murine IgG1, IgG2a, IgG2, or IgG3) or a fragment thereof. In this regard, if the molecule of interest is a fragment of an IgG antibody, then the fragment preferably comprises the Fc region and/or the Fab region. Also in this regard, if the molecule of interest is a fragment of an antibody belonging to the human VH3 family, then it preferably comprises the Fab region.

Protein G is another immunoglobulin-binding protein found in group G Streptococci. It consists of three Fc-binding domains (C1, C2 and C3) as well as an albumin-binding portion and binds to antibodies, particularly to the Fc region of IgG (Cao 2013 Biotechnol. Lett. 35: 1441-1447), but also to the Fab fragment. Native protein G also binds albumin, but because serum albumin is a major contaminant of antibody sources, the albumin-binding site has been removed from several recombinant forms of protein G. The amino acid sequences of the domains C1, C2 and C3 of protein G are provided herein as SEQ ID NOs: 17, 18 and 19, respectively. The sequences of SEQ ID NOs: 17, 18 and 19 correspond to positions 223-357, 373-427 and 443-497, respectively, in UniProt database entry P19909. Amino acid positions within the sequence of each domain C1, C2 and C3 suitable for incorporation of a light-responsive element, e.g. 4′-carboxyphenylazophenylalanine or 3′-carboxyphenylazophenylalanine, are Lys3, Val5 or Ile5, Thr10, Thr16, Val28 or Ala28, Tyr32, and/or Asp35 of SEQ ID NO: 18 (corresponding to positions 375, 377, 382, 388, 400, 404 and 407, respectively, in UniProt database entry P19909). The light-responsive element may be incorporated into protein G at one or more of these amino acid positions.

Thus, if the light-switchable polypeptide provided herein is protein G (or a variant, mutein, fusion protein, or fragment thereof) comprising a light-responsive element, then the molecule of interest is preferably an antibody or a fragment thereof, for example Fab of Fc, and more preferably an IgG or a fragment thereof. In this regard, if the molecule of interest is a fragment of an IgG antibody, then the fragment preferably comprises the Fc and/or Fab region.

Protein L is expressed on the surface of Peptostreptococcus magnus and was found to bind to immunoglobulin light chains. Full length protein L consists of 719 amino acids. The gene for protein L encodes five regions: a signal sequence with 18 amino acids; the aminoterminal region “A” with 79 residues; five homologous “B” repeats with 72-76 amino acids each; a carboxyterminal region with two additional “C” repeats of 52 amino acids each; a hydrophilic, proline-rich putative cell wall-spanning region “W”; a hydrophobic membrane anchor “M”. The B repeat region (36 kDa) is responsible for the interaction with 1 g light chains. The fragment of protein L used for antibody purification is denoted as domain B1 and comprises 78 amino acid residues (Wikstroem 1995 J. Mol. Biol. 250: 128-133). The 78 amino acids of domain B1 correspond to positions 324-389 in UniProt database entry Q51918. Since no part of the immunoglobulin heavy chain is involved in the binding interaction, protein L binds a wider range of antibody classes than protein A or G, including IgG, IgM, IgA, IgE and IgD and their subclasses. Protein L also binds single chain variable fragments (scFv) and Fab fragments of antibodies. In particular, protein L binds to antibodies that contain kappa light chains. The amino acid sequence of the domain B1 of protein L is provided herein as SEQ ID NO: 20. Amino acid positions within the sequence of domain B1 suitable for incorporation of a light-responsive element, e.g. 4′-carboxyphenylazophenylalanine or 3′-carboxyphenylazophenylalanine, are Thr5, Asn9, Ile11, Phe12, Lys16, Phe26, Lys32, Ala35, Glu43, and/or Tyr47 of SEQ ID NO: 20 (corresponding to positions 330, 334, 336, 337, 341, 351, 357, 360, 368 and 372, respectively, in UniProt database entry Q51918). Further positions are Phe22, Leu39, and/or Asn44 of SEQ ID NO: 20 (corresponding to positions 347, 364 and 369, respectively, in UniProt database entry Q51918). Further amino acid positions within the sequence of domain B1 suitable for incorporation of a light-responsive element, e.g. 4′-carboxyphenylazophenylalanine or 3′-carboxyphenylazophenylalanine, are Phe22, Leu39 and/or Asn44 of SEQ ID NO: 20 (corresponding to positions 347, 364 and 369, respectively, in UniProt database entry Q51918). Among these positions considered for introduction of a light-responsive element, e.g. 4′-carboxyphenylazophenylalanine (Caf), into the domain B1 of protein L, Phe22, Ala35, Leu39, Glu43 and Asn44 are less preferred.

The light-responsive element may be incorporated into protein L at one or more of these amino acid positions. Preferably, the light-switchable polypeptide provided herein comprises the domain B1 of protein L (SEQ ID NO: 20), wherein the light-responsive element, e.g. 4′-carboxyphenylazophenylalanine (Caf), is incorporated at the position corresponding to position Phe12 of SEQ ID NO: 20. Such a light-switchable polypeptide may also have a mutation at position 36 of SEQ ID NO: 20 and a further mutation at position 40 of SEQ ID NO: 20. For example, Tyr36 of SEQ ID NO: 20 may be mutated to Ala, Arg, Asn, Asp, Cys, Glu, Gln, Gly, His, Ile, Leu, Lys, Met, Phe, Pro, Ser, Thr, Trp or Val. Preferably, Tyr36 of SEQ ID NO: 20 is mutated to Asn. Leu40 of SEQ ID NO: 20 may be mutated to Ala, Arg, Asn, Asp, Cys, Glu, Gln, Gly, His, Ile, Lys, Met, Phe, Pro, Ser, Thr, Trp, Tyr or Val. Preferably, Leu40 of SEQ ID NO: 20 is mutated to Ser.

Therefore, if the light-switchable polypeptide provided herein is protein L or a variant or mutein or fragment or fusion protein thereof, then the molecule of interest is preferably an antibody or a fragment thereof, more preferably a human or mouse antibody or fragment thereof, even more preferably an IgG, even more preferably an antibody or fragment (e.g. Fab or scFv) thereof comprising a kappa light chain, even more preferably an antibody or fragment thereof comprising a human VκI, VκIII and/or VκIV light chain and/or a mouse VκI light chain.

As mentioned above, the light-switchable polypeptide provided herein may be a fusion protein of protein L or a fragment thereof. For example, the fusion protein may comprise a codon optimized protein L domain B1 (herein referred to as ProtL; SEQ ID NO: 20) which is fused to a human albumin-binding domain (ABD; SEQ ID NO: 59) via a short linker sequence. Such a protein L-ABD fusion protein is shown herein as SEQ ID NO: 61 (and is also called ProtL-ABD herein). Preferably, such a fusion protein carries a light-responsive element, e.g. 4′-carboxyphenylazophenylalanine or 3′-carboxyphenylazophenylalanine, preferably 4′-carboxyphenylazophenylalanine (Caf), at position 13 of SEQ ID NO: 61. Such a fusion protein may also have a mutation at position 37 of SEQ ID NO: 61 and a further mutation at position 41 of SEQ ID NO: 61. For example, Tyr37 of SEQ ID NO: 61 may be mutated to Ala, Arg, Asn, Asp, Cys, Glu, Gln, Gly, His, Ile, Leu, Lys, Met, Phe, Pro, Ser, Thr, Trp or Val. Preferably, Tyr37 of SEQ ID NO: 61 is mutated to Asn. Leu41 of SEQ ID NO: 61 may be mutated to Ala, Arg, Asn, Asp, Cys, Glu, Gln, Gly, His, Ile, Lys, Met, Phe, Pro, Ser, Thr, Trp, Tyr or Val. Preferably, Leu41 of SEQ ID NO: 61 is mutated to Ser. For example, the light-switchable polypeptide provided herein may comprise or consist of the amino acid sequence of SEQ ID NO: 86.

Protein A, protein G and protein L, or fragments or fusion proteins thereof are popular tools for antibody purification because they bind to many subclasses of antibodies from humans and animals, allowing antibodies produced via biotechnology to be captured on corresponding affinity matrices (see, e.g., Nilsson et al. 1997 Protein Expr. Purif. 11: 1-16). However, the common elution by means of chaotropic salts or low pH conditions may lead to chemical modification or denaturation of the target protein and, thus, affect functionality. Modifying protein A, protein G or protein L to show light-sensitive binding activity toward antibodies and applying them to the generation of an affinity matrix would diminish the disadvantages of this conventional purification technique.

Anti-myc-tag antibodies are commonly known in the art. For example, the anti-MYC antibody clone 9E10 (DrMAB-150) is a monoclonal mouse antibody which selectively binds to a myc-tag, i.e. a peptide (SEQ ID NO: 15) corresponding to a stretch of amino acids in the C-terminal region of human c-MYC (Schiweck et al. 1997 FEBS Lett. 414: 33-38). Therefore, this antibody is used for isolating and/or purifying molecules, in particular recombinant proteins comprising a myc-tag. The recombinant Fab fragment of the 9E10 antibody can be easily produced in Escherichia coli. However, when using a conventional anti-myc-tag antibody or its Fab or its variants or muteins immobilized to a solid support in affinity chromatography, the molecule of interest is eluted via low pH conditions from the affinity matrix, which may affect the properties of the target molecule. This disadvantage can be overcome by producing a light-switchable anti-myc-tag antibody (or anti-myc-tag antibody fragment, such as Fab 9E10) according to the present invention. Chemical coupling of a light-switchable anti-myc-tag antibody or anti-my-tag Fab fragment to a chromatography matrix advantageously allows the light-controlled elution of a molecule carrying the myc-tag. The anti-MYC antibody clone 9E10 as well as its Fab fragment (Fab 9E10) are described, e.g., in Krauss, (2008 Proteins 73: 552-565). The amino acid sequences of the mature (devoid of a signal sequence) heavy and light chains of the murine IgG1/κ antibody 9E10 are provided herein as SEQ ID NOs: 21 and 22, respectively. The Fab fragment of the antibody 9E10 comprises the same light chain and the aminoterminal region of the heavy chain, that is residues 19-228 in SEQ ID NO: 21 (optionally equipped with a His₆-tag). If the light-switchable polypeptide of the invention is a light-switchable anti-myc-tag antibody (or a variant thereof, such as a light-switchable Fab 9E10), then the light-responsive element is preferably introduced at a position within at least one of the complementarity-determining regions (CDRs). Amino acid positions within the sequence of the 9E10 heavy chain suitable for incorporation of a light-responsive element, e.g. 4′-carboxyphenylazophenylalanine or 3′-carboxyphenylazophenylalanine, are Tyr76, Phe121, Tyr122, Tyr123, Tyr124, Tyr128, and/or Tyr129 of SEQ ID NO: 21. A further position is Tyr130 of SEQ ID NO: 21. The light-responsive element may be incorporated into the sequence of the 9E10 heavy chain at one or more of these amino acid positions.

As described above, the light-switchable polypeptide provided herein may be streptavidin comprising a light-responsive element, protein A comprising a light-responsive element, protein G comprising a light-responsive element, protein L comprising a light-responsive element or the anti-myc-tag antibody or Fab 9E10 comprising a light-responsive element. However, the light-switchable polypeptide provided herein may also be a “variant” (e.g. a fragment) or “mutein” or “fusion protein” of any of the polypeptides mentioned above. Herein, a variant or mutein of a given polypeptide is any modified version of the polypeptide (such as a fragment), provided that the polypeptide is still functional. Preferably, such a mutein of the light-switchable polypeptide may comprise one or more amino acid substitution(s) at positions different from the position carrying the light-responsive element which modify/ies or enhance(s) the effect of the light-switchable configuration on the conformation and binding activity of said polypeptide to a ligand.

For example, in a light-switchable domain B1 of protein L carrying Caf as a light-responsive element at amino acid position 13 of SEQ ID NO: 61 a Tyr to Asn mutation at position 37 of SEQ ID NO: 61 and a Leu to Ser mutation at position 41 of SEQ ID NO: 61 enhance the effect of the light-switchable configuration of Caf on the conformation and binding activity of said polypeptide to an Immunoglobulin ligand. Thus, the light-switchable polypeptide of the present invention may comprise (in addition to the light-responsive element) one, two or more (e.g. 1 to 10, 1 to 5, preferably 2) further mutations which enhance the effect of light on the binding activity of the light-switchable polypeptide to a ligand (e.g. to the molecule of interest).

For example, in the ground state (e.g. in the dark or under visible light having wavelengths of about 400 to 530 nm) the light-switchable polypeptide may have a certain binding activity to the molecule of interest. Irradiating said light-switchable polypeptide with light having (a) different wavelength(s) (e.g. with UV light having wavelengths of 300 to 390 nm) may result in a decreased or increased (preferably decreased) binding activity of said light-switchable polypeptide to said molecule of interest. The effect of said light having (a) different wavelength(s) on the binding activity of the light-switchable polypeptide may be enhanced by mutations within the light-switchable polypeptide. Therefore, the light-switchable polypeptide of the present invention may comprise, in addition to the light-responsive element, mutations enhancing the degree to which the light-switchable polypeptide is controllable by light.

Accordingly, the present invention provides a method for identifying a mutation which enhances the degree to which the light-switchable polypeptide of the present invention is controllable by light, wherein the method comprises:

(a) analyzing the three-dimensional (3D) structure or tertiary structure or conformation of the light-switchable polypeptide (e.g. by using a computer program for graphical display known in the art, e.g. PyMOL or Chimera; see Jarasch 2016 Protein Eng. Des. Sel. 29: 263-270); and
(b) selecting an amino acid side chain in the vicinity of (e.g. within 15 Å, preferably 10 Å, more preferably 5 Å distance from) the light-responsive element that sterically overlaps (e.g. sharing at least one pair of atoms with closer distance than the sum of their van der Waals radii) with the configurational state of the light-responsive element (e.g. Caf) corresponding to the conformation of the light-switchable polypeptide that is associated with high binding affinity to the ligand (e.g. the trans configuration); and
(c) preparing a mutated light-switchable polypeptide by replacing the amino acid which corresponds to the selected amino acid side chain with another amino acid; and
(d) analyzing the binding activity of the mutated light-switchable protein in all possible configurations of the light-responsive element (e.g. in the cis and the trans configuration).

In step (c) above, the amino acid which corresponds to the selected amino acid side chain is preferably substituted with an amino acid which decreases the sterical overlap with the light-responsive element (e.g. an amino acid having a smaller side chain), or which results in favorable interactions (e.g. an amino acid resulting in one or more hydrogen bond(s), a salt bridge, or van der Waals contacts).

For example, a mutation which enhances the degree to which the light-switchable polypeptide is controllable by light may by a mutation which results in:

(i) an increased binding activity of the mutated light-switchable polypeptide to the ligand in the binding conformation (e.g. in the dark or under visible light having wavelengths of about 400 to 530 nm) as compared to the corresponding binding activity of the non-mutated light-switchable polypeptide;
(ii) a decreased binding activity of the mutated light-switchable polypeptide to the ligand in the non-binding conformation (e.g. at UV light having wavelengths of 300 to 390 nm) as compared to the corresponding binding activity of the non-mutated light-switchable polypeptide; or
(iii) a combination of (i) and (ii).

Accordingly, as mentioned above, enhancing additional mutations within the light-switchable polypeptide of the invention can be identified by searching for amino acid side chains in the vicinity of (e.g. within 15 Å, preferably 10 Å, more preferably 5 Å distance from) the light-responsive element that would sterically overlap with the configurational state of the light-responsive element (e.g. Caf) corresponding to the high affinity conformation of the light-switchable polypeptide (e.g. the trans configuration), e.g. by using a computer program for graphical display known in the art (e.g. PyMOL or Chimera, see: Jarasch et al. 2016 Protein Eng. Des. Sel. 29: 263-270). Then, an amino acid replacement is chosen at such a position that the sterical overlap is avoided (e.g. by using a smaller side chain) or that even favorable interactions may occur (such as one or more hydrogen bond(s), a salt bridge, or van der Waals contacts).

The light-switchable polypeptide provided herein is functional if the configuration of its light-responsive element can be switched by irradiating the polypeptide with (a) particular wavelength(s) of light, and if the switch of said configuration alters the binding activity (preferably affinity) of the polypeptide to a ligand (e.g. a molecule of interest). A variant or mutein of a given polypeptide may be the given polypeptide wherein one to several amino acids are substituted, added or deleted and wherein the polypeptide is still functional. For example, a variant or mutein of a given polypeptide may be a polypeptide having at least 80%, preferably at least 85%, more preferably at least 90%, even more preferably at least 95%, even more preferably at least 96%, even more preferably at least 97%, even more preferably at least 98% or most preferably at least 99% identity to the given polypeptide, provided that the variant or mutein is functional. A known mutein of streptavidin, which is preferably applied in the context of the present invention, is Strep-Tactin.

A variant of a given polypeptide may also be a fragment of the polypeptide provided that the fragment is still functional. For example, a variant of the anti-myc-tag antibody clone 9E10 is the Fab 9E10 as described herein.

A variant of a given polypeptide may also be a fusion protein comprising the given polypeptide and another protein. The other protein may, e.g., be a marker protein, such as green fluorescent protein (GFP), enhanced GFP (eGFP), or yellow fluorescent protein (YFP). Other fusion partners for the light-switchable polypeptide provided herein may comprise enzymes, proteins that enhance solubility, oligomerization domains or proteins having another binding function like the ABD. A variant of a given polypeptide may also be a conjugate comprising the given polypeptide and a non-proteinous compound, for example DNA.

Accordingly, one aspect of the present invention relates to the light-switchable polypeptide, use, or method provided herein, wherein the light-switchable polypeptide comprises or consists of
(i) the amino acid sequence of SEQ ID NO: 2;
(ii) the amino acid sequence of SEQ ID NO: 4;
(iii) the amino acid sequence of SEQ ID NO: 6; or
(iv) an amino acid sequence having at least 80%, preferably at least 85%, more preferably at least 90%, even more preferably at least 95%, even more preferably at least 96%, even more preferably at least 97%, even more preferably at least 98% or most preferably at least 99% identity to the amino acid sequence according to any one of (i)-(iii),
   wherein the polypeptide comprises a light-responsive element, wherein the configuration of the light-responsive element can be switched by irradiating the polypeptide with (a) particular wavelength(s) of light, and wherein the switch of said configuration alters the binding activity (preferably affinity) of the polypeptide to a ligand.

Another aspect of the present invention relates to the light-switchable polypeptide, use, or method provided herein, wherein the light-switchable polypeptide comprises or consists of
(i) the amino acid sequence of SEQ ID NO: 20, wherein the residue at position 12 of SEQ ID NO: 20 is replaced by a light-responsive element;
(ii) the amino acid sequence of SEQ ID NO: 86;
(iii) the amino acid sequence of SEQ ID NO: 61, wherein the residue at position 13 of SEQ ID NO: 61 is replaced by a light-responsive element; or
(iv) an amino acid sequence having at least 80%, preferably at least 85%, more preferably at least 90%, even more preferably at least 95%, even more preferably at least 96%, even more preferably at least 97%, even more preferably at least 98% or most preferably at least 99% identity to the amino acid sequence according to any one of (i)-(iii),
   wherein the polypeptide comprises a light-responsive element, wherein the configuration of the light-responsive element can be switched by irradiating the polypeptide with particular wavelengths of light, and wherein the switch of said configuration alters the binding activity of the polypeptide to a ligand.

The light-switchable polypeptide as defined in (i) above may also have a mutation at position 36 of SEQ ID NO: 20 and a mutation at position 40 of SEQ ID NO: 20. For example, Tyr36 of SEQ ID NO: 20 may be mutated to Ala, Arg, Asn, Asp, Cys, Glu, Gln, Gly, His, Ile, Leu, Lys, Met, Phe, Pro, Ser, Thr, Trp or Val. Preferably, Tyr36 of SEQ ID NO: 20 is mutated to Asn. Leu40 of SEQ ID NO: 20 may be mutated to Ala, Arg, Asn, Asp, Cys, Glu, Gln, Gly, His, Ile, Lys, Met, Phe, Pro, Ser, Thr, Trp, Tyr or Val. Preferably, Leu40 of SEQ ID NO: 20 is mutated to Ser.

The light-switchable polypeptide as defined in (iii) above may also have a mutation at position 37 of SEQ ID NO: 61 and a mutation at position 41 of SEQ ID NO: 61. For example, Tyr37 of SEQ ID NO: 61 may be mutated to Ala, Arg, Asn, Asp, Cys, Glu, Gln, Gly, His, Ile, Leu, Lys, Met, Phe, Pro, Ser, Thr, Trp or Val. Preferably, Tyr37 of SEQ ID NO: 61 is mutated to Asn. Leu41 of SEQ ID NO: 61 may be mutated to Ala, Arg, Asn, Asp, Cys, Glu, Gln, Gly, His, Ile, Lys, Met, Phe, Pro, Ser, Thr, Trp, Tyr or Val. Preferably, Leu41 of SEQ ID NO: 61 is mutated to Ser. For example, the light-switchable polypeptide provided herein may comprise or consist of the amino acid sequence of SEQ ID NO: 86.

The light-switchable polypeptide as defined in (iv), which has at least 80%, 85%, 90%, 95%, 96%, 97%, 98%, or 99% sequence identity to the amino acid sequence according to (i), may also have a mutation at the position which is homologous to (i.e. corresponds to) position 36 of SEQ ID NO: 20, and a mutation at the position which is homologous to (i.e. corresponds to) position 40 of SEQ ID NO: 20. For example, the position which is homologous to (i.e. corresponds to) position 36 of SEQ ID NO: 20 may be mutated to Ala, Arg, Asn, Asp, Cys, Glu, Gln, Gly, His, Ile, Leu, Lys, Met, Phe, Pro, Ser, Thr, Trp or Val, preferably to Asn. The position which is homologous to (i.e. corresponds to) position 40 of SEQ ID NO: 20 may be mutated to Ala, Arg, Asn, Asp, Cys, Glu, Gln, Gly, His, Ile, Lys, Met, Phe, Pro, Ser, Thr, Trp, Tyr or Val, preferably to Ser.

The light-switchable polypeptide as defined in (iv), which has at least 80%, 85%, 90%, 95%, 96%, 97%, 98%, or 99% sequence identity to the amino acid sequence according to (iii), may also have a mutation at the position which is homologous to (i.e. corresponds to) position 37 of SEQ ID NO: 61, and a mutation at the position which is homologous to (i.e. corresponds to) position 41 of SEQ ID NO: 61. For example, the position which is homologous to (i.e. corresponds to) position 37 of SEQ ID NO: 61 may be mutated to Ala, Arg, Asn, Asp, Cys, Glu, Gln, Gly, His, Ile, Leu, Lys, Met, Phe, Pro, Ser, Thr, Trp or Val, preferably to Asn. The position which is homologous to (i.e. corresponds to) position 41 of SEQ ID NO: 61 may be mutated to Ala, Arg, Asn, Asp, Cys, Glu, Gln, Gly, His, Ile, Lys, Met, Phe, Pro, Ser, Thr, Trp, Tyr or Val, preferably to Ser.

Accordingly, the light-switchable polypeptide provided herein may comprise or consist of a fusion protein comprising domain B1 of protein L and ABD, e.g. having the amino acid sequence of SEQ ID NO: 86; or an amino acid sequence having at least 80%, 85%, 90%, 95%, 96%, 97%, 98%, or 99% sequence identity to SEQ ID NO: 86 and comprising a light-responsive element, e.g. at the position which is homologous to position 13 of SEQ ID NO: 86. However, as also described below, for application in an affinity matrix a light-switchable domain B1 of protein L is preferably applied without an ABD fusion partner, in particular in cases were co-purification of albumin is to be avoided. Accordingly, in a preferred embodiment the light-switchable polypeptide provided herein comprises or consists of domain B1 of protein L, e.g. having the amino acid sequence of SEQ ID NO: 20, wherein residue 12 is replaced with a light-response element such as Caf; or having an amino acid sequence which has at least 80%, 85%, 90%, 95%, 96%, 97%, 98%, or 99% sequence identity to SEQ ID NO: 20, wherein the residue which is homologous to residue 12 of SEQ ID NO: 12 is replaced with a light-responsive element such as Caf.

As mentioned above, it is also envisaged that the light-switchable polypeptide of the present invention is protein A (or a variant, mutein, fusion protein, or fragment thereof) comprising a light-responsive element, protein G (or a variant, mutein, fusion protein, or fragment thereof) comprising a light-responsive element, protein L (or a variant, mutein, fusion protein, or fragment thereof) comprising a light-responsive element, or an anti-myc-tag antibody (or a variant, mutein, fusion protein, or fragment thereof) comprising a light-responsive element. The amino acid sequences of protein A, protein G, protein L and an anti-myc-tag antibody, as well as amino acid positions within theses sequences that are suitable for the incorporation of a light-responsive element (e.g. 4′-carboxyphenylazophenylalanine or 3′-carboxyphenylazophenylalanine) are provided herein above and below.

In the appended Examples a light-responsive element (i.e. a light-responsive amino acid side chain) is exemplary introduced into a mutein of streptavidin. Therefore, in one aspect of the invention, the light-switchable polypeptide comprises or consists of
(i) the amino acid sequence of SEQ ID NO: 2;
(ii) the amino acid sequence of SEQ ID NO: 4; or
(iii) the amino acid sequence of SEQ ID NO: 6.

In one particular example of the present invention the light-switchable polypeptide comprises or consists of the amino acid sequence of SEQ ID NO: 2.

In the appended Examples a light-responsive element (i.e. a light-responsive amino acid side chain) is also introduced into a fusion protein comprising a codon optimized domain B1 of protein L which is fused to an albumin-binding domain (ABD). Therefore, in one aspect of the invention, the light-switchable polypeptide comprises or consists of the amino acid sequence of SEQ ID NO: 61, wherein the residue at position 13 of SEQ ID NO: 61 is replaced by a light-responsive element, such as Caf. Such a fusion protein may also have a mutation at position 37 of SEQ ID NO: 61 (e.g. a Tyr to Asn mutation) and a mutation at position 41 of SEQ ID NO: 61 (e.g. a Leu to Ser mutation). For example, the light-switchable polypeptide of the present invention may comprise or consist of the amino acid sequence of SEQ ID NO: 86.

However, the principle provided herein can be applied to any protein that is used as affinity molecule in affinity chromatography. An overview of commonly used tags and corresponding affinity molecules is given in Table 1, below. According to the present invention any of the affinity molecules described therein may be modified in order to be light-controllable. For example, the generation and use of a light-switchable anti-HA antibody, anti-FLAG-tag antibody, or anti-T7-tag antibody is also comprised by the present invention.

TABLE 1
Overview of some commonly used tags for affinity chromatography

| | Affinity | | | |
|---|---|---|---|---|
| TAG | matrix | Elution | Comment | Reference |
| Strep-tag | Strep-Tactin | biotin or | Short, linear recognition motif; | Schmidt & Skerra |
| | (modified | desthiobiotin | matrix regenerable; one-step | 2007 Nat. Protoc. 2: |
| | streptavidin) | | purification of relatively pure | 1528-1535; Schmidt |
| | | | protein, used for pro- and | & Skerra 1994 J. |
| | | | eukaryotic cell surface display, | Chromatogr. A 676: |
| | | | immobilization to streptavadin- | 337-345 |
| | | | coated surfaces (e.g., SPR chips); | |
| | | | specific binding conditions may be | |
| | | | unsuitable for some fusions | |
| HA-tag | mAb based | synthetic HA | Anti-HA antibodies specific; useful | Hage 1999 Clin. |
| | affinity matrix | peptide or | in mammalian expression systems; | Chem. 45: 593-615 |
| | | low pH | low pH elution may irreversibly | |
| | | | affect protein properties; matrix is of | |
| | | | limited reusability | |
| FLAG-tag | mAb based | synthetic | Short, linear recognition motif; | Einhauer & |
| | affinity matrix | FLAG | moderately pure protein in one- | Jungbauer 2001 J. |
| | | peptide or | step; enterokinase cleaves after C- | Biochem. Biophys. |
| | | low pH, | term Lys to completely remove tag, | Methods 49: 455- |
| | | EDTA | depending on identity of first amino | 465; Knappik 1994 |
| | | | acid of fusion; M1 antibody can only | Biotechniques 17: |
| | | | bind tag at N-term; low pH elution | 754-761 |
| | | | may irreversibly affect protein | |
| | | | properties; matrix is of limited | |
| | | | reusability | |
| myc-tag | mAb based | synthetic | Short, linear recognition motif; anti- | Kolodziej & Young |
| | affinity matrix | myc peptide | myc antibody somewhat | 1991 Methods |
| | | or low pH | promiscuous; low pH elution may | Enzymol. 194: 508- |
| | | | irreversibly affect protein properties; | 519; Terpe 2003 |
| | | | matrix is of limited reusability | Appl. Microbiol. |
| | | | | Biotechnol. 60: 523- |
| | | | | 533 |
| T7-tag | mAb based | synthetic T7 | May increase expression of fusion | Chatterjee & |
| | affinity matrix | peptide or | proteins; low pH elution may | Esposito 2006 |
| | | low pH | irreversibly affect protein properties; | Protein Expr. Purif. |
| | | | matrix is of limited reusability | 46: 122-129 |

One aspect of the present invention relates to the light-switchable polypeptide, use, or method provided herein wherein the switch of one configuration (i.e. configurational state) to the other configuration (configurational state) of the light-responsive element changes the conformation or shape of the ligand-binding pocket or site of the polypeptide (i.e. of the light-switchable polypeptide). Herein, the term “changes the conformation” or grammatical variations thereof, is used synonymously with the term “changes the shape” or grammatical variations thereof. In particular, herein a conformational change of the ligand-binding pocket is a change in the shape of the ligand-binding pocket or ligand-binding site. As defined herein, each possible shape of the ligand-binding pocket is a “conformation” of the ligand-binding pocket. Herein, a transition between different conformations is a conformational change.

According to the present invention, a switch of the configuration of the light-responsive element of the light-switchable polypeptide provided herein alters the binding activity of the light-switchable polypeptide to a ligand. Or, in other words, the configuration of the light-responsive element determines whether the light-switchable polypeptide provided herein has binding activity to its ligand. It is envisaged that the light-responsive element contributes to the shape of the ligand-binding pocket or site of the light-switchable polypeptide. Accordingly, one aspect of the invention relates to the light-switchable polypeptide, use, or method provided herein wherein the light-responsive element is in or in the vicinity of the ligand-binding pocket or site of the polypeptide. If a ligand is bound to the light-switchable polypeptide of the present invention, then the light-responsive element has preferably a distance to said ligand which is less than 25 Å, more preferably less than 20 Å, even more preferably less than 15 Å, even more preferably less than 10 Å, and most preferably less than 5 Å. Also, the light-responsive element may be involved in the binding of a ligand to the affinity molecule (i.e. to the light-switchable polypeptide).

The position Trp108 of Strep-Tactin in the amino acid sequence of mature (devoid of the signal sequence) wild type streptavidin (UniProt Entry: P22629) is situated at the bottom of the binding cavity of Strep-Tactin. Trp108 corresponds to position 132 in pre-streptavidin, which comprises full length streptavidin with an aminoterminal signal sequence (SEQ ID NO: 12), and to position 96 in recombinant core streptavidin (SEQ ID NO: 10). Recombinant core streptavidin including its muteins and variants (SEQ ID NOs: 2, 4, 8 and 10) is devoid of the signal sequence and truncated at the aminoterminus as well as the carboxyterminus, optionally carrying an additional start-methionine residue as published (Schmidt & Skerra 1994 J. Chromatogr. A 676: 337-345). In the context of the present invention it has surprisingly been found that a change of the configurational state of a light-responsive element introduced at this position affects the affinity of Strep-Tactin to its ligand (i.e. Strep-tag or Strep-tag II). Accordingly, this position is particularly suitable as location for the light-responsive element within a light-switchable polypeptide of the invention. Thus, one aspect of the present invention relates to the light-switchable polypeptide, use, or method provided herein wherein the light-responsive element is
(i) at amino acid position 96 of any one of SEQ ID NOs: 2, 4, 8, and 10;
(ii) at amino acid position 132 of any one of SEQ ID NOs: 6 and 12;
(iii) in an amino acid sequence having at least 80%, preferably at least 85%, more preferably at least 90%, even more preferably at least 95%, even more preferably at least 96%, even more preferably at least 97%, even more preferably at least 98%, or most preferably at least 99% identity to the amino acid sequence of any one of SEQ ID NOs: 2, 4, 8 and 10 at the amino acid position that is homologous to amino acid position 96 of SEQ ID NO: 2, 4, 8 or 10, respectively; or
(iv) in an amino acid sequence having at least 80%, preferably at least 85%, more preferably at least 90%, even more preferably at least 95%, even more preferably at least 96%, even more preferably at least 97%, even more preferably at least 98%, or most preferably at least 99% identity to the amino acid sequence of any one of SEQ ID NOs: 6 and 12 at the amino acid position that is homologous to amino acid position 132 of SEQ ID NO: 6 or 12, respectively.

In the context of the present invention it has also been found that if a light-responsive element is introduced in the domain B1 of protein L at the position corresponding to position Phe12 of SEQ ID NO: 20, the affinity to its ligand (such as an immunoglobulin or antibody) can be regulated by irradiation with light. More specifically, in the context of the present invention a fusion protein comprising a protein L domain B1 and an albumin-binding domain has been prepared, and a light-responsive element has been incorporated in this fusion protein at the position corresponding to position 13 of SEQ ID NO: 61, The affinity of the resulting protein L domain B1 fusion protein to its ligand can be regulated by irradiation with light. Thus, one aspect of the present invention relates to the light-switchable polypeptide, use, or method provided herein wherein the light-responsive element is
(i) at position 12 of SEQ ID NO: 20;
(ii) at position 13 of any one of SEQ ID NOs: 61 and 86;
(iii) in an amino acid sequence having at least 80%, preferably at least 85%, more preferably at least 90%, even more preferably at least 95%, even more preferably at least 96%, even more preferably at least 97%, even more preferably at least 98%, or most preferably at least 99% identity to the amino acid sequence of SEQ ID NO: 20, at the amino acid position that is homologous to amino acid position 12 of SEQ ID NO: 20; or
(iv) in an amino acid sequence having at least 80%, preferably at least 85%, more preferably at least 90%, even more preferably at least 95%, even more preferably at least 96%, even more preferably at least 97%, even more preferably at least 98%, or most preferably at least 99% identity to the amino acid sequence of any one of SEQ ID NOs: 61 and 86, at the amino acid position that is homologous to amino acid position 13 of SEQ ID NO: 61.

The light-switchable polypeptide according to (i) may have a mutation at position 36 of SEQ ID NO: 20 (e.g. Tyr to Asn) as defined above, and/or a mutation at position 40 of SEQ ID NO: 20 (e.g. Leu to Ser) as defined above; preferably the light-switchable polypeptide has both mutations. The light-switchable polypeptide according to (ii) may have a mutation at position 37 of SEQ ID NO: 61 (e.g. Tyr to Asn) as defined above, and/or a mutation at position 41 of SEQ ID NO: 61 (e.g. Leu to Ser) as defined above; preferably the light-switchable polypeptide has both mutations. In one embodiment the light-switchable polypeptide according to (iii) has a mutation at the position which is homologous to (i.e. corresponds to) position 36 of SEQ ID NO: 20 (e.g. Tyr to Asn) as defined above, and/or a mutation at the position which is homologous to (i.e. corresponds to) position 40 of SEQ ID NO: 20 (e.g. Leu to Ser) as defined above; preferably the light-switchable polypeptide has both mutations. In another embodiment the light-switchable polypeptide according to (iii) has a mutation at the position which is homologous to (i.e. corresponds to) position 37 of SEQ ID NO: 61 (e.g. Tyr to Asn) as defined above, and/or a mutation at the position which is homologous to (i.e. corresponds to) position 41 of SEQ ID NO: 61 (e.g. Leu to Ser) as defined above; preferably the light-switchable polypeptide has both mutations.

The skilled person can easily assess whether a particular amino acid position of a given sequence that has at least 80%, at least 85%, at least 90%, at least 95%, at least 96%, at least 97%, at least 98%, or at least 99% identity to the amino acid sequence of SEQ ID NO: 2, 4, 6, 8, 10, 12, 20 or 61 is homologous (i.e. corresponds or is equivalent) to amino acid position 96, 132, 12 or 13 of the amino acid sequence of SEQ ID NO: 2, 4, 6, 8, 10, 12, 20 or 61, respectively. For example, such homologous positions can easily be identified by performing a sequence alignment between the given sequence and the amino acid sequence of SEQ ID NO: 2, 4, 6, 8, 10, 12, 20 or 61. Aligned amino acid sequences are typically represented as rows within a matrix. In these rows homologous (i.e. corresponding) amino acids lie below each other. Gaps are inserted between the residues so that identical or similar characters are aligned in successive columns. A variety of computational algorithms exist that can be used for performing a sequence alignment in order to identify an amino acid position that is homologous to an amino acid position of another sequence. For example, by using the NCBI BLAST algorithm (Altschul et al. 1997 Nucleic Acids Res. 25: 3389-3402) or CLUSTALW software (Sievers & Higgins 2014 Methods Mol. Biol. 1079: 105-116.) sequence alignments may be performed. However, sequences can also be aligned manually.

One aspect of the present invention relates to the light-switchable polypeptide, use, or method provided herein wherein the polypeptide comprising the first configuration of the light-responsive element has higher affinity to a ligand as compared to the polypeptide comprising a second configuration of the light-responsive element. Preferably, the polypeptide comprising a first configuration of the light-responsive element has high affinity to a ligand and the polypeptide comprising a second configuration of the light responsive element has low affinity to said ligand.

The term “affinity” is commonly known in the art and refers to the intrinsic binding strength of one molecule to another. Or, in other words, the affinity is the tendency of a molecule to associate with another. In particular, herein a polypeptide has a “high affinity” to a ligand if the polypeptide is capable of retaining at least 60%, preferably at least 70% more preferably at least 80%, and most preferably at least 90% of the molecule of interest within an affinity chromatography column. It is envisaged that the polypeptide having “high affinity” to a ligand is even capable of retaining at least 60%, preferably at least 70% more preferably at least 80%, and most preferably at least 90% of the molecule of interest within an affinity chromatography column if the affinity chromatography column is washed with an appropriate buffer such as phosphate-buffered saline (PBS) or tris-buffered saline (TBS).

On the other hand, herein a polypeptide has “low affinity” to a ligand if, by using an appropriate elution buffer (e.g. PBS or TBS), at least 60%, preferably at least 70%, more preferably at least 80%, and most preferably at least 90% of the molecule of interest is eluted from the affinity chromatography column.

For example, herein “high affinity” includes an affinity with a dissociation constant (K_{d}) value of <10 μM, preferably of ≤1 μM, more preferably of 100 nM, even more preferably of 510 nM, and most preferably of ≤1 nM. On the other hand, herein “low affinity” includes an affinity with a K_{d} value of >10 μM, preferably of ≥100 μM, more preferably of ≥1 mM, even more preferably of ≥10 mM, and most preferably of ≥100 mM.

Thus, in the context of the present invention a polypeptide which has “low affinity” to a ligand includes a polypeptide which has an affinity with a K_{d} value that is fold, preferably ≥100 fold, more preferably ≥1000 fold, and most preferably ≥10000 fold larger than the K_{d} value of a polypeptide which has “high affinity” to the ligand. Or, in other words, herein the light-switchable polypeptide comprising a second configuration of the light-responsive element has an affinity with a K_{d} value that is fold, preferably ≥100 fold, more preferably ≥1000 fold, and most preferably ≥10000 fold higher than the K_{d} value of a light-switchable polypeptide comprising a first configuration of the light-responsive element.

The K_{d} value with which a polypeptide binds to a given ligand can be determined by well known methods including, without being limiting, fluorescence titration, ELISA or competition ELISA, calorimetric methods, such as isothermal titration calorimetry (ITC), flow cytometric titration analysis (FACS titration) and surface plasmon resonance (BIAcore). Preferably, the K_{d} value with which a polypeptide binds to a given ligand is determined with an ELISA. Such methods are well known in the art and have been described e.g. in (De Jong 2005 J. Chromatogr. B 829: 1-25; Heinrich 2010 J. Immunol. Methods 352: 13-22; Williams & Daviter (Eds.) 2013 Protein-Ligand Interactions, Methods and Applications, Springer, New York, N.Y.).

As described in the appended Examples, a light-switchable polypeptide can be obtained by incorporating a photo-isomerizable group into an affinity molecule (e.g. streptavidin, protein A, protein G, protein L, an anti-myc-tag antibody, or a variant, fusion protein, mutein or fragment thereof). For example, one aspect of the present invention relates to the light-switchable polypeptide, use, or method provided herein, wherein the light-responsive element comprises a hydrophilic compound or molecular moiety comprising an azo group. Accordingly, the light-responsive element of the light-switchable polypeptide, use, or method provided herein may comprise an azo group. The term “azo group” is commonly known in the art and refers to an N═N group. In accordance with the present invention the light-responsive element may comprise an azo compound. An azo compound is any derivative of diazene (diimide), HN═NH, wherein both hydrogens are substituted by hydrocarbyl groups, e.g. PhN═NPh azobenzene or diphenyldiazene, which themselves may carry substituents. Thus, herein an azo compound is any compound bearing the functional group R—N═N—R′, in which R and R′ can be either aryl or alkyl. Preferred are such hydrocarbyl groups which carry hydrophilic or polar substituents, e.g. —COOH, —SO₃H, —B(OH)₂, —CONH₂, —CONR″R′″, —NH₂, —NR″R′″, wherein R″ and R′″ can be either aryl or alkyl.

The chemical modification of proteins with a photoactive ligand such as azobenzene has been described in the art (Kramer et al. 2005 Nat. Chem. Biol. 1: 360-365). The photochromic properties of azobenzenes have attracted great interest due to the stereochemical cis/trans isomerization of the N═N double bond that is readily triggered by a light source (Merino & Ribagorda 2012 Beilstein J. Org. Chem. 8: 1071-1090). The trans-azobenzene, which is energetically favored in the ground state, isomerizes to the cis isomer by irradiation with a wavelength between 300 and 390 nm. This photoreaction is reversible and the trans isomer is recovered when the cis isomer is irradiated with light of 400 to 530 nm (Merino & Ribagorda 2012 Beilstein J. Org. Chem. 8: 1071-1090) or by way of thermal relaxation.

For many azobenzenes, both types of photochemical conversion (trans to cis and cis to trans) occur within picoseconds while the thermal relaxation of the cis isomer to the trans isomer (ground state) is much slower (milliseconds to days at ambient temperature or faster if heated). The photo-induced isomerization of azobenzenes leads to a change in their physical properties, in particular molecular geometry, dipole moment and light absorption (Henzl et al. 2006 Angew. Chem. Int. Ed. Engl. 45: 603-606). In azobenzene and its derivatives, the isomerization process involves a pronounced decrease in the distance between the two para carbon atoms of the aromatic rings on both sides of the azo group, from 9.0 Å in the trans form to 5.5 Å in the cis form (Koshima et al. 2009 J. Am. Chem. Soc. 131: 6890-6891).

To biosynthetically incorporate the azobenzene moiety into proteins, an unnatural amino acid dubbed AzoPhe was generated in the prior art and its genetic incorporation into a recombinant protein via the amber suppression technique has been described (Bose et al. 2006 J. Am. Chem. Soc. 128: 388-389). However, this photo-responsive amino acid suffers from extremely poor solubility in water as well as culture media, which limits is use for biosynthetic purposes. Later, photo-switchable amino acids based on tetra-o-fluoro-substituted azobenzenes were also investigated (John et al. 2015 Org. Lett. 17: 6258-6261), but showed inferior trans to cis photo-switching properties.

Other derivatives of azobenzene are the non-natural amino acids 4′-carboxyphenylazophenylalanine (i.e. 4-[(4-carboxyphenyl)azo]-L-phenylalanine) and 3′-carboxyphenylazophenylalanine (i.e. 4-[(3-Carboxyphenyl)azo]-L-phenylalanine). These non-natural amino acids still have the ability that the cis and trans configurational isomers can be switched with particular wavelengths of light. In addition, these artificial amino acids can be incorporated into a polypeptide, thereby generating a light-switchable polypeptide. Surprisingly, in the context of the present invention it was found that 4′-carboxyphenylazophenylalanine and 3′-carboxyphenylazophenylalanine have good solubility in water and LB culture medium at physiological pH, which constitutes a considerable advantage. Moreover, these compounds have the further advantage that they have a physiological structure (the biochemical carboxylate moiety instead of fluoro atoms) resulting in a reduced risk of toxicity and immunogenicity. Thus, one aspect of the present invention relates to the light-switchable polypeptide, use, or method provided herein wherein the light-responsive element comprises
(i) 3′-carboxyphenylazophenylalanine or a derivative thereof; or
(ii) 4′-carboxyphenylazophenylalanine or a derivative thereof.

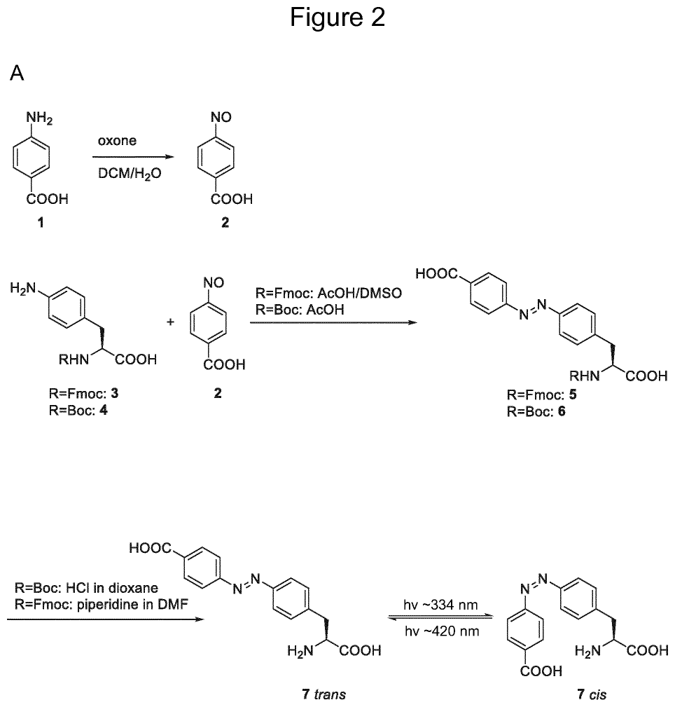

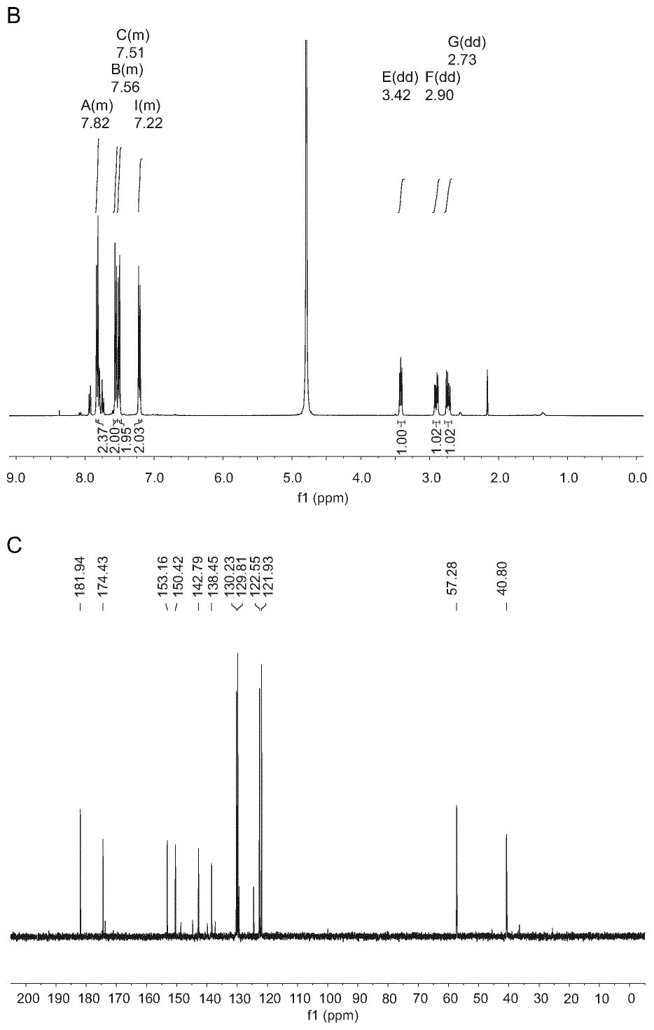

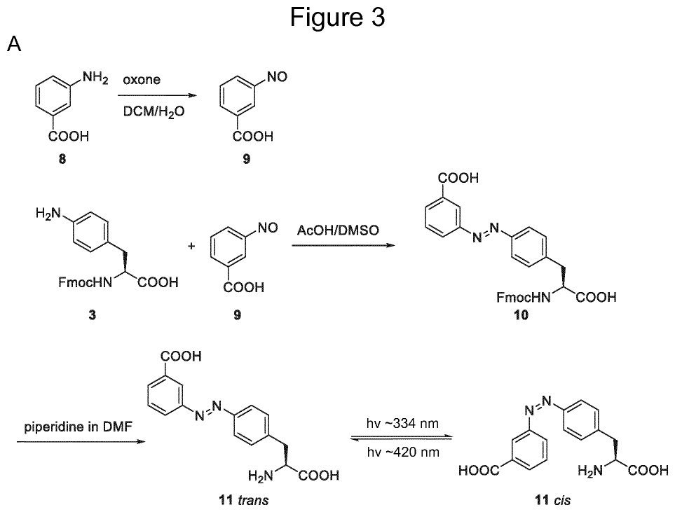

The formulae of 3′-carboxyphenylazophenylalanine and 4′-carboxyphenylazophenylalanine are shown herein in FIGS. 3 and 2, respectively.

It is most preferred that the light-responsive element of the light-switchable polypeptide of the present invention comprises 4′-carboxyphenylazophenylalanine (abbreviated: Caf).

As demonstrated in the appended Examples, the light-induced modification of the binding properties of the inventive light-switchable polypeptide can be achieved, e.g., by site-directed incorporation of a non-natural (in particular, non-proteinogenic) light-switchable amino acid. This non-natural amino acid has a light-switchable side chain. Or, in other words, the configuration of the side chain of the non-natural amino acid can be changed by irradiating it with (a) particular wavelength(s) of light. This configurational change advantageously results in a change of the conformation and/or binding activity of the corresponding polypeptide (affinity molecule). Therefore, according to the present invention the light-responsive element may comprise a light-switchable amino acid side chain. For example, the light-responsive element may comprise or consist of a non-natural (i.e. non-proteinogenic) amino acid wherein two configurational isomers of the non-natural amino acid can be switched by applying (a) particular wavelength(s) of light.

The biosynthesis of proteins with non-natural (i.e. non-proteinogenic) amino acids has been established since several years and has opened the way to novel biomolecular reagents for biophysical, structural and biochemical research as well as biotechnological and biopharmaceutical applications (Wals & Ovaa 2014 Front. Chem. 2: 15). A versatile method for the site-specific incorporation of non-natural (i.e. non-proteinogenic) amino acids exploits a nucleic acid codon that is not actively used by the genetic code of the host cell. Thus, the amber stop codon (UAG), which also is subject to natural nonsense suppression mechanisms, has been recruited as an additional coding triplet for novel amino acids to provide new side chain chemistries in recombinant proteins. Initially developed for in vitro translation systems employing synthetic aminoacyl-tRNAs, this general approach has been adapted to the heterologous overexpression of proteins in live cells by utilizing an artificial aminoacyl-tRNA synthetase (aaRS) with the desired amino acid substrate specificity (Young & Schultz 2010 J. Biol. Chem. 285: 11039-11044). Importantly, such an aaRS must not aminoacylate any endogenous cellular tRNA, whereas the cognate suppressor tRNA, which is co-overexpressed in vivo, must not be aminoacylated with a natural amino acid by any endogenous aminoacyl-tRNA synthetase. In other words, suppressor tRNA and the foreign or engineered aaRS must be orthogonal to their endogenous counterparts in the host cell of choice.

The first efficient orthogonal pair of tRNA and aaRS suitable for in vivo translation in E. coli was found in the tyrosyl-tRNA synthetase (TyrRS) from the archaebacterium Methanococcus jannaschii (Mj) and its cognate tRNA^{Tyr}, which was mutated to specifically recognize and suppress the amber stop codon (Wang & Schultz 2001 Chem. Biol. 8: 883-890). Later, the toolbox for incorporation of non-natural amino acids was expanded by a system based on the 22nd proteinogenic amino acid, L-pyrrolysine (Pyl), which is translated in response to an amber stop codon by the action of pyrrolysyl-tRNA synthetase (PylRS) together with its cognate natural suppressor tRNA^{Pyl} (Fekner & Chan 2011 Curr. Opin. Chem. Biol. 15: 387-391). This system was originally found in the methanogenic archaeons Methanosarcina barkeri (Mb) and Methanosarcina mazei (James et al. 2001 J. Biol. Chem. 276: 34252-34258) and is now increasingly used as a genetic code expansion tool (Wan at al. 2014 Biochem. Biophys. Acta 1844: 1059-1070). Due to its rather low selectivity towards the natural substrate Pyl, PylRS has (in part after protein engineering) permitted the genetic incorporation of more than 100 non-natural amino acids (Wan et al. 2014 Biochem. Biophys. Acta 1844: 1059-1070).

Thus, several well-established systems exist for realizing the biosynthesis of proteins with non-proteinogenic amino acids. As documented in the appended Examples, these methods clearly enable the production of the light-switchable polypeptide provided herein. More specifically, the light-switchable polypeptide of the invention can be prepared by incorporating a photo-isomerizable amino acid into into a protein (such as streptavidin, protein A, protein G, protein L, an anti-myc-tag antibody, or a variant, fusion protein, mutein or fragment thereof) which in turn is used as affinity molecule in affinity chromatography.

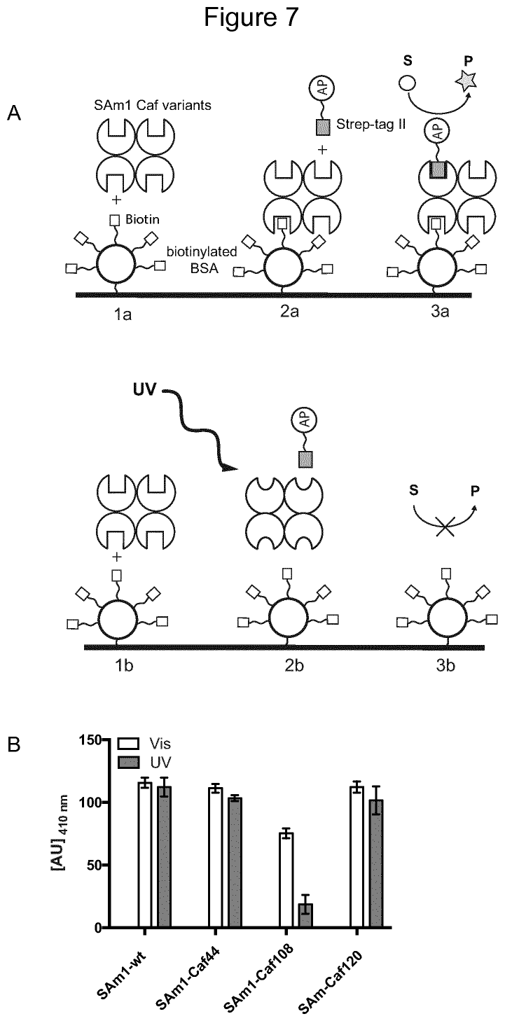

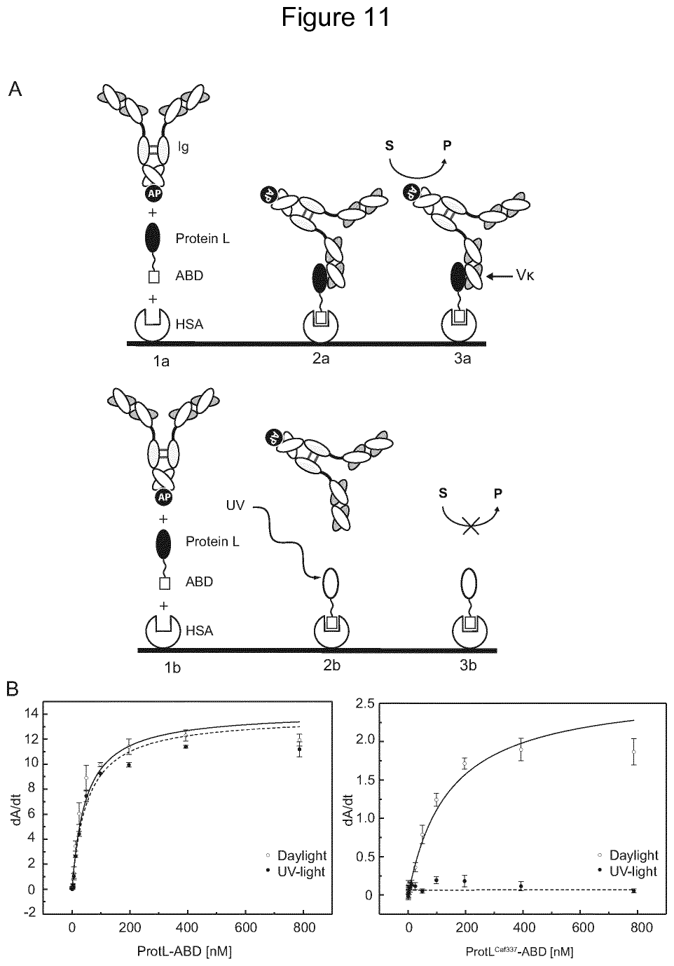

In order to test whether a newly designed polypeptide shows a light-induced change of the affinity to a ligand (i.e. in order to test whether it is a light-switchable polypeptide in accordance with the present invention), an enzyme-linked immunosorbent assay (ELISA) may be performed. An ELISA that may be used in this regard is exemplified in FIG. 7(A). More specifically, FIG. 7(A) shows a schematic representation of an ELISA that may be used for the detection of the interaction between a ligand (e.g. a protein of interest comprising a suitable affinity tag) and a given polypeptide (affinity molecule). Such an ELISA set-up in principle corresponds to a simple version of an affinity chromatography procedure. Another ELISA that may preferably be used in this regard is exemplified in FIG. 11.

In particular, such an ELISA may be performed as follows. A plate (e.g. a microtiter plate) may be coated with the potential light-switchable polypeptide. Subsequently, a reporter enzyme (e.g. an alkaline phosphatase), which is fused with a peptide ligand of the potential light-switchable polypeptide (e.g. an affinity tag such as the Strep-tag or Strep-tag II) may be added. Then, washing steps without or with exposure to light having (a) particular wavelength(s) (e.g. UV light having a wavelength of 300 to 390 nm) may be carried out. Afterwards, the remaining bound enzyme may be detected via biocatalytic conversion of a chromogenic substrate (e.g., p-nitrophenylphosphate) and quantified, e.g., as absorbance in a photometer.

In this ELISA a potential light-switchable polypeptide can be considered to be a light-switchable polypeptide according to the present invention
(1) if a decrease in remaining enzyme activity is detected upon exposure of the potential light-switchable polypeptide to light having (a) particular wavelength(s) (e.g. UV light having a wavelength of 300 to 390 nm); and
(2) if no (or less) decrease in remaining enzyme activity is detected when the potential light-switchable polypeptide is exposed to light having (a) different particular wavelength(s) (e.g. with visible light having a wavelength of about 400 to 530 nm, e.g., 400 to 500 nm) or kept in the dark.

As mentioned above, the present invention provides a polypeptide comprising a light-responsive element (e.g. 3′-carboxyphenylazophenylalanine or 4′-carboxyphenylazophenylalanine), wherein the switch of the configuration of the light-responsive element alters the binding activity of the polypeptide to a ligand. As commonly known in the art, “isomers” are compounds that have the same molecular formula or composition but a different structure. “Stereoisomers” only differ in the spatial orientation of their component atoms. Therefore, stereoisomers require that an additional nomenclature prefix be added to the IUPAC name in order to indicate their spatial orientation. Commonly used prefixes that are used to distinguish stereoisomers are cis (Latin, meaning “on this side”) and trans (Latin, meaning “across”). More specifically, in organic chemistry “cis” means that the substituents are on the same side of a pair of atoms, often carbon but also nitrogen such as in the case of azo compounds, which are linked by a non-rotatable bond, e.g. a double bond, whereas “trans” means that the substituents (e.g. functional groups) are on opposite sides of said pair of atoms. Such isomeric states are commonly referred to as configurations or configurational isomers or states.

For some compounds it is not clear which isomer should be called cis and which trans. Therefore, an unambiguous system of rules to define such stereoisomers has been proposed by the International Union of Pure and Applied Chemistry (IUPAC). This system is based on a set of group priority rules on the substituents (known as the Cahn-Ingold-Prelog or CIP rules) assigning a Z (German, “zusammen” for “together”) or an E (German, “entgegen” for “opposite”) to designate the stereoisomers. Often, Z is equivalent to cis and E is equivalent to trans for isomers for which the cis-trans notation is adequate.

According to the present invention the isomers of the light-responsive element may be a trans isomer and a cis isomer. In addition or alternatively, the isomers of the light-responsive element may be an E isomer and a Z isomer. Accordingly, herein, the switch of the configuration of the light-responsive element may be the conversion from the trans (or E) isomer of the light-responsive element to the corresponding cis (or Z) isomer, and vice versa. The cis and trans isomers of 3′-carboxyphenylazophenylalanine and 4′-carboxyphenylazophenylalanine are shown herein in FIGS. 3 and 2, respectively.

One aspect of the invention relates to the light-switchable polypeptide, use, or method provided herein wherein the polypeptide comprising a trans isomer of 3′-carboxyphenylazophenylalanine or 4′-carboxyphenylazophenylalanine has an increased affinity to a ligand as compared to the polypeptide comprising a cis isomer of 3′-carboxyphenylazophenylalanine or 4′-carboxyphenylazophenylalanine, respectively. For example, the polypeptide comprising a trans isomer of 3′-carboxyphenylazophenylalanine or 4′-carboxyphenylazophenylalanine may have “high affinity” to a ligand; and the polypeptide comprising a cis isomer of 3′-carboxyphenylazophenylalanine or 4′-carboxyphenylazophenylalanine may have “low affinity” to the same ligand. The terms “high affinity” and “low affinity” are defined herein above.

However, the light-switchable polypeptide of the present invention may also be constructed in a way that its cis isomer has higher affinity to the ligand as compared to the trans isomer. Thus, one embodiment of the present invention relates to the light-switchable polypeptide, use, or method provided herein wherein the polypeptide comprising a cis isomer of 3′-carboxyphenylazophenylalanine or 4′-carboxyphenylazophenylalanine has an increased affinity to a ligand as compared to the polypeptide comprising a trans isomer of 3′-carboxyphenylazophenylalanine or 4′-carboxyphenylazophenylalanine, respectively. In this embodiment the polypeptide comprising a cis isomer of 3′-carboxyphenylazophenylalanine or 4′-carboxyphenylazophenylalanine may have “high affinity” to a ligand; and the polypeptide comprising a trans isomer of 3′-carboxyphenylazophenylalanine or 4′-carboxyphenylazophenylalanine may have “low affinity” to the same ligand. As mentioned, an explicit definition of the terms “high affinity” and “low affinity” is given herein above.

As an example for a potential light-switchable polypeptide, a light-switchable streptavidin mutant is prepared and characterized in the appended Examples. At visible light having (a) wavelength(s) around 400 to 530 nm, 80-90% of this light-switchable streptavidin mutant comprises the trans isomer of the light-responsive element (e.g. the trans isomer of 3′-carboxyphenylazophenylalanine or 4′-carboxyphenylazophenylalanine). At UV light having a wavelength around 300 to 390 nm, 80-90% of the exemplary light-switchable streptavidin mutant comprises the cis isomer of the light-responsive element (i.e. the cis isomer of 3′-carboxyphenylazophenylalanine or 4′-carboxyphenylazophenylalanine).

Usually, visible light covers wavelengths from 400 to 780 nm. This light is also commonly referred to as daylight.

The appended Examples demonstrate that, if the light-switchable polypeptide of the present invention is applied for an affinity chromatography procedure, then the highest degree of binding and the highest degree of elution of the molecule of interest takes place at around 430 nm and at around 330 nm, respectively. Therefore, light having (a) wavelength(s) around 430 nm (visible light) and 330 nm (UV light) may be applied in the context of the present invention. However, conventional light sources usually provide light having wavelengths that are around 530 nm (visible light) and 365 nm (UV light). Therefore, also light providing these wavelengths (i.e. around 530 nm and/or around 365 nm) may be used in accordance with the present invention.

Therefore, one aspect of the present invention relates to the light-switchable polypeptide, use, or method provided herein wherein at visible light having about 400 to 530 nm, e.g., 400 to 500 nm, preferably 405 to 470 nm, more preferably 410 to 450 nm, and most preferably about 430 nm, at least 60%, preferably at least 70%, more preferably at least 75%, even more preferably at least 80%, even more preferably at least 90%, and most preferably at least 95% of the light-switchable polypeptide comprises a trans isomer of the light-responsive element.

Another aspect of the present invention relates to the light-switchable polypeptide, use, or method provided herein wherein at UV light having 300 to 390 nm, preferably 310 to 370 nm, even more preferably 320 to 350 nm, and most preferably about 330 nm, at least 60%, preferably at least 70%, more preferably at least 75%, even more preferably at least 80%, even more preferably at least 85%, even more preferably at least 90%, and most preferably at least 95% of the light-switchable polypeptide comprises a cis isomer of the light-responsive element. As mentioned above, conventional light sources usually provide UV light having wavelengths around 365 nm. Therefore, an alternative aspect of the present invention relates to the light-switchable polypeptide, use, or method provided herein, wherein at UV light having about 365 nm at least 60%, preferably at least 70%, more preferably at least 75%, even more preferably at least 80%, even more preferably at least 85%, even more preferably at least 90%, and most preferably at least 95% of the light-switchable polypeptide comprises a cis isomer of the light-responsive element.

It is known to the skilled person in the art that the degree (e.g. proportion, fraction or yield) of isomerization or configurational switch of the light-responsive element not only depends on the wavelength but also on the intensity of light used for irradiation. Useful light intensities according to this invention are achieved when a conventional light source such as an LED or several LEDs with a combined electric power of at least 0.1 mW, preferably, at least 1 mW, more preferably at least 10 mW, more preferably at least 100 mW, most preferably at least 1000 mW are applied for irradiating 1 mL (wet) volume of an affinity matrix or chromatography matrix that carries the light-switchable polypeptide (affinity molecule) and placed at a distance of less than 1 m, preferentially less than 10 cm, more preferentially less than 2 cm and most preferentially less than 1 cm to said matrix. For larger volumes of affinity matrix or chromatography matrix proportionally larger values of electric power are applied. Alternatively, other light sources providing similar light intensities and wavelengths as said LEDs may be used, for example (a) tubular fluorescent lamp(s). For larger volumes of the affinity matrix the light source may also be placed within the bed of the chromatography column, e.g. using a fiberoptic.

The UV light according to the present invention falls into a region of the spectrum of electromagnetic radiation which is commonly referred to as the near ultraviolet (UV) light. The wavelengths of the UV light according to the present invention are essentially not absorbed by many biomolecules of interest, including proteins, nucleic acids and carbohydrates. Hence, said UV light can be considered mild as the risk of radiation damage is low if compared with the use of far UV light, having shorter wavelengths and higher energy, for example.

As described above, the inventive light-switchable polypeptide can be used for separating and/or purifying a molecule of interest, e.g. during an affinity chromatography procedure. Therefore, the light-switchable polypeptide is preferably comprised in a solid phase (such as a solid carrier or adsorbed to a solid surface or to a swollen polymer gel).

Said solid phase is preferably hydrophilic. The terms “solid phase” and “liquid phase” are commonly known in the art and refer to solid material and liquid material, respectively. The liquid phase can be any solution, mixture of solutions or suspension. For example, the liquid phase can comprise a cell extract or culture supernatant, optionally mixed with a buffer solution. In accordance with the present invention the solid phase may be any suitable carrier. For example, the solid phase may be a matrix (e.g. a polymer of an organic or biomolecular substance potentially including cross-links), a hydrogel (usually formed through the cross-linking of hydrophilic polymer chains within an aqueous microenvironment), a bead, a magnetic bead, a chip, a glass surface, a plastic surface, a gold surface, a silver surface or a plate. The matrix, the hydrogel, the bead, the chip, the glass surface, the plastic surface, or the plate is preferably light-transmissive. The matrix, the hydrogel or the bead may be the solid phase of an affinity chromatography column. The matrix may be, for example, N-hydroxysuccinimidyl (NHS) activated CH-sepharose. The plate may be a microtiter well plate. An overview of some activated chromatography materials suitable for coupling of the light-switchable polypeptide of the present invention is given in Table 2, below.

TABLE 2
Overview of some activated chromatography materials suitable
for coupling of the light-switchable polypeptide.

| Material | Vendor | Comment |
|---|---|---|
| NHS-Activated | GE Healthcare | NHS pre-activated medium for coupling of small amino- |
| Sepharose 4 Fast Flow | Life Sciences | containing proteins and peptides in process-scale |
| | | applications |
| NHS-ACT Sepharose | GE Healthcare | NHS-activated Sepharose High Performance |
| High Performance | Life Sciences | |
| Activated Thiol | GE Healthcare | Activated Thiol Sepharose 4B medium is a medium |
| Sepharose 4B | Life Sciences | used for reversible immobilization of molecules |
| | | containing thiol groups under mild conditions. Optimized |
| | | for immobilization of large molecules |
| CNBr-Activated | GE Healthcare | CNBr-activated Sepharose 4 Fast Flow is a well |
| Sepharose 4 Fast Flow | Life Sciences | established, pre-activated chromatography medium for |
| | | coupling of large amino-containing ligands. |
| CNBr-Activated | GE Healthcare | CNBr-activated Sepharose 4B is a pre-activated media |
| Sepharose 4B | Life Sciences | used for coupling antibodies or other large proteins |
| | | containing —NH₂ groups to the Sepharose media, by the |
| | | cyanogen bromide method, without an intermediate |
| | | spacer arm |
| EAH Sepharose 4B | GE Healthcare | EAH Sepharose pre-activated media is used for |
| | Life Sciences | coupling compounds containing carboxyl groups to |
| | | Sepharose 4B through carbodiimide- based coupling via |
| | | an 11-atom spacer arm |
| Epoxy-Activated | GE Healthcare | Epoxy-activated Sepharose 6B is a pre-activated |
| Sepharose 6B | Life Sciences | medium for immobilization of various ligands including |
| | | sugars through coupling of hydroxy, amino or thiol |
| | | groups on the ligand to Sepharose 6B via a 12-atom |
| | | hydrophilic spacer arm |
| NHS Mag Sepharose | GE Healthcare | NHS Mag Sepharose are magnetic beads designed for |
| | Life Sciences | pull-down techniques enabling rapid capture and |
| | | enrichment of selected proteins based on affinity |
| Aldehyde Agarose | Sigma-Aldrich | Aldehyde Agarose is used in affinity chromatography. It |
| | | has been used in research for the immobilization and |
| | | stabilization of enzymes. |
| Cyanogen bromide- | Sigma-Aldrich | Cyanogen bromide-activated Agarose is lyophilized |
| activated Agarose | | powder stabilized with lactose used in affinity |
| | | chromatography, protein chromatography, protein |
| | | interactions, antibody labeling, antibody modification |
| | | and attaching antibodies to agarose beads. |
| Epoxy-activated- | Sigma-Aldrich | Epoxy-activated-Agarose is a lyophilized powder, |
| Agarose | | stabilized with lactose, which is used in affinity |
| | | chromatography, protein chromatography and |
| | | activated/functionalized matrices. Epoxy-activated |
| | | agarose has been used in studies informing anti- |
| | | proliferative activity on human-derived cancer cells as |
| | | well as cancer prevention. |
| TOYOPEARL ® AF- | Sigma-Aldrich | Toyopearl AF-Amino-650 resin is a reactive resin used |
| Amino-650M | | for the coupling of specific ligands for affinity |
| amine-activated | | chromatography. Ligands are immobilized by either |
| | | peptide bond formation or reductive amination through |
| | | their respective carboxylate or aldehyde groups. |
| TOYOPEARL ® AF- | Sigma-Aldrich | Toyopearl AF-Epoxy-650 resin is an activated resin |
| Epoxy-650M expoxy- | | provided in dry form for the immobilization of protein |
| activated | | ligands for affinity chromatography. It is used when high |
| | | densities of low molecular weight molecules need to be |
| | | attached. It is also useful when a conversion to other |
| | | special functional groups is required prior to ligand |
| | | immobilization. For instance, its hydrazide form is very |
| | | useful for carbohydrates or glycoprotein ligands. |
| TOYOPEARL ® AF- | Sigma-Aldrich | Toyopearl AF-Tresyl-650 resin is an activated resin |
| Tresyl-650M tresyl- | | which readily binds to amine and thiol groups. |
| activated | | |
| Pierce NHS-Activated | Thermo Scientific | Amine-reactive, beaded-agarose resin for rapid and |
| Agarose | | stable immobilization of proteins, peptides and other |
| | | ligands via primary amines. |
| AminoLink Coupling | Thermo Scientific | Crosslinked 4% beaded agarose that has been |
| Resin | | activated with aldehyde groups to enable covalent |
| | | immobilization of antibodies and other proteins through |
| | | primary amines. |
| AminoLink Plus Coupling | Thermo Scientific | Aldehyde-activated agarose beads for high-yield |
| Resin | | covalent coupling of antibodies (proteins) via primary |
| | | amines to prepare columns for affinity purification. |
| SulfoLink Coupling | Thermo Scientific | Crosslinked, 6% beaded agarose that has been |
| Resin | | activated with iodoacetyl groups for covalent |
| | | immobilization of cysteine-peptides and other sulfhydryl |
| | | molecules. |
| CarboxyLink Coupling | Thermo Scientific | For covalent immobilization of peptides or other |
| | | carboxyl-containing (—COOH) molecules to a porous, |
| | | beaded resin for use in affinity purification procedures. |

In accordance with the present invention, the light-switchable polypeptide may be covalently or non-covalently attached to the solid phase. It is most preferred that the light-switchable polypeptide is covalently attached to the solid phase. This has the advantage that the light-switchable polypeptide is fixed on the solid phase so that it is not eluted together with the molecule of interest. Thus, by covalently attaching the light-switchable polypeptide to the solid phase (e.g. affinity chromatography matrix) contamination of the eluted molecule of interest is avoided.

However, the present invention also comprises non-covalent binding of the light-switchable polypeptide to the solid phase. For example, the light-switchable polypeptide may be a part of a fusion protein. The other part of the fusion protein may bind, covalently or non-covalently, to the solid phase (e.g. to the matrix of the affinity chromatography column).

In one embodiment of the present invention the carrier is covalently or non-covalently attached to biotin, a biotinylated protein or molecule and/or a peptide ligand of the light-switchable polypeptide (e.g. a Step-tag). In this embodiment, the light-switchable polypeptide may be attached to the carrier via non-covalent binding to biotin, a biotinylated protein or molecule and/or the peptide ligand of the polypeptide. In an alternative embodiment of the present invention the carrier is covalently or non-covalently attached to albumin, e.g. human serum albumin (HSA). In this embodiment, the light-switchable polypeptide may be attached to the carrier via non-covalent binding to HSA, e.g. as part of a fusion protein with the ABD. For example, a light-switchable domain B1 of protein L carrying a light-responsive element can be conveniently produced as fusion protein with the ABD and tested for light-controllable affinity towards an immunoglobulin in an ELISA, as demonstrated in the appended Examples. However, for application in an affinity matrix comprising the light-switchable polypeptide as defined herein the light-switchable domain B1 of protein L carrying a light-responsive element is preferably applied without an ABD fusion partner, in particular in cases were copurification of albumin, e.g. from a cell culture medium, is to be avoided.

The light-switchable polypeptide provided herein has the advantage that its binding activity can be controlled simply by irradiating the light-switchable polypeptide with (a) particular wavelength(s) of light. Therefore, it is desirable in the context of the present invention that the used solid phase (e.g. the carrier) is light resistant. Preferably, the carrier is light resistant at least in the wavelength range from 300 nm to 500 nm, preferably from 330 nm to 450 nm.

The switch of the configuration of the light-responsive element alters the binding activity of the light-switchable polypeptide to a ligand. Herein, the “ligand” can be any molecule that has affinity to the light-switchable polypeptide provided herein in one of its configurational states (for example, the trans ground state). If the molecule of interest has affinity to the light-switchable polypeptide per se, then the ligand may be the molecule of interest itself, i.e. without further modification. For example, if the light-switchable polypeptide is a light-switchable protein A, protein G, or protein L (or a light-switchable variant, mutein, fusion protein, or fragment thereof), then an immunoglobulin, an antibody or a fragment of an antibody may be the ligand and molecule of interest. However, if the molecule of interest does not have affinity to the light-switchable polypeptide per se, then the ligand is preferably a fusion molecule comprising the molecule of interest and an affinity tag. For example, if the light-switchable polypeptide is a light-switchable streptavidin or anti-myc-tag antibody (or a light-switchable variant, mutein, fusion protein, or fragment thereof), then the ligand is preferably the molecule of interest that is fused with a Strep-tag/Strep-tag II, or a myc-tag, respectively.

Preferably, the ligand is a biomolecular ligand including a molecule selected from the group consisting of a peptide, an oligopeptide, a polypeptide, a protein, an antibody or a fragment thereof, an immunoglobulin or a fragment thereof, an enzyme, a hormone, a cytokine, a complex, an oligonucleotide, a polynucleotide, a nucleic acid, a carbohydrate, a liposome, a nanoparticle, a cell, a biomacromolecule, a biomolecule, and a small molecule. For example, the ligand may be a polypeptide, a complex, a polynucleotide, a nucleic acid, a carbohydrate, a liposome, a nanoparticle, a cell, or a small molecule. As mentioned above, the ligand may also be a fusion molecule comprising any one of the molecules mentioned above (a molecule of interest) and an affinity tag (such as a Strep-tag, a Strep-tag II, or a myc-tag). It is most preferred that the ligand is a protein or a peptide. If the light-switchable polypeptide is streptavidin (or a mutein or variant thereof, such as Strep-Tactin) comprising a light-responsive element, then the ligand may be a Strep-tag (i.e. Strep-tag or Strep-tag II) or biotin, preferably a Strep-tag. Thus, one aspect of the invention relates to the light-switchable polypeptide, use, or method provided herein wherein the peptide ligand comprises or consists of
(i) the amino acid sequence of SEQ ID NO: 13 [Strep-tag];
(ii) the amino acid sequence of SEQ ID NO: 14 [Strep-tag II]; or
(iii) an amino acid sequence having at least 80%, preferably at least 85%, more preferably at least 90%, even more preferably at least 95%, even more preferably at least 96%, even more preferably at least 97%, even more preferably at least 98%, or most preferably at least 99% identity to SEQ ID NO: 13 or 14 and having affinity to streptavidin or its mutants or variants. As mentioned above, a known mutant of streptavidin which is widely used in research and industry is Strep-Tactin. Thus, in the context of the present invention, the streptavidin mutant may be a tetramer of the protein having the amino acid sequence of SEQ ID NO: 7.

If the light-switchable polypeptide is an anti-myc-tag antibody (e.g. clone 9E10) or a fragment, a mutein or variant thereof (e.g. Fab 9E10) comprising a light-responsive element, then the ligand may be a myc-tag. The amino acid sequence of the myc-tag is shown herein as SEQ ID NO: 15.

As mentioned above, protein A as well as protein G bind to the Fc region of antibodies, particularly of IgGs including human and mouse IgGs as well as Igs from other species. protein L binds to Igs or antibodies as well, e.g. to antibodies or fragments thereof comprising a kappa light chain. Thus, if the light-switchable polypeptide is protein A comprising a light-responsive element, or protein G comprising a light-responsive element, then the ligand is preferably an antibody or a fragment thereof, preferably an IgG or a variant, mutein or fragment thereof, wherein said variant, mutein or fragment comprises the Fc region of an IgG antibody and/or the Fab region of an IgG antibody. If the light-switchable polypeptide is protein L comprising a light-responsive element, then the ligand is preferably an antibody (or a fragment thereof such as an Fab fragment, an Fv fragment, an scFv fragment or a single domain fragment), comprising a kappa light chain, such as a human VκI, VκIII and/or VκIV light chain; and/or a mouse VκI light chain. Thus, various different antibodies may be purified by using a light-switchable protein A, protein G, or protein L according to the present invention. For example, beyond others, the therapeutic antibodies described in Reichert 2017 mAbs 9: 167-181 may be isolated or purified by applying the means and methods described herein.

In accordance with the present invention, the term “% sequence identity” or “% identity” describes the number of matches (“hits”) of identical amino acids of two or more aligned amino acid sequences as compared to the number of amino acid residues making up the overall length of the amino acid sequences (or the overall part thereof that is used for the comparison). Percent identity is determined by dividing the number of identical residues by the total number of residues of the longest sequence used for the comparison and multiplying the product by 100. In other terms, using an alignment, the percentage of amino acid residues that are the same (e.g., 80% identity) may be determined for two or more sequences or sub-sequences when these (sub)sequences are compared and aligned for maximum correspondence over a sequence window used for the comparison or over a designated region as measured using a sequence comparison algorithm as known in the art or when manually aligned and visually inspected.

Those having skill in the art know how to determine percent sequence identity between/among sequences using, for example, algorithms such as those based on the NCBI BLAST algorithm (Altschul 1997 Nucleic Acids Res. 25: 3389-3402), the CLUSTALW computer program (Tompson 1994 Nucleic Acids Res. 22: 4673-4680) or FASTA (Pearson 1988 Proc. Natl. Acad. Sci. U.S.A. 85: 2444-2448). The NCBI BLAST algorithm is preferably employed in accordance with this invention. For amino acid sequences, the BLASTP program uses as default a word length (W) of 3 and an expectation (E) of 10. Accordingly, all the (poly)peptides having a sequence identity of at least 80%, preferably at least 85%, more preferably at least 90%, even more preferably at least 95%, even more preferably at least 96%, even more preferably at least 97%, even more preferably at least 98% or most preferably at least 99% identity as determined with the NCBI BLAST or BLASTP program fall under the scope of the invention.

As described above, one embodiment of the invention relates to a method for isolating and/or purifying a molecule of interest by employing the light-switchable polypeptide provided herein. In step (i) of this method, the molecule of interest binds to the light-switchable polypeptide of the invention. Therefore, during this step the light-responsive element (e.g. 3′-carboxyphenylazophenylalanine or 4′-carboxyphenylazophenylalanine) is in a configuration that results in a polypeptide which has high binding affinity to the molecule of interest. This can be achieved by irradiating the light-switchable polypeptide with (a) particular wavelength(s) of light. For example, in the method provided herein, before and/or during step (i) the light-switchable polypeptide may be irradiated with visible light having about 400 to 530 nm, e.g., 400 to 500 nm, preferably 405 to 470 nm, more preferably 410 to 450 nm, and most preferably about 430 nm.

After binding of the molecule of interest to the light-switchable polypeptide, the solid phase (e.g. the affinity chromatography matrix) may be washed in order to remove unbound material from the column. Thus, in one aspect of the invention the method provided herein further comprises the step of
(i′) washing the solid phase with an appropriate buffer.

It is envisaged that the molecule of interest stays within the column or bound to the solid phase during this washing step. Thus, during this washing step the light-responsive element of the light-switchable polypeptide provided herein is preferably in a configuration resulting in binding activity of the light-switchable polypeptide to the molecule of interest. During step (i′) of the method provided herein (i.e. during the washing step) the light-switchable polypeptide may be irradiated with visible light having about 400 to 530 nm, e.g., 400 to 500 nm, preferably 405 nm to 470 nm, more preferably 410 nm to 450 nm, and most preferably about 430 nm.

The exemplified light-switchable Strep-Tactin that has been produced in the appended Examples has binding activity to its ligand in the trans configuration of the light-responsive element, i.e. trans-3′-carboxyphenylazophenylalanine or trans-4′-carboxyphenylazophenylalanine. For this light-responsive element the trans configuration is the state with the most favorable (i.e. lowest) energy. Therefore, this exemplified light-switchable Strep-Tactin binds to its ligand even in the dark or under irradiation with wavelengths longer than 500 nm. Therefore, if the light-switchable polypeptide provided herein binds to its ligand in the conformation or configuration with the lowest energy, then step (i) (e.g. loading of the column) and (i′) (e.g. washing of the column) can also be performed in the dark or under irradiation with wavelengths that are longer than 500 nm.

In order to elute the molecule of interest, the light-switchable polypeptide has to be converted into a conformation with lower binding activity to the molecule of interest. The herein exemplarily designed light-switchable Strep-Tactin has low binding activity when its light-response element (i.e. 3′-carboxyphenylazophenylalanine or 4′-carboxyphenylazophenylalanine) is in the cis configuration. The cis configuration of this light-responsive element (i.e. cis-3′-carboxyphenylazophenylalanine or cis-4′-carboxyphenylazophenylalanine) can be obtained by irradiation with UV light. Thus, one aspect of the present invention relates to the method provided herein wherein during step (ii) (i.e. the elution step) the light-switchable polypeptide is irradiated with UV light having 300 to 390 nm, preferably 310 to 370 nm, more preferably 320 to 350 nm, or most preferably about 330 nm. Alternatively, the light-switchable polypeptide may be irradiated with UV light having about 365 nm during this step.

However, if desired, also light having a lower wavelength than near UV light can be used in order to convert the trans configuration of the light-responsive element (e.g. trans-3′-carboxyphenylazophenylalanine or trans-4′-carboxyphenylazophenylalanine) into a cis configuration. Thus, during step (ii) (i.e. the elution step) of the method provided herein, the light-switchable polypeptide may also be irradiated with light having a shorter wavelength than 300 nm, e.g. with light having (a) wavelength(s) between 300 and 200 nm. However, as described above, it is preferred in the context of the present invention that mild UV light having (a) wavelength(s) from 300 to 390 nm is used.

After elution, the light-switchable polypeptide is usually converted back to the conformation which has binding activity to the ligand. Thus, one aspect of the present invention relates to the method provided herein wherein the method further comprises the step of
(iii) regenerating the light-switchable polypeptide to the first conformation having affinity to the molecule of interest.

During this step (iii) the light-responsive element may be regenerated by irradiating the light-switchable polypeptide with visible light having about 400 to 530 nm, e.g., 400 to 500 nm, preferably 405 to 470 nm, more preferably 410 to 450 nm, and most preferably about 430 nm. Also, during step (iii) the solid phase may be washed with an appropriate buffer, for example PBS or TBS.

In the method provided herein the liquid phase comprising the molecule of interest may be a cell extract or a culture supernatant. For example, the cell extract may be an extract of the periplasm or a whole cell extract. Before the liquid phase comprising the molecule of interest is contacted with the light-switchable polypeptide, the liquid phase may be dialyzed or diluted with a buffer.

According to the present invention any molecule of interest may be isolated (and/or separated or purified) by using the light-switchable polypeptide provided herein. Preferably, the molecule of interest is a molecule selected from the group consisting of a peptide, an oligopeptide, a polypeptide, a protein, an antibody or a fragment thereof, an immunoglobulin or a fragment thereof, an enzyme, a hormone, a cytokine, a complex, an oligonucleotide, a polynucleotide, a nucleic acid, a carbohydrate, a liposome, a nanoparticle, a cell, a biomacromolecule, a biomolecule and a small molecule. For example, the molecule of interest may be a polypeptide, a complex, a polynucleotide, a carbohydrate, a liposome, a nanoparticle, a cell, or a small molecule. It is preferred that the molecule of interest is a natural (i.e. native/endogenous) protein or a recombinantly produced protein. For example, the molecule of interest may be a therapeutic protein.

A particularly preferred molecule of interest is an antibody or an antibody fragment; e.g. if the inventive light-switchable polypeptide is a light-switchable version of protein A, protein G or protein L. The antibody may be a monoclonal antibody or a polyclonal antibody. The antibody fragment may be, e.g., a nanobody, a Fab fragment, a Fab′ fragment, a Fab′-SH fragment, a F(ab′)2 fragment, a Fd fragment, a Fv fragment, a scFv fragment, a single domain antibody or an isolated complementarity determining region (CDR). Preferably, the antibody fragment is a Fab fragment, a F(ab′)2 fragment, a Fd fragment, a Fv fragment, a scFv fragment, or a single domain antibody. The antibody or antibody fragment may be derived from human or from other species such as mouse, rat, rabbit, hamster, goat, guinea pig, ferret, cat, dog, chicken, sheep, goat, cattle, horse, camel, llama or monkey. It is prioritized that the antibody or antibody fragment is humanized or fully human. The antibody may also be a chimeric and/or bispecific antibody. The antibody may be, for example, trastuzumab.

Herein, the terms “polypeptide”, “peptide”, “oligopeptide” and “protein” are used interchangeably and relate to a molecule that encompasses at least one chain of amino acids, wherein the amino acid residues are linked by peptide (amide) bonds. Herein the terms “peptide”, “oligopeptide”, “polypeptide” and “protein” also include molecules with modifications, such as phosphorylation, ubiquitination, sumolyation, a midation, acetylation, acylation, covalent attachment of fatty acids (e.g., C6-C18), attachment of proteins such as albumin, glycosylation, biotinylation, PEGylation, addition of an acetomidomethyl (Acm) group, ADP-ribosylation, alkylation, carbamoylation, carboxyethylation, esterification, covalent attachment of flavin, covalent attachment of a heme moiety, covalent attachment of a nucleotide or nucleotide derivative, covalent attachment of a drug or toxin, covalent attachment of a marker (e.g., a fluorescent or radioactive marker), covalent attachment of a lipid or lipid derivative, covalent attachment of phosphatidylinositol, demethylation, formation of covalent crosslinks, formation of cystine, formation of a disulfide bond, formation of pyroglutamate, formylation, gamma-carboxylation, GPI anchor formation, hydroxylation, iodination, methylation, myristoylation, oxidation, proteolytic processing, prenylation, racemization, selenoylation, or sulfation.

Herein, the terms “peptide”, “oligopeptide”, “polypeptide” and “protein” also comprise “peptide analogs” (also called “peptidomimetics” or “peptide mimetics”). Peptide analogs/peptidomimetics replicate the backbone geometry and physico-chemical properties of biologically active peptides. Generally, peptide analogs are structurally similar to the template peptide, i.e. a peptide that has biological or pharmacological activity and that comprises naturally occurring amino acids, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc. Such peptide analogs can be prepared by methods well known in the art.

The term “amino acid” or “residue” as used herein includes both, L- and D-isomers of the naturally occurring amino acids that are encoded by nucleic acid sequences as well as of other amino acids (e.g., non-naturally-occurring amino acids, amino acids which are not encoded by nucleic acid sequences, synthetic amino acids, non-proteinogenic amino acids etc.). Examples of naturally occurring amino acids are alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y) and valine (Val; V). Naturally occurring non-genetically encoded amino acids and synthetic amino acids include, for example, selenocysteine, 3′-carboxyphenylazophenylalanine, 4′-carboxyphenylazophenylalanine, β-alanine, 3-aminopropionic acid, 2,3-diamino propionic acid, α-aminoisobutyric acid (Aib), 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine (Nie), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, β-2-thienylalanine, methionine sulfoxide, L-homoarginine (Harg), N-acetyl lysine, 2-amino butyric acid, 2-amino butyric acid, 2,4,-diaminobutyric acid, p-aminophenylalanine, p-acetylphenylalanine, N-methylvaline, homocysteine, homoserine, cysteic acid, ε-amino hexanoic acid, δ-amino valeric acid, 2,3-diaminobutyric acid etc. Further non-natural amino acids are β-amino acids (β3 and β2), homo-amino acids, 3β-substituted alanine derivatives, ring-substituted phenylalanine and tyrosine derivatives, linear core amino acids, and N-methyl amino acids.

In accordance with the present invention, the terms “nucleic acid molecule”, “oligonucleotide”, and “polynucleotide” are used interchangeably and include DNA, such as cDNA, genomic DNA, plasmid DNA, viral DNA, fragments of DNA prepared by restriction digest, synthetic DNA prepared e.g. by automated DNA synthesis or by amplification via polymerase chain reaction (PCR), and RNA. It is understood that the term “RNA” as used herein comprises all forms of RNA including mRNA, rRNA, tRNA, siRNA, muRNA, viral RNA, synthetic RNA and the like.

Both single-strand as well as double-strand nucleic acid molecules are encompassed by the terms “nucleic acid molecule”, “oligonucleotide”, and “polynucleotide”. Further included are nucleic acid mimicking molecules known in the art such as synthetic or semi-synthetic derivatives of DNA or RNA and mixed polymers. Such nucleic acid mimicking molecules or nucleic acid derivatives according to the invention include a phosphorothioate nucleic acid, a phosphoramidate nucleic acid, a 2′-O-methoxyethyl ribonucleic acid, a morpholino nucleic acid, a hexitol nucleic acid (HNA), a peptide nucleic acid (PNA) and a locked nucleic acid (LNA).

Herein, the term “small molecule” relates to any molecule with a molecular weight of 2000 Daltons or less, preferably of 900 Daltons or less, more preferably of 500 Daltons or less. Herein, a small molecule may be organic or inorganic, preferably organic. It is further preferred that the small molecule can diffuse across cell membranes so that it can reach intracellular sites of action. In addition, the small molecule as defined herein may have oral bioavailability.

The term “complex” is commonly known in the field of biochemistry and relates to an entity composed of molecules in which the constituents maintain much of their chemical identity. For example, typical complexes are the antibody/antigen complex, receptor/hormone complex, receptor/cytokine complex, enzyme/substrate complex, metal/chelate complex streptavidin/biotin complex or the Strep-Tactin/Strep-tag complex.

In accordance with the present invention, if the molecule of interest is an immunoglobulin (i.e. an antibody) or a fragment thereof, then it can be isolated and/or purified simply by using a light-switchable version of protein A, protein G or protein L. However, binding of the molecule of interest to the light-switchable polypeptide can also be achieved by fusing the molecule of interest with an affinity tag. For example, the molecule of interest may be fused with a Strep-tag, a Strep-tag II and/or a myc-tag.

A further aspect of the present invention relates to an affinity matrix comprising the light-switchable polypeptide as defined herein. For example, the affinity chromatography matrix of the present invention may be prepared by coupling the light-switchable polypeptide provided herein to a conventional affinity chromatography matrix (e.g. NHS-activated Sepharose 4B). For example, 0.1 to 50 mg, preferably 0.5 to 40 mg, more preferably 1 to 25 mg, even more preferably 2.5 to 10 mg, and most preferably about 5 mg or about 10 mg of the light-switchable polypeptide per mL of swollen gel may be applied. Preparing a conventional affinity chromatography matrix is commonly known in the art and described, e.g., in Schmidt & Skerra 1994 J. Chromatogr. A 676: 337-345.

Another aspect of the present invention relates to an affinity chromatography column comprising the affinity matrix of the invention. In this affinity chromatography column the matrix may be contained in a light-transmissible tube or vessel; and/or in a tube or vessel comprising at least one fiberoptic. Thus, the light may reach the matrix either by passing through the wall of the light-transmissible tube or vessel or via at least one fibreoptic. The light-transmissible tube or vessel may be made of glass or plastic. The affinity chromatography column of the present invention may, for example, be prepared by packing a UV-transparent column in a glass capillary (e.g. 0.7 mm inner-diameter), optionally equipped with a fritted glass or plastic base at one or both ends, with the chromatography matrix (e.g. 20 μL). Also, the affinity chromatography column of the present invention may be prepared by packing a UV-transparent column in a larger glass or plastic tube, e.g. having a 5 mm to 50 mm (such as 7 mm or about 10 mm or about 25 mm) inner-diameter.

The affinity chromatography column provided herein comprising the light-switchable polypeptide of the invention can form part of an affinity chromatography apparatus. Thus, a further aspect of the invention relates to an affinity chromatography apparatus comprising
(i) the affinity chromatography column of the invention;
(ii) a light source;
(iii) a housing; and
(iv) an electronic interface.

In addition, the affinity chromatography apparatus comprises elements that are commonly found in commercial chromatography systems such as a controllable pump, tubing and, optionally, a UV detector (or e.g. light scattering detector or refractive index detector) and fraction collector.

This affinity chromatography apparatus may be configured for use at the laboratory scale or for automated high throughput isolation and/or purification of a desired molecule of interest. For example, such automated high throughput processes are of particular relevance for the isolation of recombinantly produced biological drug candidates or therapeutic proteins. Also, the isolation of biomolecules, in particular proteins, nucleic acids, carbohydrates, and live cells for purposes of research or biomedical application is envisaged.

The light source of the affinity chromatography apparatus provided herein enables irradiation of the light-switchable polypeptide with the desired wavelength(s) of light. For example, the light source may comprise or consist of one, two or more light-emitting diode(s), LED(s), fluorescent tube(s), and/or laser(s). The wavelength of the light that is emitted by the light source may be controlled electronically. It is envisaged in the context of the inventive affinity chromatography apparatus that the wavelength(s) of the light that is emitted by the light source is switchable. For example, the wavelength(s) may easily be changed by means of the same or a second set of LED(s), fluorescent tube(s), and/or laser(s).

One aspect of the present invention relates to the affinity chromatography apparatus provided herein wherein the wavelength(s) of the light that is emitted by the one, two or more light source(s) is switchable from visible light (having about 400 to 530 nm, e.g., 400 to 500 nm, preferably 405 to 470 nm, more preferably 410 to 450 nm, and most preferably about 430 nm) to UV light (having 300 to 390 nm, preferably 310 to 370 nm, more preferably 320 to 350 nm, and most preferably about 330 nm; or alternatively about 365 nm) and vice versa.

An affinity chromatography procedure according to the present invention may, for example, be performed as follows. At first, the column may be equilibrated with running buffer, e.g. PBS or TBS (100 mM Tris-HCl pH 8.0, 100 mM NaCl), once (optionally, to elute remaining bound ligand from a previous application) under UV irradiation (e.g. 300 to 390 nm, such as about 365 nm) and once under irradiation with visible light (e.g. 400 to 500 nm or >500 nm or daylight, to trigger the trans configuration). Then, the liquid phase comprising the molecule of interest (e.g. a cell extract or a culture supernatant) may be applied to the column and the column may be washed with running buffer. Sample (i.e. liquid phase) application and washing steps are preferably conducted under irradiation with visible light (e.g. 400 to 500 nm or >500 nm or daylight). Elution of the bound molecule of interest is preferably triggered by irradiation with UV light (e.g. 300 to 390 nm, such as about 365 nm, to trigger the cis configuration). To this end, buffer flow may be stopped for a certain period of time (e.g. 0 to 60 min) while applying UV light; then the molecule of interest may be eluted with running buffer. Alternatively, the molecule of interest may be eluted with running buffer under continuous irradiation with UV light.

In another aspect of the invention the affinity chromatography procedure may be performed as follows. At first, the column may be equilibrated with running buffer, e.g. PBS or TBS, once (optionally, to elute remaining bound ligand from a previous application) under irradiation with visible light (e.g. 400 to 500 nm or >500 nm or daylight), and once under UV irradiation (e.g. 300 to 390 nm, such as about 365 nm, to trigger the cis configuration). Then, the liquid phase comprising the molecule of interest (e.g. a cell extract or a culture supernatant) may be applied to the column and the column may be washed with running buffer. Sample (i.e. liquid phase) application and washing steps may be conducted under irradiation with UV light (e.g. 300 to 390 nm, such as about 365 nm). Elution of the bound molecule of interest may be triggered by irradiation with visible light (e.g. 400 to 500 nm or >500 nm or daylight, to trigger the trans configuration). To this end, buffer flow may be stopped for a certain period of time (e.g. 0 to 60 min) while applying visible light; then the molecule of interest may be eluted with running buffer (either under visible light or in the dark). Alternatively, the molecule of interest may be eluted with running buffer. In this regard, elution may either be performed under continuous irradiation with visible light; or elution may be started under visible light (to trigger the trans configuration) and subsequently performed in the dark.

As described herein above and below in the appended Examples, using the light-switchable polypeptide provided herein for affinity chromatography entails a variety of advantages, as it decreases costs and time while increasing purity of the isolated molecule of interest.

However, there are also other areas of application of the light-switchable polypeptide of the present invention. For example, the light-switchable polypeptide may be used in analytical tests, e.g. in an ELISA, or in connection with (para)magnetic beads or plastic particles coated with the light-switchable polypeptide. In addition, the light-switchable polypeptide of the present invention may be used in a surface plasmon resonance (SPR) assay in order to test the binding properties of a compound of interest (e.g. a newly designed drug) towards its target (or vice versa). Therefore, an SPR chip comprising the light-switchable polypeptide (e.g. within a matrix) may be used. Such an SPR chip can be used several times and has short regeneration times when using UV light for the desorption of the compound of interest and/or target.

All publications cited herein, including all scientific and patent literature, are incorporated by reference in their entirety.

The present invention is further described by reference to the following non-limiting Figures and Examples.

The Figures show:

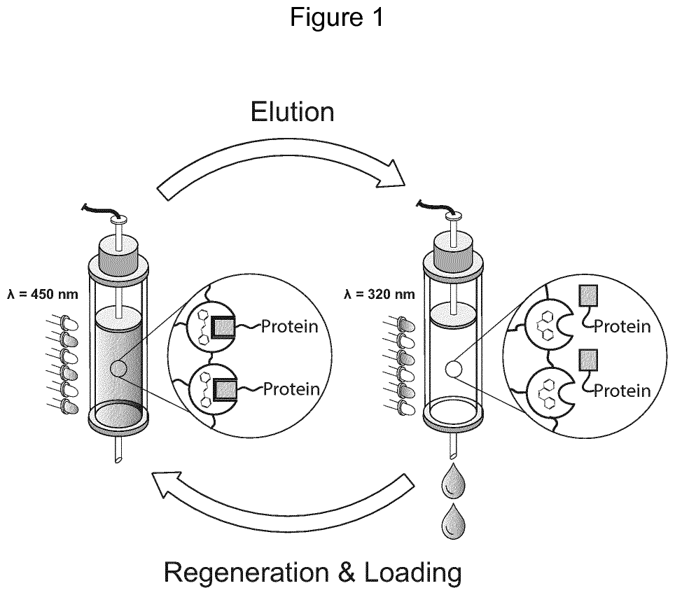

FIG. 1: Principle of light-controlled affinity chromatography for protein purification.

The affinity column contains a chromatography matrix with an immobilized light-switchable binding protein (affinity molecule). A protein solution (e.g. a cell extract) is applied to the column and, once the protein of interest (e.g. carrying an affinity tag such as the Strep-tag II) has bound to the affinity matrix, contaminating proteins and biomolecules (possibly including host cell and/or buffer components of any kind) are washed away. By irradiation with mild UV light at 365 nm the conformation of the binding protein in the affinity matrix is changed in such a way as to lose binding activity towards the protein of interest and/or affinity tag, thus effecting instant elution (under constant buffer flow). To regenerate the column afterwards, green light >530 nm is applied, which relaxes the affinity matrix to the ground state.

FIG. 2: Synthesis of the photo-switchable non-natural amino acid 4′-carboxyphenylazophenylalanine alias 4-[(4-carboxyphenyl)azo]-L-phenylalanine based on azo-benzene.

(A) Preparation of 4-[(4-carboxyphenyl)azo]-L-phenylalanine (Caf; 7) via Boc- or Fmoc-protected intermediates, also illustrating the reversible isomerization from the trans to the cis configuration triggered by light at different wavelengths. (B)¹H-NMR spectrum of 4-[(4-carboxyphenyl)azo]-L-phenylalanine (7) in D₂O. (C)¹³C-NMR spectrum of 4-[(4-carboxyphenyl)azo]-L-phenylalanine (7) in D₂O.

FIG. 3: Synthesis of the photo-switchable non-natural amino acid 3′-carboxyphenylazophenylalanine alias 4-[(3-carboxyphenyl)azo]-L-phenylalanine based on azo-benzene.

(A) Preparation of 4-[(3-carboxyphenyl)azo]-L-phenylalanine (11), also illustrating the reversible isomerization from the trans to the cis configuration triggered by light at different wavelengths.

(B) ¹H-NMR spectrum of 4-[(3-carboxyphenyl)azo]-L-phenylalanine (11) in D₂O. (C) ¹³C-NMR spectrum of 4-[(3-carboxyphenyl)azo]-L-phenylalanine (11) in D₂O.

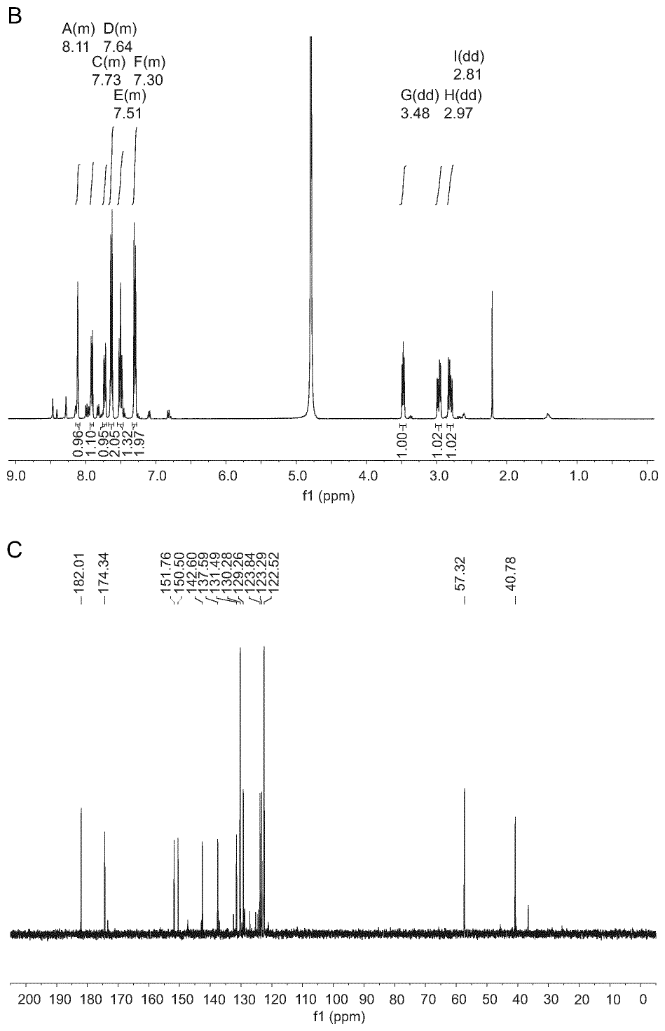

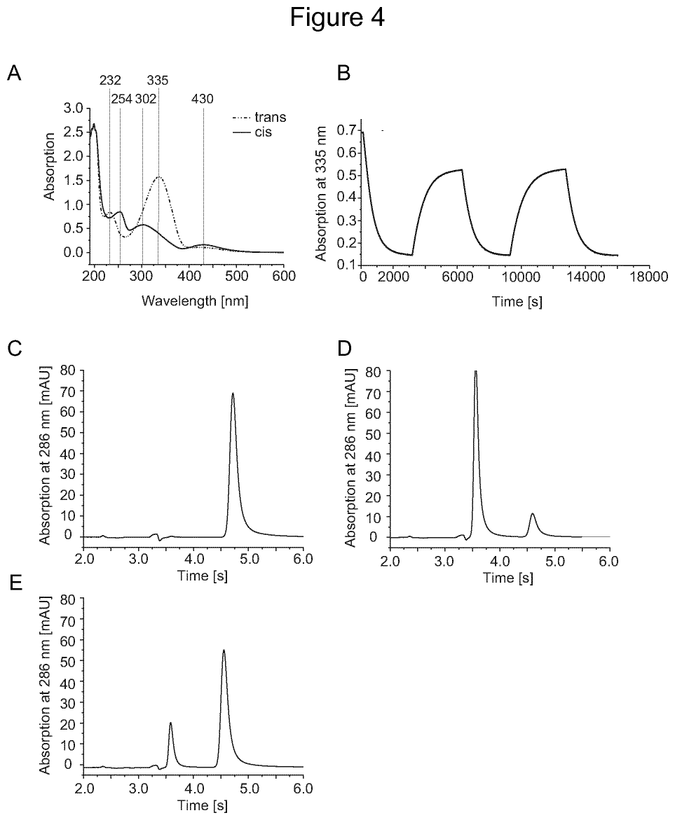

FIG. 4: Reversible photo-switching (isomerization) of Caf with alternating 365 nm (UV) versus 530 nm (green) LED photo-irradiation cycles.

(A) UV spectrum of the non-natural amino acid Caf in water (solid line: trans isomer; dotted line: cis isomer). (B) Reversible photo-switching between trans and cis configurations as visualized via changes in absorbance at approximately 340 nm (transition π→π*) over 3 cycles. High absorption at 335 nm indicates the trans configuration whereas low absorption at 335 nm indicates the cis configuration of Caf, cf. panel (A). (C-D) HPLC chromatograms of 7, absorption at λ=286 nm before irradiation (C), after irradiation with UV light (D) and after irradiation with green light (E). The chromatogram in panel (C) reveals essentially pure trans isomer; the chromatogram in panel (D) reveals mostly cis isomer, with the trans isomer as minor species; the chromatogram in panel (E) reveals mostly trans isomer, with the cis isomer as minor species.

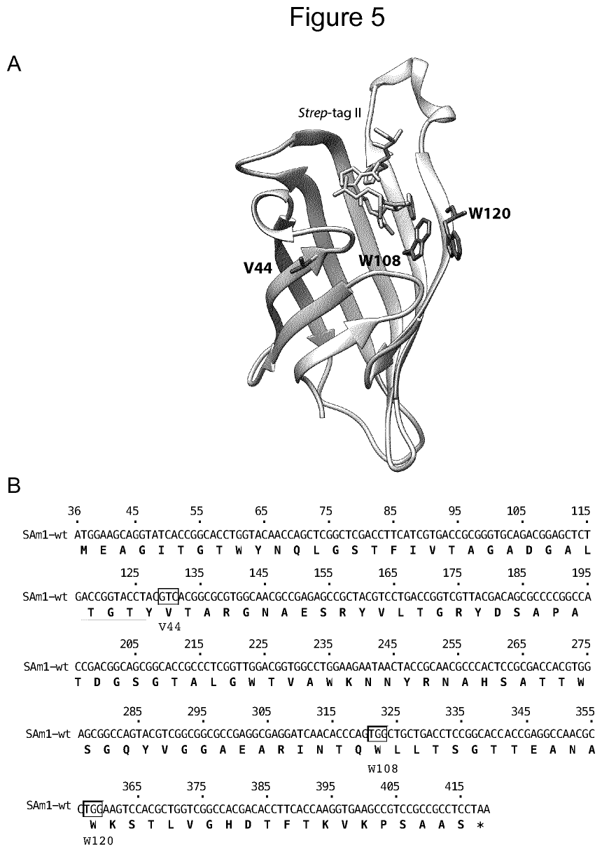

FIG. 5: Structural and sequence overview of SAm1^{Caf} variants.

(A) Crystal structure of the complex between streptavidin mutant 1 (SAm1, Strep-Tactin) and the Strep-tag II with highlighted residues V44, W108 and W120 (PDB entry 1KL3). All positions substituted with Caf were investigated for their potential to interfere with binding (reduce affinity) of the Strep-tag II in the cis configuration of the non-natural amino acid Caf but preserve binding in its (trans) ground state. Among these positions investigated for introduction of Caf as a light-responsive element, V44 and W120 are less preferred. (B) Nucleic and amino acid sequence of SAm1 with positions for Caf incorporation (in translation/suppression of an amber stop codon) highlighted.

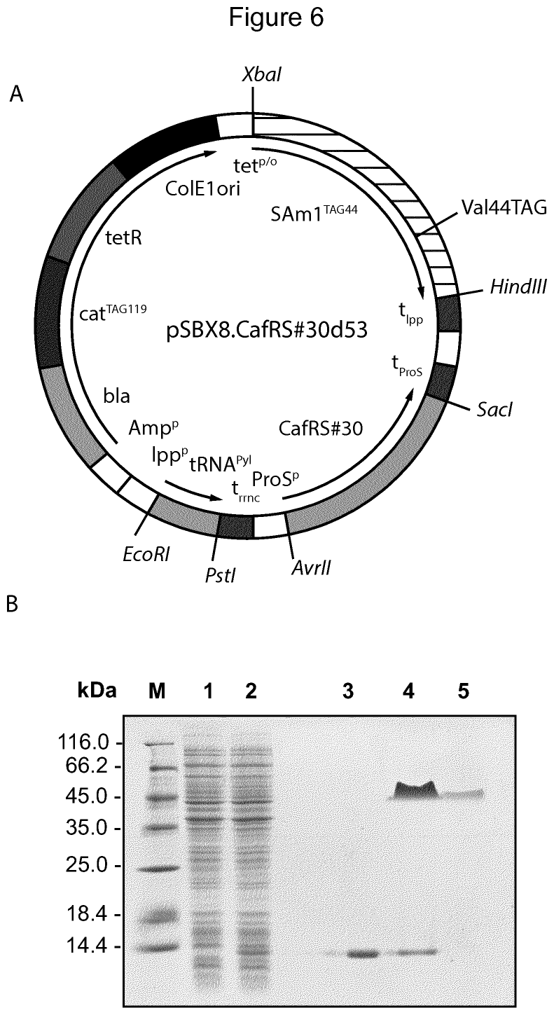

FIG. 6: Expression, purification and refolding of SAm1^{Caf108}.

(A) Plasmid map of pSBX8.CafRS#30d53 (SEQ ID NO: 55). (B) Purification and refolding of the recombinant core streptavidin mutant SAm1 carrying Caf at position 108. An SDS-PAGE (15%) gel stained with Coomassie brilliant blue is shown with samples from different stages during preparation of the recombinant protein. Lanes: 1, total E. coli protein before induction of gene expression; 2, total cell protein 12 h after induction; 3, protein solution after renaturation of the inclusion bodies and CEX purification; 4, same sample as in 3, but without heat treatment prior to SDS-PAGE. Under these conditions the core streptavidin tetramer remains intact (Bayer et al. 1990 Methods Enzymol. 184: 80-89). Thus the correctly folded state of the recombinant mutant streptavidin in the final preparation was confirmed whereas small amounts of monomeric (likely non-functional) streptavidin were still present after refolding (lane 4). Lane 5 shows the same sample as lane 4, but at lower concentration.

FIG. 7: Reversible binding of the PhoA/Strep-tag II fusion protein to streptavidin mutants/variants modified with a light-switchable amino acid in an ELISA.

(A) ELISA setup for screening streptavidin mutants having reversible binding activity toward the Strep-tag II peptide in response to UV light. (B) Screening for light-induced desorption of purified PhoA/Strep-tag II from SAm1 and its variants Caf44, Caf108, Caf120. All tested streptavidin mutants showed good affinity for the PhoA/Strep-tag II fusion protein, giving rise to comparable signals as obtained with SAm1 for those samples illuminated with visible light. In contrast, a clear decrease in remaining enzyme activity was observed after irradiation with UV light at 365 nm for the streptavidin variant SAm1^{Caf108}. This indicates reduced affinity of the streptavidin variant SAm1^{Caf108} for PhoA carrying the Strep-tag II upon light-induced switching of Caf to the cis configuration.

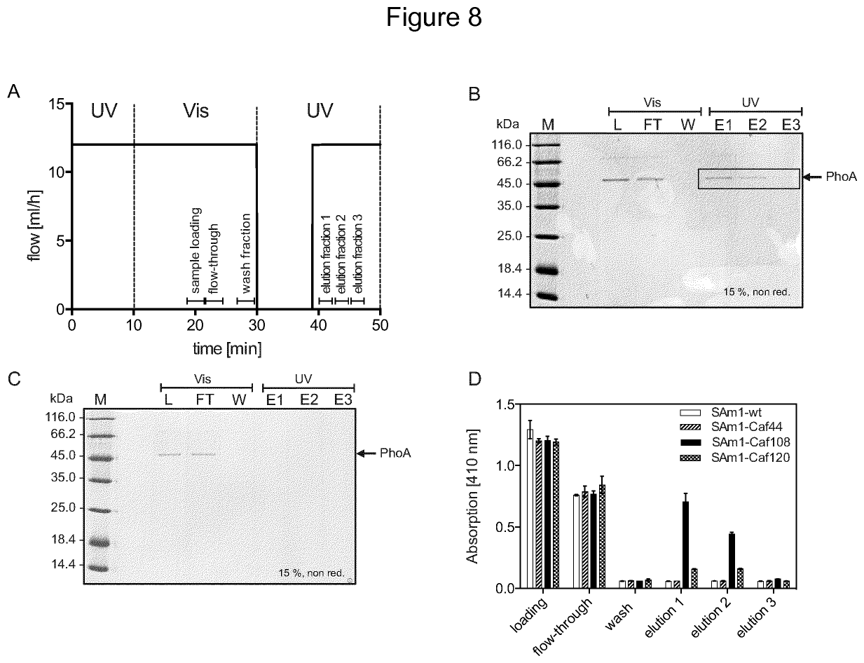

FIG. 8: Light-induced desorption of PhoA/Strep-tag II from a functionalized affinity matrix.

(A) Flow profile observed for the chromatography column containing 20 μL sepharose with immobilized SAm1^{Caf108}. Irradiation with green LED light (530 nm) or mild UV light (365 nm) was performed as indicated. (B) Samples of each fraction (10 μL) collected from the SAm1^{Caf108} column were analyzed by SDS-PAGE. Lanes: M, molecular size standard; L, loaded sample; FT, flow-trough; W, wash; E1-E3, elution fractions. (C) 15% SDS-PAGE of samples from the SAm1 column. (D) Quantification of PhoA/Strep-tag II fusion protein in the collected fractions (loaded sample, flow-through, wash, elution 1-3) via PhoA enzyme assay. In contrast to the unmodified streptavidin mutein (SAm1), the affinity column comprising SAm1^{Caf108} reveals light-dependent elution of the bound PhoA/Strep-tag II fusion protein.

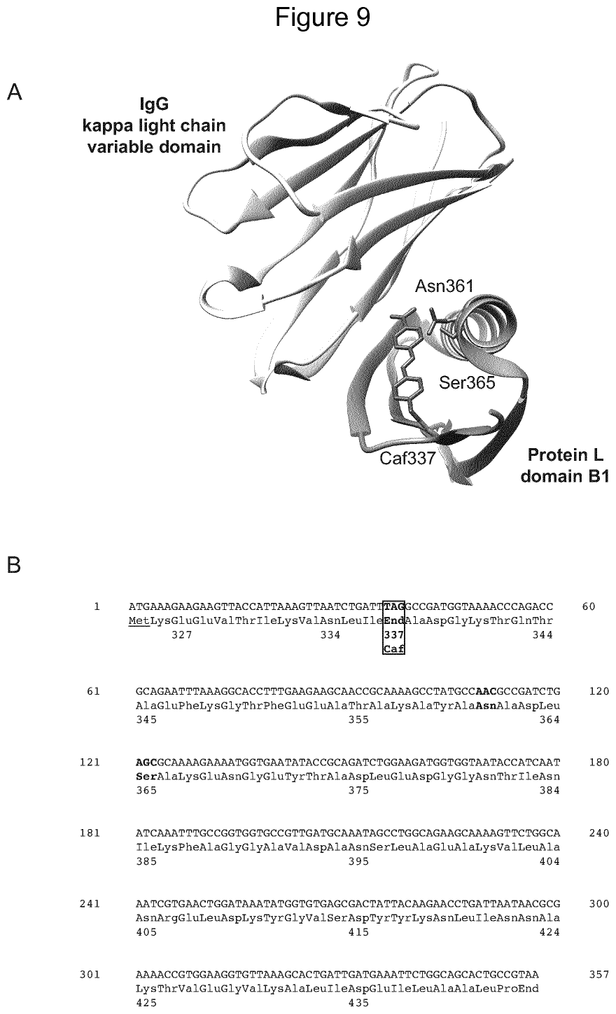

FIG. 9: Structural and sequence overview of ProtL^{Caf} variants.

(A) Crystal structure of the complex between the trastuzumab Fab fragment and the B1 domain of protein L with Caf337 as well as mutated residues Asn361 and Ser365 shown as sticks (UniProt accession code Q51918; this corresponds to positions 29, 53 and 57 in the PDB entry 4 HKZ). Position 337 is suitable for substitution with Caf with the goal of achieving a different affinity towards an immunoglobulin depending on the cis or trans configuration of the light-responsive non-natural amino acid. (B) Nucleic acid and amino acid sequence of the ProtL^{Caf}-ABD fusion protein with position 337 for Caf incorporation (in translation/suppression of an amber stop codon) highlighted. Methionine (underlined) was added as a start codon in comparison to SEQ ID NO: 20.

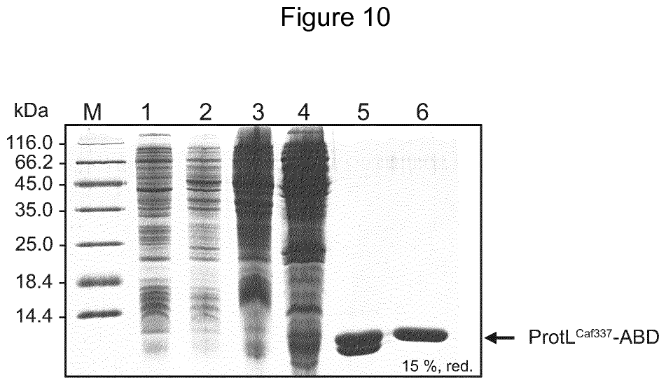

FIG. 10: Expression and purification of the ProtL^{Caf337}-ABD fusion protein

An SDS-PAGE (15%) gel stained with Coomassie brilliant blue shows samples from different stages during preparation of the recombinant protein. Lanes: 1, total E. coli protein before induction of gene expression; 2, total cell protein 12 h after induction; 3, insoluble fraction of the whole cell extract; 4, soluble supernatant of the whole cell extract; 5, elution fraction from HSA affinity chromatography; 6, ProtL^{Caf337}-ABD after CEX purification.

FIG. 11: Reversible binding of an immunoglobulin to a ProtL^{Caf}-ABD fusion protein modified with a light-switchable amino acid in an ELISA.

(A) Schematic ELISA setup for screening of ProtL^{Caf} variants having reversible binding activity towards immunoglobulins in response to UV light. (B) Exemplary assay for light-induced desorption of a mouse anti-6×His antibody (immunoglobulin) conjugated with alkaline phosphatase (AP) from protein L domain B1 and its variant Caf337 (both fused with the ABD and adsorbed to an HSA-coated microtiter plate). In its ground state, the tested Caf337 variant showed high affinity for the IgG (right, hollow circles), even though with lower signals than observed for the unmodified Protein L domain (left, hollow circles). In contrast, a clear decrease in remaining activity of bound Ig-AP conjugate was observed after irradiation with UV light at 365 nm (solid circles) only for the ProtL^{Caf337} variant, indicating that the light-induced formation of the cis isomer of Caf leads to specific dissociation between the light-switchable Protein L domain and the immunoglobulin (Ig).

Error bars indicate standard deviations from triplicate measurements. Curve fit of the ELISA data (Voss & Skerra 1997 Protein Eng 10:975-82) for the ProtL^{Caf337} in its ground state revealed a dissociation constant of approximately 140 nM for the complex with the anti-6×His antibody, corresponding to a high affinity. The signal intensities observed after irradiation with UV light were too low to deduce a dissociation constant, indicating strong loss in affinity of the light-switchable polypeptide (hence, these data were fitted by a straight line).

The Examples illustrate the invention.

### EXAMPLE 1: SYNTHESIS OF 4-[(4-CARBOXYPHENYL)AZO]-L-PHENYLALANINE (CAF)

The preparation of 4-[(4-carboxyphenyl)azo]-L-phenylalanine (Caf; 7) (herein also called 4′-carboxyphenylazophenylalanine) was previously reported (Nakayama et al. 2005 Bioconjug. Chem. 16: 1360-1366). However, here a more convenient protocol for the synthesis of Caf which is illustrated in FIG. 2A is provided. Commercially available Fmoc- or Boc-protected 4-amino-L-phenylalanine (3 and 4) was reacted with 4-nitrosobenzoic acid (2), which was prepared from 4-aminobenzoic acid (1) by oxidation with oxone (2 KHSO₅+KHSO₄+K₂SO₄). The resulting diazo intermediate 5 was deprotected with piperidine, whereas the alternative intermediate 6 was deprotected with HCl in dioxane, in both cases yielding the desired amino acid 7.

### Step 1: Synthesis of 4-Nitrosobenzoic Acid (2)

Compound 2 was prepared according to a published procedure (Priewisch & Ruck-Braun 2005 J. Org. Chem. 70: 2350-2352). 4-Aminobenzoic acid (15 g, 109 mmol) was suspended in 180 ml dichloromethane. A solution of oxone (134.5 g, 219 mmol) in 675 ml H₂O was added and the mixture was stirred for 1.5 h at room temperature. The precipitate was filtered off, washed thoroughly with H₂O, dried at air and then over P₂O₅. 4-Nitrosobenzoic acid (2) was obtained as a yellow solid (16 g, 106 mmol), containing a small amount of 4-nitrobenzoic acid, and was further used without purification.

¹H NMR (400 MHz, DMSO-d6) δ=13.50 (s, 1H, COOH), 8.29-8.22 (m, 2H, aromat.), 8.05-8.00 (m, 2H, aromat.).

¹³C NMR (101 MHz, DMSO) δ=166.19 (CO), 165.00 (C aromat.), 136.53 (C aromat.), 131.02 (2× C aromat.), 120.62 (2× C aromat.).

Analytical HPLC: Column Purospher RP-8e 250×3 mm (Merck KgaA, Darmstadt, Germany), gradient 10-100% ACN in water+0.1% TFA in 30 min, flow rate 0.6 ml/min; t_{R}=14.43 min.

### Step 2a: Synthesis of N-Fmoc-4-[(4-carboxyphenyl)azo]-L-phenylalanine (5)

Compound 5 was prepared analogously to a published procedure (Priewisch & Ruck-Braun 2005 J. Org. Chem. 70: 2350-2352). 4-Nitrosobenzoic acid (2) (3 g, 19.9 mmol) was suspended in 320 ml DMSO/AcOH 1:1 (v/v) with ultrasonification, followed by addition of Fmoc-Phe(4-NH₂)—OH (3) (4 g, 9.94 mmol; Iris Biotech, Marktredwitz, Germany). The mixture was stirred for 2 d at room temperature. Then 700 ml H₂O was added and the resulting precipitate was filtered, washed with H₂O, dried at air and then over P₂O₅. The desired product 5 was obtained as a brown solid and further used without purification.

¹H NMR (400 MHz, DMSO-d6) δ=13.16 (s, 2H, 2×COOH), 8.17-8.13 (m, 2H, aromat.), 7.97-7.90 (m, 2H, aromat.), 7.88-7.81 (m, 5H, aromat., NH), 7.68-7.58 (m, 2H, aromat.), 7.56-7.48 (m, 2H, aromat.), 7.42-7.33 (m, 2H, aromat.), 7.33-7.23 (m, 2H, aromat.), 4.30 (ddd, J=10.6, 8.5, 4.5 Hz, 1H, C^{α}H), 4.25-4.19 (m, 2H, Fmoc-CH₂), 4.19-4.12 (m, 1H, Fmoc-CH), 3.23 (dd, J=13.9, 4.4 Hz, 1H, C^{β}H), 3.01 (dd, J=13.8, 10.7 Hz, 1H, C^{β}H).

¹³C NMR (101 MHz, DMSO) δ=173.14 (CO), 166.75 (CO), 155.99 (CO), 154.37 (C aromat.), 150.69 (C aromat.), 143.78 (C aromat.), 143.73 (C aromat.), 142.83 (C aromat.), 140.71 (C aromat.), 140.69 (C aromat.), 132.71 (C aromat.), 131.03 (2×C aromat.), 130.66 (2×C aromat.), 130.35 (2×C aromat.), 127.61 (2×C aromat.), 127.05 (2×C aromat.), 122.79 (2×C aromat.), 122.47 (2×C aromat.), 120.09 (2×C aromat.), 65.64 (Fmoc-CH₂), 55.19 (C^{α}), 46.61 (Fmoc-CH), 36.42 (C^{β}).

MS analysis: calc. [M-H⁺]=534.16706; found [M-H⁺]=534.15320.

Analytical HPLC: Column Purospher RP-8e 250×3 mm (Merck KgaA, Darmstadt, Germany), gradient 10-100% ACN in water+0.1% TFA over 30 min, flow rate 0.6 ml/min; t_{R}=21.16 min.

### Step 2b: Synthesis of N-Boc-4-[(4-carboxyphenyl)azo]-L-phenylalanine (6)

Compound 6 was prepared according to a published procedure (Bose et al. 2006 J. Am. Chem. Soc. 128: 388-389) Boc-Phe(4-NH₂)—OH (4) (1 g, 3.6 mmol; Bachem, Bubendorf, Switzerland) was dissolved in 50 ml AcOH. After addition of 4-nitrosobenzoic acid (2) (0.8 g, 5.4 mmol) the mixture was stirred for 24 h. The solvent was removed at reduced pressure and the remaining material was dissolved in 100 ml each of 1 M HCl (aq.) and ethyl acetate. The aqueous phase was extracted four times with 50 ml ethyl acetate. The combined organic phases were washed once with brine and dried over MgSO₄. After evaporation of the solvent 6 was obtained as a brown solid (638 mg, 1.54 mmol, 43%), which was further used without purification.

¹H NMR (500 MHz, DMSO-d6) δ=8.14 (d, J=8.3 Hz, 2H, aromat.), 7.94 (d, J=8.4 Hz, 2H, aromat.), 7.85 (d, J=7.9 Hz, 2H, aromat.), 7.49 (d, J=8.1 Hz, 2H, aromat.), 7.11 (d, J=8.4 Hz, 1H, NH), 4.22-4.13 (m, 1H, C^{α}H), 3.15 (dd, J=13.9, 4.6 Hz, 1H, C^{β}H), 2.95 (dd, J=13.8, 10.2 Hz, 1H, C^{β}H), 1.31 (s, 9H, C(CH₃)₃).

Analytical HPLC: Column Purospher RP-8e 250×3 mm (Merck KgaA, Darmstadt, Germany), gradient 10-100% ACN in water+0.1% TFA over 30 min, flow rate 0.6 ml/min; t_{R}=18.33 min.

### Step 3a: Synthesis of 4-[(4-Carboxyphenyl)azo]-L-phenylalanine (7) (Fmoc Cleavage)

Compound 5 (5 g, 9.34 mmol) was dissolved in 40 ml DMF, then 10 ml piperidine was added dropwise and the mixture was stirred for 30 min at room temperature. Addition of 450 ml 0.5 M NaHCO₃ (aq.) caused formation of a colorless precipitate, which was removed by filtration. The filtrate was acidified to pH 1-2 by addition of 6 M HCl (aq.). The precipitate was filtered off and dried at air, then over P₂O₅. Compound 7 was obtained as a brown solid (2.42 g, 7.72 mmol, 98% over 2 steps) which was used for biophysical and biochemical experiments described in Example 3 and 6 without further purification.

¹H NMR (400 MHz, D₂O) δ=7.86-7.80 (m, 2H, aromat.), 7.59-7.53 (m, 2H, aromat.), 7.53-7.47 (m, 2H, aromat.), 7.24-7.18 (m, 2H, aromat.), 3.42 (dd, J=7.5, 5.6 Hz, 1H, C^{α}H), 2.90 (dd, J=13.5, 5.6 Hz, 1H, C_{β}H), 2.73 (dd, J=13.4, 7.6 Hz, 1H, C^{β}H).

¹³C NMR (101 MHz, D₂O) δ=181.94 (CO), 174.43 (CO), 153.16 (C aromat.), 150.42 (C aromat.), 142.79 (C aromat.), 138.45 (C aromat.), 130.23 (2×C aromat.), 129.81 (2× C aromat.), 122.55 (2× C aromat.), 121.93 (2× C aromat.), 57.28 (V), 40.80 (C^{β}).

MS analysis: calc. [M-H⁺]⁻=312.09898; found [M-H⁺]⁻=312.09380.

Analytical HPLC: Column Purospher RP-8e 250×3 mm (Merck KgaA, Darmstadt, Germany), gradient 10-100% ACN in water+0.1% TFA over 30 min, flow rate 0.6 ml/min; t_{R}=10.7 min.

### Step 3b: Synthesis of 4-[(4-Carboxyphenyl)azo]-L-phenylalanine (7) (Boc Cleavage)

Compound 6 (638 mg, 1.5 mmol) was dissolved in 20 ml of approx. 2 M HCl in dioxane and stirred over night at room temperature. The precipitate was filtered off, washed with diethyl ether and dried at vacuum. Compound 7 was obtained as a brown solid (236 mg, 0.67 mmol, 44%), which was used for biophysical and biochemical experiments described in Example 3 and 6 without further purification. Analytical data were in agreement with those described in Step 3a.

### EXAMPLE 2: SYNTHESIS OF 4-[(3-CARBOXYPHENYL)AZO]-L-PHENYLALANINE (11)

4-[(3-Carboxyphenyl)azo]-L-phenylalanine (11) (herein also called 3′-carboxyphenylazophenylalanine) was synthesized in 3 steps as shown in FIG. 3A. Fmoc-protected 4-aminophenylalanine (3) was reacted with 3-nitrosobenzoic acid (9), which was prepared from 3-aminobenzoic acid (8) by oxidation with axone. Intermediate 10 was deprotected with piperidine to yield 4-[(3-carboxyphenyl)azo]-L-phenylalanine (11).

### Step 1: Synthesis of 3-Nitrosobenzoic Acid (9)

Compound 9 was prepared according to a published procedure (Priewisch & Ruck-Braun 2005 J. Org. Chem. 70: 2350-2352). 3-Aminobenzoic acid (8) (5 g, 36.5 mmol) was suspended in 100 ml DCM. After addition of a solution of oxone (44.9 g, 73 mmol) in 400 ml H₂O, the mixture was stirred for 1 h at room temperature. The precipitate was filtered off, washed thoroughly with H₂O, and dried over P₂O₅. 3-Nitrosobenzoic acid (9) was obtained as a brown solid (4.1 g, 27 mmol, 76%), containing a small amount of 3-nitrobenzoic acid, and was further used without purification.

¹H NMR (400 MHz, DMSO-d6) δ=13.52 (s, 1H, COOH), 8.41-8.35 (m, 1H, aromat.), 8.35-8.33 (m, 1H, aromat.), 8.19-8.11 (m, 1H, aromat.), 7.91-7.84 (m, 1H, aromat.).

¹³C NMR (101 MHz, DMSO) δ=166.08, 165.19, 136.26, 132.45, 130.47, 124.25, 120.98. Analytical HPLC: Column Purospher RP-8e 250×3 mm (Merck KgaA, Darmstadt, Germany), gradient 10-100% ACN in water+0.1% TFA over 30 min, flow rate 0.6 ml/min; t_{R}=14.05 min.

### Step 2: Synthesis of N-Fmoc-4-[(3-carboxyphenyl)azo]-L-phenylalanine (10)

Compound 10 was prepared analogously to a published procedure (Priewisch & Ruck-Braun 2005 J. Org. Chem. 70: 2350-2352). 3-Nitrosobenzoic acid (9) (378 mg, 2.5 mmol) was suspended in 40 ml DMSO/AcOH 1:1 with ultrasonification, followed by addition of Fmoc-Phe(4-NH₂)—OH (3) (500 mg, 1.24 mmol). The mixture was stirred for 2 d at room temperature and then 200 ml H₂O was added. The resulting precipitate was filtered, washed with H₂O, and dried over P₂O₅. Fmoc-protected amino acid 10 was obtained as a brown solid and further used without purification.

¹H NMR (400 MHz, DMSO-d6) δ=13.15 (s, 2H, 2×COOH), 8.38-8.33 (m, 1H, aromat.), 8.11 (dd, J=7.8, 1.8 Hz, 2H, aromat.), 7.93-7.79 (m, 5H, aromat., NH), 7.77-7.69 (m, 1H, aromat.), 7.63 (t, 2H, aromat.), 7.54-7.48 (m, 2H, aromat.), 7.43-7.33 (m, 2H, aromat.), 7.33-7.22 (m, 2H, aromat.), 4.28 (ddd, J=10.8, 8.5, 4.5 Hz, 1H, C^{α}H), 4.24-4.10 (m, 3H, Fmoc-CH, CH₂), 3.26-3.17 (m, 1H, C^{β}H), 3.00 (dd, J=13.8, 10.7 Hz, 1H, C^{β}H).

¹³C NMR (101 MHz, DMSO) δ=173.11 (CO), 166.72 (CO), 155.96 (C aromat.), 151.94 (CO), 150.55 (C aromat.), 143.77 (2× C aromat.), 143.71 (2× C aromat.), 142.51 (C aromat.), 140.67 (C aromat.), 136.26 (C aromat.), 132.15 (C aromat.), 130.48 (C aromat.), 130.30 (2× C aromat.), 129.95 (C aromat.), 127.59 (C aromat.), 127.04 (2× C aromat.), 125.23 (C aromat.), 125.18 (C aromat.), 122.67 (2× C aromat.), 122.22 (C aromat.), 120.08 (2× C aromat.), 65.62 (Fmoc-CH2), 55.18 (Fmoc-CH), 46.57 (C^{α}), 36.36 (C^{β}).

MS analysis: calc. [M-H⁺]⁻=534.16706; found [M-H⁺]⁻=534.15493.

Analytical HPLC: Column Purospher RP-8e 250×3 mm (Merck KgaA, Darmstadt, Germany), gradient 10-100% ACN in water+0.1% TFA over 30 min, flow rate 0.6 ml/min; t_{R}=21.6 min.

### Step 3: Synthesis of 4-[(3-Carboxyphenyl)azo]-L-phenylalanine (11)

The Fmoc-protected amino acid 10 (650 mg, 1.21 mmol) was dissolved in 12 ml DMF. After dropwise addition of 3 ml piperidine the mixture was stirred for 30 min at room temperature. Addition of 35 ml 0.5 M NaOH caused formation of a colorless precipitate, which was removed by filtration. The filtrate was acidified to pH 1-2 using 6 M HCl (aq.). The resulting precipitate was removed by filtration and dried at air, then over P₂O₅. Amino acid 11 was obtained as a brown solid (361 mg, 1.15 mmol, 83% over 2 steps) and was used for biophysical experiments described in Example 3 without further purification.

¹H NMR (400 MHz, D₂O) δ=8.14-8.08 (m, 1H, aromat.), 7.94-7.89 (m, 1H, aromat.), 7.76-7.70 (m, 1H, aromat.), 7.67-7.60 (m, 2H, aromat.), 7.55-7.47 (m, 1H, aromat.), 7.34-7.27 (m, 2H, aromat.), 3.48 (dd, J=7.4, 5.6 Hz, 1H, C^{α}H), 2.97 (dd, J=13.5, 5.6 Hz, 1H, C^{β}H₂), 2.81 (dd, J=13.5, 7.5 Hz, 1H, C^{β}H₂).

¹³C NMR (101 MHz, D₂O) δ=182.01 (CO), 174.34 (CO), 151.76 (C aromat.), 150.50 (C aromat.), 142.60 (C aromat.), 137.59 (C aromat.), 131.49 (C aromat.), 130.28 (2× C aromat.), 129.26 (C aromat.), 123.84 (C aromat.), 123.29 (C aromat.), 122.52 (2× C aromat.), 57.32)(C°, 40.78 (C^{β}).

MS analysis: calc. [M-H⁺]⁻=312.09898; found [M-H⁺]⁻=312.09760.

Analytical HPLC: Column Purospher RP-8e 250×3 mm (Merck, Darmstadt, Germany), gradient 10-100% ACN in water+0.1% TFA over 30 min, flow rate 0.6 ml/min; t_{R}=11.3 min.

### EXAMPLE 3: LIGHT-INDUCED ISOMERIZATION OF 4-[(4-CARBOXYPHENYL)AZO]-I-PHENYLALANINE (CAF)

### Analysis by Spectroscopy

The UV-VIS absorption spectrum of azobenzene reveals two characteristic absorption bands corresponding to π→π* and n→π* electronic transitions, which differ in amplitude and precise location of the absorption maximum (λ) for the trans and cis configuration. The electronic transition π→π* is usually in the near UV region around 340 nm (Sension et al. 1993 J. Chem. Phys. 98: 6291-6315) whereas the electronic transition n→π* is usually located in the visible (VIS) region around 420 nm and is due to the presence of unshared electron pairs of the nitrogen atoms (Naegele et al. 1997 Chem. Phys. Lett. 272: 489-495). To examine whether the synthesized non-natural amino acid Caf (7) can respond to photoswitching induced by UV light, the compound was subjected to alternating irradiation cycles. In a typical experiment, 0.5 ml of a 30 μM aqueous solution was placed in a quartz cuvette with 1 cm optical pathlength. Then the sample was irradiated for 30 min from the top using a UV LED (NS355L-5RLO; Nitride Semiconductors, Tokushima, Japan) with 353 nm or a green LED (LL-504PGC2E-G5-2CC; Lucky Light Electronics, Hongkong, China) with 520 nm emitting wavelength. The change in intensity of the π-π* band at around 340 nm corresponding to the trans/cis isomerization (FIG. 4A) was monitored with a computer controlled photometer (Ultrospec 2100 pro, Amersham Biosciences). Closer examination revealed reproducible changes in absorbance at about 340 nm over 3 cycles, consistent with reversible photoswitching between the trans (high absorbance at 340 nm) and cis (low absorbance at 340 nm) configuration of the azo compound (FIG. 4B).

### Analysis by HPLC

500 μl of a 60 μM solution of Caf (7) in water was placed in a 1.5 ml HPLC vial (Screw neck vial N9, amber glass, 11.6×32 mm; Macherey Nagel, Duren, Germany) and irradiated with a UV LED (λ=353 nm, NS355L-5RLO; Nitride Semiconductors, Tokushima, Japan) for 30 min directly from the top. Before and after irradiation, a 20 μl sample of the solution was withdrawn and analyzed by HPLC on a Purospher RP-8e 250×3 mm column (Merck), applying a concentration gradient of 10-12% acetonitrile (ACN) in 50 mM NH₄OAc buffer pH 8 over 10 min (flow rate 0.6 ml/min). Another sample was analyzed in the same manner after irradiation with green LED light (λ=520 nm, LL-504PGC2E-G5-2CC, Lucky Light Electronics, Hongkong, China). FIG. 4 shows the corresponding chromatograms with absorbance at λ=286 nm (wavelength at which trans-(7) and cis-(7) show the same molar extinction coefficient, allowing direct comparison of peak integrals). The chromatograms reveal that the cis and trans isomers of (7) can be separated by HPLC (cis-(7) t_{R}=3.6 min, trans-(7) t_{R}=4.6 min). Prior to irradiation in the ground state, only energetically favored trans-(7) occurs (FIG. 4C). Irradiation with UV light (365 nm) causes an increase in the proportion of cis-(7), here up to 86% (FIG. 4D), which can be reversed by irradiation with green light (λ=520 nm), thus recovering the ground state (FIG. 4E) via photochemical reisomerization. However, it should be taken into account that also during HPLC analysis reisomerization of cis-(7) to trans-(7) takes place, so the proportion of cis-(7) after irradiation with UV light might actually be higher than indicated by HPLC chromatograms. Thus, if the light-switchable polypeptide of the present invention is applied for an affinity chromatography procedure, and the trans configuration corresponds to the high affinity state whereas the cis configuration corresponds to the low affinity conformation, then the highest degree of binding and the highest degree of elution of the molecule of interest takes place at 430 nm and 330 nm, respectively. However, conventional light sources usually provide light having wavelengths that are around 530 nm (visible light) and 365 nm (UV light). Therefore, also light providing these wavelengths (i.e. around 530 nm and/or around 365 nm) may be used in accordance with the present invention.

### EXAMPLE 4: SELECTION OF A PYLRS VARIANT SPECIFIC FOR 4-[(4-CARBOXYPHENYL)AZO]-L-PHENYLALANINE (CAF)

The biosynthesis of proteins containing a photo-switchable non-natural amino acid such as 4-[(4-carboxyphenyl)azo]-L-phenylalanine (Caf) opens the way to novel light-controllable biomolecular reagents for biophysical, structural or biochemical research as well as biotechnological and biopharmaceutical applications. To develop an orthogonal pair of suppressor tRNA and amino-acyl tRNA synthetase (aaRS) for the co-translational site-specific incorporation of Caf in a recombinant protein produced in E. coli, the pyrrolysyl-tRNA synthetase (PylRS) from the methanogenic archaeon Methanosarcina barkeri (Mb) (James et al. 2001 J. Biol. Chem. 276: 34252-34258) and its cognate tRNA^{Pyl} that specifically recognizes and suppresses the amber stop codon (Fekner & Chan 2011 Curr. Opin. Chem. Biol. 15:387-91) were employed.

To select a mutant aaRS specific for the non-natural amino acid substrate Caf, a previously described one-plasmid system (Kuhn et al. 2010 J. Mol. Biol. 404: 70-87) encoding both the aaRS and the cognate tRNA was adapted to PylRS. The modified plasmid, pSBX8.101d58 (SEQ ID NO: 23), encodes a PylRS derived from Mb and the cognate suppressor tRNA^{Pyl} (FIG. 5A). Cloned on the same plasmid, a chloramphenicol-resistance reporter gene equipped with an amber stop codon (cat^{UAG112}; SEQ ID NO: 24) served to select highly active aaRS variants (conferring Cam resistance), and a fluorescent reporter gene equipped with another amber stop codon (eGFP^{UAG39}; SEQ ID NO: 25) was used in conjunction with fluorescence-activated cell sorting (FACS) to screen for variants exhibiting the desired amino acid specificity. By applying alternating cycles of positive and negative FACS combined with dead/live selection on LB agar plates supplemented with Cam in the presence or in the absence of the foreign amino acid, respectively, a mutated aaRS (dubbed CafRS) with high specificity for Caf incorporation was selected.

The mutation Tyr349F has been described to increase the in vivo suppression activity of Mb PylRS for non-natural amino acids (Yanagisawa et al. 2008 Chem. Biol. 15: 1187-1197) and, therefore, this position was fixed to Phe in all libraries. The mutation was introduced into the PylRS wild-type gene (SEQ ID NO: 26) using the QuikChange site-directed mutagenesis kit (Agilent, Waldbronn, Germany) with a pair of suitable PCR primers (SEQ ID NO: 27 and 28), resulting in the variant PylRS#1 (SEQ ID NO: 29).

To evolve a mutant synthetase specific for the non-natural amino acid Caf, a first synthetase library (CafRS#0-R5) based on PylRS#1 was generated by fully randomizing five positions (M309, Asn311, Cys313, Met315 and Trp382) in the active site using NNS degenerate primers in a two-step assembly PCR approach. Site-directed saturation mutagenesis was carried out using the Q5 DNA polymerase PCR kit (New England Biolabs, Ipswich, Mass., USA) with the PylRS#1 gene (SEQ ID NO: 29) as template. First, two overlapping PCR fragments were prepared, each using a pair of forward and reverse primers (forward primer 1: SEQ ID NO: 30; forward primer 2: SEQ ID NO: 31; reverse primer 1: SEQ ID NO: 32; reverse primer 2: SEQ ID NO: 33). All primers were supplied by MWG Eurofins (Ebersberg, Germany).

The two randomization reactions were performed under the same conditions in a 50 μL reaction mixture comprising 1× Q5 buffer, 200 μM of each dNTP and 0.5 U Q5 DNA polymerase. The mixture was denatured for 10 s at 98° C., annealed for 30 s at 64° C., and a linear polymerase reaction was then performed for 30 s at 72° C. After 35 cycles, an enzymatic digest with DpnI was performed at 37° C. for 2 h to remove the bacterial template. Both amplified DNA fragments were purified via agarose gel purification using the Gel Extraction Kit (Qiagen, Hilden, Germany) and assembled in a second PCR reaction. To this end, 200 ng of both fragments were mixed in a 50 μL Q5 DNA polymerase reaction mixture comprising 1× Q5 buffer, 200 μM of each dNTP and 0.5 U Q5 DNA polymerase. The mixture was denatured for 10 s at 98° C., annealed for 30 s at 64° C., and a linear polymerase reaction was then performed for 30 s at 72° C. After 10 cycles the flanking primers (SEQ ID NOs: 30 and 34) were added, followed by 30 thermocycles of 10 s at 98° C., 30 s at 64° C. and 30 s at 72° C. with a final incubation at 72° C. for 5 min.

After agarose gel purification of the PCR product using the Qiagen Gel Extraction Kit reamplification was performed in 100 μL Q5 DNA polymerase reaction mixture using primers SEQ ID NOs: 30 and 34 by applying the thermocycles described above. A pair of mutually non-compatible type IIS restriction sites (BsaI) in the flanking primers used in the preceding assembly step (SEQ ID NOs: 30 and 34) allowed unidirectional insertion of the central coding region into pSBX8.101d58 (SEQ ID NO: 23). After application of the Qiagen PCR purification Kit the resulting DNA fragment carrying random mutations in the targeted regions was doubly cut with BsaI, again purified using the Qiagen PCR purification Kit and cloned on the plasmid pSBX8.101d58. Transformation (Dower et al. 1988 Nucleic Acids Res. 16: 6127-6145) of electrocompetent E. coli NEB10beta cells (New England Biolabs) yielded a library of 3×10⁹ transformants (according to colony count of a sample fraction), which were plated on 10 square LB agar plates (114 cm²) supplemented with 100 mg/L ampicillin.

Colonies were scraped from the plates and resuspended in each 5 mL LB medium (Sambrook & Russell 2001 Molecular Cloning: A Laboratory Manual, 3rd Ed. Cold Spring Harbor Laboratory Press, New York, N.Y.), then combined and adjusted to a volume of 1 L with fresh medium. After incubation at 30° C. under shaking for 30 min, plasmid DNA was prepared from this pooled culture by means of the Qiagen Plasmid Midi Kit and subsequently used for transformation of electrocompetent E. coli BL21 (Studier & Moffatt 1986 J. Mol. Biol. 189: 113-130). Randomization of the targeted positions in the PylRS#1 gene cloned on pSBX8.101d58 was confirmed by DNA sequencing.

Directly after transformation (Dower et al. 1988 Nucleic Acids Res. 16: 6127-6145) of electrocompetent E. coli BL21 with the CafRS#0-R5 library prepared above, 4 mL of the transfected cell suspension were diluted in 50 mL LB medium supplemented with phosphate buffer (17 mM KH₂PO₄, 72 mM K₂HPO₄) and 1 mM Caf (100 mM stock solution in 300 mM NaOH). After incubation for 2 h at 37° C., cells were sedimented by centrifugation and washed with 10 mL fresh LB medium without additives. After another centrifugation step, cells were resuspended in 2 mL LB medium and plated on four square LB agar plates (114 cm²) supplemented with 100 mg/L ampicillin, 60 mg/L chloramphenicol and 1 mM Caf. Colonies obtained after incubation for 48 h at 37° C. were scraped from the plates and resuspended in 5 mL LB medium each, then combined and diluted into 1 L LB medium containing 100 mg/L ampicillin and grown at 37° C. to 0D₅₅₀=0.4 in a 3 L shake flask. From this culture, triplicates of 2 mL cultures were transferred into plastic tubes and supplemented in parallel with or without 1 mM Caf, freshly dissolved as a 100 mM solution in 300 mM NaOH. Bacteria were grown under shaking at 37° C. for 30 min, then expression of eGFP was induced by addition of 200 ng/mL anhydrotetracycline (aTc; Acros Organics, Geel, Belgium) dissolved at 2 mg/mL in DMF, followed by shaking at 37° C. for another 9-12 h. 1 mL of each culture was centrifuged in a 1.5 mL Eppendorf tube for 3 min and the bacterial pellet was carefully resuspended by repeated pipetting with 1 mL filter-sterilised PBS (4 mM KH₂PO₄, 16 mM Na₂HPO₄, 115 mM NaCl). After washing twice according to this procedure, the bacteria were finally resuspended in the same volume of PBS.

Flow cytofluorimetric analysis as well as bacterial cell sorting were performed on a FACSAria instrument (BD Biosciences, Heidelberg, Germany) which was operated with filter-sterilised PBS as sheath fluid, using a 488 nm LASER for excitation and a 502 nm long-pass filter with a 530/30 band-pass filter for specific detection of eGFP fluorescence. After selecting intact bacterial cells via an appropriate FSC/SSC gate, the final sort gates for each population were dynamically set to select those cells belonging to the fraction of 1 to 5% of total cells with the highest eGFP signal intensities in the presence of Caf for “positive selection” cycles. For “negative selection”, cells with low eGFP signal, comparable to that of uninduced bacteria, were sorted. Bacteria were directly collected in LB medium supplemented with 100 mg/L ampicillin. For reamplification, the sorted cells were plated on LB agar containing 100 mg/L ampicillin and incubated at 37° C. over night. The lawn of colonies was collectively resuspended in LB medium as described further above. A 2 mL aliquot of this dense bacterial cell suspension was used to inoculate 100 mL freshly prepared LB medium supplemented with 100 mg/L ampicillin to be directly used for the next selection cycle.

To enrich CafRS variants with high fidelity and to eliminate those accepting any natural amino acid, two successive negative FACS selection steps were initially performed. Following five alternating FACS selection rounds of positive (i.e. with addition of 1 mM Caf) and negative selection (i.e. in the absence of Caf), a fluorescence response indicating specific incorporation of Caf into the reporter protein eGFP clearly developed. After the final positive selection cycle, bacteria were plated on LB agar and plasmid DNA was prepared from recovered cells by means of the Qiagen Plasmid Midi Kit. After transformation of calcium competent E. coli BL21 cells, followed by plating on LB agar supplemented with 100 mg/L ampicillin in a rectangular plastic dish (Nunc, Langenselbold, Germany), the resulting bacterial population was subjected to single-clone analysis in 96-well microcultures using a robotic platform as previously described in detail (Reichert et al. 2015 Protein Eng. Des. Sel. 28: 553-565). In this assay, 190 randomly chosen colonies were propagated and analyzed individually for eGFP fluorescence.

After incubation over night at 37° C., colonies were automatically picked and used to inoculate 100 μL TB medium (Sambrook & Russell 2001 Molecular Cloning: A Laboratory Manual, 3rd Ed. Cold Spring Harbor Laboratory Press, New York, N.Y.) supplemented with 100 mg/L ampicillin in 96-well round bottom microtiter plates (Sarstedt, Nürnbrecht, Germany). The microtiter plates were sealed with a gas-permeable Breathseal 80/140 mm membrane (Greiner Bio-One, Frickenhausen, Germany) and incubated overnight at 37° C. to stationary phase under 300 rpm agitation using an orbital shaking Minitran incubator with 25 mm amplitude (Infors, Eisenbach, Germany). Then, fresh 1 mL cultures in TB medium containing 100 mg/L ampicillin were inoculated in Masterblock 2 mL V-shape deep well microtiter plates (Greiner Bio-One), each with 20 μL of the pre-culture, and incubated for approximately 2 h at 37° C. to reach OD₅₅₀≈0.5 as monitored with the Synergy 2 SLFA microplate reader (BioTek Instruments, Bad Friedrichshall, Germany). This inoculation step was done in duplicate using two equivalent 96 deep-well plates, one to be supplemented with 1 mM Caf and the other without the non-natural amino acid. After further shaking for 30 min the cells were induced with 200 ng/mL aTc (by adding 20 μL from a 10 μg/mL stock solution in LB medium). Bacterial growth was continued at 37° C. for 12 h; then, the cultures were centrifuged (3857×g; 15 min) and resuspended in 1 mL PBS by repeated pipetting on the robotic platform. Washing in PBS was repeated once. Finally, eGFP^{Caf39} fluorescence of a 100 μL aliquot was measured in the cell suspension using Maxisorb black 96-well assay plates (Nunc) under excitation at 395 nm, detecting emission at 510 nm with cutoff at 495 nm. Fluorescence readings of each well were normalised to 0D₅₅₀ of the same cell suspension, diluted 1:5 (20 μL aliquot plus 80 μL PBS), in a 96-well Mikrotest plate F (Sarstedt). The normalised background fluorescence of two wells with cells harboring only empty pSBX8.100d backbone (encoding no eGFP) was averaged and subtracted from all other fluorescence readings. Final values were determined as fluorescence ratio aaRS^{+Caf}/aaRS^{-Caf} for each clone.

The best clone in terms of efficiency and fidelity, dubbed CafRS#7 (SEQ ID NO: 35) showed already some increase in mean eGFP fluorescence, which indicated the need for randomization of further positions. Sequence analysis of CafRS#7 indicated three amino acid substitutions compared to PylRS#1 (Met309Gln, Asn311Ser and Cys313Gly).

CafRS#7 (SEQ ID NO: 35) was used as starting point for a second focused aaRS library (CafRS#7-R6; SEQ ID NO: 36) with six fully randomized positions (Ala267, Leu270, Tyr271, Leu274, Ile285 and Ile287). Two PCR fragments were generated using two sets of degenerate NNS-primers and assembled. The first PCR fragment was generated with a forward primer (SEQ ID NO: 30) and a NNS reverse primer (SEQ ID NO: 37) to introduce variations for the residues of interest, generating the upstream portion of the gene. The second PCR fragment was generated with another NNS-degenerate forward primer (SEQ ID NO: 38) and a reverse primer (SEQ ID NO: 34), having an overlap of the forward primer to the 3′ end of the first PCR product, providing the downstream portion of the gene. These PCR fragments were generated according to the experimental procedure described above with the CafRS#7 gene serving as template. After agarose gel purification, 200 ng of each fragment was used in an assembly PCR reaction with primers for the 5′ (SEQ ID NO: 30) and 3′ ends (SEQ ID NO: 34) of the gene, also comprising the BsaI restriction sites. The library was cloned on pSBX8.101.d58, yielding 1×10¹⁰ transformants, and subjected to an initial dead/alive selection for viable colonies on LB agar plates supplemented with 100 mg/mL ampicillin as well as 30 mg/mL chloramphenicol and 1 mM Oaf, followed by 2 negative selection rounds using FACS. After five alternative FACS selections (three positive and two negative) bacterial cells were recovered on LB agar supplemented with 100 mg/L ampicillin, followed by single-clone analysis of 189 colonies in a 96-well microculture format as described above. Sequence analysis of the mutated aaRS gene cassettes revealed that the clone with the highest specific fluorescence ratio, dubbed CafRS#29 (SEQ ID NO: 39), carried four additional amino acid substitutions (Ala267Thr, Leu274Ala, Ile285Asn, Ile287Ser) as compared to CafRS#7.

Judged from the crystal structure of the Methanosarcina mazei (Mz) PylRS (PBD entry 2ZCE) and from the results of the two prior library screenings, two residues located at the entry (Gln309 and Ser311), which had already been targeted in the first library, and three residues located at the rear part of the active site (Ala274, Asn285 and Ser287), which had been targeted in the second library, appeared as promising candidates for constructing a third CafRS library. The library CafRS#29-R5 (SEQ ID NO: 40) based on CafRS#29 was generated again via assembly PCR using degenerate NNS primers.

Three PCR fragments were generated and assembled using a set of forward and reverse primers. The first PCR fragment was generated with a forward primer (SEQ ID NO: 30) and a NNS-degenerate reverse primer (SEQ ID NO: 41) to yield the randomized upstream portion of the gene. The second PCR fragment providing the middle part of the gene was generated with a set of two NNS-primers (SEQ ID NO: 38 and SEQ ID NO: 42) having an overlap with the 3′ end of the first and the 5′ end of the third PCR fragment. The third PCR fragment providing the downstream portion of the gene was generated with an NNS forward primer (SEQ ID NO: 31) and a reverse primer (SEQ ID NO: 34). The PCR fragments were generated and assembled according to the experimental procedure described above with the CafRS#29 gene serving as template. The gene library was digested with the restriction enzyme BsaI, gel purified, and ligated with the pSBX8.101d58 vector, after digestion with BsaI, to yield the CafRS#29-R5 library (SEQ ID NO: 40). 10 μg of the ligation products were then electroporated into E. coli NEB10beta cells. Electroporated cells were recovered and plated on LB agar plates with 100 mg/mL ampicillin, yielding 1×10¹⁰ independent transformants. Selection from the CafRS#20-R5 library followed the procedure described for the selections from the first and the second CafRS-library. The finally selected mutant synthetase, CafRS#30 (SEQ ID NO: 43), carries in total 7 amino acid substitutions compared with wild-type Mb PylRS (Ala276Thr, Leu274Ser, Ile285Ser, IIle287Val, Asn311Val, Met315Gly and Tyr349Phe).

### EXAMPLE 5: GENERATION OF SAM1^{CAF} VARIANTS

For a proof of concept, the streptavidin mutant 1, SAm1 (also called “Strep-Tactin”) (Voss & Skerra 1997 Protein Eng. 10:975-82) (SEQ ID NOs: 7 and 8), was modified with Caf at either position V44, W108 or W120. To this end, an amber stop codon (TAG) was introduced into the coding region at each of these sequence positions by site-directed mutagenesis using the plasmid pSAm1 (SEQ ID NO: 44) as template together with the QuikChange site-directed mutagenesis kit and a suitable pair of forward and reverse PCR primers: SEQ ID NO: 45 and 46 resulting in SAm1^{UAG44} (SEQ ID NO: 47), SEQ ID NO: 48 and 49 resulting in SAm1^{UAG108} (SEQ ID NOs: 1 and 2) and SEQ ID NO: 50 and 51 resulting in SAm1^{UAG120} (SEQ ID NO: 52) (FIG. 5B). After transformation of calcium-competent E. coli XL1-blue cells, plasmid preparation (Plasmid Miniprep Kit, Qiagen) and sequencing (Mix2Seq, MWG Eurofins, Ebersberg, Germany), the SAm1 variants were subcloned via XbaI and HindIII on the vector pSBX8.CafRS#30d58 (SEQ ID NO: 53), yielding pSBX8.CafRS#30d47 (V44TAG; SEQ ID NO: 54), pSBX8.CafRS#30d53 (W108TAG; SEQ ID NO: 55) and pSBX8.CafRS#30d51 (W120TAG; SEQ ID NO: 56), respectively.

All positions substituted with Caf were intended to disturb binding of the Strep-tag II if the side chain adopts the cis configuration (i.e., after illumination at 340 or 365 nm) but preserve binding activity in the trans configuration (FIG. 5A). Position Val44 is located on the N-terminal side of the flexible loop region comprising positions 44-53. Caf isomerization was supposed to change the loop conformation. Position Trp108 is located at the bottom of the binding pocket for biotin and, therefore, cis-Caf was supposed to clash with neighboring side chains. Position Trp120 is located at the top of the binding site extending from a neighboring tetramer subunit, thus changing the overall geometry upon isomerization of Caf into the cis state.

### EXAMPLE 6: EXPRESSION AND PURIFICATION OF SAM1 VARIANTS

Both SAm1 (SEQ ID NOs: 7 and 8) and the SAm1^{Caf} variants were produced as cytoplasmic inclusion bodies in E. coli, solubilized, refolded, purified by anion-exchange chromatography (AEX) and analyzed by SDS-PAGE.

A single colony of E. coli BL21 transformed with plasmid pSBX8.CafRS#30d53 coding for SAm1^{Caf108} (SEQ ID NOs: 1 and 2) (FIG. 6A) was used for inoculating 50 mL LB medium supplemented with 100 mg/L ampicillin. After incubation overnight at 30° C. the 20 mL culture was transferred to 2 L LB medium in a baffled shake flask, again supplemented with 100 mg/L ampicillin as well as phosphate buffer (17 mM KH₂PO₄, 72 mM K₂HPO₄) and 1 mM Caf (from a 100 mM stock solution in 300 mM NaOH). The culture was incubated at 37° C. to OD₅₅₀=0.5. Then, SAm1^{Caf108} gene expression (under control of the tet^{o/o}; Skerra 1994 Gene 151: 131-135) was induced with 200 ng/mL aTc and growth was continued at 37° C. for 12 h. The CafRS gene was under the control of E. coli proS promotor and proM terminator. Cells were harvested by centrifugation (10,000×g, 20 min, 4° C.) and washed twice with 100 mL 100 mM Na-borate pH 9.0, 150 mM NaCl to remove precipitated Caf. The bacteria were resuspended in 3 mL per mg wet weight of cold 100 mM Tris-HCl pH 8.0, 150 mM NaCl, 1 mM EDTA and disrupted in 3 runs, using a French Pressure homogenizer (SLM Aminco, Urbana, Ill., USA). The homogenate was centrifuged (20.000 g, 30 min, 4° C.) to sediment the streptavidin inclusion bodies. After washing the protein pellet twice with 50 mM Tris-HCl pH 8.0, 2 M urea, 2% v/v Triton X-100 (3 mL/g cell wet weight) to remove impurities, followed by a washing step with 50 mM Tris-HCl to deplete residual Triton X-100. The inclusion bodies were dissolved in 8 M urea pH 2.5 (3 mL/g cell wet weight). After centrifugation (20.000 g, 30 min, 4° C.), the cleared supernatant was subjected to refolding, which was accomplished by rapid dilution. The unfolded protein was pipetted dropwise into a 25-fold volume of 50 mM Tris-HCl pH 8.0 at 4° C. using a Pasteur pipette. The mixture was incubated over night at 4° C., cleared by centrifugation (10.000×g, 20 min, 4° C.) and purified by AEX on a 6 mL Resource Q column (GE Healthcare, Freiburg, Germany) equilibrated with 20 mM Tris-HCl pH 8.0. Protein fractions eluted in a linear salt concentration gradient of 0-500 mM NaCl at ˜80 mM NaCl in a pure state as analyzed by SDS-PAGE (Fling & Gregerson 1986 Anal. Biochem. 155: 83-88) using staining with Coomassie brilliant blue R-250 (FIG. 6B).

### EXAMPLE 7: PREPARATION OF THE ALKALINE PHOSPHATASE/STREP-TAG II FUSION PROTEIN

Preparative protein expression of the PhoA/Strep-tag II fusion protein using E. coli JM83 transformed with the plasmid pASK75-PhoA-strepII (SEQ ID NO: 57) was accomplished in 2 L LB medium supplemented with 100 mg/mL ampicillin essentially as described by Voss & Skerra 1997 Protein Eng. 10: 975-982. Cultures were grown at 22° C. to OD₅₅₀=0.5, then phoA gene expression was induced by addition of 200 ng/mL aTc. Incubation was continued at 22° C. for 4 h. Cells were harvested via centrifugation, resuspended in 20 mL ice-cold periplasmic fractionation buffer (0.5 M sucrose, 2 mg/mL polymyxcin B sulfate and 100 mM Tris-HCl, pH 8.0) containing 100 μg/mL lysozyme and incubated for 30 min on ice. Due to the presence of metal ions in the active site of the enzyme, periplasmic protein preparation was carried out in the presence of 2 mg/mL polymyxin B sulfate instead of EDTA. The spheroplasts were removed by repeated centrifugation (Skerra & Schmidt 2000 Methods Enzymol. 326: 271-304) and the supernatant was recovered as periplasmic cell fraction. The PhoA/Strep-tag II fusion protein was purified from the periplasmic cell fraction by streptavidin affinity chromatography, using StrepTactin Sepharose (IBA, Göttingen, Germany) and D-desthiobiotin for elution according to a published procedure (Schmidt & Skerra 2007 Nat. Protoc. 2: 1528-1535). To avoid loss of metal ions in the active site of PhoA, EDTA was omitted from the chromatography buffer (150 mM NaCl, 100 mM Tris-HCl pH 8.0). Finally, the PhoA/Strep-tag II fusion protein was dialyzed twice against 2 L buffer (1 mM ZnSO₄, 5 mM MgCl₂, 100 mM Tris-HCl, pH 8.0) for removal of D-desthiobiotin prior to ELISA measurements or binding experiments with Caf-modified streptavidin variants immobilized on a chromatography matrix.

### EXAMPLE 8: DETECTION OF REVERSIBLE BINDING FOR THE PHOA/STREP-TAG II FUSION PROTEIN IN AN ELISA

The light-induced reversible binding of streptavidin mutants carrying the light-switchable amino acid Caf at certain positions was first tested in an ELISA (enzyme-linked immunosorbent assay) using the purified PhoA/Strep-tag II fusion enzyme as a model ligand (FIG. 7).

ELISA was performed at ambient temperature in 96-well microtiter plates (Nunc, Langenselbold, Germany). Each well was coated over night with 100 μL biotinylated bovine serum albumin (BSA) in PBS (4 mM KH₂PO₄, 16 mM Na₂HPO₄, 115 mM NaCl) at a concentration of 1 mg/mL (FIG. 7 A). Biotinylation of 2 mL BSA (10 mg/mL in PBS) was conducted using 20× molar excess of biotin NHS ester. After incubation for 2 h at room temperature, the reaction was quenched by addition of 2 mL 100 mM NaCl, 100 mM Tris-HCl pH 8.0 and purified using a PD-10 desalting column (GE Healthcare) equilibrated with the same buffer. The wells were blocked with 3% w/v BSA, 0.5% v/v Tween in PBS for 2.5 h and washed three times with PBS-Tween. 100 μL of the SAm1 or its Caf-variants were applied at 100 μg/mL in PBS to effect immobilization via complex formation of the pre-adsorbed biotin-BSA. After incubation for 1 h, the wells were washed three times with PBS-Tween. Then, 100 μL of PhoA/Strep-tag II in 1 mM ZnSO₄, 5 mM MgCl₂: 100 mM Tris-HCl pH 8.0 was applied to each well. After incubation for 1 h, the liquid was removed and the wells were washed twice with PBS-Tween and twice with PBS. Between each of these washing steps, the microtiter plate was illuminated with UV light at a wavelength of 365 nm (UV hand lamp, NU-6 KL, Benda Laborgeräte, Wiesloch, Germany; FIG. 7A, lower panel) with 2 mm distance, or with visible light (day light; FIG. 7A, upper panel), for 5 min, whereas buffer exchange was performed in the dark. Finally, 100 μL 0.5 mg/mL p-nitrophenyl phosphate in 1 mM ZnSO₄, 5 mM MgCl₂, 1 M Tris-HCl, pH 8.0 was added to each well and remaining enzymatic PhoA activity was measured as the change in light absorption at 410 nm using a Synergy 2 SLFA microplate reader.

As a result, it appeared that all tested streptavidin mutants showed good affinity for the PhoA/Strep-tag II fusion protein, giving rise to comparable signals as obtained with SAm1 for those samples illuminated with visible light (FIG. 7B). In contrast, a clear decrease in remaining enzyme activity was observed after irradiation with UV light at 365 nm for the streptavidin variant SAm1^{Caf108}. SAm1 as well as the mutants SAm1^{Caf44} and SAm1^{Caf120} showed no or much less signal decrease, respectively, under these circumstances. Hence, the streptavidin mutant SAm1^{Caf108} shows light-inducible (light-switchable) reversible binding of a target protein equipped with an affinity tag.

### EXAMPLE 9: TEST OF A LIGHT-CONTROLLABLE AFFINITY MATRIX

Purified SAm1 or its Caf-variants, encoded on the corresponding derivative of vector pSBX8CAFRS#30 (see Examples 4 and 5), was coupled to NHS-activated Sepharose 4B (Pharmacia, Stockholm, Sweden) at 5 mg protein per mL of swollen gel as described (Schmidt & Skerra 1994 J. Chromatogr. A 676: 337-345). To this end, NHS-activated CH-Sepharose 4B was swollen and washed in ice-cold 1 mM HCl as recommended by the manufacturer. The supernatant was drained and the gel was mixed with twice its volume of a 2.5 mg/mL solution of the streptavidin variant which had been dialyzed against 100 mM NaHCO₃ pH 8.0, 500 mM NaCl. After 2 h of gentle shaking at room temperature the supernatant was decanted and the gel was mixed with 5 volumes of 100 mM Tris-HCl pH 8.0 to achieve blocking of residual activated groups, followed by shaking overnight at 4° C.

A UV-transparent column was packed in a glass capillary (0.7 mm inner diameter) with 20 μL of the chromatography matrix from above, each for Sam1^{Caf44}, SAm1^{Caf108}, SAm1^{Caf120} and SAm1, respectively. At first, the column was equilibrated twice with 2 mL running buffer (100 mM Tris-HCl pH 8.0, 100 mM NaCl) at a constant flow rate of 12 mL/h using a syringe pump (kdScientific, Holliston, Mass., USA), once under UV irradiation at 365 nm (UV hand lamp, NU-6 KL, Benda Laborgeräte, Wiesloch, Germany) and once under irradiation using an LED light table (FG-08, Nippon Genetics, Düren, Germany) with an emitting wavelength of >530 nm (FIG. 8A). Then 25 μL of the purified PhoA/Strep-tag II fusion protein with a concentration of 0.1 mg/mL in 100 mM Tris-HCl pH 8.0, 100 mM NaCl was applied and the column was washed with 2 mL running buffer while unbound protein was collected in the flow through fraction. Sample application and washing steps were conducted under irradiation with visible light using the LED light table. Subsequently, elution of bound protein was triggered by irradiation with UV light at 365 nm using the UV hand lamp. At first, buffer flow was stopped for 10 min while applying UV light. Then the flow rate was set to 12 mL/h again and three elution fractions (25 μL each) were collected. The protein band visible on the Coomassie-stained gel corresponding to the PhoA/Strep-tag II fusion protein in the elution fractions of the chromatography matrix based on SAm1^{Caf108} indicates that the protein was specifically eluted by irradiation at 365 nm (FIG. 8B). No band was observed in case of streptavidin (FIG. 80) or its variants SAm1^{Caf44} and SAm1^{Caf120} (data not shown).

To increase the detection limit of affinity-purified protein in the elution fractions, PhoA enzyme activity was measured. Therefore, 10 μL of each fraction (loaded sample, flow-through, washing and elution fractions 1-3) were applied to single wells of a 96-well plate (Nunc). 90 μL 0.5 mg/mL p-nitrophenyl phosphate in 1 mM ZnSO₄, 5 mM MgCl₂, 1 M Tris-HCl, pH 8.0 was added. After incubation for 30 min at RT the enzymatic activity was determined by measuring time-dependent absorbance at 410 nm using a Synergy 2 SLFA microplate reader. In line with the SDS-PAGE analysis, the elution fractions of the chromatography matrix based on SAm1^{Caf108} showed the highest protein concentration (enzyme activity) eluted under UV irradiation (FIG. 8D).

### EXAMPLE 10: GENERATION OF PROTL^{CAF}-ABD VARIANTS

Protein L is a surface protein originally found in cell wall of Finegoldia magna (formerly known as Peptostreptococcus magnus) with a high affinity and specificity to immunoglobulins (Igs) from many mammalian species, most notably IgGs, and therefore has gained use for antibody purification (Rodrigo et al., 2015 Antibodies 4:259-277). While other IgG binding proteins like protein A and protein G from Staphylococcus aureus and group G Streptococci bind to the Fc region of Igs, protein L binds to the kappa light chain variable region without interfering with the antigen binding site. Natural protein L (UniProt accession number Q51918) essentially comprises the following domains (in analogy to Kaster et al. 1992 J. Biol. Chem. 267: 12820-12825): signal peptide (1-26); three protein G-related albumin-binding domains (77-116; 129-177; 190-238); four homologous B1 domains (254-317; 326-389; 399-436; 474-538); two C-repeats (610-660; 668-722) and a transmembrane region (969-991).

To engineer a light-switchable affinity matrix for the purification of antibodies as well as fragments or related formats (such as antibody fusion proteins, bispecific antibodies and the like), a recombinant protein L comprising a single domain without non-essential domains was designed. The codon optimized protein L domain B1 (herein referred to as ProtL; SEQ ID NO: 20) was fused to a human albumin-binding domain (ABD; SEQ ID NO: 59) derived from protein G via a short linker sequence. The protein L-ABD fusion protein (ProtL-ABD; SEQ ID NO: 61) was modified with Caf at either of the positions 337, 347, 360, 364, 368 or 369 (referring to the numbering scheme in UniProt accession number Q51918). The positions 337, 347, 360, 364, 368 and 369 correspond to positions 13, 23, 36, 40, 44, and 45, respectively, of SEQ ID NO: 61.

To this end, an amber stop codon (TAG) was introduced (via substitution of the original amino acid codon) into the coding region at each of these sequence positions by site-directed mutagenesis using the plasmid pASK75-ProtL-ABD (SEQ ID NO: 62) as template with the help of the QuikChange site-directed mutagenesis kit and a suitable pair of forward and reverse primers: SEQ ID NO: 63 and 66 for ProtL^{UAG337}-ABD (SEQ ID NO: 67), SEQ ID NO: 68 and 69 for ProtL^{UAG347}-ABD (SEQ ID NO: 72), SEQ ID NO: 73 and 74 for ProtL^{UAG360}-ABD (SEQ ID NO: 75), SEQ ID NO: 76 and 77 for ProtL^{UAG364}-ABD (SEQ ID NO: 78), SEQ ID NO: 79 and 80 for ProtL^{UAG368}-ABD (SEQ ID NO: 81) and SEQ ID NO: 82 and 83 for ProtL^{UAG369}-ABD (SEQ ID NO: 84) (FIG. 9).

After transformation of calcium-competent E. coli XL1-blue (Bullock et al., 1987 Biotechniques 5:376-378) cells, plasmid preparation and sequencing, the unmodified ProtL-ABD and the ProtL^{UAG}-ABD variants were subcloned via XbaI and HindIII restriction sites on the vector pSBX8.CafRS#30d58 (SEQ ID NO: 53), yielding the plasmids pSBX8.CafRS#30d70 (no amber-stop codon), pSBX8.CafRS#30d71 (337TAG), pSBX8.CafRS#30d72 (347TAG), pSBX8.CafRS#30d73 (360TAG), pSBX8.CafRS#30d74 (364TAG), pSBX8.CafRS#30d75 (368TAG) and pSBX8.CafRS#30d76 (369TAG), respectively.

Positions 337 and 347 substituted with Caf were intended to disturb binding of Ig if the side chain adopts the cis configuration (i.e., after illumination at about 340 or about 365 nm) but retain binding activity in the trans configuration. Positions 360, 364, 368 and 369 substituted with Caf were intended to disturb Ig binding if the side chain adopts the trans configuration (i.e., after illumination >420 nm) but retain binding activity in the cis configuration. After isomerization the Caf side chain was supposed to clash with neighboring side chains within protein L (thus altering the conformation of its binding site) and/or the Ig ligand (thus changing the geometry of the protein/protein interface) and hence disturb binding.

To provide sufficient space for the large Caf side chain without sterical overlap (particularly in the extended trans configuration) within the binding interface of protein L and to preserve IgG binding activity, additional amino acid exchanges were introduced into the mutated ProtL-ABD as appropriate. For example, the mutation Tyr361Ala was introduced into the coding region of ProtL^{Caf347}-ABD using the QuikChange site-directed mutagenesis kit and the forward and reverse primers SEQ ID NO: 70 and 73. The two additional mutations Tyr361Asn and Leu365Ser were simultaneous introduced into ProtL^{Caf337}-ABD using the primers SEQ ID NO: 65 and 68. These positions 361 and 365 correspond to positions 37 and 41, respectively, of SEQ ID NO: 61 and 86.

### EXAMPLE 11: EXPRESSION AND PURIFICATION OF PROTL^{CAF}-ABD VARIANTS

ProtL (SEQ ID NO: 60) and the ProtL^{Caf} variants (SEQ ID NOs: 69, 74, 77, 80, 83 and 86) were produced as ABD-fusion proteins in the cytoplasm of E. coli and purified by human serum albumin (HSA) affinity chromatography and anion-exchange chromatography (AEX).

For example, a single colony of E. coli MG1655 (Guyer et al., 1981 Cold Spring Harb Symp Quant Biol 45:135-40) transformed with plasmid pSBX8.CafRS#30d71, coding for ProtL^{Caf337}-ABD (SEQ ID NO: 85), was used for inoculating 50 mL LB medium supplemented with 100 mg/L ampicillin. After incubation overnight at 30° C., 20 mL of the culture was transferred to 2 L LB medium supplemented with 100 mg/L ampicillin as well as phosphate buffer (17 mM KH₂PO₄, 72 mM K₂HPO₄) and 1 mM Caf (from a 100 mM stock solution in 300 mM NaOH) in a baffled shake flask. The culture was incubated at 37° C. to OD₅₅₀=0.5 under agitation. Then, ProtL^{Caf337}-ABD gene expression (under control of the tet^{o/o}) was induced with 200 ng/mL aTc and growth was continued at 37° C. for 12-16 h. The CafRS gene was under the constitutive control of the E. coli proS promotor in combination with the proM terminator. Cells were harvested by centrifugation (10,000×g, 20 min, 4° C.), resuspended in 3 mL per g wet weight of cold 50 mM Tris-HCl pH 8.0, 100 mM NaCl, 5 mM EDTA and disrupted using a French Pressure homogenizer. The homogenate was centrifuged (20.000 g, 30 min, 4° C.) to sediment the cell debris, and the cleared supernatant was subjected to affinity chromatography using a HSA affinity column.

The HSA affinity matrix was prepared using NHS-activated Sepharose 4B (GE Healthcare, Freiburg, Germany) according to a published protocol (Schmidt & Skerra 1994 J. Chromatogr. A 676: 337-345). To this end, NHS-activated CH-Sepharose 4B was first swollen and washed in ice-cold 1 mM HCl as recommended by the manufacturer. The supernatant was drained and the gel was mixed with twice its volume of a 5 mg/mL solution of recombinant HSA produced in rice (Sigma-Aldrich, St. Louis, Mo., USA) in 100 mM NaHCO₃ pH 8.0, 500 mM NaCl. After 2 h of gentle shaking at room temperature the supernatant was decanted and the gel was mixed with 5 volumes of 100 mM Tris-HCl pH 8.0 followed by shaking overnight at 4° C. in order to block residual activated groups. The HSA affinity matrix was packed into a 2 ml column housing connected to an ÄKTA Purifier chromatography system.

After equilibration of the HSA column with running buffer (50 mM Tris-HCl pH 8.0, 100 mM NaCl) the cleared supernatant from E. coli containing ProL^{Caf337}-ABD was loaded onto the column. Then, the column was washed with five volumes (10 mL) of running buffer and the bound protein was eluted with 150 mM glycine-HCl pH 2.8, 100 mM NaCl. Peak fractions were collected into neutralization buffer (100 μl of 1 M Tris-HCl pH 9.0 per ml fraction), such that the final pH of the fractions became approximately neutral. Pooled fractions were immediately dialyzed against 20 mM Tris-HCl pH 8.0 at 4° C. over night. ProtL^{Caf337}-ABD was further purified by AEX on a 1 mL Resource Q column (GE Healthcare) equilibrated with 20 mM Tris-HCl pH 8.0. Protein fractions were eluted in a linear salt concentration gradient of 0-200 mM NaCl at ˜100 mM NaCl in a pure state as analyzed by SDS-PAGE (Fling & Gregerson 1986 Anal. Biochem. 155: 83-88) as visualized by staining with Coomassie brilliant blue R-250 (FIG. 10). Other Caf variants as well as the unmodified ProtL-ABD fusion protein were prepared in the same manner.

### EXAMPLE 12: DETECTION OF REVERSIBLE BINDING FOR THE PROTL^{CAF}-ABD FUSION PROTEIN IN AN ELISA

The light-induced reversible binding of ProtL^{Caf}-ABD mutants carrying the light-switchable amino acid Caf at certain positions was tested in an ELISA using a mouse anti-6×His antibody alkaline phosphatase (AP) conjugate (Arigo Biolaboratories, Hsinchu City, Taiwan) as a model Ig ligand (FIG. 11 A). ELISA was performed at ambient temperature in a 96-well Maxisorb microtiter plate (Nunc, Langenselbold, Germany).

To this end, each well was first coated with 50 μl of recombinant HSA produced in rice (Sigma-Aldrich) at a concentration of 10 μg/ml in PBS (4 mM KH₂PO₄, 16 mM Na₂HPO₄, 115 mM NaCl) for 1 h at room temperature. Then, the wells were blocked with 200 ml Roti-Block (Carl Roth, Karlsruhe, Germany) diluted 1:10 in ddH₂O for 1 h and washed three times with PBS containing 0.1% v/v Tween 20 (PBS/T). After that, the purified ProtL^{Caf}-ABD fusion protein from Example 11 was applied in a dilution series in PBS/T and incubated for 1 h to effect complex formation between the ABD moiety and the pre-adsorbed HSA. The wells were then washed three times with PBS/T and incubated with 50 μl of a 1:1000 dilution in PBS/T of the aforementioned mouse anti-6×His Ig-AP conjugate.

After 1 h the microtiter plate was protected from daylight and illuminated with UV light at a wavelength of 365 nm (UV hand lamp NU-6 KL) with 2 mm distance for 5 min. All subsequent washing steps were performed in the dark. The microtiter plate was washed twice with PBS/T and twice with PBS, and then the enzymatic activity was detected using p-nitrophenyl phosphate (0.5 mg/mL in 5 mM MgCl₂, 1 M Tris-HCl pH 8.0) as chromogenic substrate to quantify the remaining bound phosphatase reporter enzyme. After 5 min at 25° C., the absorbance at 405 nm was measured using a SpectraMax 250 microtiter plate reader (Molecular Devices, Sunnyvale, Calif., USA).

As result, the ProtL^{Caf337} variant (SEQ ID NO: 86) illuminated with visible light showed affinity for the IgG, even though with a lower signal than observed for ProtL without Caf (FIG. 11 B). In contrast, a clear decrease in enzyme activity was observed after irradiation with UV light at 365 nm for the ProtL^{Caf337} variant, whereas the unmodified ProtL-ABD fusion protein did not reveal any change in binding activity under the different illumination conditions. The mutants ProtL^{Caf347}, ProtL^{Caf360}, ProtL^{Caf364}, ProtL^{Caf368} and ProtL^{Caf369} showed much less signal decrease under these circumstances. Hence, ProtL^{Caf337} shows light-switchable reversible binding of an IgG.

These experiments demonstrate that a chromatography matrix carrying an immobilized binding protein (engineered streptavidin or protein L) with the non-natural amino acid Caf incorporated at a suitable position in the polypeptide sequence can be used for the reversible binding and light-driven elution of a target protein (here equipped with and without an affinity tag) under typical conditions of an affinity chromatography, but without the need for application of a competing ligand or buffer shift.

The present invention refers to the following nucleotide and amino acid sequences:

SEQ ID NO: 1: Nucleic acid sequence of
Strep-Tactin comprising Caf.
The codon of Caf is in bold face and underlined.
ATGGAAGCAGGTATCACCGGCACCTGGTACAACCAGCTCGGCTCGACCTT
CATCGTGACCGCGGGTGCAGACGGAGCTCTGACCGGTACCTACGTCACGG
CGCGTGGCAACGCCGAGAGCCGCTACGTCCTGACCGGTCGTTACGACAGC
GCCCCGGCCACCGACGGCAGCGGCACCGCCCTCGGTTGGACGGTGGCCTG
GAAGAATAACTACCGCAACGCCCACTCCGCGACCACGTGGAGCGGCCAGT
ACGTCGGCGGCGCCGAGGCGAGGATCAACACCCAGTAGCTGCTGACCTCC
GGCACCACCGAGGCCAACGCCTGGAAGTCCACGCTGGTCGGCCACGACAC
CTTCACCAAGGTGAAGCCGTCCGCCGCCTCCTAA
SEQ ID NO: 2: Amino acid sequence of Strep-Tactin
comprising Caf.
The position of Caf is in bold face and underlined.
MetGluAlaGlyIleThrGlyThrTrpTyrAsnGlnLeuGlySerThr
PheIleValThrAlaGlyAlaAspGlyAlaLeuThrGlyThrTyrVal
ThrAlaArgGlyAsnAlaGluSerArgTyrValLeuThrGlyArgTyr
AspSerAlaProAlaThrAspGlySerGlyThrAlaLeuGlyTrpThr
ValAlaTrpLysAsnAsnTyrArgAsnAlaHisSerAlaThrThrTrp
SerGlyGlnTyrValGlyGlyAlaGluAlaArgIleAsnThrGlnCaf
LeuLeuThrSerGlyThrThrGluAlaAsnAlaTrpLysSerThrLeu
ValGlyHisAspThrPheThrLysValLysProSerAlaAlaSer

In the following, for illustration purposes, the amino acid sequence of Strep-Tactin comprising Caf (SEQ ID NO: 2) is shown below the corresponding nucleic acid sequence (SEQ ID NO: 1). The position of Caf is in bold face and underlined.

```
                          10 20 30 40 50 60
                            + + + + + +
      1 ATGGAAGCAGGTATCACCGGCACCTGGTACAACCAGCTCGGCTCGACCTTCATCGTGACC 60
          MetGluAlaGlyIleThrGlyThrTrpTyrAsnGlnLeuGlySerThrPheIleValThr
                        70 80 90 100 110 120
                          + + + + + +
  61 GCGGGTGCAGACGGAGCTCTGACCGGTACCTACGTCACGGCGCGTGGCAACGCCGAGAGC 120
        AlaGlyAlaAspGlyAlaLeuThrGlyThrTyrValThrAlaArgGlyAsnAlaGluSer
                        130 140 150 160 170 180
                          + + + + + +
121 CGCTACGTCCTGACCGGTCGTTACGACAGCGCCCCGGCCACCGACGGCAGCGGCACCGCC 180
        ArgTyrValLeuThrGlyArgTyrAspSerAlaProAlaThrAspGlySerGlyThrAla
                        190 200 210 220 230 240
                          + + + + + +
181 CTCGGTTGGACGGTGGCCTGGAAGAATAACTACCGCAACGCCCACTCCGCGACCACGTGG 240
        LeuGlyTrpThrValAlaTrpLysAsnAsnTyrArgAsnAlaHisSerAlaThrThrTrp
                        250 260 270 280 290 300
                          + + + + + +
241 AGCGGCCAGTACGTCGGCGGCGCCGAGGCGAGGATCAACACCCAGTAGCTGCTGACCTCC 300
        SerGlyGlnTyrValGlyGlyAlaGluAlaArgIleAsnThrGlnCafLeuLeuThrSer
                        310 320 330 340 350 360
                          + + + + + +
301 GGCACCACCGAGGCCAACGCCTGGAAGTCCACGCTGGTCGGCCACGACACCTTCACCAAG 360
        GlyThrThrGluAlaAsnAlaTrpLysSerThrLeuValGlyHisAspThrPheThrLys
                        370 380
                          + +
361 GTGAAGCCGTCCGCCGCCTCCTAA 384
        ValLysProSerAlaAlaSerEnd
SEQ ID NO: 3: Nucleic acid sequence of core streptavidin comprising Caf.
The codon of Caf is in bold face and underlined.
ATGGAAGCAGGTATCACCGGCACCTGGTACAACCAGCTCGGCTCGACCTTCATCGTGACC
GCGGGCGCCGACGGCGCCCTGACCGGAACCTACGAGTCGGCCGTCGGCAACGCCGAGA
GCCGCTACGTCCTGACCGGTCGTTACGACAGCGCCCCGGCCACCGACGGCAGCGGCACC
GCCCTCGGTTGGACGGTGGCCTGGAAGAATAACTACCGCAACGCCCACTCCGCGACCAC
GTGGAGCGGCCAGTACGTCGGCGGCGCCGAGGCGAGGATCAACACCCAGTAGCTGCTGA
CCTCCGGCACCACCGAGGCCAACGCCTGGAAGTCCACGCTGGTCGGCCACGACACCTTC
ACCAAGGTGAAGCCGTCCGCCGCCTCCTAA
SEQ ID NO: 4: Amino acid sequence of core streptavidin comprising Caf.
The position of Cafisinbddfaceandundedined.
MetGluAlaGlyIleThrGlyThrTrpTyrAsnGlnLeuGlySerThrPheIleValThr
AlaGlyAlaAspGlyAlaLeuThrGlyThrTyrGluSerAlaValGlyAsnAlaGluSer
ArgTyrValLeuThrGlyArgTyrAspSerAlaProAlaThrAspGlySerGlyThrAla
LeuGlyTrpThrValAlaTrpLysAsnAsnTyrArgAsnAlaHisSerAlaThrThrTrp
SerGlyGlnTyrValGlyGlyAlaGluAlaArgIleAsnThrGlnCafLeuLeuThrSer
GlyThrThrGluAlaAsnAlaTrpLysSerThrLeuValGlyHisAspThrPheThrLys
ValLysProSerAlaAlaSer
```

In the following, for illustration purposes, the amino acid sequence of core streptavidin comprising Caf (SEQ ID NO: 4) is shown below the corresponding nucleic acid sequence (SEQ ID NO: 3). The position of Caf is in bold face and underlined.

```
                          10 20 30 40 50 60
                            + + + + + +
      1 ATGGAAGCAGGTATCACCGGCACCTGGTACAACCAGCTCGGCTCGACCTTCATCGTGACC 60
          MetGluAlaGlyIleThrGlyThrTrpTyrAsnGlnLeuGlySerThrPheIleValThr
                        70 80 90 100 110 120
                          + + + + + +
  61 GCGGGCGCCGACGGCGCCCTGACCGGAACCTACGAGTCGGCCGTCGGCAACGCCGAGAGC 120
        AlaGlyAlaAspGlyAlaLeuThrGlyThrTyrGluSerAlaValGlyAsnAlaGluSer
                        130 140 150 160 170 180
                          + + + + + +
121 CGCTACGTCCTGACCGGTCGTTACGACAGCGCCCCGGCCACCGACGGCAGCGGCACCGCC 180
        ArgTyrValLeuThrGlyArgTyrAspSerAlaProGlyThrAspGlySerGlyThrAla
                        190 200 210 220 230 240
                          + + + + + +
181 CTCGGTTGGACGGTGGCCTGGAAGAATAACTACCGCAACGCCCACTCCGCGACCACGTGG 240
        LeuGlyTrpThrValAlaTrpLysAsnAsnTyrArgAsnAlaHisSerAlaThrThrTrp
                        250 260 270 280 290 300
                          + + + + + +
241 AGCGGCCAGTACGTCGGCGGCGCCGAGGCGAGGATCAACACCCAGTAGCTGCTGACCTCC 300
        SerGlyGlnTyrValGlyGlyAlaGluAlaArgIleAsnThrGlnCafLeuLeuThrSer
                        310 320 330 340 350 360
                          + + + + + +
301 GGCACCACCGAGGCCAACGCCTGGAAGTCCACGCTGGTCGGCCACGACACCTTCACCAAG 360
        GlyThrThrGluAlaAsnAlaTrpLysSerThrLeuValGlyHisAspThrPheThrLys
                          370 380
                            + +
  361 GTGAAGCCGTCCGCCGCCTCCTAA 384
          ValLysProSerAlaAlaSerEnd
SEQ ID NO: 5: Nucleic acid sequence of unprocessed streptavidin
(i.e. pre-streptavidin) comprising Caf.
The codon of Caf is in bold face and underlined.
ATGCGCAAGATCGTCGTTGCAGCCATCGCCGTTTCCCTGACCACGGTCTCGATTACGGCC
AGCGCTTCGGCAGACCCCTCCAAGGACTCGAAGGCCCAGGTCTCGGCCGCCGAGGCCGG
CATCACCGGCACCTGGTACAACCAGCTCGGCTCGACCTTCATCGTGACCGCGGGCGCCG
ACGGCGCCCTGACCGGAACCTACGAGTCGGCCGTCGGCAACGCCGAGAGCCGCTACGTC
CTGACCGGTCGTTACGACAGCGCCCCGGCCACCGACGGCAGCGGCACCGCCCTCGGTTG
GACGGTGGCCTGGAAGAATAACTACCGCAACGCCCACTCCGCGACCACGTGGAGCGGCC
AGTACGTCGGCGGCGCCGAGGCGAGGATCAACACCCAGTAGCTGCTGACCTCCGGCACC
ACCGAGGCCAACGCCTGGAAGTCCACGCTGGTCGGCCACGACACCTTCACCAAGGTGAA
GCCGTCCGCCGCCTCCATCGACGCGGCGAAGAAGGCCGGCGTCAACAACGGCAACCCGC
TCGACGCCGTTCAGCAGTAG
SEQ ID NO: 6: Amino acid sequence of unprocessed streptavidin
(i.e. pre-streptavidin) comprising Caf.
The signal sequence which directs secretion of streptavidin is
underlined. The position of Caf is in bold face and underlined.
MetArgLvsIleValValAlaAlaIleAlaValSerLeuThrThrValSerIleThrAla
SerAlaSerAlaAspProSerLysAspSerLysAlaGlnValSerAlaAlaGluAlaGly
IleThrGlyThrTrpTyrAsnGlnLeuGlySerThrPheIleValThrAlaGlyAlaAsp
GlyAlaLeuThrGlyThrTyrGluSerAlaValGlyAsnAlaGluSerArgTyrValLeu
ThrGlyArgTyrAspSerAlaProAlaThrAspGlySerGlyThrAlaLeuGlyTrpThr
Va1AlaTrpLysAsnAsnTyrArgAsnAlaHisSerAlaThrThrTrpSerGlyGinTyr
ValGlyGlyAlaGluAlaArgIleAsnThrGlnCafLeuLeuThrSerGlyThrThrGlu
AlaAsnAlaTrpLysSerThrLeuValGlyHisAspThrPheThrLysValLysProSer
AlaAlaSerIleAspAlaAlaLysLysAlaGlyValAsnAsnGlyAsnProLeuAspAla
ValGlnGln
```

In the following, for illustration purposes, the amino acid sequence of unprocessed streptavidin (i.e. pre-streptavidin) comprising Caf (SEQ ID NO: 6) is shown below the corresponding nucleic acid sequence (SEQ ID NO: 5). The signal sequence which directs secretion of streptavidin is underlined. The position of Caf is in bold face and underlined. The sequence of core streptavidin begins with Glu²⁵ and ends with Ser¹⁶³.

```
                          10 20 30 40 50 60
                            + + + + + +
      1 ATGCGCAAGATCGTCGTTGCAGCCATCGCCGTTTCCCTGACCACGGTCTCGATTACGGCC 60
          MetArgLysIlevalValAlaAlaIleAlaValSerLeuThrThrValSerIleThrAla
                        70 80 90 100 110 120
                          + + + + + +
  61 AGCGCTTCGGCAGACCCCTCCAAGGACTCGAAGGCCCAGGTCTCGGCCGCCGAGGCCGGC 120
        SerAlaSerAlaAspProSerLysAspSerLysAlaGlnValSerAlaAlaGluAlaGly
                                                                                                              14
                        130 140 150 160 170 180
                          + + + + + +
121 ATCACCGGCACCTGGTACAACCAGCTCGGCTCGACCTTCATCGTGACCGCGGGCGCCGAC 180
        IleThrGlyThrTrpTyrAsnGlnLeuGlySerThrPheIleValThrAlaGlyAlaAsp
                        190 200 210 220 230 240
                          + + + + + +
181 GGCGCCCTGACCGGAACCTACGAGTCGGCCGTCGGCAACGCCGAGAGCCGCTACGTCCTG 240
        GlyAlaLeuThrGlyThrTyrGluSerAlaValGlyAsnAlaGluSerArgTyrValLeu
                        250 260 270 280 290 300
                          + + + + + +
241 ACCGGTCGTTACGACAGCGCCCCGGCCACCGACGGCAGCGGCACCGCCCTCGGTTGGACG 300
        ThrGlyArgTyrAspSerAlaProAlaThrAspGlySerGlyThrAlaLeuGlyTrpThr
                        310 320 330 340 350 360
                          + + + + + +
301 GTGGCCTGGAAGAATAACTACCGCAACGCCCACTCCGCGACCACGTGGAGCGGCCAGTAC 360
        ValAlaTrpLysAsnAsnTyrArgAsnAlaHisSerAlaThrThrTrpSerGlyGlnTyr
                        370 380 390 400 410 420
                          + + + + + +
361 GTCGGCGGCGCCGAGGCGAGGATCAACACCCAGTAGCTGCTGACCTCCGGCACCACCGAG 420
        ValGlyGlyAlaGluAlaArgIleAsnThrGlnCafLeuLeuThrSerGlyThrThrGlu
                        430 440 450 460 470 480
                          + + + + + +
421 GCCAACGCCTGGAAGTCCACGCTGGTCGGCCACGACACCTTCACCAAGGTGAAGCCGTCC 480
        AlaAsnAlaTrpLysSerThrLeuValGlyHisAspThrPheThrLysValLysProSer
                        490 500 510 520 530 540
                          + + + + + +
481 GCCGCCTCCATCGACGCGGCGAAGAAGGCCGGCGTCAACAACGGCAACCCGCTCGACGCC 540
        AlaAlaSerIleAspAlaAlaLysLysAlaGlyValAsnAsnGlyAsnProLeuAspAla
                      163
                        550
                          +
541 GTTCAGCAGTAG 552
        ValGlnGlnEnd
SEQ ID NO: 7: Nucleic acid sequence of streptactin.
ATGGAAGCAGGTATCACCGGCACCTGGTACAACCAGCTCGGCTCGACCTTCATCGTGACC
GCGGGTGCAGACGGAGCTCTGACCGGTACCTACGTCACGGCGCGTGGCAACGCCGAGAG
CCGCTACGTCCTGACCGGTCGTTACGACAGCGCCCCGGCCACCGACGGCAGCGGCACCG
CCCTCGGTTGGACGGTGGCCTGGAAGAATAACTACCGCAACGCCCACTCCGCGACCACGT
GGAGCGGCCAGTACGTCGGCGGCGCCGAGGCGAGGATCAACACCCAGTGGCTGCTGAC
CTCCGGCACCACCGAGGCCAACGCCTGGAAGTCCACGCTGGTCGGCCACGACACCTTCA
CCAAGGTGAAGCCGTCCGCCGCCTCCTAA
SEQ ID NO: 8: Amino acid sequence of Strep-Tactin.
Trp96 is in bold face and underlined.
MetGluAlaGlyIleThrGlyThrTrpTyrAsnGlnLeuGlySerThrPheIleValThr
AlaGlyAlaAspGlyAlaLeuThrGlyThrTyrValThrAlaArgGlyAsnAlaGluSer
ArgTyrValLeuThrGlyArgTyrAspSerAlaProAlaThrAspGlySerGlyThrAla
LeuGlyTrpThrValAlaTrpLysAsnAsnTyrArgAsnAlaHisSerAlaThrThrTrp
SerGlyGlnTyrValGlyGlyAlaGluAlaArgIleAsnThrGlnTrpLeuLeuThrSer
GlyThrThrGluAlaAsnAlaTrpLysSerThrLeuValGlyHisAspThrPheThrLys
ValLysProSerAlaAlaSer
```

In the following, for illustration purposes, the amino acid sequence of streptactin (SEQ ID NO: 8) is shown below the corresponding nucleic acid sequence (SEQ ID NO: 7). The position of Trp is in bold face and underlined.

```
                          10 20 30 40 50 60
                            + + + + + +
      1 ATGGAAGCAGGTATCACCGGCACCTGGTACAACCAGCTCGGCTCGACCTTCATCGTGACC 60
          MetGluAlaGlyIleThrGlyThrTrpTyrAsnGlnLeuGlySerThrPheIleValThr
                14
                        70 80 90 100 110 120
                          + + + + + +
  61 GCGGGTGCAGACGGAGCTCTGACCGGTACCTACGTCACGGCGCGTGGCAACGCCGAGAGC 120
        AlaGlyAlaAspGlyAlaLeuThrGlyThrTyrValThrAlaArgGlyAsnAlaGluSer
                        130 140 150 160 170 180
                          + + + + + +
121 CGCTACGTCCTGACCGGTCGTTACGACAGCGCCCCGGCCACCGACGGCAGCGGCACCGCC 180
        ArgTyrValLeuThrGlyArgTyrAspSerAlaProAlaThrAspGlySerGlyThrAla
                        190 200 210 220 230 240
                          + + + + + +
181 CTCGGTTGGACGGTGGCCTGGAAGAATAACTACCGCAACGCCCACTCCGCGACCACGTGG 240
        LeuGlyTrpThrValAlaTrpLysAsnAsnTyrArgAsnAlaHisSerAlaThrThrTrp
                        250 260 270 280 290 300
                          + + + + + +
241 AGCGGCCAGTACGTCGGCGGCGCCGAGGCGAGGATCAACACCCAGTGGCTGCTGACCTCC 300
        SerGlyGlnTyrValGlyGlyAlaGluAlaArgIleAsnThrGlnTrpLeuLeuThrSer
                        310 320 330 340 350 360
                          + + + + + +
301 GGCACCACCGAGGCCAACGCCTGGAAGTCCACGCTGGTCGGCCACGACACCTTCACCAAG 360
        GlyThrThrGluAlaAsnAlaTrpLysSerThrLeuValGlyHisAspThrPheThrLys
                        370 380
                          + +
361 GTGAAGCCGTCCGCCGCCTCCTAA 384
        ValLysProSerAlaAlaSerEnd
                                              139
SEQ ID NO: 9: Nucleic acid sequence of core streptavidin.
ATGGAAGCAGGTATCACCGGCACCTGGTACAACCAGCTCGGCTCGACCTTCATCGTGACC
GCGGGCGCCGACGGCGCCCTGACCGGAACCTACGAGTCGGCCGTCGGCAACGCCGAGA
GCCGCTACGTCCTGACCGGTCGTTACGACAGCGCCCCGGCCACCGACGGCAGCGGCACC
GCCCTCGGTTGGACGGTGGCCTGGAAGAATAACTACCGCAACGCCCACTCCGCGACCAC
GTGGAGCGGCCAGTACGTCGGCGGCGCCGAGGCGAGGATCAACACCCAGTGGCTGCTG
ACCTCCGGCACCACCGAGGCCAACGCCTGGAAGTCCACGCTGGTCGGCCACGACACCTT
CACCAAGGTGAAGCCGTCCGCCGCCTCCTAA
SEQ ID NO: 10: Amino acid sequence of core streptavidin (residues
2-127 correspond to residues 38-163 in UniProt database entry P22629;
residue 1 is a start methionine). Trp96 is in bold face and underlined.
MetGluAlaGlyIleThrGlyThrTrpTyrAsnGlnLeuGlySerThrPheIleValThr
AlaGlyAlaAspGlyAlaLeuThrGlyThrTyrGluSerAlaValGlyAsnAlaGluSer
ArgTyrValLeuThrGlyArgTyrAspSerAlaProAlaThrAspGlySerGlyThrAla
LeuGlyTrpThrValAlaTrpLysAsnAsnTyrArgAsnAlaHisSerAlaThrThrTrp
SerGlyGlnTyrValGlyGlyAlaGluAlaArgIleAsnThrGlnTrpLeuLeuThrSer
GlyThrThrGluAlaAsnAlaTrpLysSerThrLeuValGlyHisAspThrPheThrLys
ValLysProSerAlaAlaSer
```

In the following, for illustration purposes, the amino acid sequence of core streptavidin (SEQ ID NO: 10) is shown below the corresponding nucleic acid sequence (SEQ ID NO: 9). The position of Trp96 is in bold face and underlined.

```
                          10 20 30 40 50 60
                            + + + + + +
      1 ATGGAAGCAGGTATCACCGGCACCTGGTACAACCAGCTCGGCTCGACCTTCATCGTGACC 60
          MetGluAlaGlyIleThrGlyThrTrpTyrAsnGlnLeuGlySerThrPheIleValThr
                        70 80 90 100 110 120
                          + + + + + +
  61 GCGGGCGCCGACGGCGCCCTGACCGGAACCTACGAGTCGGCCGTCGGCAACGCCGAGAGC 120
        AlaGlyAlaAspGlyAlaLeuThrGlyThrTyrGluSerAlaValGlyAsnAlaGluSer
                        130 140 150 160 170 180
                          + + + + + +
121 CGCTACGTCCTGACCGGTCGTTACGACAGCGCCCCGGCCACCGACGGCAGCGGCACCGCC 180
        ArgTyrValLeuThrGlyArgTyrAspSerAlaProAlaThrAspGlySerGlyThrAla
                        190 200 210 220 230 240
                          + + + + + +
181 CTCGGTTGGACGGTGGCCTGGAAGAATAACTACCGCAACGCCCACTCCGCGACCACGTGG 240
        LeuGlyTrpThrValAlaTrpLysAsnAsnTyrArgAsnAlaHisSerAlaThrThrTrp
                        250 260 270 280 290 300
                          + + + + + +
241 AGCGGCCAGTACGTCGGCGGCGCCGAGGCGAGGATCAACACCCAGTGGCTGCTGACCTCC 300
        SerGlyGlnTyrValGlyGlyAlaGluAlaArgIleAsnThrGlnTrpLeuLeuThrSer
                        310 320 330 340 350 360
                          + + + + + +
301 GGCACCACCGAGGCCAACGCCTGGAAGTCCACGCTGGTCGGCCACGACACCTTCACCAAG 360
        GlyThrThrGluAlaAsnAlaTrpLysSerThrLeuValGlyHisAspThrPheThrLys
                        370 380
                          + +
361 GTGAAGCCGTCCGCCGCCTCCTAA 384
        ValLysProSerAlaAlaSerEnd
SEQ ID NO: 11: Nucleic acid sequence of unprocessed streptavidin
(pre-streptavidin).
ATGCGCAAGATCGTCGTTGCAGCCATCGCCGTTTCCCTGACCACGGTCTCGATTACGGCC
AGCGCTTCGGCAGACCCCTCCAAGGACTCGAAGGCCCAGGTCTCGGCCGCCGAGGCCGG
CATCACCGGCACCTGGTACAACCAGCTCGGCTCGACCTTCATCGTGACCGCGGGCGCCG
ACGGCGCCCTGACCGGAACCTACGAGTCGGCCGTCGGCAACGCCGAGAGCCGCTACGTC
CTGACCGGTCGTTACGACAGCGCCCCGGCCACCGACGGCAGCGGCACCGCCCTCGGTTG
GACGGTGGCCTGGAAGAATAACTACCGCAACGCCCACTCCGCGACCACGTGGAGCGGCC
AGTACGTCGGCGGCGCCGAGGCGAGGATCAACACCCAGTGGCTGCTGACCTCCGGCACC
ACCGAGGCCAACGCCTGGAAGTCCACGCTGGTCGGCCACGACACCTTCACCAAGGTGAA
GCCGTCCGCCGCCTCCATCGACGCGGCGAAGAAGGCCGGCGTCAACAACGGCAACCCGC
TCGACGCCGTTCAGCAGTAG
SEQ ID NO: 12: Amino acid sequence of pre-streptavidin.
Trp132 is in bold face and underlined.
MetArgLysIleValValAlaAlaIleAlaValSerLeuThrThrValSerIleThrAla
SerAlaSerAlaAspProSerLysAspSerLysAlaGlnValSerAlaAlaGluAlaGly
IleThrGlyThrTrpTyrAsnGlnLeuGlySerThrPheIleValThrAlaGlyAlaAsp
GlyAlaLeuThrGlyThrTyrGluSerAlaValGlyAsnAlaGluSerArgTyrValLeu
ThrGlyArgTyrAspSerAlaProAlaThrAspGlySerGlyThrAlaLeuGlyTrpThr
ValAlaTrpLysAsnAsnTyrArgAsnAlaHisSerAlaThrThrTrpSerGlyGlnTyr
ValGlyGlyAlaGluAlaArgIleAsnThrGlnTrpLeuLeuThrSerGlyThrThrGlu
AlaAsnAlaTrpLysSerThrLeuValGlyHisAspThrPheThrLysValLysProSer
AlaAlaSerIleAspAlaAlaLysLysAlaGlyValAsnAsnGlyAsnProLeuAspAla
VaiGlnGln
```

In the following, for illustration purposes, the amino acid sequence of pre-streptavidin (SEQ ID NO: 12) is shown below the corresponding nucleic acid sequence (SEQ ID NO: 11). The position of Trp132 is in bold face and underlined.

```
                          10 20 30 40 50 60
                            + + + + + +
      1 ATGCGCAAGATCGTCGTTGCAGCCATCGCCGTTTCCCTGACCACGGTCTCGATTACGGCC 60
          MetArgLysIleValValAlaAlaIleAlaValSerLeuThrThrValSerIleThrAla
                        70 80 90 100 110 120
                          + + + + + +
  61 AGCGCTTCGGCAGACCCCTCCAAGGACTCGAAGGCCCAGGTCTCGGCCGCCGAGGCCGGC 120
        SerAlaSerAlaAspProSerLysAspSerLysAlaGlnValSerAlaAlaGluAlaGly
                        130 140 150 160 170 180
                          + + + + + +
121 ATCACCGGCACCTGGTACAACCAGCTCGGCTCGACCTTCATCGTGACCGCGGGCGCCGAC 180
        IleThrGlyThrTrpTyrAsnGlnLeuGlySerThrPheIleValThrAlaGlyAlaAsp
                        190 200 210 220 230 240
                          + + + + + +
181 GGCGCCCTGACCGGAACCTACGAGTCGGCCGTCGGCAACGCCGAGAGCCGCTACGTCCTG 240
        GlyAlaLeuThrGlyThrTyrGluSerAlaValGlyAsnAlaGluSerArgTyrValLeu
                        250 260 270 280 290 300
                          + + + + + +
241 ACCGGTCGTTACGACAGCGCCCCGGCCACCGACGGCAGCGGCACCGCCCTCGGTTGGACG 300
        ThrGlyArgTyrAspSerAlaProAlaThrAspGlySerGlyThrAlaLeuGlyTrpThr
                        310 320 330 340 350 360
                          + + + + + +
301 GTGGCCTGGAAGAATAACTACCGCAACGCCCACTCCGCGACCACGTGGAGCGGCCAGTAC 360
        ValAlaTrpLysAsnAsnTyrArgAsnAlaHisSerAlaThrThrTrpSerGlyGlnTyr
                        370 380 390 400 410 420
                          + + + + + +
361 GTCGGCGGCGCCGAGGCGAGGATCAACACCCAGTGGCTGCTGACCTCCGGCACCACCGAG 420
        ValGlyGlyAlaGluAlaArgIleAsnThrGlnTrpLeuLeuThrSerGlyThrThrGlu
                        430 440 450 460 470 480
                          + + + + + +
421 GCCAACGCCTGGAAGTCCACGCTGGTCGGCCACGACACCTTCACCAAGGTGAAGCCGTCC 480
        AlaAsnAlaTrpLysSerThrLeuValGlyHisAspThrPheThrLysValLysProSer
                        490 500 510 520 530 540
                          + + + + + +
481 GCCGCCTCCATCGACGCGGCGAAGAAGGCCGGCGTCAACAACGGCAACCCGCTCGACGCC 540
        AlaAlaSerIleAspAlaAlaLysLysAlaGlyValAsnAsnGlyAsnProLeuAspAla
                        550
                          +
541 GTTCAGCAGTAG 552
        ValGlnGlnEnd
SEQ ID NO: 13: Amino acid sequence of Strep-tag
AWRHPQFGG
SEQ ID NO: 14: Amino acid sequence of Strep-tag II
WSHPQFEK
SEQ ID NO: 15 Amino acid sequence of the myc-tag (corresponding to residues 410-419
in UniProt database entry P01106).
EQKLISEEDL
SEQ ID NO: 16: Amino acid sequence of the domain Z of protein A (corresponding to
residues 212-269 in UniProt database entry P38507). Suitable positions for Caf
incorporation Phe5, Gln9, Phe13, Tyr14, Glu25, Gln26, Arg27, Asn28 Ala29, Phe30, Ile31,
Gln32, Lys35, Asp36, Asp37, Gln40, Asn43, Leu45, Glu47, Leu51, Asn52 shown in bold
face and underlined.
VDNKFNKEQQNAFYEILHLPNLNEEQRNAFIQSLKDDPSQSANLLAEAKKLNDAQAPK
SEQ ID NO: 17 Amino acid sequence of the C1 domain of protein G (corresponding to
residues 303-357 in UniProt database entry P19909). Suitable positions for Caf
incorporation are Lys3, Ile5, Thr10, Thr16, Val28, Tyr32, Asp35 shown in bold face and
underlined.
TYKLILNGKTLKGETTTEAVDAATAEKVFKQYANDNGVDGEWTYDDATKTFTVTE
SEQ ID NO: 18 Amino acid sequence of the C2 domain of protein G (corresponding to
residues 373-427 in UniProt database entry P19909). Suitable positions for Caf
incorporation are Lys3, Val5, Thr10, Thr16, Val28, Tyr32, Asp35 shown in bold face and
underlined.
TYKLVINGKTLKGETTTEAVDAATAEKVFKQYANDNGVDGEWTYDDATKTFTVTE
SEQ ID NO: 19 Amino acid sequence of the C3 domain of protein G (corresponding to
residues 443-497 in UniProt database entry P19909). Suitable positions for Caf
incorporation are Lys3, Val5, Thr10, Thr16, Ala28, Tyr32, Asp35 shown in bold face and
underlined.
TYKLVINGKTLKGETTTKAVDAETAEKAFKQYANDNGVDGVWTYDDATKTFTVTE
SEQ ID NO: 20 Amino acid sequence of the domain B1 of protein L (corresponding to
residues 326-389 in UniProt database entry Q51918). Suitable positions for Caf
incorporation are Thr5 (330), Asn9 (334), Ile (336), Phe12 (337), Lys16 (341), Phe 22
(347) Phe26 (351), Lys32 (357), Ala35 (360), Leu39 (364), Glu43 (368), Asn44 (369) Tyr47
(372) shown in bold face and underlined.
KEEVTIKVNLIFADGKTQTAEFKGTFEEATAKAYAYADLLAKENGEYTADLEDGGNTINIKFAG
SEQ ID NO: 21 Amino acid sequence of the heavy chain of the anti-myc-tag monoclonal
antibody clone 9E10 (corresponding to residues 20-470 in GenBank database entry
CAN87018). Suitable positions for Caf incorporation are the residues corresponding to
Tyr76, Phe121, Tyr122, Tyr123, Tyr124, Tyr128, Tyr129 and Tyr130 of GenBank database
entry CAN87018, which are shown in bold face and underlined. More specifically, since the
sequence below starts with residue 20 of GenBank database entry CAN87018, the positions
which correspond to Tyr76, Phe121, Tyr122, Tyr123, Tyr124, Tyr128, Tyr129 and Tyr130 of
GenBank database entry CAN87018 are the positions Tyr57, Phe102, Tyr103, Tyr104, Tyr105,
Tyr109, Tyr110 and Tyr111, respectively, in the sequence below.
EVHLVESGGDLVKPGGSLKLSCAASGFTFSHYGMSWVRQTPDKRLEWVATIGSRGTYTHYPD
SVKGRFTISRDNDKNALYLQMNSLKSEDTAMYYCARRSEFYYYGNTYYYSAMDYWGQGASVT
VSSAKTTPPSVYPLAPGSAAQTNSMVTLGCLVKGYFPEPVTVTWNSGSLSSGVHTFPAVLQSD
LYTLSSSVTVPSSTWPSETVTCNVAHPASSTKVDKKIVPRDCGCKPCICTVPEVSSVFIFPPKPK
DVLTITLTPKVTCVVVDISKDDPEVQFSWFVDDVEVHTAQTQPREEQFNSTFRSVSELPIMHQD
WPNGKEFKCRVNSAAFPAPIEKTISKTKGRPKAPQVYTIPPPKEQMAKDKVSLTCMITDFFPEDI
TVEWQWNGQPAENYKNTQPIMNTNGSYFVYSKLNVQKSNWEAGNTFTCSVLHEGLHNHHTE
KSLSHSPGK
SEQ ID NO: 22 Amino acid sequence of the light chain of the anti-myc-tag monoclonal
antibody clone 9E10 (corresponding to residues 21-238 in GenBank database entry
CAN87019).
DIVLTQSPASLAVSLGQRATISCRASESVDNYGFSFMNWFQQKPGQPPKLLIYAISNRGSGVPA
RFSGSGSGTDFSLNIHPVEEDDPAMYFCQQTKEVPWTFGGGTKLEIKRADAAPTVSIFPPSSE
QLTSGGASVVCFLNNLYPKDINVKWKIDGSERQNGVLNSWTDQDSKDSTYSMSSTLTLTKDEY
ERHNSYTCEATHKTSTSPIVKSFNRNEC
SEQ ID NO: 23: Nucleic acid sequence of pSBX8.101d58
TTTTTACGGTTCCTGGCCTTTTGCTGGCCTTTTGCTCACATGACCCGACACCATAACGCTC
GGTTGCCGCCGGGCGTTTTTTATTGGCCAGATGATTAATTCCTAATTTTTGTTGACACTCTA
TCATTGATAGAGTTATTTTACCACTCCCTATCAGTGATAGAGAAAAGTGAAATGAATAGTTC
GACAAAATCTAGATAACGAGGGCAAAAAATGTCTAAAGGTGAAGAACTTTTCACTGGAGTT
GTCCCAATTCTTGTTGAATTAGATGGTGATGTTAATGGGCACAAATTTTCTGTCAGTGGAGA
GGGTGAAGGTGATGCAACATAGGGAAAACTTACCCTTAAATTTATTTGCACTACTGGAAAA
CTACCTGTTCCATGGCCAACACTTGTCACTACTTTGACTTATGGTGTTCAATGCTTTTCAAG
ATACCCGGATCATATGAAACGGCATGACTTTTTCAAGAGTGCCATGCCCGAAGGTTATGTA
CAGGAAAGAACTATATTTTTCAAAGATGACGGGAACTACAAGACACGTGCTGAAGTCAAGT
TTGAAGGTGATACCCTTGTTAATAGAATCGAGTTAAAAGGTATTGATTTTAAAGAAGATGGA
AACATTCTTGGACACAAATTGGAATACAACTATAACTCACACAATGTATACATCATGGCAGA
CAAACAAAAGAATGGAATCAAAGTTAACTTCAAAATTAGACACAACATTGAAGATGGAAGCG
TTCAACTAGCAGACCATTATCAACAAAATACTCCAATTGGCGATGGCCCTGTCCTTTTACCA
GACAACCATTACCTGTCCACACAATCTGCCCTTTCGAAAGATCCCAACGAAAAGAGGGACC
ACATGGTCCTTCTTGAGTTTGTAACAGCTGCTGGGATTACACATGGCATGGATGAACTGTA
CCAAAGCGCTTGGAGCCACCCGCAGTTCGAAAAATAATAAGCTTGACCTGTGAAGTGAAAA
ATGGCGCACATTGTGCGACATTTTTTTTGTCTGCCGTTTACCGCTACTGCGCGGCAGTACG
CCTTTGGTTTATCCATTTTATACAATCCATGTAAAAAAGGGCCCTGAAATTCAGGACCCTTT
CTGAGCTCATTACAGGTTGGTGCTAATACCATTATAGTAGCTCTCGGAACGGCTTGCACGT
TTAATGTTTTTGAAACCGTGCATTACTTTCAGCAGACGTTCGAGACCAAAACCTGCACCAAT
CCAGGGTTTATCAATACCCCATTCACGATCCAGGCTAACCGGACCAACAACTGCGCTACTC
AGTTCCAGATCACCGTGCATAATATCCAGGGTATCACCATAAACCATGCAGCTATCACCAA
CAATTTCAAAGTCGATTTCCAGGTAATCCAGAAACTCTTTAATCAGTGCTTCCAGATTTTCA
CGGGTACAACCGCTACCCATCTGACAAAAGTTCACCATTGTAAATTCTTCCAGGTGTTCTTT
ACCATCACTTTCTTTACGATAGCACGGACCAACTTCAAAGATTTTGATAGGACCAGGCAGA
ATACGATCCAGTTTCCGCAGGTAGTTATACAGTGTCGGTGCCAGCATAGGACGCAGACAC
AGGTTTTTATCAACGCGAAAGATTTGTTTGCTCAGTTCGGTATCATTGTTAATGCCCATACG
TTCAACATATTCTGCCGGAATCAGAATCGGGCTTTTGATTTCCAGAAAACCGCGATCCACG
AAAAATTTGGTAATATCACGTTCCAGTTTACCCAGATAATCTTCGCGGTCGTTGGTATATAA
GCGTTGAAAATCATTTTTACGACGAGTAACCAGTTCCGGTTCCAGTTCACGAAACGGTTTT
GCCATATTCAGGCTGATTTTATCTTCAGGACTCAGCAGTGCTTCAACACGATCTAACTGAGA
CCGGGTTAAGCTCGGTGCCGGTGCGCTTGCCGGAACGCTGCTATTCGGGGTGCTTTTTGC
CGGACTCGGAACGCTACGGCTGGTATTGGTGCTTGCTTTTGCGCTAACGCTATTTTCCAGA
GGTTTCGGTGCGCGACTAACGCTTTTCGGCATTGCTTTTTTAACTTTAGGTGCGCTCACAA
CACGAACTTTAACTGAATTTTTGCTTTCGGTGCTGCGTGTCAGAAAATTGTTAATATCTTCAT
CACTCACACGGCAGCGTTTACAGGTTTTACGATATTTGTGATGACGAAATGCGCGTGCGGT
ACGACAGCTACGGCTATTATTCACAACCAGATGATCGCCACAGGCCATTTCAATATAGATTT
TGCTGCGGCTAACTTCGTGATGTTTGATTTTATGCAGGGTGCCGGTACGGCTCATCCACAG
ACCTGTTGCGCTAATCAGAACATCCAGCGGTTTTTTATCCATATCGTACCTCCTTAAATTTC
TAGGTTGTGACCTAGGTGATTTAGTTTACCAGTGCAAAAGAAATGTCAAAAGAGAAGGGCG
TGAATTTAACGCGGTTCCAGCGCAAAGACTTCAAAACCTGCGTCGGTGCCGATTTCGGCCT
ATTGGTTAAAAAATGAGCTGAGTTCTAGTAAAAAAAATCCTTAGCTTTCGCTAAGGATCTGC
AGTGGCGGAAACCCCGGGAATCTAACCCGGCTGAACGGATTTAGAGTCCATTCGATCTAC
ATGATCAGGTTTCCGAATTCAGCGTTACAAGTATTACACAAAGTTTTTTATGTTGAGAATATT
TTTTTGATGGGGCATGGCGCAAAACCTTTCGCGGTATGGCATGCAGGTGGCACTTTTCGG
GGAAATGTGCGCGGAACCCCTATTTGTTTATTTTTCTAAATACATTCAAATATGTATCCGCT
CATGAGACAATAACCCTGATAAATGCTTCAATAATATTGAAAAAGGAAGAGTATGAGTATTC
AACATTTCCGTGTCGCCCTTATTCCCTTTTTTGCGGCATTTTGCCTTCCTGTTTTTGCTCAC
CCAGAAACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAACGTTTTC
CAATGATGAGCACTTTTAAAGTTCTGCTATGTGGCGCGGTATTATCCCGTATTGACGCCGG
GCAAGAGCAACTCGGTCGCCGCATACACTATTCTCAGAATGACTTGGTTGAGTACTCACCA
GTCACAGAAAAGCATCTTACGGATGGCATGACAGTAAGAGAATTATGCAGTGCTGCCATAA
CCATGAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAACCGGA
GCTGAATGAAGCCATACCAAACGACGAGCGTGACACCACGATGCCTGTAGCAATGGCAAC
AACGTTGCGCAAACTATTAACTGGCGAACTACTTACTCTAGCTTCCCGGCAACAATTGATA
GACTGGATGGAGGCGGATAAAGTTGCAGGACCACTTCTGCGCTCGGCCCTTCCGGCTGG
CTGGTTTATTGCTGATAAATCTGGAGCCGGTGAGCGTGGCTCTCGCGGTATCATTGCAGCA
CTGGGGCCAGATGGTAAGCCCTCCCGTATCGTAGTTATCTACACGACGGGGAGTCAGGCA
ACTATGGATGAACGAAATAGACAGATCGCTGAGATAGGTGCCTCACTGATTAAGCATTGGT
AAGAATTAATGATGTCTCGTTTAGATAAAAGTAAAGTGATTAACAGCGCATTAGAGCTGCTT
AATGAGGTCGGAATCGAAGGTTTAACAACCCGTAAACTCGCCCAGAAGCTAGGTGTAGAG
CAGCCTACATTGTATTGGCATGTAAAAAATAAGCGGGCTTTGCTCGACGCCTTAGCCATTG
AGATGTTAGATAGGCACCATACTCACTTTTGCCCTTTAGAAGGGGAAAGCTGGCAAGATTT
TTTACGTAATAACGCTAAAAGTTTTAGATGTGCTTTACTAAGTCATCGCGATGGAGCAAAAG
TACATTTAGGTACACGGCCTACAGAAAAACAGTATGAAACTCTCGAAAATCAATTAGCCTTT
TTATGCCAACAAGGTTTTTCACTAGAGAATGCATTATATGCACTCAGCGCAGTGGGGCATTT
TACTTTAGGTTGCGTATTGGAAGATCAAGAGCATCAAGTCGCTAAAGAAGAAAGGGAAACA
CCTACTACTGATAGTATGCCGCCATTATTACGACAAGCTATCGAATTATTTGATCACCAAGG
TGCAGAGCCAGCCTTCTTATTCGGCCTTGAATTGATCATATGCGGATTAGAAAAACAACTTA
AATGTGAAAGTGGGTCTTAATGAGAATATTCGTTTTCACCCAAGGAATAGAGGATATGGAG
AAAAAAATCACTGGATATACCACCGTTGATATATCCCAATGGCATCGTAAAGAACATTTTGA
GGCATTTCAGTCAGTTGCTCAATGTACCTATAACCAGACCGTTCAGCTGGATATTACGGCC
TTTTTAAAGACCGTAAAGAAAAATAAGCACAAGTTTTATCCGGCCTTTATTCACATTCTTGCC
CGCCTGATGAATGCTCATCCGGAGTTCCGTATGGCAATGAAAGACGGTGAGCTGGTGATA
TGGGATAGTGTTCACCCTTGTTACACCGTTTTCCATGAGCAAACTGAAACGTTTTCATCGCT
CTGGAGTGAATACCACGACTAGTTCCGGCAGTTTCTACACATATATTCGCAAGATGTGGCG
TGTTACGGTGAAAACCTGGCCTATTTCCCTAAAGGGTTTATTGAGAATATGTTTTTCGTCTC
AGCCAATCCCTGGGTGAGTTTCACCAGTTTTGATTTAAACGTGGCCAATATGGACAACTTCT
TCGCCCCCGTTTTCACTATGGGCAAATATTATACGCAAGGCGACAAGGTGCTGATGCCGCT
GGCGATTCAGGTTCATCATGCCGTTTGTGATGGCTTCCATGTCGGCAGAATGCTTAATGAA
TTACAACAGTACTGCGATGAGTGGCAGGGCGGGGCGTAATAGCTTCACTAGTTTAAAAGG
ATCTAGGTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGATAAGTCGTGTC
TTACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTTGTGATG
CTCGTCAGGGGGGCGGAGCCTATGGAAAAACGCCAGCAACGCGGCC
SEQ ID NO: 24: Nucleic acid sequence of cat^{UAG119}
ATGGAGAAAAAAATCACTGGATATACCACCGTTGATATATCCCAATGGCATCGTAAAGAACA
TTTTGAGGCATTTCAGTCAGTTGCTCAATGTACCTATAACCAGACCGTTCAGCTGGATATTA
CGGCCTTTTTAAAGACCGTAAAGAAAAATAAGCACAAGTTTTATCCGGCCTTTATTCACATT
CTTGCCCGCCTGATGAATGCTCATCCGGAGTTCCGTATGGCAATGAAAGACGGTGAGCTG
GTGATATGGGATAGTGTTCACCCTTGTTACACCGTTTTCCATGAGCAAACTGAAACGTTTTC
ATCGCTCTGGAGTGAATACCACGACTAGTTCCGGCAGTTTCTACACATATATTCGCAAGAT
GTGGCGTGTTACGGTGAAAACCTGGCCTATTTCCCTAAAGGGTTTATTGAGAATATGTTTTT
CGTCTCAGCCAATCCCTGGGTGAGTTTCACCAGTTTTGATTTAAACGTGGCCAATATGGAC
AACTTCTTCGCCCCCGTTTTCACTATGGGCAAATATTATACGCAAGGCGACAAGGTGCTGA
TGCCGCTGGCGATTCAGGTTCATCATGCCGTTTGTGATGGCTTCCATGTCGGCAGAATGCT
TAATGAATTACAACAGTACTGCGATGAGTGGCAGGGCGGGGCGTAA
SEQ ID NO: 25: Nucleic acid sequence of eGFP^{UAG39}
ATGTCTAAAGGTGAAGAACTTTTCACTGGAGTTGTCCCAATTCTTGTTGAATTAGATGGTGA
TGTTAATGGGCACAAATTTTCTGTCAGTGGAGAGGGTGAAGGTGATGCAACATAGGGAAAA
CTTACCCTTAAATTTATTTGCACTACTGGAAAACTACCTGTTCCATGGCCAACACTTGTCAC
TACTTTGACTTATGGTGTTCAATGCTTTTCAAGATACCCGGATCATATGAAACGGCATGACT
TTTTCAAGAGTGCCATGCCCGAAGGTTATGTACAGGAAAGAACTATATTTTTCAAAGATGAC
GGGAACTACAAGACACGTGCTGAAGTCAAGTTTGAAGGTGATACCCTTGTTAATAGAATCG
AGTTAAAAGGTATTGATTTTAAAGAAGATGGAAACATTCTTGGACACAAATTGGAATACAAC
TATAACTCACACAATGTATACATCATGGCAGACAAACAAAAGAATGGAATCAAAGTTAACTT
CAAAATTAGACACAACATTGAAGATGGAAGCGTTCAACTAGCAGACCATTATCAACAAAATA
CTCCAATTGGCGATGGCCCTGTCCTTTTACCAGACAACCATTACCTGTCCACACAATCTGC
CCTTTCGAAAGATCCCAACGAAAAGAGGGACCACATGGTCCTTCTTGAGTTTGTAACAGCT
GCTGGGATTACACATGGCATGGATGAACTGTACCAA
SEQ ID NO: 26: Nucleic acid sequence of wt PylRS
ATGGATAAAAAACCGCTGGATGTTCTGATTAGCGCAACAGGTCTGTGGATGAGCCGTACC
GGCACCCTGCATAAAATCAAACATCACGAAGTTAGCCGCAGCAAAATCTATATTGAAATGG
CCTGTGGCGATCATCTGGTTGTGAATAATAGCCGTAGCTGTCGTACCGCACGCGCATTTCG
TCATCACAAATATCGTAAAACCTGTAAACGCTGCCGTGTGAGTGATGAAGATATTAACAATT
TTCTGACACGCAGCACCGAAAGCAAAAATTCAGTTAAAGTTCGTGTTGTGAGCGCACCTAA
AGTTAAAAAAGCAATGCCGAAAAGCGTTAGTCGCGCACCGAAACCTCTGGAAAATAGCGTT
AGCGCAAAAGCAAGCACCAATACCAGCCGTAGCGTTCCGAGTCCGGCAAAAAGCACCCCG
AATAGCAGCGTTCCGGCAAGCGCACCGGCACCGAGCTTAACCCGGTCTCAGTTAGATCGT
GTTGAAGCACTGCTGAGTCCTGAAGATAAAATCAGCCTGAATATGGCAAAACCGTTTCGTG
AACTGGAACCGGAACTGGTTACTCGTCGTAAAAATGATTTTCAACGCTTATATACCAACGAC
CGCGAAGATTATCTGGGTAAACTGGAACGTGATATTACCAAATTTTTCGTGGATCGCGGTT
TTCTGGAAATCAAAAGCCCGATTCTGATTCCGGCAGAATATGTTGAACGTATGGGCATTAA
CAATGATACCGAACTGAGCAAACAAATCTTTCGCGTTGATAAAAACCTGTGTCTGCGTCCTA
TGCTGGCACCGACACTGTATAACTACCTGCGGAAACTGGATCGTATTCTGCCTGGTCCTAT
CAAAATCTTTGAAGTTGGTCCGTGCTATCGTAAAGAAAGTGATGGTAAAGAACACCTGGAA
GAATTTACAATGGTGAACTTTTGTCAGATGGGTAGCGGTTGTACCCGTGAAAATCTGGAAG
CACTGATTAAAGAGTTTCTGGATTACCTGGAAATCGACTTTGAAATTGTTGGTGATAGCTGC
ATGGTTTATGGTGATACCCTGGATATTATGCACGGTGATCTGGAACTGAGTAGCGCAGTTG
TTGGTCCGGTTAGCCTGGATCGTGAATGGGGTATTGATAAACCCTGGATTGGTGCAGGTTT
TGGTCTCGAACGTCTGCTGAAAGTAATGCACGGTTTCAAAAACATTAAACGTGCAAGCCGT
TCCGAGAGCTACTATAATGGTATTAGCACCAACCTG
SEQ ID NO: 27:
TAGCTGCATGGTTTTTGGTGATACCCTGG
SEQ ID NO: 28:
CCAGGGTATCACCAAAAACCATGCAGCTA
SEQ ID NO: 29: Nucleic acid sequence of PylRS#1 (Y349F)
ATGGATAAAAAACCGCTGGATGTTCTGATTAGCGCAACAGGTCTGTGGATGAGCCGTACC
GGCACCCTGCATAAAATCAAACATCACGAAGTTAGCCGCAGCAAAATCTATATTGAAATGG
CCTGTGGCGATCATCTGGTTGTGAATAATAGCCGTAGCTGTCGTACCGCACGCGCATTTCG
TCATCACAAATATCGTAAAACCTGTAAACGCTGCCGTGTGAGTGATGAAGATATTAACAATT
TTCTGACACGCAGCACCGAAAGCAAAAATTCAGTTAAAGTTCGTGTTGTGAGCGCACCTAA
AGTTAAAAAAGCAATGCCGAAAAGCGTTAGTCGCGCACCGAAACCTCTGGAAAATAGCGTT
AGCGCAAAAGCAAGCACCAATACCAGCCGTAGCGTTCCGAGTCCGGCAAAAAGCACCCCG
AATAGCAGCGTTCCGGCAAGCGCACCGGCACCGAGCTTAACCCGGTCTCAGTTAGATCGT
GTTGAAGCACTGCTGAGTCCTGAAGATAAAATCAGCCTGAATATGGCAAAACCGTTTCGTG
AACTGGAACCGGAACTGGTTACTCGTCGTAAAAATGATTTTCAACGCTTATATACCAACGAC
CGCGAAGATTATCTGGGTAAACTGGAACGTGATATTACCAAATTTTTCGTGGATCGCGGTT
TTCTGGAAATCAAAAGCCCGATTCTGATTCCGGCAGAATATGTTGAACGTATGGGCATTAA
CAATGATACCGAACTGAGCAAACAAATCTTTCGCGTTGATAAAAACCTGTGTCTGCGTCCTA
TGCTGGCACCGACACTGTATAACTACCTGCGGAAACTGGATCGTATTCTGCCTGGTCCTAT
CAAAATCTTTGAAGTTGGTCCGTGCTATCGTAAAGAAAGTGATGGTAAAGAACACCTGGAA
GAATTTACAATGGTGAACTTTTGTCAGATGGGTAGCGGTTGTACCCGTGAAAATCTGGAAG
CACTGATTAAAGAGTTTCTGGATTACCTGGAAATCGACTTTGAAATTGTTGGTGATAGCTGC
ATGGTTTTTGGTGATACCCTGGATATTATGCACGGTGATCTGGAACTGAGTAGCGCAGTTG
TTGGTCCGGTTAGCCTGGATCGTGAATGGGGTATTGATAAACCCTGGATTGGTGCAGGTTT
TGGTCTCGAACGTCTGCTGAAAGTAATGCACGGTTTCAAAAACATTAAACGTGCAAGCCGT
TCCGAGAGCTACTATAATGGTATTAGCACCAACCTG
SEQ ID NO: 30:
GAGCTTAACCCGGTCTCAGTTAGATCG
SEQ ID NO: 31:
GGTAGCGGTTGTACCCGTG
SEQ ID NO: 32:
GGGTACAACCGCTACCSNNCTGSNNAAASNNCACSNNTGTAATTCTTCCAGGTGTT
SEQ ID NO: 33:
CTTTCAGCAGACGTTCGAGACCAAAACCTGCACCAATSNNGGGTTTATCAATACCCCATTC
SEQ ID NO: 34:
CTTTCAGCAGACGTTCGAGAC
SEQ ID NO: 35: Nucleic acid sequence of CafRS#7
ATGGATAAAAAACCGCTGGATGTTCTGATTAGCGCAACAGGTCTGTGGATGAGCCGTACC
GGCACCCTGCATAAAATCAAACATCACGAAGTTAGCCGCAGCAAAATCTATATTGAAATGG
CCTGTGGCGATCATCTGGTTGTGAATAATAGCCGTAGCTGTCGTACCGCACGCGCATTTCG
TCATCACAAATATCGTAAAACCTGTAAACGCTGCCGTGTGAGTGATGAAGATATTAACAATT
TTCTGACACGCAGCACCGAAAGCAAAAATTCAGTTAAAGTTCGTGTTGTGAGCGCACCTAA
AGTTAAAAAAGCAATGCCGAAAAGCGTTAGTCGCGCACCGAAACCTCTGGAAAATAGCGTT
AGCGCAAAAGCAAGCACCAATACCAGCCGTAGCGTTCCGAGTCCGGCAAAAAGCACCCCG
AATAGCAGCGTTCCGGCAAGCGCACCGGCACCGAGCTTAACCCGGTCTCAGTTAGATCGT
GTTGAAGCACTGCTGAGTCCTGAAGATAAAATCAGCCTGAATATGGCAAAACCGTTTCGTG
AACTGGAACCGGAACTGGTTACTCGTCGTAAAAATGATTTTCAACGCTTATATACCAACGAC
CGCGAAGATTATCTGGGTAAACTGGAACGTGATATTACCAAATTTTTCGTGGATCGCGGTT
TTCTGGAAATCAAAAGCCCGATTCTGATTCCGGCAGAATATGTTGAACGTATGGGCATTAA
CAATGATACCGAACTGAGCAAACAAATCTTTCGCGTTGATAAAAACCTGTGTCTGCGTCCTA
TGCTGGCACCGACACTGTATAACTACCTGCGGAAACTGGATCGTATTCTGCCTGGTCCTAT
CAAAATCTTTGAAGTTGGTCCGTGCTATCGTAAAGAAAGTGATGGTAAAGAACACCTGGAA
GAATTTACACAGGTGTCCTTTGGCCAGATGGGTAGCGGTTGTACCCGTGAAAATCTGGAAG
CACTGATTAAAGAGTTTCTGGATTACCTGGAAATCGACTTTGAAATTGTTGGTGATAGCTGC
ATGGTTTTTGGTGATACCCTGGATATTATGCACGGTGATCTGGAACTGAGTAGCGCAGTTG
TTGGTCCGGTTAGCCTGGATCGTGAATGGGGTATTGATAAACCCTGGATTGGTGCAGGTTT
TGGTCTCGAACGTCTGCTGAAAGTAATGCACGGTTTCAAAAACATTAAACGTGCAAGCCGT
TCCGAGAGCTACTATAATGGTATTAGCACCAACCTG
SEQ ID NO: 36: Nucleic acid sequence of CafRS#7-R6
ATGGATAAAAAACCGCTGGATGTTCTGATTAGCGCAACAGGTCTGTGGATGAGCCGTACC
GGCACCCTGCATAAAATCAAACATCACGAAGTTAGCCGCAGCAAAATCTATATTGAAATGG
CCTGTGGCGATCATCTGGTTGTGAATAATAGCCGTAGCTGTCGTACCGCACGCGCATTTCG
TCATCACAAATATCGTAAAACCTGTAAACGCTGCCGTGTGAGTGATGAAGATATTAACAATT
TTCTGACACGCAGCACCGAAAGCAAAAATTCAGTTAAAGTTCGTGTTGTGAGCGCACCTAA
AGTTAAAAAAGCAATGCCGAAAAGCGTTAGTCGCGCACCGAAACCTCTGGAAAATAGCGTT
AGCGCAAAAGCAAGCACCAATACCAGCCGTAGCGTTCCGAGTCCGGCAAAAAGCACCCCG
AATAGCAGCGTTCCGGCAAGCGCACCGGCACCGAGCTTAACCCGGTCTCAGTTAGATCGT
GTTGAAGCACTGCTGAGTCCTGAAGATAAAATCAGCCTGAATATGGCAAAACCGTTTCGTG
AACTGGAACCGGAACTGGTTACTCGTCGTAAAAATGATTTTCAACGCTTATATACCAACGAC
CGCGAAGATTATCTGGGTAAACTGGAACGTGATATTACCAAATTTTTCGTGGATCGCGGTT
TTCTGGAAATCAAAAGCCCGATTCTGATTCCGGCAGAATATGTTGAACGTATGGGCATTAA
CAATGATACCGAACTGAGCAAACAAATCTTTCGCGTTGATAAAAACCTGTGTCTGCGTCCTA
TGCTGNNNCCGACANNSNNSAACTACNNNCGGAAACTGGATCGTATTCTGCCTGGTCCTN
NSAAANNSTTTGAAGTTGGTCCGTGCTATCGTAAAGAAAGTGATGGTAAAGAACACCTGGA
AGAATTTACACAGGTGTCCTTTGGCCAGATGGGTAGCGGTTGTACCCGTGAAAATCTGGAA
GCACTGATTAAAGAGTTTCTGGATTACCTGGAAATCGACTTTGAAATTGTTGGTGATAGCTG
CATGGTTTTTGGTGATACCCTGGATATTATGCACGGTGATCTGGAACTGAGTAGCGCAGTT
GTTGGTCCGGTTAGCCTGGATCGTGAATGGGGTATTGATAAACCCTGGATTGGTGCAGGT
TTTGGTCTCGAACGTCTGCTGAAAGTAATGCACGGTTTCAAAAACATTAAACGTGCAAGCC
GTTCCGAGAGCTACTATAATGGTATTAGCACCAACCTG
SEQ ID NO: 37:
GGCAGAATACGATCCAGTTTCCGSNNGTAGTTSNNSNNTGTCGGSNNCAGCATAGGACGC
AGACAC
SEQ ID NO: 38:
CGGAAACTGGATCGTATTCTGCCTGGTCCTNNSAAANNSTTTGAAGTTGGTCCGTGCTATC
GT
SEQ ID NO: 39: Nucleic acid sequence of CafRS#29
ATGGATAAAAAACCGCTGGATGTTCTGATTAGCGCAACAGGTCTGTGGATGAGCCGTACC
GGCACCCTGCATAAAATCAAACATCACGAAGTTAGCCGCAGCAAAATCTATATTGAAATGG
CCTGTGGCGATCATCTGGTTGTGAATAATAGCCGTAGCTGTCGTACCGCACGCGCATTTCG
TCATCACAAATATCGTAAAACCTGTAAACGCTGCCGTGTGAGTGATGAAGATATTAACAATT
TTCTGACACGCAGCACCGAAAGCAAAAATTCAGTTAAAGTTCGTGTTGTGAGCGCACCTAA
AGTTAAAAAAGCAATGCCGAAAAGCGTTAGTCGCGCACCGAAACCTCTGGAAAATAGCGTT
AGCGCAAAAGCAAGCACCAATACCAGCCGTAGCGTTCCGAGTCCGGCAAAAAGCACCCCG
AATAGCAGCGTTCCGGCAAGCGCACCGGCACCGAGCTTAACCCGGTCTCAGTTAGATCGT
GTTGAAGCACTGCTGAGTCCTGAAGATAAAATCAGCCTGAATATGGCAAAACCGTTTCGTG
AACTGGAACCGGAACTGGTTACTCGTCGTAAAAATGATTTTCAACGCTTATATACCAACGAC
CGCGAAGATTATCTGGGTAAACTGGAACGTGATATTACCAAATTTTTCGTGGATCGCGGTT
TTCTGGAAATCAAAAGCCCGATTCTGATTCCGGCAGAATATGTTGAACGTATGGGCATTAA
CAATGATACCGAACTGAGCAAACAAATCTTTCGCGTTGATAAAAACCTGTGTCTGCGTCCTA
TGCTGACCCCGACATTGTTCAACTACGCGCGGAAACTGGATCGTATTCTGCCTGGTCCTAA
CAAGAGCTTTGAAGTTGGTCCGTGCTATCGTAAAGAAAGTGATGGTAAAGAACACCTGGAA
GAATTTACACAGGTGTCCTTTGGCCAGATGGGTAGCGGTTGTACCCGTGAAAATCTGGAAG
CACTGATTAAAGAGTTTCTGGATTACCTGGAAATCGACTTTGAAATTGTTGGTGATAGCTGC
ATGGTTTTTGGTGATACCCTGGATATTATGCACGGTGATCTGGAACTGAGTAGCGCAGTTG
TTGGTCCGGTTAGCCTGGATCGTGAATGGGGTATTGATAAACCCTGGATTGGTGCAGGTTT
TGGTCTCGAACGTCTGCTGAAAGTAATGCACGGTTTCAAAAACATTAAACGTGCAAGCCGT
TCCGAGAGCTACTATAATGGTATTAGCACCAACCTGTAATGAGCTCAGAGAGGGTCCTGAT
TTTCAGGGCCCTTTTTTTACGTGGTATTGTATAAAATGGATAAACCAAAGGCGTACTGCCGC
GCAGTAGCGGTAAACGGCAGACAAAAAAAATGTCGCACAGTG
SEQ ID NO: 40: Nucleic acid sequence of CafRS#29-R5
ATGGATAAAAAACCGCTGGATGTTCTGATTAGCGCAACAGGTCTGTGGATGAGCCGTACC
GGCACCCTGCATAAAATCAAACATCACGAAGTTAGCCGCAGCAAAATCTATATTGAAATGG
CCTGTGGCGATCATCTGGTTGTGAATAATAGCCGTAGCTGTCGTACCGCACGCGCATTTCG
TCATCACAAATATCGTAAAACCTGTAAACGCTGCCGTGTGAGTGATGAAGATATTAACAATT
TTCTGACACGCAGCACCGAAAGCAAAAATTCAGTTAAAGTTCGTGTTGTGAGCGCACCTAA
AGTTAAAAAAGCAATGCCGAAAAGCGTTAGTCGCGCACCGAAACCTCTGGAAAATAGCGTT
AGCGCAAAAGCAAGCACCAATACCAGCCGTAGCGTTCCGAGTCCGGCAAAAAGCACCCCG
AATAGCAGCGTTCCGGCAAGCGCACCGGCACCGAGCTTAACCCGGTCTCAGTTAGATCGT
GTTGAAGCACTGCTGAGTCCTGAAGATAAAATCAGCCTGAATATGGCAAAACCGTTTCGTG
AACTGGAACCGGAACTGGTTACTCGTCGTAAAAATGATTTTCAACGCTTATATACCAACGAC
CGCGAAGATTATCTGGGTAAACTGGAACGTGATATTACCAAATTTTTCGTGGATCGCGGTT
TTCTGGAAATCAAAAGCCCGATTCTGATTCCGGCAGAATATGTTGAACGTATGGGCATTAA
CAATGATACCGAACTGAGCAAACAAATCTTTCGCGTTGATAAAAACCTGTGTCTGCGTCCTA
TGCTGACCCCGACATTGTTCAACTACNNSCGGAAACTGGATCGTATTCTGCCTGGTCCTNN
SAAGNNSTTTGAAGTTGGTCCGTGCTATCGTAAAGAAAGTGATGGTAAAGAACACCTGGAA
GAATTTACANNSGTGNNSTTTGGCCAGATGGGTAGCGGTTGTACCCGTGAAAATCTGGAAG
CACTGATTAAAGAGTTTCTGGATTACCTGGAAATCGACTTTGAAATTGTTGGTGATAGCTGC
ATGGTTTTTGGTGATACCCTGGATATTATGCACGGTGATCTGGAACTGAGTAGCGCAGTTG
TTGGTCCGGTTAGCCTGGATCGTGAATGGGGTATTGATAAACCCTGGATTGGTGCAGGTTT
TGGTCTCGAACGTCTGCTGAAAGTAATGCACGGTTTCAAAAACATTAAACGTGCAAGCCGT
TCCGAGAGCTACTATAATGGTATTAGCACCAACCTGTAATGAGCTCAGAGAGGGTCCTGAT
TTTCAGGGCCCTTTTTTTACGTGGTATTGTATAAAATGGATAAACCAAAGGCGTACTGCCGC
GCAGTAGCGGTAAACGGCAGACAAAAAAAATGTCGCACAGTG
SEQ ID NO: 41:
CAGAATACGATCCAGTTTCCGSNNGTAGTTATACAGTGTCGG
SEQ ID NO: 42:
GGGTACAACCGCTACCCATCTGGCCAAASNNCACSNNTGTAAATTCTTCCAGGTGTT
SEQ ID NO: 43: Nucleic acid sequence of CafRS#30
ATGGATAAAAAACCGCTGGATGTTCTGATTAGCGCAACAGGTCTGTGGATGAGCCGTACC
GGCACCCTGCATAAAATCAAACATCACGAAGTTAGCCGCAGCAAAATCTATATTGAAATGG
CCTGTGGCGATCATCTGGTTGTGAATAATAGCCGTAGCTGTCGTACCGCACGCGCATTTCG
TCATCACAAATATCGTAAAACCTGTAAACGCTGCCGTGTGAGTGATGAAGATATTAACAATT
TTCTGACACGCAGCACCGAAAGCAAAAATTCAGTTAAAGTTCGTGTTGTGAGCGCACCTAA
AGTTAAAAAAGCAATGCCGAAAAGCGTTAGTCGCGCACCGAAACCTCTGGAAAATAGCGTT
AGCGCAAAAGCAAGCACCAATACCAGCCGTAGCGTTCCGAGTCCGGCAAAAAGCACCCCG
AATAGCAGCGTTCCGGCAAGCGCACCGGCACCGAGCTTAACCCGGTCTCAGTTAGATCGT
GTTGAAGCACTGCTGAGTCCTGAAGATAAAATCAGCCTGAATATGGCAAAACCGTTTCGTG
AACTGGAACCGGAACTGGTTACTCGTCGTAAAAATGATTTTCAACGCTTATATACCAACGAC
CGCGAAGATTATCTGGGTAAACTGGAACGTGATATTACCAAATTTTTCGTGGATCGCGGTT
TTCTGGAAATCAAAAGCCCGATTCTGATTCCGGCAGAATATGTTGAACGTATGGGCATTAA
CAATGATACCGAACTGAGCAAACAAATCTTTCGCGTTGATAAAAACCTGTGTCTGCGTCCTA
TGCTGACCCCGACATTGTATAACTACAGCCGGAAACTGGATCGTATTCTGCCTGGTCCTTC
CAAAGTCTTTGAAGTTGGTCCGTGCTATCGTAAAGAAAGTGATGGTAAAGAACACCTGGAA
GAATTTACAATGGTGGTGTTTGGCCAGATGGGTAGCGGTTGTACCCGTGAAAATCTGGAAG
CACTGATTAAAGAGTTTCTGGATTACCTGGAAATCGACTTTGAAATTGTTGGTGATAGCTGC
ATGGTTTTTGGTGATACCCTGGATATTATGCACGGTGATCTGGAACTGAGTAGCGCAGTTG
TTGGTCCGGTTAGCCTGGATCGTGAATGGGGTATTGATAAACCCIGGATTGGTGCAGGTTT
TGGTCTCGAACGTCTGCTGAAAGTAATGCACGGTTTCAAAAACATTAAACGTGCAAGCCGT
TCCGAGAGCTACTATAATGGTATTAGCACCAACCTGTAA
SEQ ID NO: 44: Nucleic acid sequence pSAm1
GCACTTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAGGAAGAG
TATGAGTATTCAACATTTCCGTGTCGCCCTTATTCCCTTTTTTGCGGCATTTTGCCTTCCTGT
TTTTGCTCACCCAGAAACGCTGGTGAAAGTAAAAGATGCTGAAGATCAGTTGGGTGCACGA
GTGGGTTACATCGAACTGGATCTCAACAGCGGTAAGATCCTTGAGAGTTTTCGCCCCGAAG
AACGTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATGCAGTG
CTGCCATAACCATGAGTGATAACACTGCGGCCAACTTACTTCTGACAACGATCGGAGGACC
GAAGGAGCTAACCGCTTTTTTGCACAACATGGGGGATCATGTAACTCGCCTTGATCGTTGG
GAACCGGAGCTGAATGAAGCCATACCAAACGACGAGCGTGACACCACGATGCCTGTAGCA
ATGGCAACAACGTTGCGCAAACTATTAACTGGCGAACTACTTACTCTAGCTTCCCGGCAAC
AATTAATAGACTGGATGGAGGCGGATAAAGTTGCAGGACCACTTCTGCGCTCGGCCCTTC
CGGCTGGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGACGGGGA
GTCAGGCAACTATGGATGAACGAAATAGACAGATCGCTGAGATAGGTGCCTCACTGATTAA
GCATTGGTAACTGTCAGACCAAGTTTACTCATATATACTTTAGATTGATTTAAAACTTCATTT
TTAATTTAAAAGGATCTAGGTGAAGATCCTTTTTGATAATCTCATGACCAAAATCCCTTAACG
TGAGTTTTCGTTCCACTGAGCGTCAGACCCCGTAGAAAAGATCAAAGGATCTTCTTGAGAT
CCTTTTTTTCTGCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGCTTCAGCAGAGC
GCAGATACCAAATACTGTCCTTCTAGTGTAGCCGTAGTTAGGCCACCACTTCAAGAACTCT
GTAGCACCGCCTACATACCTCGCTCTGCTAATCCTGTTACCAGTGGCTGCTGCCAGTGGC
GATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGCAACGCGGCCT
TTTTACGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAGAGCGCCCAATACGCAAACC
GCCTCTCCCCGCGCGTTGGCCGATTCATTAATGCAGGATCTCGATCCCGCGAAATTAATAC
GACTCACTATAGGGAGACCACAACGGTTTCCCTCTAGAAATAATTTTGTTTAACTTTAAGAA
GGAGATATACATATGGAAGCAGGTATCACCGGCACCTGGTACAACCAGCTCGGCTCGACC
TTCATCGTGACCGCGGGTGCAGACGGAGCTCTGACCGGTACCTACGTCACGGCGCGTGG
CAACGCCGAGAGCCGCTACGTCCTGACCGGTCGTTACGACAGCGCCCCGGCCACCGACG
GCAGCGGCACCGCCCTCGGTTGGACGGTGGCCTGGAAGAATAACTACCGCAACGCCCAC
TCCGCGACCACGTGGAGCGGCCAGTACGTCGGCGGCGCCGAGGCGAGGATCAACACCC
AGTGGCTGCTGACCTCCGGCACCACCGAGGCCAACGCCTGGAAGTCCACGCTGGTCGGC
CACGACACCTTCACCAAGGTGAAGCCGTCCGCCGCCTCCTAATAAGCTTGATCCGGCTGC
TAACAAAGCCCGAAAGGAAGCTGAGTTGGCTGCTGCCACCGCTGAGCAATAACTAGCATA
ACCCCTTGGGGCCTCTAAACGGGTCTTGAGGGGTTTTTTGCTGAAAGGAGGAACTATATCC
GGATCTGGCGTAATAGCGAAGAGGCCCGCACCGATCGCCCTTCCCAACAGTTGCGCAGC
CTGAATGGCGAATGGGACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCTTTCTT
CCCTTCCTTTCTCGCCACGTTCGCCGGCTTTCCCCGTCAAGCTCTAAATCGGGGGCTCCCT
TTAGGGTTCCGATTTAGTGCTTTACGGCACCTCGACCCCAAAAAACTTGATTAGGGTGATG
GTTCACGTAGTGGGCCATCGCCCTGATAGACGGTTTTTCGCCCTTTGACGTTGGAGTCCAC
GTTCTTTAATAGTGGACTCTTGTTCCAAACTGGAACAACACTCAACCCTATCTCGGTCTATT
CTTTTGATTTATAAGGGATTTTGCCGATTTCGGCCTATTGGTTAAAAAATGAGCTGATTTAAC
AAAAATTTAACGCGAATTTTAACAAATATTAACGCTTACAATTTAGGTG
SEQ ID NO: 45:
GCAGACGGaGCCCTGACCGGtACCTACTAGacggcgcgtG
SEQ ID NO: 46:
CacgcgccgtCTAGTAGGTaCCGGTCAGGGCtCCGTCTGC
SEQ ID NO: 47: Nucleic acid sequence SAm1^{UAG44}
ATGGAAGCAGGTATCACCGGCACCTGGTACAACCAGCTCGGCTCGACCTTCATCGTGACC
GCGGGTGCAGACGGAGCCCTGACCGGTACCTACTAGACGGCGCGTGGCAACGCCGAGA
GCCGCTACGTCCTGACCGGTCGTTACGACAGCGCCCCGGCCACCGACGGCAGCGGCACC
GCCCTCGGTTGGACGGTGGCCTGGAAGAATAACTACCGCAACGCCCACTCCGCGACCAC
GTGGAGCGGCCAGTACGTCGGCGGCGCCGAGGCGAGGATCAACACCCAGTGGCTGCTG
ACCTCCGGCACCACCGAGGCCAACGCCTGGAAGTCCACGCTGGTCGGCCACGACACCTT
CACCAAGGTGAAGCCGTCCGCCGCCTCCTAA
SEQ ID NO: 48:
GATCAACACCCAGTAGCTGCTGACCTCC
SEQ ID NO: 49:
GGAGGTCAGCAGCTACTGGGTGTTGATC
SEQ ID NO: 50:
GAGGCCAACGCCTAGAAGTCCACGCTGG
SEQ ID NO: 51:
CCAGCGTGGACTTCTAGGCGTTGGCCTC
SEQ ID NO: 52: Nucleic acid sequence SAm1^{UAG120}
ATGGAAGCAGGTATCACCGGCACCTGGTACAACCAGCTCGGCTCGACCTTCATCGTGACC
GCGGGTGCAGACGGAGCTCTGACCGGTACCTACGTCACGGCGCGTGGCAACGCCGAGAG
CCGCTACGTCCTGACCGGTCGTTACGACAGCGCCCCGGCCACCGACGGCAGCGGCACCG
CCCTCGGTTGGACGGTGGCCTGGAAGAATAACTACCGCAACGCCCACTCCGCGACCACGT
GGAGCGGCCAGTACGTCGGCGGCGCCGAGGCGAGGATCAACACCCAGTGGCTGCTGAC
CTCCGGCACCACCGAGGCCAACGCCTAGAAGTCCACGCTGGTCGGCCACGACACCTTCA
CCAAGGTGAAGCCGTCCGCCGCCTCCTAA
SEQ ID NO: 53: Nucleic acid sequence of pSBX8.CafRS#30.d58
TTTTTACGGTTCCTGGCCTTTTGCTGGCCTTTTGCTCACATGACCCGACACCATAACGCTC
GGTTGCCGCCGGGCGTTTTTTATTGGCCAGATGATTAATTCCTAATTTTTGTTGACACTCTA
TCATTGATAGAGTTATTTTACCACTCCCTATCAGTGATAGAGAAAAGTGAAATGAATAGTTC
GACAAAATCTAGATAACGAGGGCAAAAAATGTCTAAAGGTGAAGAACTTTTCACTGGAGTT
GTCCCAATTCTTGTTGAATTAGATGGTGATGTTAATGGGCACAAATTTTCTGTCAGTGGAGA
GGGTGAAGGTGATGCAACATAGGGAAAACTTACCCTTAAATTTATTTGCACTACTGGAAAA
CTACCTGTTCCATGGCCAACACTTGTCACTACTTTGACTTATGGTGTTCAATGCTTTTCAAG
ATACCCGGATCATATGAAACGGCATGACTTTTTCAAGAGTGCCATGCCCGAAGGTTATGTA
CAGGAAAGAACTATATTTTTCAAAGATGACGGGAACTACAAGACACGTGCTGAAGTCAAGT
TTGAAGGTGATACCCTTGTTAATAGAATCGAGTTAAAAGGTATTGATTTTAAAGAAGATGGA
AACATTCTTGGACACAAATTGGAATACAACTATAACTCACACAATGTATACATCATGGCAGA
CAAACAAAAGAATGGAATCAAAGTTAACTTCAAAATTAGACACAACATTGAAGATGGAAGCG
TTCAACTAGCAGACCATTATCAACAAAATACTCCAATTGGCGATGGCCCTGTCCTTTTACCA
GACAACCATTACCTGTCCACACAATCTGCCCTTTCGAAAGATCCCAACGAAAAGAGGGACC
ACATGGTCCTTCTTGAGTTTGTAACAGCTGCTGGGATTACACATGGCATGGATGAACTGTA
CCAAAGCGCTTGGAGCCACCCGCAGTTCGAAAAATAATAAGCTTGACCTGTGAAGTGAAAA
ATGGCGCACATTGTGCGACATTTTTTTTGTCTGCCGTTTACCGCTACTGCGCGGCAGTACG
CCTTTGGTTTATCCATTTTATACAATCCATGTAAAAAAGGGCCCTGAAATTCAGGACCCTTT
CTGAGCTCATTACAGGTTGGTGCTAATACCATTATAGTAGCTCTCGGAACGGCTTGCACGT
TTAATGTTTTTGAAACCGTGCATTACTTTCAGCAGACGTTCGAGACCAAAACCTGCACCAAT
CCAGGGTTTATCAATACCCCATTCACGATCCAGGCTAACCGGACCAACAACTGCGCTACTC
AGTTCCAGATCACCGTGCATAATATCCAGGGTATCACCAAAAACCATGCAGCTATCACCAA
CAATTTCAAAGTCGATTTCCAGGTAATCCAGAAACTCTTTAATCAGTGCTTCCAGATTTTCA
CGGGTACAACCGCTACCCATCTGGCCAAACACCACCATTGTAAATTCTTCCAGGTGTTCTT
TACCATCACTTTCTTTACGATAGCACGGACCAACTTCAAAGACTTTGGAAGGACCAGGCAG
AATACGATCCAGTTTCCGGCTGTAGTTATACAATGTCGGGGTCAGCATAGGACGCAGACAC
AGGTTTTTATCAACGCGAAAGATTTGTTTGCTCAGTTCGGTATCATTGTTAATGCCCATACG
TTCAACATATTCTGCCGGAATCAGAATCGGGCTTTTGATTTCCAGAAAACCGCGATCCACG
AAAAATTTGGTAATATCACGTTCCAGTTTACCCAGATAATCTTCGCGGTCGTTGGTATATAA
GCGTTGAAAATCATTTTTACGACGAGTAACCAGTTCCGGTTCCAGTTCACGAAACGGTTTT
GCCATATTCAGGCTGATTTTATCTTCAGGACTCAGCAGTGCTTCAACACGATCTAACTGAGA
CCGGGTTAAGCTCGGTGCCGGTGCGCTTGCCGGAACGCTGCTATTCGGGGTGCTTTTTGC
CGGACTCGGAACGCTACGGCTGGTATTGGTGCTTGCTTTTGCGCTAACGCTATTTTCCAGA
GGTTTCGGTGCGCGACTAACGCTTTTCGGCATTGCTTTTTTAACTTTAGGTGCGCTCACAA
CACGAACTTTAACTGAATTTTTGCTTTCGGTGCTGCGTGTCAGAAAATTGTTAATATCTTCAT
CACTCACACGGCAGCGTTTACAGGTTTTACGATATTTGTGATGACGAAATGCGCGTGCGGT
ACGACAGCTACGGCTATTATTCACAACCAGATGATCGCCACAGGCCATTTCAATATAGATTT
TGCTGCGGCTAACTTCGTGATGTTTGATTTTATGCAGGGTGCCGGTACGGCTCATCCACAG
ACCTGTTGCGCTAATCAGAACATCCAGCGGTTTTTTATCCATATCGTACCTCCTTAAATTTC
TAGGTTGTGACCTAGGTGATTTAGTTTACCAGTGCAAAAGAAATGTCAAAAGAGAAGGGCG
TGAATTTAACGCGGTTCCAGCGCAAAGACTTCAAAACCTGCGTCGGTGCCGATTTCGGCCT
ATTGGTTAAAAAATGAGCTGAGTTCTAGTAAAAAAAATCCTTAGCTTTCGCTAAGGATCTGC
AGTGGCGGAAACCCCGGGAATCTAACCCGGCTGAACGGATTTAGAGTCCATTCGATCTAC
ATGATCAGGTTTCCGAATTCAGCGTTACAAGTATTACACAAAGTTTTTTATGTTGAGAATATT
TTTTTGATGGGGCATGGCGCAAAACCTTTCGCGGTATGGCATGCAGGTGGCACTTTTCGG
GGAAATGTGCGCGGAACCCCTATTTGTTTATTTTTCTAAATACATTCAAATATGTATCCGCT
CATGAGACAATAACCCTGATAAATGCTTCAATAATATTGAAAAAGGAAGAGTATGAGTATTC
AACATTTCCGTGTCGCCCTTATTCCCTTTTTTGCGGCATTTTGCCTTCCTGTTTTTGCTCAC
CCAGAAACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAACGTTTTC
CAATGATGAGCACTTTTAAAGTTCTGCTATGTGGCGCGGTATTATCCCGTATTGACGCCGG
GCAAGAGCAACTCGGTCGCCGCATACACTATTCTCAGAATGACTTGGTTGAGTACTCACCA
GTCACAGAAAAGCATCTTACGGATGGCATGACAGTAAGAGAATTATGCAGTGCTGCCATAA
CCATGAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAACCGGA
GCTGAATGAAGCCATACCAAACGACGAGCGTGACACCACGATGCCTGTAGCAATGGCAAC
AACGTTGCGCAAACTATTAACTGGCGAACTACTTACTCTAGCTTCCCGGCAACAATTGATA
GACTGGATGGAGGCGGATAAAGTTGCAGGACCACTTCTGCGCTCGGCCCTTCCGGCTGG
CTGGTTTATTGCTGATAAATCTGGAGCCGGTGAGCGTGGCTCTCGCGGTATCATTGCAGCA
CTGGGGCCAGATGGTAAGCCCTCCCGTATCGTAGTTATCTACACGACGGGGAGTCAGGCA
ACTATGGATGAACGAAATAGACAGATCGCTGAGATAGGTGCCTCACTGATTAAGCATTGGT
AAGAATTAATGATGTCTCGTTTAGATAAAAGTAAAGTGATTAACAGCGCATTAGAGCTGCTT
AATGAGGTCGGAATCGAAGGTTTAACAACCCGTAAACTCGCCCAGAAGCTAGGTGTAGAG
CAGCCTACATTGTATTGGCATGTAAAAAATAAGCGGGCTTTGCTCGACGCCTTAGCCATTG
AGATGTTAGATAGGCACCATACTCACTTTTGCCCTTTAGAAGGGGAAAGCTGGCAAGATTT
TTTACGTAATAACGCTAAAAGTTTTAGATGTGCTTTACTAAGTCATCGCGATGGAGCAAAAG
TACATTTAGGTACACGGCCTACAGAAAAACAGTATGAAACTCTCGAAAATCAATTAGCCTTT
TTATGCCAACAAGGTTTTTCACTAGAGAATGCATTATATGCACTCAGCGCAGTGGGGCATTT
TACTTTAGGTTGCGTATTGGAAGATCAAGAGCATCAAGTCGCTAAAGAAGAAAGGGAAACA
CCTACTACTGATAGTATGCCGCCATTATTACGACAAGCTATCGAATTATTTGATCACCAAGG
TGCAGAGCCAGCCTTCTTATTCGGCCTTGAATTGATCATATGCGGATTAGAAAAACAACTTA
AATGTGAAAGTGGGTCTTAATGAGAATATTCGTTTTCACCCAAGGAATAGAGGATATGGAG
AAAAAAATCACTGGATATACCACCGTTGATATATCCCAATGGCATCGTAAAGAACATTTTGA
GGCATTTCAGTCAGTTGCTCAATGTACCTATAACCAGACCGTTCAGCTGGATATTACGGCC
TTTTTAAAGACCGTAAAGAAAAATAAGCACAAGTTTTATCCGGCCTTTATTCACATTCTTGCC
CGCCTGATGAATGCTCATCCGGAGTTCCGTATGGCAATGAAAGACGGTGAGCTGGTGATA
TGGGATAGTGTTCACCCTTGTTACACCGTTTTCCATGAGCAAACTGAAACGTTTTCATCGCT
CTGGAGTGAATACCACGACTAGTTCCGGCAGTTTCTACACATATATTCGCAAGATGTGGCG
TGTTACGGTGAAAACCTGGCCTATTTCCCTAAAGGGTTTATTGAGAATATGTTTTTCGTCTC
AGCCAATCCCTGGGTGAGTTTCACCAGTTTTGATTTAAACGTGGCCAATATGGACAACTTCT
TCGCCCCCGTTTTCACTATGGGCAAATATTATACGCAAGGCGACAAGGTGCTGATGCCGCT
GGCGATTCAGGTTCATCATGCCGTTTGTGATGGCTTCCATGTCGGCAGAATGCTTAATGAA
TTACAACAGTACTGCGATGAGTGGCAGGGCGGGGCGTAATAGCTTCACTAGTTTAAAAGG
ATCTAGGTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGATAAGTCGTGTC
TTACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTTGTGATG
CTCGTCAGGGGGGCGGAGCCTATGGAAAAACGCCAGCAACGCGGCC
SEQ ID NO: 54: Nucleic acid sequence of pSBX8.CafRS#30.d47
TTTTTACGGTTCCTGGCCTTTTGCTGGCCTTTTGCTCACATGACCCGACACCATAACGCTC
GGTTGCCGCCGGGCGTTTTTTATTGGCCAGATGATTAATTCCTAATTTTTGTTGACACTCTA
TCATTGATAGAGTTATTTTACCACTCCCTATCAGTGATAGAGAAAAGTGAAATGAATAGTTC
GACAAAATCTAGATAACGAGGGCAAAAAATGGAAGCAGGTATCACCGGCACCTGGTACAA
CCAGCTCGGCTCGACCTTCATCGTGACCGCGGGTGCAGACGGAGCCCTGACCGGTACCT
ACTAGACGGCGCGTGGCAACGCCGAGAGCCGCTACGTCCTGACCGGTCGTTACGACAGC
GCCCCGGCCACCGACGGCAGCGGCACCGCCCTCGGTTGGACGGTGGCCTGGAAGAATAA
CTACCGCAACGCCCACTCCGCGACCACGTGGAGCGGCCAGTACGTCGGCGGCGCCGAG
GCGAGGATCAACACCCAGTGGCTGCTGACCTCCGGCACCACCGAGGCCAACGCCTGGAA
GTCCACGCTGGTCGGCCACGACACCTTCACCAAGGTGAAGCCGTCCGCCGCCTCCTAATA
AGCTTGACCTGTGAAGTGAAAAATGGCGCACATTGTGCGACATTTTTTTTGTCTGCCGTTTA
CCGCTACTGCGCGGCAGTACGCCTTTGGTTTATCCATTTTATACAATCCATGTAAAAAAGG
GCCCTGAAATTCAGGACCCTTTCTGAGCTCATTACAGGTTGGTGCTAATACCATTATAGTAG
CTCTCGGAACGGCTTGCACGTTTAATGTTTTTGAAACCGTGCATTACTTTCAGCAGACGTTC
GAGACCAAAACCTGCACCAATCCAGGGTTTATCAATACCCCATTCACGATCCAGGCTAACC
GGACCAACAACTGCGCTACTCAGTTCCAGATCACCGTGCATAATATCCAGGGTATCACCAA
AAACCATGCAGCTATCACCAACAATTTCAAAGTCGATTTCCAGGTAATCCAGAAACTCTTTA
ATCAGTGCTTCCAGATTTTCACGGGTACAACCGCTACCCATCTGGCCAAACACCACCATTG
TAAATTCTTCCAGGTGTTCTTTACCATCACTTTCTTTACGATAGCACGGACCAACTTCAAAG
ACTTTGGAAGGACCAGGCAGAATACGATCCAGTTTCCGGCTGTAGTTATACAATGTCGGGG
TCAGCATAGGACGCAGACACAGGTTTTTATCAACGCGAAAGATTTGTTTGCTCAGTTCGGT
ATCATTGTTAATGCCCATACGTTCAACATATTCTGCCGGAATCAGAATCGGGCTTTTGATTT
CCAGAAAACCGCGATCCACGAAAAATTTGGTAATATCACGTTCCAGTTTACCCAGATAATCT
TCGCGGTCGTTGGTATATAAGCGTTGAAAATCATTTTTACGACGAGTAACCAGTTCCGGTT
CCAGTTCACGAAACGGTTTTGCCATATTCAGGCTGATTTTATCTTCAGGACTCAGCAGTGCT
TCAACACGATCTAACTGAGACCGGGTTAAGCTCGGTGCCGGTGCGCTTGCCGGAACGCTG
CTATTCGGGGTGCTTTTTGCCGGACTCGGAACGCTACGGCTGGTATTGGTGCTTGCTTTTG
CGCTAACGCTATTTTCCAGAGGTTTCGGTGCGCGACTAACGCTTTTCGGCATTGCTTTTTTA
ACTTTAGGTGCGCTCACAACACGAACTTTAACTGAATTTTTGCTTTCGGTGCTGCGTGTCAG
AAAATTGTTAATATCTTCATCACTCACACGGCAGCGTTTACAGGTTTTACGATATTTGTGAT
GACGAAATGCGCGTGCGGTACGACAGCTACGGCTATTATTCACAACCAGATGATCGCCAC
AGGCCATTTCAATATAGATTTTGCTGCGGCTAACTTCGTGATGTTTGATTTTATGCAGGGTG
CCGGTACGGCTCATCCACAGACCTGTTGCGCTAATCAGAACATCCAGCGGTTTTTTATCCA
TATCGTACCTCCTTAAATTTCTAGGTTGTGACCTAGGTGATTTAGTTTACCAGTGCAAAAGA
AATGTCAAAAGAGAAGGGCGTGAATTTAACGCGGTTCCAGCGCAAAGACTTCAAAACCTGC
GTCGGTGCCGATTTCGGCCTATTGGTTAAAAAATGAGCTGAGTTCTAGTAAAAAAAATCCTT
AGCTTTCGCTAAGGATCTGTCAGTGGCGGAAACCCCGGGAATCTAACCCGGCTGAACGGAT
TTAGAGTCCATTCGATCTACATGATCAGGTTTCCGAATTCAGCGTTACAAGTATTACACAAA
GTTTTTTATGTTGAGAATATTTTTTTGATGGGGCATGGCGCAAAACCTTTCGCGGTATGGCA
TGCAGGTGGCACTTTTCGGGGAAATGTGCGCGGAACCCCTATTTGTTTATTTTTCTAAATAC
ATTCAAATATGTATCCGCTCATGAGACAATAACCCTGATAAATGCTTCAATAATATTGAAAAA
GGAAGAGTATGAGTATTCAACATTTCCGTGTCGCCCITATTCCCTTTTTTGCGGCATTTTGC
CTTCCTGTTTTTGCTCACCCAGAAACGCTGGTGAAAGTAAAAGATGCTGAAGATCAGTTGG
GTGCACGAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCGGTATTAT
CCCGTATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCACGATG
CCTGTAGCAATGGCAACAACGTTGCGCAAACTATTAACTGGCGAACTACTTACTCTAGCTT
CCCGGCAACAATTGATAGACTGGATGGAGGCGGATAAAGTTGCAGGACCACTTCTGCGCT
CGGCCCTTCCGGCTGGCTGGTTTATTGCTGATAAATCTGGAGCCGGTGAGCGTGGCTCTC
GCGGTATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAGGTGCCT
CACTGATTAAGCATTGGTAAGAATTAATGATGTCTCGTTTAGATAAAAGTAAAGTGATTAAC
AGCGCATTAGAGCTGCTTAATGAGGTCGGAATCGAAGGTTTAACAACCCGTAAACTCGCCC
AGAAGCTAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTAGAAGGG
GAAAGCTGGCAAGATTTTTTACGTAATAACGCTAAAAGTTTTAGATGTGCTTTACTAAGTCA
TCGCGATGGAGCAAAAGTACATTTAGGTACACGGCCTACAGAAAAACAGTATGAAACTCTC
GAAAATCAATTAGCCTTTTTATGCCAACAAGGTTTTTCACTAGAGAATGCATTATATGCACTC
AGCGCAGTGGGGCATTTTACTTTAGGTTGCGTATTGGAAGATCAAGAGCATCAAGTCGCTA
AAGAAGAAAGGGAAACACCTACTACTGATAGTATGCCGCCATTATTACGACAAGCTATCGA
ATTATTTGATCACCAAGGTGCAGAGCCAGCCTTCTTATTCGGCCTTGAATTGATCATATGCG
GATTAGAAAAACAACTTAAATGTGAAAGTGGGTCTTAATGAGAATATTCGTTTTCACCCAAG
GAATAGAGGATATGGAGAAAAAAATCACTGGATATACCACCGTTGATATATCCCAATGGCA
TCGTAAAGAACATTTTGAGGCATTTCAGTCAGTTGCTCAATGTACCTATAACCAGACCGTTC
AGCTGGATATTACGGCCTTTTTAAAGACCGTAAAGAAAAATAAGCACAAGTTTTATCCGGCC
TTTATTCACATTCTTGCCCGCCTGATGAATGCTCATCCGGAGTTCCGTATGGCAATGAAAG
ACGGTGAGCTGGTGATATGGGATAGTGTTCACCCTTGTTACACCGTTTTCCATGAGCAAAC
TGAAACGTTTTCATCGCTCTGGAGTGAATACCACGACTAGTTCCGGCAGTTTCTACACATAT
ATTCGCAAGATGTGGCGTGTTACGGTGAAAACCTGGCCTATTTCCCTAAAGGGTTTATTGA
GAATATGTTTTTCGTCTCAGCCAATCCCTGGGTGAGTTTCACCAGTTTTGATTTAAACGTGG
CCAATATGGACAACTTCTTCGCCCCCGTTTTCACTATGGGCAAATATTATACGCAAGGCGA
CAAGGTGCTGATGCCGCTGGCGATTCAGGTTCATCATGCCGTTTGTGATGGCTTCCATGTC
GGCAGAATGCTTAATGAATTACAACAGTACTGCGATGAGTGGCAGGGCGGGGCGTAATAG
CTTCACTAGTTTAAAAGGATCTAGGTGAAGATCCTTTTTGATAATCTCATGACCAAAATCCC
TTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCTGCCAG
TGGCGATAAGTCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGAGCT
TCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCAACGCG
GCC
SEQ ID NO: 55: Nucleic acid sequence of pSBX8.CafRS#30.d53
TTTTTACGGTTCCTGGCCTTTTGCTGGCCTTTTGCTCACATGACCCGACACCATAACGCTC
GGTTGCCGCCGGGCGTTTTTTATTGGCCAGATGATTAATTCCTAATTTTTGTTGACACTCTA
TCATTGATAGAGTTATTTTACCACTCCCTATCAGTGATAGAGAAAAGTGAAATGAATAGTTC
GACAAAATCTAGATAACGAGGGCAAAAAATGGAAGCAGGTATCACCGGCACCTGGTACAA
CCAGCTCGGCTCGACCTTCATCGTGACCGCGGGTGCAGACGGAGCTCTGACCGGTACCT
ACGTCACGGCGCGTGGCAACGCCGAGAGCCGCTACGTCCTGACCGGTCGTTACGACAGC
GCCCCGGCCACCGACGGCAGCGGCACCGCCCTCGGTTGGACGGTGGCCTGGAAGAATAA
CTACCGCAACGCCCACTCCGCGACCACGTGGAGCGGCCAGTACGTCGGCGGCGCCGAG
GCGAGGATCAACACCCAGTAGCTGCTGACCTCCGGCACCACCGAGGCCAACGCCTGGAA
GTCCACGCTGGTCGGCCACGACACCTTCACCAAGGTGAAGCCGTCCGCCGCCTCCTAATA
AGCTTGACCTGTGAAGTGAAAAATGGCGCACATTGTGCGACATTTTTTTTGTCTGCCGTTTA
CCGCTACTGCGCGGCAGTACGCCTTTGGTTTATCCATTTTATACAATCCATGTAAAAAAGG
GCCCTGAAATTCAGGACCCTTTCTGAGCTCATTACAGGTTGGTGCTAATACCATTATAGTAG
CTCTCGGAACGGCTTGCACGTTTAATGTTTTTGAAACCGTGCATTACTTTCAGCAGACGTTC
GAGACCAAAACCTGCACCAATCCAGGGTTTATCAATACCCCATTCACGATCCAGGCTAACC
GGACCAACAACTGCGCTACTCAGTTCCAGATCACCGTGCATAATATCCAGGGTATCACCAA
AAACCATGCAGCTATCACCAACAATTTCAAAGTCGATTTCCAGGTAATCCAGAAACTCTTTA
ATCAGTGCTTCCAGATTTTCACGGGTACAACCGCTACCCATCTGGCCAAACACCACCATTG
TAAATTCTTCCAGGTGTTCTTTACCATCACTTTCTTTACGATAGCACGGACCAACTTCAAAG
ACTTTGGAAGGACCAGGCAGAATACGATCCAGTTTCCGGCTGTAGTTATACAATGTCGGGG
TCAGCATAGGACGCAGACACAGGTTTTTATCAACGCGAAAGATTTGTTTGCTCAGTTCGGT
ATCATTGTTAATGCCCATACGTTCAACATATTCTGCCGGAATCAGAATCGGGCTTTTGATTT
CCAGAAAACCGCGATCCACGAAAAATTTGGTAATATCACGTTCCAGTTTACCCAGATAATCT
TCGCGGTCGTTGGTATATAAGCGTTGAAAATCATTTTTACGACGAGTAACCAGTTCCGGTT
CCAGTTCACGAAACGGTTTTGCCATATTCAGGCTGATTTTATCTTCAGGACTCAGCAGTGCT
TCAACACGATCTAACTGAGACCGGGTTAAGCTCGGTGCCGGTGCGCTTGCCGGAACGCTG
CTATTCGGGGTGCTTTTTGCCGGACTCGGAACGCTACGGCTGGTATTGGTGCTTGCTTTTG
CGCTAACGCTATTTTCCAGAGGTTTCGGTGCGCGACTAACGCTTTTCGGCATTGCTTTTTTA
ACTTTAGGTGCGCTCACAACACGAACTTTAACTGAATTTTTGCTTTCGGTGCTGCGTGTCAG
AAAATTGTTAATATCTTCATCACTCACACGGCAGCGTTTACAGGTTTTACGATATTTGTGAT
GACGAAATGCGCGTGCGGTACGACAGCTACGGCTATTATTCACAACCAGATGATCGCCAC
AGGCCATTTCAATATAGATTTTGCTGCGGCTAACTTCGTGATGTTTGATTTTATGCAGGGTG
CCGGTACGGCTCATCCACAGACCTGTTGCGCTAATCAGAACATCCAGCGGTTTTTTATCCA
TATCGTACCTCCTTAAATTTCTAGGTTGTGACCTAGGTGATTTAGTTTACCAGTGCAAAAGA
AATGTCAAAAGAGAAGGGCGTGAATTTAACGCGGTTCCAGCGCAAAGACTTCAAAACCTGC
GTCGGTGCCGATTTCGGCCTATTGGTTAAAAAATGAGCTGAGTTCTAGTAAAAAAAATCCTT
AGCTTTCGCTAAGGATCTGCAGTGGCGGAAACCCCGGGAATCTAACCCGGCTGAACGGAT
TTAGAGTCCATTCGATCTACATGATCAGGTTTCCGAATTCAGCGTTACAAGTATTACACAAA
GTTTTTTATGTTGAGAATATTTTTTTGATGGGGCATGGCGCAAAACCTTTCGCGGTATGGCA
TGCAGGTGGCACTTTTCGGGGAAATGTGCGCGGAACCCCTATTTGTTTATTTTTCTAAATAC
ATTCAAATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATTTTGC
CTTCCTGTTTTTGCTCACCCAGAAACGCTGGTGAAAGTAAAAGATGCTGAAGATCAGTTGG
GTGCACGAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCGGTATTAT
CCCGTATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCACGATG
CCTGTAGCAATGGCAACAACGTTGCGCAAACTATTAACTGGCGAACTACTTACTCTAGCTT
CCCGGCAACAATTGATAGACTGGATGGAGGCGGATAAAGTTGCAGGACCACTTCTGCGCT
CGGCCCTTCCGGCTGGCTGGTTTATTGCTGATAAATCTGGAGCCGGTGAGCGTGGCTCTC
GCGGTATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAGGTGCCT
CACTGATTAAGCATTGGTAAGAATTAATGATGTCTCGTTTAGATAAAAGTAAAGTGATTAAC
AGCGCATTAGAGCTGCTTAATGAGGTCGGAATCGAAGGTTTAACAACCCGTAAACTCGCCC
AGAAGCTAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTAGAAGGG
GAAAGCTGGCAAGATTTTTTACGTAATAACGCTAAAAGTTTTAGATGTGCTTTACTAAGTCA
TCGCGATGGAGCAAAAGTACATTTAGGTACACGGCCTACAGAAAAACAGTATGAAACTCTC
GAAAATCAATTAGCCTTTTTATGCCAACAAGGTTTTTCACTAGAGAATGCATTATATGCACTC
AGCGCAGTGGGGCATTTTACTTTAGGTTGCGTATTGGAAGATCAAGAGCATCAAGTCGCTA
AAGAAGAAAGGGAAACACCTACTACTGATAGTATGCCGCCATTATTACGACAAGCTATCGA
ATTATTTGATCACCAAGGTGCAGAGCCAGCCTTCTTATTCGGCCTTGAATTGATCATATGCG
GATTAGAAAAACAACTTAAATGTGAAAGTGGGTCTTAATGAGAATATTCGTTTTCACCCAAG
GAATAGAGGATATGGAGAAAAAAATCACTGGATATACCACCGTTGATATATCCCAATGGCA
TCGTAAAGAACATTTTGAGGCATTTCAGTCAGTTGCTCAATGTACCTATAACCAGACCGTTC
AGCTGGATATTACGGCCTTTTTAAAGACCGTAAAGAAAAATAAGCACAAGTTTTATCCGGCC
TTTATTCACATTCTTGCCCGCCTGATGAATGCTCATCCGGAGTTCCGTATGGCAATGAAAG
ACGGTGAGCTGGTGATATGGGATAGTGTTCACCCTTGTTACACCGTTTTCCATGAGCAAAC
TGAAACGTTTTCATCGCTCTGGAGTGAATACCACGACTAGTTCCGGCAGTTTCTACACATAT
ATTCGCAAGATGTGGCGTGTTACGGTGAAAACCTGGCCTATTTCCCTAAAGGGTTTATTGA
GAATATGTTTTTCGTCTCAGCCAATCCCTGGGTGAGTTTCACCAGTTTTGATTTAAACGTGG
CCAATATGGACAACTTCTTCGCCCCCGTTTTCACTATGGGCAAATATTATACGCAAGGCGA
CAAGGTGCTGATGCCGCTGGCGATTCAGGTTCATCATGCCGTTTGTGATGGCTTCCATGTC
GGCAGAATGCTTAATGAATTACAACAGTACTGCGATGAGTGGCAGGGCGGGGCGTAATAG
CTTCACTAGTTTAAAAGGATCTAGGTGAAGATCCTTTTTGATAATCTCATGACCAAAATCCC
TTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCTGCCAG
TGGCGATAAGTCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGAGCT
TCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCAACGCG
GCC
SEQ ID NO: 56: Nucleic acid sequence of pSBX8.CafRS#30.d51
TTTTTACGGTTCCTGGCCTTTTGCTGGCCTTTTGCTCACATGACCCGACACCATAACGCTC
GGTTGCCGCCGGGCGTTTTTTATTGGCCAGATGATTAATTCCTAATTTTTGTTGACACTCTA
TCATTGATAGAGTTATTTTACCACTCCCTATCAGTGATAGAGAAAAGTGAAATGAATAGTTC
GACAAAATCTAGATAACGAGGGCAAAAAATGGAAGCAGGTATCACCGGCACCTGGTACAA
CCAGCTCGGCTCGACCTTCATCGTGACCGCGGGTGCAGACGGAGCTCTGACCGGTACCT
ACGTCACGGCGCGTGGCAACGCCGAGAGCCGCTACGTCCTGACCGGTCGTTACGACAGC
GCCCCGGCCACCGACGGCAGCGGCACCGCCCTCGGTTGGACGGTGGCCTGGAAGAATAA
CTACCGCAACGCCCACTCCGCGACCACGTGGAGCGGCCAGTACGTCGGCGGCGCCGAG
GCGAGGATCAACACCCAGTGGCTGCTGACCTCCGGCACCACCGAGGCCAACGCCTAGAA
GTCCACGCTGGTCGGCCACGACACCTTCACCAAGGTGAAGCCGTCCGCCGCCTCCTAATA
AGCTTGACCTGTGAAGTGAAAAATGGCGCACATTGTGCGACATTTTTTTTGTCTGCCGTTTA
CCGCTACTGCGCGGCAGTACGCCTTTGGTTTATCCATTTTATACAATCCATGTAAAAAAGG
GCCCTGAAATTCAGGACCCTTTCTGAGCTCATTACAGGTTGGTGCTAATACCATTATAGTAG
CTCTCGGAACGGCTTGCACGTTTAATGTTTTTGAAACCGTGCATTACTTTCAGCAGACGTTC
GAGACCAAAACCTGCACCAATCCAGGGTTTATCAATACCCCATTCACGATCCAGGCTAACC
GGACCAACAACTGCGCTACTCAGTTCCAGATCACCGTGCATAATATCCAGGGTATCACCAA
AAACCATGCAGCTATCACCAACAATTTCAAAGTCGATTTCCAGGTAATCCAGAAACTCTTTA
ATCAGTGCTTCCAGATTTTCACGGGTACAACCGCTACCCATCTGGCCAAACACCACCATTG
TAAATTCTTCCAGGTGTTCTTTACCATCACTTTCTTTACGATAGCACGGACCAACTTCAAAG
ACTTTGGAAGGACCAGGCAGAATACGATCCAGTTTCCGGCTGTAGTTATACAATGTCGGGG
TCAGCATAGGACGCAGACACAGGTTTTTATCAACGCGAAAGATTTGTTTGCTCAGTTCGGT
ATCATTGTTAATGCCCATACGTTCAACATATTCTGCCGGAATCAGAATCGGGCTTTTGATTT
CCAGAAAACCGCGATCCACGAAAAATTTGGTAATATCACGTTCCAGTTTACCCAGATAATCT
TCGCGGTCGTTGGTATATAAGCGTTGAAAATCATTTTTACGACGAGTAACCAGTTCCGGTT
CCAGTTCACGAAACGGTTTTGCCATATTCAGGCTGATTTTATCTTCAGGACTCAGCAGTGCT
TCAACACGATCTAACTGAGACCGGGTTAAGCTCGGTGCCGGTGCGCTTGCCGGAACGCTG
CTATTCGGGGTGCTTTTTGCCGGACTCGGAACGCTACGGCTGGTATTGGTGCTTGCTTTTG
CGCTAACGCTATTTTCCAGAGGTTTCGGTGCGCGACTAACGCTTTTCGGCATTGCTTTTTTA
ACTTTAGGTGCGCTCACAACACGAACTTTAACTGAATTTTTGCTTTCGGTGCTGCGTGTCAG
AAAATTGTTAATATCTTCATCACTCACACGGCAGCGTTTACAGGTTTTACGATATTTGTGAT
GACGAAATGCGCGTGCGGTACGACAGCTACGGCTATTATTCACAACCAGATGATCGCCAC
AGGCCATTTCAATATAGATTTTGCTGCGGCTAACTTCGTGATGTTTGATTTTATGCAGGGTG
CCGGTACGGCTCATCCACAGACCTGTTGCGCTAATCAGAACATCCAGCGGTTTTTTATCCA
TATCGTACCTCCTTAAATTTCTAGGTTGTGACCTAGGTGATTTAGTTTACCAGTGCAAAAGA
AATGTCAAAAGAGAAGGGCGTGAATTTAACGCGGTTCCAGCGCAAAGACTTCAAAACCTGC
GTCGGTGCCGATTTCGGCCTATTGGTTAAAAAATGAGCTGAGTTCTAGTAAAAAAAATCCTT
AGCTTTCGCTAAGGATCTGCAGTGGCGGAAACCCCGGGAATCTAACCCGGCTGAACGGAT
TTAGAGTCCATTCGATCTACATGATCAGGTTTCCGAATTCAGCGTTACAAGTATTACACAAA
GTTTTTTATGTTGAGAATATTTTTTTGATGGGGCATGGCGCAAAACCTTTCGCGGTATGGCA
TGCAGGTGGCACTTTTCGGGGAAATGTGCGCGGAACCCCTATTTGTTTATTTTTCTAAATAC
ATTCAAATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATTTTGC
CTTCCTGTTTTTGCTCACCCAGAAACGCTGGTGAAAGTAAAAGATGCTGAAGATCAGTTGG
GTGCACGAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCGGTATTAT
CCCGTATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCACGATG
CCTGTAGCAATGGCAACAACGTTGCGCAAACTATTAACTGGCGAACTACTTACTCTAGCTT
CCCGGCAACAATTGATAGACTGGATGGAGGCGGATAAAGTTGCAGGACCACTTCTGCGCT
CGGCCCTTCCGGCTGGCTGGTTTATTGCTGATAAATCTGGAGCCGGTGAGCGTGGCTCTC
GCGGTATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAGGTGCCT
CACTGATTAAGCATTGGTAAGAATTAATGATGTCTCGTTTAGATAAAAGTAAAGTGATTAAC
AGCGCATTAGAGCTGCTTAATGAGGTCGGAATCGAAGGTTTAACAACCCGTAAACTCGCCC
AGAAGCTAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTAGAAGGG
GAAAGCTGGCAAGATTTTTTACGTAATAACGCTAAAAGTTTTAGATGTGCTTTACTAAGTCA
TCGCGATGGAGCAAAAGTACATTTAGGTACACGGCCTACAGAAAAACAGTATGAAACTCTC
GAAAATCAATTAGCCTTTTTATGCCAACAAGGTTTTTCACTAGAGAATGCATTATATGCACTC
AGCGCAGTGGGGCATTTTACTTTAGGTTGCGTATTGGAAGATCAAGAGCATCAAGTCGCTA
AAGAAGAAAGGGAAACACCTACTACTGATAGTATGCCGCCATTATTACGACAAGCTATCGA
ATTATTTGATCACCAAGGTGCAGAGCCAGCCTTCTTATTCGGCCTTGAATTGATCATATGCG
GATTAGAAAAACAACTTAAATGTGAAAGTGGGTCTTAATGAGAATATTCGTTTTCACCCAAG
GAATAGAGGATATGGAGAAAAAAATCACTGGATATACCACCGTTGATATATCCCAATGGCA
TCGTAAAGAACATTTTGAGGCATTTCAGTCAGTTGCTCAATGTACCTATAACCAGACCGTTC
AGCTGGATATTACGGCCTTTTTAAAGACCGTAAAGAAAAATAAGCACAAGTTTTATCCGGCC
TTTATTCACATTCTTGCCCGCCTGATGAATGCTCATCCGGAGTTCCGTATGGCAATGAAAG
ACGGTGAGCTGGTGATATGGGATAGTGTTCACCCTTGTTACACCGTTTTCCATGAGCAAAC
TGAAACGTTTTCATCGCTCTGGAGTGAATACCACGACTAGTTCCGGCAGTTTCTACACATAT
ATTCGCAAGATGTGGCGTGTTACGGTGAAAACCTGGCCTATTTCCCTAAAGGGTTTATTGA
GAATATGTTTTTCGTCTCAGCCAATCCCTGGGTGAGTTTCACCAGTTTTGATTTAAACGTGG
CCAATATGGACAACTTCTTCGCCCCCGTTTTCACTATGGGCAAATATTATACGCAAGGCGA
CAAGGTGCTGATGCCGCTGGCGATTCAGGTTCATCATGCCGTTTGTGATGGCTTCCATGTC
GGCAGAATGCTTAATGAATTACAACAGTACTGCGATGAGTGGCAGGGCGGGGCGTAATAG
CTTCACTAGTTTAAAAGGATCTAGGTGAAGATCCTTTTTGATAATCTCATGACCAAAATCCC
TTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCTGCCAG
TGGCGATAAGTCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGAGCT
TCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCAACGCG
GCC
SEQ ID NO: 57: Nucleic acid sequence of pASK75-PhoA-strepII
CCATCGAATGGCCAGATGATTAATTCCTAATTTTTGTTGACACTCTATCATTGATAGAGTTAT
TTTACCACTCCCTATCAGTGATAGAGAAAAGTGAAATGAATAGTTCGACAAAAATCTAGAAC
ATGGAGAAAATAAAGTGAAACAAAGCACTATTGCACTGGCACTCTTACCGTTACTGTTTACC
CCTGTGACAAAAGCCCGGACACCAGAAATGCCTGTTCTGGAAAACCGGGCTGCTCAGGGC
GATATTACTGCACCCGGCGGTGCTCGCCGTTTAACGGGTGATCAGACTGCCGCTCTGCGT
GATTCTCTTAGCGATAAACCTGCAAAAAATATTATTTTGCTGATTGGCGATGGGATGGGGG
ACTCGGAAATTACTGCCGCACGTAATTATGCCGAAGGTGCGGGCGGCTTTTTTAAAGGTAT
AGATGCCTTACCGCTTACCGGGCAATACACTCACTATGCGCTGAATAAAAAAACCGGCAAA
CCGGACTACGTCACCGACTCGGCTGCATCAGCAACCGCCTGGTCAACCGGTGTCAAAACC
TATAACGGCGCGCTGGGCGTCGATATTCACGAAAAAGATCACCCAACGATTCTGGAAATG
GCAAAAGCCGCAGGTCTGGCGACCGGTAACGTTTCTACCGCAGAGTTGCAGGATGCCACG
CCCGCTGCGCTGGTGGCACATGTGACCTCGCGCAAATGCTACGGTCCGAGCGCGACCAG
TGAAAAATGTCCGGGTAACGCTCTGGAAAAAGGCGGAAAAGGATCGATTACCGAACAGCT
GCTTAACGCTCGTGCCGACGTTACGCTTGGCGGCGGCGCAAAAACCTTTGCTGAAACGGC
AACCGCTGGTGAATGGCAGGGAAAAACGCTGCGTGAACAGGCACAGGCGCGTGGTTATC
AGTTGGTGAGCGATGCTGCCTCACTGAATTCGGTGACGGAAGCGAATCAGCAAAAACCCC
TGCTTGGCCTGTTTGCTGACGGCAATATGCCAGTGCGCTGGCTAGGACCGAAAGCAACGT
ACCATGGCAATATCGATAAGCCCGCAGTCACCTGTACGCCAAATCCGCAACGTAATGACAG
TGTACCAACCCTGGCGCAGATGACCGACAAAGCCATTGAATTGTTGAGTAAAAATGAGAAA
GGCTTTTTCCTGCAAGTTGAAGGTGCGTCAATCGATAAACAGGATCATGCTGCGAATCCTT
GTGGGCAAATTGGCGAGACGGTCGATCTCGATGAAGCCGTACAACGGGCGCTGGAATTC
GCTAAAAAGGAGGGTAACACGCTGGTCATAGTCACCGCTGATCACGCCCACGCCAGCCAG
ATTGTTGCGCCGGATACCAAAGCTCCGGGCCTCACCCAGGCGCTAAATACCAAAGATGGC
GCAGTGATGGTGATGAGTTACGGGAACTCCGAAGAGGATTCACAAGAACATACCGGCAGT
CAGTTGCGTATTGCGGCGTATGGCCCGCATGCCGCCAATGTTGTTGGACTGACCGACCAG
ACCGATCTCTTCTACACCATGAAAGCCGCTCTGGGGCTGAAACCGCCTAGCGCTTGGTCT
CACCCGCAGTTCGAAAAATAATAAGCTTGACCTGTGAAGTGAAAAATGGCGCACATTGTGC
GACATTTTTTTTGTCTGCCGTTTACCGCTACTGCGTCACGGATCTCCACGCGCCCTGTAGC
GGCGCATTAAGCGCGGCGGGTGTGGTGGTTACGCGCAGCGTGACCGCTACACTTGCCAG
CGCCCTAGCGCCCGCTCCTTTCGCTTTCTTCCCTTCTTTCTCGCCACGTTCGCCGGCTTT
CCCCGTCAAGCTCTAAATCGGGGGCTCCCTTTAGGGTTCCGATTTAGTGCTTTACGGCACC
TCGACCCCAAAAAACTTGATTAGGGTGATGGTTCACGTAGTGGGCCATCGCCCTGATAGAC
GGTTTTTCGCCCTTTGACGTTGGAGTCCACGTTCTTTAATAGTGGACTCTTGTTCCAAACTG
GAACAACACTCAACCCTATCTCGGTCTATTCTTTTGATTTATAAGGGATTTTGCCGATTTCG
GCCTATTGGTTAAAAAATGAGCTGATTTAACAAAAATTTAACGCGAATTTTAACAAAATATTA
ACGTTTACAATTTCAGGTGGCACTTTTCGGGGAAATGTGCGCGGAACCCCTATTTGTTTATT
TTTCTAAATACATTCAAATATGTATCCGCTCATGAGACAATAACCCTGATAAATGCTTCAATA
ATATTGAAAAAGGAAGAGTATGAGTATTCAACATTTCCGTGTCGCCCTTATTCCCTTTTTTG
CGGCATTTTGCCTTCCTGTTTTTGCTCACCCAGAAACGCTGGTGAAAGTAAAAGATGCTGA
AGATCAGTTGGGTGCACGAGTGGGTTACATCGAACTGGATCTCAACAGCGGTAAGATCCTT
GAGAGTTTTCGCCCCGAAGAACGTTTTCCAATGATGAGCACTTTTAAAGTTCTGCTATGTG
GCGCGGTATTATCCCGTATTGACGCCGGGCAAGAGCAACTCGGTCGCCGCATACACTATT
CTCAGAATGACTTGGTTGAGTACTCACCAGTCACAGAAAAGCATCTTACGGATGGCATGAC
AGTAAGAGAATTATGCAGTGCTGCCATAACCATGAGTGATAACACTGCGGCCAACTTACTT
CTGACAACGATCGGAGGACCGAAGGAGCTAACCGCTTTTTTGCACAACATGGGGGATCAT
GTAACTCGCCTTGATCGTTGGGAACCGGAGCTGAATGAAGCCATACCAAACGACGAGCGT
GACACCACGATGCCTGTAGCAATGGCAACAACGTTGCGCAAACTATTAACTGGCGAACTAC
TTACTCTAGCTTCCCGGCAACAATTGATAGACTGGATGGAGGCGGATAAAGTTGCAGGACC
ACTTCTGCGCTCGGCCCTTCCGGCTGGCTGGTTTATTGCTGATAAATCTGGAGCCGGTGA
GCGTGGCTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCGCTGA
GATAGGTGCCTCACTGATTAAGCATTGGTAGGAATTAATGATGTCTCGTTTAGATAAAAGTA
AAGTGATTAACAGCGCATTAGAGCTGCTTAATGAGGTCGGAATCGAAGGTTTAACAACCCG
TAAACTCGCCCAGAAGCTAGGTGTAGAGCAGCCTACATTGTATTGGCATGTAAAAAATAAG
CGGGCTTTGCTCGACGCCTTAGCCATTGAGATGTTAGATAGGCACCATACTCACTTTTGCC
CTTTAGAAGGGGAAAGCTGGCAAGATTTTTTACGTAATAACGCTAAAAGTTTTAGATGTGCT
TTACTAAGTCATCGCGATGGAGCAAAAGTACATTTAGGTACACGGCCTACAGAAAAACAGT
ATGAAACTCTCGAAAATCAATTAGCCTTTTTATGCCAACAAGGTTTTTCACTAGAGAATGCA
TTATATGCACTCAGCGCAGTGGGGCATTTTACTTTAGGTTGCGTATTGGAAGATCAAGAGC
ATCAAGTCGCTAAAGAAGAAAGGGAAACACCTACTACTGATAGTATGCCGCCATTATTACG
ACAAGCTATCGAATTATTTGATCACCAAGGTGCAGAGCCAGCCTTCTTATTCGGCCTTGAAT
TGATCATATGCGGATTAGAAAAACAACTTAAATGTGAAAGTGGGTCTTAAAAGCAGCATAAC
CTTTTTCCGTGATGGTAACTTCACTAGTTTAAAAGGATCTAGGTGAAGATCCTTTTTGATAAT
CTCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTTTTCCGA
AGGTAACTGGCTTCAGCAGAGCGCAGATACCAAATACTGTCCTTCTAGTGTAGCCGTAGTT
AGGCCACCACTTCAAGAACTCTGTAGCACCGCCTACATACCTCGCTCTGCTAATCCTGTTA
CCAGTGGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCAC
GCTTCCCGAAGGGAGAAAGGCGGACAGGTATCCGGTAAGCGGCAGGGTCGGAACAGGAG
AGCGCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCTTTTGCTCACAT
GACCCGACA
SEQ ID NO: 58: Nucleic acid sequence of human albumin binding domain (ABD)
CTGGCAGAAGCAAAAGTTCTGGCAAATCGTGAACTGGATAAATATGGTGTGAGCGACTATT
ACAAGAACCTGATTAATAACGCGAAAACCGTGGAAGGTGTTAAAGCACTGATTGATGAAAT
TCTGGCAGCACTGCCG
SEQ ID NO: 59: Amino acid sequence of human albumin binding domain (ABD)
LAEAKVLANRELDKYGVSDYYKNLINNAKTVEGVKALIDEILAALP
SEQ ID NO: 60: Nucleic acid sequence of ProtL-ABD fusion protein
ATGAAAGAAGAAGTTACCATTAAAGTTAATCTGATTTTCGCCGATGGTAAAACCCAGACCGC
AGAATTTAAAGGCACCTTTGAAGAAGCAACCGCAAAAGCCTATGCCTATGCCGATCTGCTG
GCAAAAGAAAATGGTGAATATACCGCAGATCTGGAAGATGGTGGTAATACCATCAATATCA
AATTTGCCGGTGGTGCCGTTGATGCAAATAGCCTGGCAGAAGCAAAAGTTCTGGCAAATC
GTGAACTGGATAAATATGGTGTGAGCGACTATTACAAGAACCTGATTAATAACGCGAAAAC
CGTGGAAGGTGTTAAAGCACTGATTGATGAAATTCTGGCAGCACTGCCGTAA
SEQ ID NO: 61: Amino acid sequence of ProtL-ABD fusion protein.
Methionine (underlined) was added as a start codon.
MKEEVTIKVNLIFADGKTQTAEFKGTFEEATAKAYAYADLLAKENGEYTADLEDGGNTINIKFAG
GAVDANSLAEAKVLANRELDKYGVSDYYKNLINNAKTVEGVKALIDEILAALP
```

In the following, for illustration purposes, the amino acid sequence of ProtL-ABD (SEQ ID NO: 61) is shown below the corresponding nucleic acid sequence (SEQ ID NO: 60). The sequence of protein L domain B1 begins with Lys³²⁶ and ends with Gly³⁸⁹ (UniProt Q51918)

The position of 337, 347, 360, 364, 368 and 369 are in bold face and underlined.

```
                          10 20 30 40 50 60
                            + + + + + +
      1 ATGAAAGAAGAAGTTACCATTAAAGTTAATCTGATTTTCGCCGATGGTAAAACCCAGACC 60
          MetLysGluGluValThrIleLysValAsnLeuIlePheAlaAspGlyLysThrG1nThr
                326
                        70 80 90 100 110 120
                          + + + + + +
  61 GCAGAATTTAAAGGCACCTTTGAAGAAGCAACCGCAAAAGCCTATGCCTATGCCGATCTG 120
        AlaGluPheLysGlyThrPheGluGluAlaThrAlaLysAlaTyrAlaTyrAlaAspLeu
                        130 140 150 160 170 180
                          + + + + + +
121 CTGGCAAAAGAAAATGGTGAATATACCGCAGATCTGGAAGATGGTGGTAATACCATCAAT 180
        LeuAlaLysGluAsnGlyGluTyrThrAlaAspLeuGluAspGlyGlyAsnThrIleAsn
                        190 200 210 220 230 240
                          + + + + + +
181 ATCAAATTTGCCGGTGGTGCCGTTGATGCAAATAGCCTGGCAGAAGCAAAAGTTCTGGCA 240
        IleLysPheAlaGlyGlyAlaValAspAlaAsnSerLeuAlaGluAlaLysValLeuAla
                                389 > albumin binding domain
                        250 260 270 280 290 300
                          + + + + + +
241 AATCGTGAACTGGATAAATATGGTGTGAGCGACTATTACAAGAACCTGATTAATAACGCG 300
        AsnArgGluLeuAspLysTyrGlyValSerAspTyrTyrLysAsnLeuIleAsnAsnAla
                        310 320 330 340 350
                          + + + + +
301 AAAACCGTGGAAGGTGTTAAAGCACTGATTGATGAAATTCTGGCAGCACTGCCGTAG 354
        LysThrValGluGlyValLysAlaLeuIleAspGluIleLeuAlaAlaLeuPro
SEQ ID NO: 62: Nucleic acid sequence of pASK75-ProtL-ABD
CCATCGAATGGCCAGATGATTAATTCCTAATTTTTGTTGACACTCTATCATTGATAGAGTTAT
TTTACCACTCCCTATCAGTGATAGAGAAAAGTGAAAATGAATAGTTCGACAAAAATCTAGAAA
TAATTTTGTTTAACTTTAAGAAGGAGATATACATATGAAAGAAGAAGTTACCATTAAAGTTAA
TCTGATTTTCGCCGATGGTAAAACCCAGACCGCAGAATTTAAAGGCACCTTTGAAGAAGCA
ACCGCAAAAGCCTATGCCTATGCCGATCTGCTGGCAAAAGAAAATGGTGAATATACCGCAG
ATCTGGAAGATGGTGGTAATACCATCAATATCAAATTTGCCGGTGGTGCCGTTGATGCAAA
TAGCCTGGCAGAAGCAAAAGTTCTGGCAAATCGTGAACTGGATAAATATGGTGTGAGCGAC
TATTACAAGAACCTGATTAATAACGCGAAAACCGTGGAAGGTGTTAAAGCACTGATTGATG
AAATTCTGGCAGCACTGCCGTAATAAGCTTGACCTGTGAAGTGAAAAATGGCGCACATTGT
GCGACATTTTTTTTGTCTGCCGTTTACCGCTACTGCGTCACGGATCTCCACGCGCCCTGTA
GCGGCGCATTAAGCGCGGCGGGTGTGGTGGTTACGCGCAGCGTGACCGCTACACTTGCC
AGCGCCCTAGCGCCCGCTCCTTTCGCTTTCTTCCCTTCCTTTCTCGCCACGTTCGCCGGCT
TTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATA
GACGGTTTTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTTGCCGATT
TCGGCCTATTGGTTAAAAAATGAGCTGATTTAACAAAAATTTAACGCGAATTTTAACAAAATA
TTAACGTTTACAATTTCAGGTGGCACTTTTCGGGGAAATGTGCGCGGAACCCCTATTTGTTT
ATTTTTCTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCCCTTTTT
TGCGGCATTTTGCCTTCCTGTTTTTGCTCACCCAGAAACGCTGGTGAAAGTAAAAGATGCT
GAAGATCAGTTGGGTGCACGAGTGGGTTACATCGAACTGGATCTCAACAGCGGTAAGATC
CTTGAGAGTTTTCGCCCCGAAGAACGTTTTCCAATGATGAGCACTTTTAAAGTTCTGCTATG
TGGCGCGGTATTATCCCGTATTGACGCCGGGCAAGAGCAACTCGGTCGCCGCATACACTA
TTCTCAGAATGACTTGGTTGAGTACTCACCAGTCACAGAAAAGCATCTTACGGATGGCATG
ACAGTAAGAGAATTATGCAGTGCTGCCATAACCATGAGTGATAACACTGCGGCCAACTTAC
TTCTGACAACGATCGGAGGACCGAAGGAGCTAACCGCTTTTTTGCACAACATGGGGGATC
ATGTAACTCGCCTTGATCGTTGGGAACCGGAGCTGAATGAAGCCATACCAAACGACGAGC
GTGACACCACGATGCCTGTAGCAATGGCAACAACGTTGCGCAAACTATTAACTGGCGAACT
ACTTACTCTAGCTTCCCGGCAACAATTgATAGACTGGATGGAGGCGGATAAAGTTGCAGGA
CCACTTCTGCGCTCGGCCCTTCCGGCTGGCTGGTTTATTGCTGATAAATCTGGAGCCGGT
GAGCGTGGCTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAGATCGCT
GAGATAGGTGCCTCACTGATTAAGCATTGGTAGGAATTAATGATGTCTCGTTTAGATAAAAG
TAAAGTGATTAACAGCGCATTAGAGCTGCTTAATGAGGTCGGAATCGAAGGTTTAACAACC
CGTAAACTCGCCCAGAAGCTAGGTGTAGAGCAGCCTACATTGTATTGGCATGTAAAAAATA
AGCGGGCTTTGCTCGACGCCTTAGCCATTGAGATGTTAGATAGGCACCATACTCACTTTTG
CCCTTTAGAAGGGGAAAGCTGGCAAGATTTTTTACGTAATAACGCTAAAAGTTTTAGATGTG
CTTTACTAAGTCATCGCGATGGAGCAAAAGTACATTTAGGTACACGGCCTACAGAAAAACA
GTATGAAACTCTCGAAAATCAATTAGCCTTTTTATGCCAACAAGGTTTTTCACTAGAGAATG
CATTATATGCACTCAGCGCaGTGGGGCATTTTACTTTAGGTTGCGTATTGGAAGATCAAGA
GCATCAAGTCGCTAAAGAAGAAAGGGAAACACCTACTACTGATAGTATGCCGCCATTATTA
CGACAAGCTATCGAATTATTTGATCACCAAGGTGCAGAGCCAGCCTTCTTATTCGGCCTTG
AATTGATCATtTGCGGATTAGAAAAACAACTTAAATGTGAAAGTGGGTCTTAAAAGCAGCAT
AACCTTTTTCCGTGATGGTAACTTCACTAGTTTAAAAGGATCTAGGTGAAGATCCTTTTTGA
TAATCTCATGACCAAAATCCCTTAACGTGAGTTTTCGTTCCACTGAGCGTCAGACCCCGTA
GAAAAGATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTCCTTCTAGTGTAGCCGT
AGTTAGGCCACCACTTCAAGAACTCTGTAGCACCGCCTACATACCTCGCTCTGCTAATCCT
GTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCA
GCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGGAGCCT
ATGGAAAAACGCCAGCAACGCGGCCTTTTTACGGTTCCTGGCCTTTTGCTGGCCTTTTGCT
CACATGACCCGACA
SEQ ID NO: 63: Nucleic acid sequence of AJR_ProtL_PHE337TAG_fw
CATTAAAGTTAATCTGATTTAGGCCGATGGTAAAAC
SEQ ID NO: 64: Nucleic acid sequence of AJR_ProtL_PHE337TAG_rv
GTTTTACCATCGGCCTAAATCAGATTAACTTTAATG
SEQ ID NO: 65: Nucleic acid sequence of AJR_ProtL_Y361N_L365S_fw
CAAAAGCCTATGCCAACGCCGATCTGAGCGCGAAAATGGTG
SEQ ID NO: 66: Nucleic acid sequence of AJR_ProtL_Y361N_L365S_rv
CACCATTTTCTTTTGCGCTCAGATCGGCGTTGGCATAGGCTTTTG
SEQ ID NO: 67: Nucleic acid sequence of ProtL^{UAG337}-ABD
ATGAAAGAAGAAGTTACCATTAAAGTTAATCTGATTTAGGCCGATGGTAAAACCCAGACCG
CAGAATTTAAAGGCACCTTTGAAGAAGCAACCGCAAAAGCCTATGCCAACGCCGATCTGAG
CGCAAAAGAAAATGGTGAATATACCGCAGATCTGGAAGATGGTGGTAATACCATCAATATC
AAATTTGCCGGTGGTGCCGTTGATGCAAATAGCCTGGCAGAAGCAAAAGTTCTGGCAAATC
GTGAACTGGATAAATATGGTGTGAGCGACTATTACAAGAACCTGATTAATAACGCGAAAAC
CGTGGAAGGTGTTAAAGCACTGATTGATGAAATTCTGGCAGCACTGCCGTAA
SEQ ID NO: 68: Nucleic acid sequence of AJR_ProtL_PHE347TAG_fw
CAGACCGCAGAATAGAAAGGCACCTTTG
SEQ ID NO: 69: Nucleic acid sequence of AJR_ProtL_PHE347TAG_rv
CAAAGGTGCCTTTCTATTCTGCGGTCTG
SEQ ID NO: 70: Nucleic acid sequence of AJR_ProtL_TYR361ALA_fw
CCGCAAAAGCCTATGCCGCGGCCGATCTGCTGGC
SEQ ID NO: 71: Nucleic acid sequence of AJR_ProtL_TYR361ALA_rv
GCCAGCAGATCGGCCGCGGCATAGGCTTTTGCGG
SEQ ID NO: 72: Nucleic acid sequence of ProtL^{UAG347}-ABD
ATGAAAGAAGAAGTTACCATTAAAGTTAATCTGATTTTCGCCGATGGTAAAACCCAGACCGC
AGAATAGAAAGGCACCTTTGAAGAAGCAACCGCAAAAGCCTATGCCGCGGCCGATCTGCT
GGCAAAAGAAAATGGTGAATATACCGCAGATCTGGAAGATGGTGGTAATACCATCAATATC
AAATTTGCCGGTGGTGCCGTTGATGCAAATAGCCTGGCAGAAGCAAAAGTTCTGGCAAATC
GTGAACTGGATAAATATGGTGTGAGCGACTATTACAAGAACCTGATTAATAACGCGAAATC
CGTGGAAGGTGTTAAAGCACTGATTGATGAAATTCTGGCAGCACTGCCGTAA
SEQ ID NO: 73: Nucleic acid sequence of AJR_ProtL_ALA360TAG_fw
CCGCAAAAGCCTATTAGTATGCCGATCTGC
SEQ ID NO: 74: Nucleic acid sequence of AJR_ProtL_ALA360TAG_rv
GCAGATCGGCATACTAATAGGCTTTTGCGG
SEQ ID NO: 75: Nucleic acid sequence of ProtL^{UAG360}-ABD
ATGAAAGAAGAAGTTACCATTAAAGTTAATCTGATTTTCGCCGATGGTAAAACCCAGACCGC
AGAATTTAAAGGCACCTTTGAAGAAGCAACCGCAAAAGCCTATTAGTATGCCGATCTGCTG
GCAAAAGAAAATGGTGAATATACCGCAGATCTGGAAGATGGTGGTAATACCATCAATATCA
AATTTGCCGGTGGTGCCGTTGATGCAAATAGCCTGGCAGAAGCAAAAGTTCTGGCAAATC
GTGAACTGGATAAATATGGTGTGAGCGACTATTACAAGAACCTGATTAATAACGCGAAAAC
CGTGGAAGGTGTTAAGCACTGATTGATGAAATTCTGGCAGCACTGCCGTAA
SEQ ID NO: 76: Nucleic acid sequence of AJR_ProtL_LEU364TAG_fw
CTATGCCTATGCCGATTAGCTGGCAAAAGAAAATGG
SEQ ID NO: 77: Nucleic acid sequence of AJR_ProtL_LEU364TAG_rv
CCATTTTCTTTTGCCAGCTAATCGGCATAGGCATAG
SEQ ID NO: 78: Nucleic acid sequence of ProtL^{UAG364}-ABD
ATGAAAGAAGAAGTTACCATTAAAGTTAATCTGATTTTCGCCGATGGTAAAACCCAGACCGC
AGAATTTAAAGGCACCTTTGAAGAAGCAACCGCAAAAGCCTATGCCTATGCCGATTAGCTG
GCAAAAGAAAATGGTGAATATACCGCAGATCTGGAAGATGGTGGTAATACCATCAATATCA
AATTTGCCGGTGGTGCCGTTGATGCAAATAGCCTGGCAGAAGCAAAAGTTCTGGCAAATC
GTGAACTGGATAAATATGGTGTGAGCGACTATTACAAGAACCTGATTAATAACGCGAAAAC
CGTGGAAGGTGTTAAAGCACTGATTGATGAAATTCTGGCAGCACTGCCGTAA
SEQ ID NO: 79: Nucleic acid sequence of AJR_ProtL_GLU368TAG_fw
GATCTGCTGGCAAAATAGAATGGTGAATATACCG
SEQ ID NO: 80: Nucleic acid sequence of AJR_ProtL_GLU368TAG_rv
CGGTATATTCACCATTCTATTTTGCCAGCAGATC
SEQ ID NO: 81: Nucleic acid sequence of ProtL^{UAG368}-ABD
ATGAAAGAAGAAGTTACCATTAAAGTTAATCTGATTTTCGCCGATGGTAAAACCCAGACCGC
AGAATTTAAAGGCACCTTTGAAGAAGCAACCGCAAAAGCCTATGCCTATGCCGATCTGCTG
GCAAAATAGAATGGTGAATATACCGCAGATCTGGAAGATGGTGGTAATACCATCAATATCA
AATTTGCCGGTGGTGCCGTTGATGCAAATAGCCTGGCAGAAGCAAAAGTTCTGGCAAATC
GTGAACTGGATAAATATGGTGTGAGCGACTATTACAAGAACCTGATTAATAACGCGAAAAC
CGTGGAAGGTGTTAAAGCACTGATTGATGAAATTCTGGCAGCACTGCCGTAA
SEQ ID NO: 82: Nucleic acid sequence of AJR_ProtL_ASN369TAG_fw
CTGCTGGCAAAAGAATAGGGTGAATATACCGC
SEQ ID NO: 83: Nucleic acid sequence of AJR_ProtL_ASN369TAG_rv
GCGGTATATTCACCCTATTCTTTTGCCAGCAG
SEQ ID NO: 84: Nucleic acid sequence of ProtL^{UAG369}-ABD
ATGAAAGAAGAAGTTACCATTAAAGTTAATCTGATTTTCGCCGATGGTAAAACCCAGACCGC
AGAATTTAAAGGCACCTTTGAAGAAGCAACCGCAAAAGCCTATGCCTATGCCGATCTGCTG
GCAAAAGAATAGGGTGAATATACCGCAGATCTGGAAGATGGTGGTAATACCATCAATATCA
AATTTGCCGGTGGTGCCGTTGATGCAAATAGCCTGGCAGAAGCAAAAGTTCTGGCAAATC
GTGAACTGGATAAATATGGTGTGAGCGACTATTACAAGAACCTGATTAATAACGCGAAAAC
CGTGGAAGGTGTTAAAGCACTGATTGATGAAATTCTGGCAGCACTGCCGTAA
SEQ ID NO: 85: Nucleic acid sequence of pSBX8.CafRS#30d71
TTTTTACGGTTCCTGGCCTTTTGCTGGCCTTTTGCTCACATGACCCGACACCATAACGCTC
GGTTGCCGCCGGGCGTTTTTTATTGGCCAGATGATTAATTCCTAATTTTTGTTGACACTCTA
TCATTGATAGAGTTATTTTACCACTCCCTATCAGTGATAGAGAAAAGTGAAATGAATAGTTC
GACAAAATCTAGAAATAATTTTGTTTAACTTTAAGAAGGAGATATACATATGAAAGAAGAAGT
TACCATTAAAGTTAATCTGATTTAGGCCGATGGTAAAACCCAGACCGCAGAATTTAAAGGCA
CCTTTGAAGAAGCAACCGCAAAAGCCTATGCCAACGCCGATCTGAGCGCAAAAGAAAATG
GTGAATATACCGCAGATCTGGAAGATGGTGGTAATACCATCAATATCAAATTTGCCGGTGG
TGCCGTTGATGCAAATAGCCTGGCAGAAGCAAAAGTTCTGGCAAATCGTGAACTGGATAAA
TATGGTGTGAGCGACTATTACAAGAACCTGATTAATAACGCGAAAACCGTGGAAGGTGTTA
AAGCACTGATTGATGAAATTCTGGCAGCACTGCCGTAATAAGCTTGACCTGTGAAGTGAAA
AATGGCGCACATTGTGCGACATTTTTTTTGTCTGCCGTTTACCGCTACTGCGCGGCAGTAC
GCCTTTGGTTTATCCATTTTATACAATCCATGTAAAAAAGGGCCCTGAAATTCAGGACCCTT
TCTGAGCTCATTACAGGTTGGTGCTAATACCATTATAGTAGCTCTCGGAACGGCTTGCACG
TTTAATGTTTTTGAAACCGTGCATTACTTTCAGCAGACGTTCGAGACCAAAACCTGCACCAA
TCCAGGGTTTATCAATACCCCATTCACGATCCAGGCTAACCGGACCAACAACTGCGCTACT
CAGTTCCAGATCACCGTGCATAATATCCAGGGTATCACCAAAAACCATGCAGCTATCACCA
ACAATTTCAAAGTCGATTTCCAGGTAATCCAGAAACTCTTTAATCAGTGCTTCCAGATTTTCA
CGGGTACAACCGCTACCCATCTGGCCAAACACCACCATTGTAAATTCTTCCAGGTGTTCTT
TACCATCACTTTCTTTACGATAGCACGGACCAACTTCAAAGACTTTGGAAGGACCAGGCAG
AATACGATCCAGTTTCCGGCTGTAGTTATACAATGTCGGGGTCAGCATAGGACGCAGACAC
AGGTTTTTATCAACGCGAAAGATTTGTTTGCTCAGTTCGGTATCATTGTTAATGCCCATACG
TTCAACATATTCTGCCGGAATCAGAATCGGGCTTTTGATTTCCAGAAAACCGCGATCCACG
AAAAATTTGGTAATATCACGTTCCAGTTTACCCAGATAATCTTCGCGGTCGTTGGTATATAA
GCGTTGAAAATCATTTTTACGACGAGTAACCAGTTCCGGTTCCAGTTCACGAAACGGTTTT
GCCATATTCAGGCTGATTTTATCTTCAGGACTCAGCAGTGCTTCAACACGATCTAACTGAGA
CCGGGTTAAGCTCGGTGCCGGTGCGCTTGCCGGAACGCTGCTATTCGGGGTGCTTTTTGC
CGGACTCGGAACGCTACGGCTGGTATTGGTGCTTGCTTTTGCGCTAACGCTATTTTCCAGA
GGTTTCGGTGCGCGACTAACGCTTTTCGGCATTGCTTTTTTAACTTTAGGTGCGCTCACAA
CACGAACTTTAACTGAATTTTTGCTTTCGGTGCTGCGTGTCAGAAAATTGTTAATATCTTCAT
CACTCACACGGCAGCGTTTACAGGTTTTACGATATTTGTGATGACGAAATGCGCGTGCGGT
ACGACAGCTACGGCTATTATTCACAACCAGATGATCGCCACAGGCCATTTCAATATAGATTT
TGCTGCGGCTAACTTCGTGATGTTTGATTTTATGCAGGGTGCCGGTACGGCTCATCCACAG
ACCTGTTGCGCTAATCAGAACATCCAGCGGTTTTTTATCCATATCGTACCTCCTTAAATTTC
TAGGTTGTGACCTAGGTGATTTAGTTTACCAGTGCAAAAGAAATGTCAAAAGAGAAGGGCG
TGAATTTAACGCGGTTCCAGCGCAAAGACTTCAAAACCTGCGTCGGTGCCGATTTCGGCCT
ATTGGTTAAAAAATGAGCTGAGTTCTAGTAAAAAAAATCCTTAGCTTTCGCTAAGGATCTGC
AGTGGCGGAAACCCCGGGAATCTAACCCGGCTGAACGGATTTAGAGTCCATTCGATCTAC
ATGATCAGGTTTCCGAATTCAGCGTTACAAGTATTACACAAAGTTTTTTATGTTGAGAATATT
TTTTTGATGGGGCATGGCGCAAAACCTTTCGCGGTATGGCATGCAGGTGGCACTTTTCGG
GGAAATGTGCGCGGAACCCCTATTTGTTTATTTTTCTAAATACATTCAAATATGTATCCGCT
CATGAGACAATAACCCTGATAAATGCTTCAATAATATTGAAAAAGGAAGAGTATGAGTATTC
AACATTTCCGTGTCGCCCTTATTCCCTTTTTTGCGGCATTTTGCCTTCCTGTTTTTGCTCAC
CCAGAAACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAACGTTTTC
CAATGATGAGCACTTTTAAAGTTCTGCTATGTGGCGCGGTATTATCCCGTATTGACGCCGG
GCAAGAGCAACTCGGTCGCCGCATACACTATTCTCAGAATGACTTGGTTGAGTACTCACCA
GTCACAGAAAAGCATCTTACGGATGGCATGACAGTAAGAGAATTATGCAGTGCTGCCATAA
CCATGAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAACCGGA
GCTGAATGAAGCCATACCAAACGACGAGCGTGACACCACGATGCCTGTAGCAATGGCAAC
AACGTTGCGCAAACTATTAACTGGCGAACTACTTACTCTAGCTTCCCGGCAACAATTGATA
GACTGGATGGAGGCGGATAAAGTTGCAGGACCACTTCTGCGCTCGGCCCTTCCGGCTGG
CTGGTTTATTGCTGATAAATCTGGAGCCGGTGAGCGTGGCTCTCGCGGTATCATTGCAGCA
CTGGGGCCAGATGGTAAGCCCTCCCGTATCGTAGTTATCTACACGACGGGGAGTCAGGCA
ACTATGGATGAACGAAATAGACAGATCGCTGAGATAGGTGCCTCACTGATTAAGCATTGGT
AAGAATTAATGATGTCTCGTTTAGATAAAAGTAAAGTGATTAACAGCGCATTAGAGCTGCTT
AATGAGGTCGGAATCGAAGGTTTAACAACCCGTAAACTCGCCCAGAAGCTAGGTGTAGAG
CAGCCTACATTGTATTGGCATGTAAAAAATAAGCGGGCTTTGCTCGACGCCTTAGCCATTG
AGATGTTAGATAGGCACCATACTCACTTTTGCCCTTTAGAAGGGGAAAGCTGGCAAGATTT
TTTACGTAATAACGCTAAAAGTTTTAGATGTGCTTTACTAAGTCATCGCGATGGAGCAAAAG
TACATTTAGGTACACGGCCTACAGAAAAACAGTATGAAACTCTCGAAAATCAATTAGCCTTT
TTATGCCAACAAGGTTTTTCACTAGAGAATGCATTATATGCACTCAGCGCAGTGGGGCATTT
TACTTTAGGTTGCGTATTGGAAGATCAAGAGCATCAAGTCGCTAAAGAAGAAAGGGAAACA
CCTACTACTGATAGTATGCCGCCATTATTACGACAAGCTATCGAATTATTTGATCACCAAGG
TGCAGAGCCAGCCTTCTTATTCGGCCTTGAATTGATCATATGCGGATTAGAAAAACAACTTA
AATGTGAAAGTGGGTCTTAATGAGAATATTCGTTTTCACCCAAGGAATAGAGGATATGGAG
AAAAAAATCACTGGATATACCACCGTTGATATATCCCAATGGCATCGTAAAGAACATTTTGA
GGCATTTCAGTCAGTTGCTCAATGTACCTATAACCAGACCGTTCAGCTGGATATTACGGCC
TTTTTAAAGACCGTAAAGAAAAATAAGCACAAGTTTTATCCGGCCTTTATTCACATTCTTGCC
CGCCTGATGAATGCTCATCCGGAGTTCCGTATGGCAATGAAAGACGGTGAGCTGGTGATA
TGGGATAGTGTTCACCCTTGTTACACCGTTTTCCATGAGCAAACTGAAACGTTTTCATCGCT
CTGGAGTGAATACCACGACTAGTTCCGGCAGTTTCTACACATATATTCGCAAGATGTGGCG
TGTTACGGTGAAAACCTGGCCTATTTCCCTAAAGGGTTTATTGAGAATATGTTTTTCGTCTC
AGCCAATCCCTGGGTGAGTTTCACCAGTTTTGATTTAAACGTGGCCAATATGGACAACTTCT
TCGCCCCCGTTTTCACTATGGGCAAATATTATACGCAAGGCGACAAGGTGCTGATGCCGCT
GGCGATTCAGGTTCATCATGCCGTTTGTGATGGCTTCCATGTCGGCAGAATGCTTAATGAA
TTACAACAGTACTGCGATGAGTGGCAGGGCGGGGCGTAATAGCTTCACTAGTTTAAAAGG
ATCTAGGTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGATAAGTCGTGTC
TTACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTTGTGATG
CTCGTCAGGGGGGCGGAGCCTATGGAAAACGCCAGCAACGCGGCC
SEQ ID NO: 86: amino acid sequence of ProtL^{UAG337-}ABD.
The position of Caf is in bold face and underlined.
MetLysGluGluValThrIleLysValAsnLeuIleCafAlaAspGlyLysThrGlnThr
AlaGluPheLysGlyThrPheGluGluAlaThrAlaLysAlaTyrAlaAsnAlaAspLeu
SerAlaLysGluAsnGlyGluTyrThrAlaAspLeuGluAspGlyGlyAsnThrIleAsn
IleLysPheAlaGlyGlyAlaValAspAlaAsnSerLeuAlaGluAlaLysValLeuAla
AsnArgGluLeuAspLysTyrGlyValSerAspTyrTyrLysAsnLeuIleAsnAsnAla
LysThrValGluGlyValLysAlaLeuIleAspGluIleLeuAlaAlaLeuPro
```

1. A polypeptide comprising a light-responsive element,
wherein the light-responsive element can be switched between two isomers by irradiating the polypeptide with a particular wavelength of light, thereby altering the binding activity of the polypeptide to a ligand.

2-3. (canceled)

4. A method for isolating and/or purifying a molecule of interest, comprising:
contacting a liquid phase comprising the molecule of interest with the polypeptide of claim 1,
wherein the polypeptide is part of a solid phase,
and wherein the light-responsive element is in a first configuration so that the polypeptide has high affinity to the molecule of interest; and
(ii) irradiating the polypeptide with a wavelength that changes the light-responsive element to a second configuration so that the polypeptide has a decreased affinity to the molecule of interest as compared to the affinity of step (i); and
(iii) eluting the molecule of interest from the solid phase.

5. The polypeptide of claim 1, wherein the polypeptide is streptavidin or a variant or mutein thereof comprising a light-responsive element.

6. The polypeptide of claim 1, wherein the polypeptide comprises or consists of
(i) the amino acid sequence of SEQ ID NO: 2;
(ii) the amino acid sequence of SEQ ID NO: 4;
(iii) the amino acid sequence of SEQ ID NO: 6;
(iv) the amino acid sequence of SEQ ID NO: 86;
(v) the amino acid sequence of SEQ ID NO: 20, wherein the residue at position 12 of SEQ ID NO: 20 is replaced by a light-responsive element;
(vi) the amino acid sequence of SEQ ID NO: 61, wherein the residue at position 13 of SEQ ID NO: 61 is replaced by a light-responsive element; or
(vii) an amino acid sequence having at least 80% identity to the amino acid sequence according to any one of (i)-(vi)
wherein irradiating the polypeptide results in a change in conformation or shape of a ligand-binding pocket of the polypeptide the polypeptide.

7. (canceled)

8. The polypeptide of claim 1, wherein the light-responsive element is in or in the vicinity of a ligand-binding pocket or site of the polypeptide or wherein the light-responsive element is involved in binding of a ligand to the polypeptide.

9. (canceled)

10. The polypeptide of claim 1, wherein the polypeptide comprises SEQ ID NO: 2, 4, 6, 8, 10, 12, 20, 61, or 86 or an amino acid sequence having at least 80% identity thereto and the light-responsive element is
(i) at amino acid position 96 of any one of SEQ ID NOs: 2, 4, 8, and 10;
(ii) at position 132 of any one of SEQ ID NOs: 6 and 12;
(iii) at position 12 of SEQ ID NO: 20;
(iv) at position 13 of any one of SEQ ID NOs: 61 and 86;
(v) in an amino acid sequence having at least 80% identity to the amino acid sequence of any one of SEQ ID NOs: 2, 4, 8 or 10, at the amino acid position that is homologous to amino acid position 96 of SEQ ID NO: 2, 4, 8 or 10, respectively;
(vi) in an amino acid sequence having at least 80% identity to the amino acid sequence of any one of SEQ ID NOs: 6 or 12, at the amino acid position that is homologous to amino acid position 132 of SEQ ID NO: 6 or 12, respectively;
(vii) in an amino acid sequence having at least 80% identity to the amino acid sequence of SEQ ID NO: 20, at the amino acid position that is homologous to amino acid position 12 of SEQ ID NO: 20; or
(viii) in an amino acid sequence having at least 80% identity to the amino acid sequence of any one of SEQ ID NOs: 61 and 86, at the amino acid position that is homologous to amino acid position 13 of SEQ ID NO: 61.

11. The polypeptide of claim 1, wherein the polypeptide has higher affinity to a ligand before being irradiated as compared to the polypeptide after it has been irradiated.

12. The polypeptide of claim 1, wherein the polypeptide has high affinity to a ligand before being irradiated and the polypeptide has low affinity to said ligand after it has been irradiated.

13. The polypeptide of claim 1, wherein the light-responsive element comprises an azo group or a light-switchable amino acid side chain.

14. (canceled)

15. The polypeptide of claim 1, wherein the light-responsive element comprises a non-natural amino acid, wherein two isomers of the non-natural amino acid can be switched with particular wavelengths of light.

16. The polypeptide of claim 1, wherein the light-responsive element comprises
(i) 3′-carboxyphenylazophenylalanine or a derivative thereof; or
(ii) 4′-carboxyphenylazophenylalanine or a derivative thereof.

17. The polypeptide of claim 1, wherein the isomers are a trans isomer and a cis isomer.

18. The polypeptide of claim 17, wherein the a trans isomer of 3′-carboxyphenylazophenylalanine or 4′-carboxyphenylazophenylalanine has an increased affinity to a ligand as compared to a cis isomer of 3′-carboxyphenylazophenylalanine or 4′-carboxyphenylazophenylalanine.

19. The polypeptide of claim 17, wherein at visible light having 405-470 nm, at least 70% of the polypeptide comprises a trans isomer of the light-responsive element or at ultraviolet (UV) light having 310 to 370 nm, at least 85% of the polypeptide comprises a cis isomer of the light-responsive element.

20.-21. (canceled)

22. A solid phase selected from the group consisting of a matrix, a hydrogel, a bead, a magnetic bead, a chip, a glass surface, a plastic surface, a gold surface, a silver surface, and a plate, wherein the solid phase comprises the polypeptide of claim 1.

23.-28. (canceled)

29. The polypeptide of claim 1, wherein the polypeptide binds to a ligand selected from the group consisting of a peptide, an oligopeptide, a polypeptide, a protein, an antibody or a fragment thereof, an immunoglobulin or a fragment thereof, an enzyme, a hormone, a cytokine, a complex, an oligonucleotide, a polynucleotide, a nucleic acid, a carbohydrate, a liposome, a nanoparticle, a cell, a biomacromolecule, a biomolecule, and a small molecule.

30. The polypeptide of claim 29, wherein the ligand comprises or consists of
(i) the amino acid sequence of SEQ ID NO: 13;
(ii) the amino acid sequence of SEQ ID NO: 14; or
(iii) an amino acid sequence having at least 80% identity to SEQ ID NO: 13 or 14 and having affinity to streptavidin or its mutants or variants.

31. The polypeptide of claim 30, wherein the polypeptide is a streptavidin mutant comprising a tetramer of the protein having the amino acid sequence of SEQ ID NO: 7.

32.-44. (canceled)

45. An affinity matrix comprising the polypeptide of claim 1.

46. An affinity chromatography column comprising the affinity matrix of claim 45.

47.-53. (canceled)
